(19) United States
(12) Patent Application Publication
(10) Pub. No.: US 2019/0374576 A1
(43) Pub. Date: Dec. 12, 2019

(54) VIRAL METHODS OF T CELL THERAPY

(71) Applicants: Intima Bioscience, Inc., New York, NY (US); Regents of the University of Minnesota, Minneapolis, MN (US); The United States of America, as represented by the Secretary,Department of Health and Human Service, Bethesda, MD (US)

(72) Inventors: Thomas HENLEY, Cambridgeshire (GB); Eric RHODES, Camino, CA (US); Modassir CHOUDHRY, New York, NY (US); Branden MORIARITY, Shoreview, MN (US); Beau WEBBER, Coon Rapids, MN (US); Steven A. ROSENBERG, Potomac, MD (US); Douglas C. PALMER, North Bethesda, MD (US); Nicholas P. RESTIFO, Chevy Chase, MD (US)

(21) Appl. No.: 16/389,586

(22) Filed: Apr. 19, 2019

Related U.S. Application Data
(63) Continuation of application No. PCT/US2017/058615, filed on Oct. 26, 2017.
(60) Provisional application No. 62/452,081, filed on Jan. 30, 2017.
(60) Provisional application No. 62/413,814, filed on Oct. 27, 2016.

(51) Int. Cl. A61K 35/17; C07K 14/725; C12N 15/90; C12N 15/86

(52) U.S. Cl. CPC A61K 35/17; C07K 14/7051; C12N 2015/8518; C12N 15/86; C12N 2310/20; C12N 15/907

(57) ABSTRACT

Methods of producing a population of genetically modified cells using viral or non-viral vectors. Disclosed are also modified viruses for producing a population of genetically modified cells and/or for the treatment of cancer.

### CROSS-REFERENCE

This application claims the benefit of U.S. Provisional Application No. 62/413,814, filed Oct. 27, 2016 and U.S. Provisional Application No. 62/452,081, filed Jan. 30, 2017, each of which is entirely incorporated herein by reference for all purposes.

### GOVERNMENT INTEREST STATEMENT

This invention was made with Government support under project numbers Z01BC010985 and Z01BC010763 by the National Institutes of Health, National Cancer Institute. The Government has certain rights in the invention.

### BACKGROUND

Despite remarkable advances in cancer therapeutics over the last 50 years, there remain many tumor types that are recalcitrant to chemotherapy, radiotherapy or biotherapy, particularly in advanced stages that cannot be addressed through surgical techniques. Recently there have been significant advances in the genetic engineering of lymphocytes to recognize molecular targets on tumors in vivo, resulting in remarkable cases of remission of the targeted tumor. However, these successes have been limited largely to hematologic tumors, and more broad application to solid tumors is limited by the lack of an identifiable molecule that is expressed by cells in a particular tumor, and lack of a molecule that can be used to specifically bind to the tumor target in order to mediate tumor destruction. Some recent advances have focused on identifying tumor-specific mutations that in some cases trigger an antitumor T cell response. For example, these endogenous mutations can be identified using a whole-exomic-sequencing approach. Tran E, et al., “Cancer immunotherapy based on mutation-specific CD4+ T cells in a patient with epithelial cancer,” Science 344: 641-644 (2014).

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein is a method of producing a population of genetically modified cells comprising: providing a population of cells from a human subject; modifying, ex vivo, at least one cell in said population of cells by introducing a break in a Cytokine Inducible SH2 Containing Protein (CISH) gene using a clustered regularly interspaced short palindromic repeats (CRISPR) system; and introducing an adeno-associated virus (AAV) vector comprising at least one exogenous transgene encoding a T cell receptor (TCR) to at least one cell in said population of cells to integrate said exogenous transgene into the genome of said at least one cell at said break; wherein using said AAV vector for integrating said at least one exogenous transgene reduces cellular toxicity compared to using a minicircle vector for integrating said at least one exogenous transgene in a comparable cell.

Disclosed herein is method of producing a population of genetically modified cells comprising: providing a population of cells from a human subject; modifying, ex vivo, at least one cell in said population of cells by introducing a break in a Cytokine Inducible SH2 Containing Protein (CISH) gene using a clustered regularly interspaced short palindromic repeats (CRISPR) system; and introducing an adeno-associated virus (AAV) vector comprising at least one exogenous transgene encoding a T cell receptor (TCR) to at least one cell in said population of cells to integrate said exogenous transgene into the genome of said at least one cell at said break; wherein said population of cells comprises at least about 90% viable cells as measured by fluorescence-activated cell sorting (FACS) at about 4 days after introducing said AAV vector.

Disclosed herein is method of producing a population of genetically modified cells comprising: providing a population of cells from a human subject; introducing a clustered regularly interspaced short palindromic repeats (CRISPR) system comprising a guide polynucleic acid to said population of cells, wherein said guide polynucleic acid specifically binds to a Cytokine Inducible SH2 Containing Protein (CISH) gene in a plurality of cells within said population of cells and said CRISPR system introduces a break in said CISH gene, thereby suppressing CISH protein function in said plurality of cells; and introducing an adeno-associated virus (AAV) vector to said plurality of cells, wherein said AAV vector integrates at least one exogenous transgene encoding a T cell receptor (TCR) into the genome of said plurality of cells at said break, thereby producing a population of genetically modified cells; wherein at least about 10% of the cells in said population of genetically modified cells expresses said at least one exogenous transgene.

Disclosed herein is a method of treating cancer in a human subject comprising: administering a therapeutically effective amount of a population of ex vivo genetically modified cells, wherein at least one of said ex vivo genetically modified cells comprises a genomic alteration in a Cytokine Inducible SH2 Containing Protein (CISH) gene that results in suppression of CISH protein function in said at least one ex vivo genetically modified cell, wherein said genomic alteration is introduced by a clustered regularly interspaced short palindromic repeats (CRISPR) system; and wherein said at least one ex vivo genetically modified cell further comprises an exogenous transgene encoding a T cell receptor (TCR), wherein said exogenous transgene is introduced into the genome of said at least one genetically modified cell in said CISH gene by an adeno-associated virus (AAV) vector; and wherein said administering treats cancer or ameliorates at least one symptom of cancer in said human subject.

Disclosed herein is a method of treating gastrointestinal cancer in a human subject comprising: administering a therapeutically effective amount of a population of ex vivo genetically modified cells, wherein at least one of said ex vivo genetically modified cells comprises a genomic alteration in a Cytokine Inducible SH2 Containing Protein (CISH) gene that results in suppression of CISH protein function in said at least one ex vivo genetically modified cell, wherein said genomic alteration is introduced by a clustered regularly interspaced short palindromic repeats (CRISPR) system; and wherein said at least one ex vivo genetically modified cell further comprises an exogenous transgene encoding a T cell receptor (TCR), wherein said exogenous transgene is introduced into the genome of said at least one genetically modified cell in said CISH gene by an adeno-associated virus (AAV) vector; and wherein said administering treats cancer or ameliorates at least one symptom of cancer in said human subject.

Disclosed herein is a method of treating cancer in a human subject comprising: administering a therapeutically effective amount of a population of ex vivo genetically modified cells, wherein at least one of said ex vivo genetically modified cells comprises a genomic alteration in a T cell receptor (TCR) gene that results in suppression of TCR protein function in said at least one ex vivo genetically modified cell and a genomic alteration in a Cytokine Inducible SH2 Containing Protein (CISH) gene that results in suppression of CISH protein function in said at least one ex vivo genetically modified cell, wherein said genomic alterations are introduced by a clustered regularly interspaced short palindromic repeats (CRISPR) system; and wherein said at least one ex vivo genetically modified cell further comprises an exogenous transgene encoding a T cell receptor (TCR), wherein said exogenous transgene is introduced into the genome of said at least one genetically modified cell in said CISH gene by an adeno-associated virus (AAV) vector; and wherein said administering treats cancer or ameliorates at least one symptom of cancer in said human subject.

Disclosed herein is an ex vivo population of genetically modified cells comprising: an exogenous genomic alteration in a Cytokine Inducible SH2 Containing Protein (CISH) gene that suppresses CISH protein function in at least one genetically modified cell, and an adeno-associated virus (AAV) vector comprising at least one exogenous transgene encoding a T cell receptor (TCR) for insertion into the genome of said at least one genetically modified cell in said CISH gene.

Disclosed herein is an ex vivo population of genetically modified cells comprising: an exogenous genomic alteration in a Cytokine Inducible SH2 Containing Protein (CISH) gene that suppresses CISH protein function in at least one genetically modified cell of said ex vivo population of genetically modified cells, and an adeno-associated virus (AAV) vector comprising at least one exogenous transgene encoding a T cell receptor (TCR) for insertion into the genome of at least one genetically modified cell of said ex vivo population of genetically modified cells in said CISH gene.

Disclosed herein is an ex vivo population of genetically modified cells comprising: an exogenous genomic alteration in a Cytokine Inducible SH2 Containing Protein (CISH) gene that suppresses CISH protein function and an exogenous genomic alteration in a T cell receptor (TCR) gene that suppresses TCR protein function in at least one genetically modified cell, and an adeno-associated virus (AAV) vector comprising at least one exogenous transgene encoding a T cell receptor (TCR) for insertion into the genome of said at least one genetically modified cell in said CISH gene.

Disclosed herein is a system for introducing at least one exogenous transgene to a cell, said system comprising a nuclease or a polynucleotide encoding said nuclease, and an adeno-associated virus (AAV) vector, wherein said nuclease or polynucleotide encoding said nuclease introduces a double strand break in a Cytokine Inducible SH2 Containing Protein (CISH) gene of at least one cell, and wherein said AAV vector introduces at least one exogenous transgene encoding a T cell receptor (TCR) into the genome of said cell at said break; wherein said system has higher efficiency of introduction of said transgene into said genome and results in lower cellular toxicity compared to a similar system comprising a minicircle and said nuclease or polynucleotide encoding said nuclease, wherein said minicircle introduces said at least one exogenous transgene into said genome.

Disclosed herein is a system for introducing at least one exogenous transgene to a cell, said system comprising a nuclease or a polynucleotide encoding said nuclease, and an adeno-associated virus (AAV) vector, wherein said nuclease or polynucleotide encoding said nuclease introduces a double strand break in a Cytokine Inducible SH2 Containing Protein (CISH) gene and in a T cell receptor (TCR) gene of at least one cell, and wherein said AAV vector introduces at least one exogenous transgene encoding a T cell receptor (TCR) into the genome of said cell at said break; wherein said system has higher efficiency of introduction of said transgene into said genome and results in lower cellular toxicity compared to a similar system comprising a minicircle and said nuclease or polynucleotide encoding said nuclease, wherein said minicircle introduces said at least one exogenous transgene into said genome.

Disclosed herein is a method of treating a cancer, comprising: modifying, ex vivo, a Cytokine Inducible SH2 Containing Protein (CISH) gene in a population of cells from a human subject using a clustered regularly interspaced short palindromic repeats (CRISPR) system, wherein said CRISPR system introduces a double strand break in said CISH gene to generate a population of engineered cells; introducing a cancer-responsive receptor into said population of engineered cells using an adeno-associated viral gene delivery system to integrate at least one exogenous transgene at said double strand break, thereby generating a population of cancer-responsive cells, wherein said adeno-associated viral gene delivery system comprises an adeno-associated virus (AAV) vector; and administering a therapeutically effective amount of said population of cancer-responsive cells to said subject.

Disclosed herein is a method of treating a gastrointestinal cancer, comprising: modifying, ex vivo, a Cytokine Inducible SH2 Containing Protein (CISH) gene in a population of cells from a human subject using a clustered regularly interspaced short palindromic repeats (CRISPR) system, wherein said CRISPR system introduces a double strand break in said CISH gene to generate a population of engineered cells; introducing a cancer-responsive receptor into said population of engineered cells using an adeno-associated viral gene delivery system to integrate at least one exogenous transgene at said double strand break, thereby generating a population of cancer-responsive cells, wherein said adeno-associated viral gene delivery system comprises an adeno-associated virus (AAV) vector; and administering a therapeutically effective amount of said population of cancer-responsive cells to said subject.

Disclosed herein is a method of making a genetically modified cell, comprising: providing a population of host cells; introducing a recombinant adeno-associated virus (AAV) vector and a clustered regularly interspaced short palindromic repeats (CRISPR) system comprising a nuclease or a polynucleotide encoding said nuclease; wherein said nuclease introduces a break in a Cytokine Inducible SH2 Containing Protein (CISH) gene, and said AAV vector introduces an exogenous nucleic acid at said break; wherein using said AAV vector for integrating said at least one exogenous transgene reduces cellular toxicity compared to using a minicircle vector for integrating said at least one exogenous transgene in a comparable cell; wherein said exogenous nucleic acid is introduced at a higher efficiency compared to a comparable population of host cells to which said CRISPR system and a corresponding wild-type AAV vector have been introduced.

Disclosed herein is a method of producing a population of genetically modified tumor infiltrating lymphocytes (TILs) comprising: providing a population of TILs from a human subject; electroporating, ex vivo, said population of TILs with a clustered regularly interspaced short palindromic repeats (CRISPR) system, wherein said CRISPR system comprises a nuclease or a polynucleotide encoding said nuclease comprising a guide ribonucleic acid (gRNA); wherein said gRNA comprises a sequence complementary to a Cytokine Inducible SH2 Containing Protein (CISH) gene and said nuclease or polynucleotide encoding said nuclease introduces a double strand break in said CISH gene of at least one TIL in said population of TILs; wherein said nuclease is Cas9 or said polynucleotide encodes Cas9; and introducing an adeno-associated virus (AAV) vector to said at least one TIL in said population of TILs about 1 hour to about 4 days after the electroporation of said CRISPR system to integrate at least one exogenous transgene encoding a T cell receptor (TCR) into said double strand break.

Disclosed herein is a method of producing a population of genetically modified tumor infiltrating lymphocytes (TILs) comprising: providing a population of TILs from a human subject; electroporating, ex vivo, said population of TILs with a clustered regularly interspaced short palindromic repeats (CRISPR) system, wherein said CRISPR system comprises a nuclease or a polynucleotide encoding said nuclease comprising a guide ribonucleic acid (gRNA); wherein said gRNA comprises a sequence complementary to a Cytokine Inducible SH2 Containing Protein (CISH) gene and said nuclease or polynucleotide encoding said nuclease introduces a double strand break in said CISH gene of at least one TIL in said population of TILs; wherein said nuclease is Cas9 or said polynucleotide encodes Cas9; and introducing an adeno-associated virus (AAV) vector to said at least one TIL in said population of TILs about 1 hour to about 3 days after the electroporation of said CRISPR system to integrate at least one exogenous transgene encoding a T cell receptor (TCR) into said double strand break.

Disclosed herein is a method of producing a population of genetically modified tumor infiltrating lymphocytes (TILs) comprising: providing a population of TILs from a human subject; electroporating, ex vivo, said population of TILs with a clustered regularly interspaced short palindromic repeats (CRISPR) system, wherein said CRISPR system comprises a nuclease or a polynucleotide encoding said nuclease and at least one guide ribonucleic acid (gRNA); wherein said at least one gRNA comprises a gRNA comprising a sequence complementary to a Cytokine Inducible SH2 Containing Protein (CISH) gene and a gRNA comprising a sequence complementary to a T cell receptor (TCR) gene; wherein, said nuclease or polynucleotide encoding said nuclease introduces a first double strand break in said CISH gene and a second double strand break in said TCR gene of at least one TIL in said population of TILs; and, wherein said nuclease is Cas9 or said polynucleotide encodes Cas9; and introducing an adeno-associated virus (AAV) vector to said at least one TIL in said population of TILs about 1 hour to about 4 days after the electroporation of said CRISPR system to integrate at least one exogenous transgene encoding a T cell receptor (TCR) into at least one of said first double strand break or said second double strand break.

Disclosed herein is a method of producing a population of genetically modified cells comprising: providing a population of cells from a human subject; modifying, ex vivo, at least one cell in said population of cells by introducing a break in a Cytokine Inducible SH2 Containing Protein (CISH) gene using a nuclease or a polypeptide encoding said nuclease and a guide polynucleic acid; and introducing an adeno-associated virus (AAV) vector comprising at least one exogenous transgene encoding a T cell receptor (TCR) to at least one cell in said population of cells to integrate said exogenous transgene into the genome of said at least one cell at said break; wherein using said AAV vector for integrating said at least one exogenous transgene reduces cellular toxicity compared to using a minicircle vector for integrating said at least one exogenous transgene in a comparable cell.

Disclosed herein is a method of producing a population of genetically modified cells comprising: providing a population of cells from a human subject; introducing a clustered regularly interspaced short palindromic repeats (CRISPR) system comprising at least one guide polynucleic acid to said population of cells, wherein said at least one guide polynucleic acid comprises a guide polynucleic acid that specifically binds to a T cell receptor (TCR) gene and a guide polynucleic acid that specifically binds to a Cytokine Inducible SH2 Containing Protein (CISH) gene in a plurality of cells within said population of cells and said CRISPR system introduces a break in said TCR gene and said CISH gene, thereby suppressing TCR protein function and CISH protein function in said plurality of cells; and introducing an adeno-associated virus (AAV) vector to said plurality of cells, wherein said AAV vector integrates at least one exogenous transgene encoding a T cell receptor (TCR) into the genome of said plurality of cells at said break, thereby producing a population of genetically modified cells; wherein at least about 10% of the cells in said population of genetically modified cells expresses said at least one exogenous transgene.

In some cases, the methods of the present disclosure can further comprise introducing a break into an endogenous TCR gene using a CRISPR system. In some cases, introducing an AAV vector to at least one cell comprises introducing an AAV vector to a cell comprising a break (e.g., a break in a CISH and/or TCR gene).

In some cases, the methods or the systems of the present disclosure can comprise electroporation and/or nucleofection. In some cases, the methods or the systems of the present disclosure can further comprise a nuclease or a polypeptide encoding said nuclease. In some cases, said nuclease or polynucleotide encoding said nuclease can introduce a break into a CISH gene and/or a TCR gene. In some cases, said nuclease or polynucleotide encoding said nuclease can comprise an inactivation or reduced expression of a CISH gene and/or a TCR gene. In some cases, said nuclease or polynucleotide encoding said nuclease is selected from a group consisting of a clustered regularly interspaced short palindromic repeats (CRISPR) system, Zinc Finger, transcription activator-like effectors (TALEN), and meganuclease to TAL repeats (MEGATAL). In some cases, said nuclease or polynucleotide encoding said nuclease is from a CRISPR system. In some cases, said nuclease or polynucleotide encoding said nuclease is from an S. pyogenes CRISPR system. In some cases, a CRISPR system comprises a nuclease or a polynucleotide encoding said nuclease. In some cases, said nuclease or polynucleotide encoding said nuclease is selected from a group consisting of Cas9 and Cas9HiFi. In some cases, said nuclease or polynucleotide encoding said nuclease is Cas9 or a polynucleotide encoding Cas9. In some cases, said nuclease or polynucleotide encoding said nuclease is catalytically dead. In some cases, said nuclease or polynucleotide encoding said nuclease is a catalytically dead Cas9 (dCas9) or a polynucleotide encoding dCas9.

In some cases, the methods of the present disclosure can comprise (or can further comprise) modifying, ex vivo, at least one cell in a population of cells by introducing a break in a Cytokine Inducible SH2 Containing Protein (CISH) gene and/or in a TCR gene. In some cases, modifying comprises modifying using a guide polynucleic acid. In some cases, modifying comprises introducing a nuclease or a polynucleotide encoding said nuclease. In some cases, a CRISPR system comprises a guide polynucleic acid. In some cases, the methods or the systems or the populations of the present disclosure can further comprise a guide polynucleic acid. In some cases, said guide polynucleic acid comprises a complementary sequence to said CISH gene. In some cases, said guide polynucleic acid comprises a complementary sequence to said TCR gene. In some cases, said guide polynucleic acid is a guide ribonucleic acid (gRNA). In some cases, said guide polynucleic acid is a guide deoxyribonucleic acid (gDNA).

In some cases, cell viability is measured. In some cases, cell viability is measured by fluorescence-activated cell sorting (FACS). In some cases, a population of genetically modified cells or a population of tumor infiltrating lymphocytes comprises at least about 90%, 92%, 93%, 94%, 95%, 96%, 97%, 98%, 99%, 99.5%, or 100% cell viability post introduction of an AAV vector as measured by fluorescence-activated cell sorting (FACS). In some cases, cell viability is measured at about 4 hours, 6 hours, 10 hours, 12 hours, 18 hours, 24 hours, 36 hours, 48 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, or longer than 240 hours post introduction of an AAV vector. In some cases, cell viability is measured at about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 45 days, 50 days, 60 days, 70 days, 90 days, or longer than 90 days post introduction of an AAV vector. In some cases, a population of genetically modified cells or a population of tumor infiltrating lymphocytes can comprise at least about 92% cell viability at about 4 days post introduction of an AAV vector as measured by fluorescence-activated cell sorting (FACS). In some cases, a population of genetically modified cells can comprise at least about 92% cell viability at about 4 days post introduction of a recombinant AAV vector as measured by fluorescence-activated cell sorting (FACS).

In some cases, an AAV vector decreases cell toxicity compared to a corresponding unmodified or wild-type AAV vector. In some cases, cellular toxicity is measured. In some cases, toxicity is measured by flow cytometry. In some cases, integrating at least one exogenous transgene using an AAV vector reduces cellular toxicity compared to integrating said at least one exogenous transgene in a comparable population of cells using a minicircle or a corresponding unmodified or wild-type AAV vector. In some cases, toxicity is reduced by about 10%, 20%, 30%, 40%, 50%, 60%, 70%, 80%, 90%, or 100%. In some cases, toxicity is measured at about 4 hours, 6 hours, 8 hours, 12 hours, 24 hours, 36 hours, 48 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, or longer than 240 hours post introduction of said AAV vector or said corresponding unmodified or wild-type AAV vector or said minicircle vector. In some cases, toxicity is measured at about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 45 days, 50 days, 60 days, 70 days, 90 days, or longer than 90 days post introduction of said AAV vector or said corresponding unmodified or wild-type AAV vector or said minicircle.

In some cases, at least about 15%, 20%, 25%, 30%, 35%, 40%, 45%, 50%, 55%, 60%, 65%, 70%, 75%, 80%, 85%, 90%, 95%, or up to 100% of a population of genetically modified cells comprises integration of at least one exogenous transgene at a break in a CISH gene of the genome of a cell. In some cases, at least about 15%, 20%, 25%, 30%, 35%, 40%, 45%, 50%, 55%, 60%, 65%, 70%, 75%, 80%, 85%, 90%, 95%, or up to 100% of a population of genetically modified cells comprises integration of at least one exogenous transgene at a break in a TCR gene of the genome of a cell.

In some cases, a population of genetically modified cells and/or a population of genetically modified tumor infiltrating lymphocytes can be prepared according to the methods of the present disclosure. In some cases, a cell or a population of cells or a population of genetically modified cells can be a tumor infiltrating lymphocyte or a population of tumor infiltrating lymphocytes (TILs). In some cases, a population of cells or a population of genetically modified cells, respectively, is a primary cell or a population of primary cells. In some cases, a primary cell or a population of primary cells is a primary lymphocyte or a population of primary lymphocytes. In some cases, a primary cell or a population of primary cells is a TIL or a population of TILs. In some cases, TILs are autologous. In some cases, TILs are natural killer (NK) cells. In some cases, TILs are B cells. In some cases, TILs are T cells.

In some cases, the AAV vector is introduced at a multiplicity of infection (MOI) from about 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶ 4×10⁶, 5×10⁶, 6×10⁶, 7×10⁶, 8×10⁶, 9×10⁶, 1×10⁷, 2×10⁷, 3×10⁷, or up to about 9×10⁹ genome copies/virus particles per cell. In some cases, the wild-type AAV vector is introduced at a multiplicity of infection (MOI) from about 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶ 4×10⁶, 5×10⁶, 6×10⁶, 7×10⁶, 8×10⁶, 9×10⁶, 1×10⁷, 2×10⁷, 3×10⁷, or up to about 9×10⁹ genome copies/virus particles per cell. In some cases, AAV vector is introduced to said cell from 1-3 hrs., 3-6 hrs., 6-9 hrs., 9-12 hrs., 12-15 hrs., 15-18 hrs., 18-21 hrs., 21-23 hrs., 23-26 hrs., 26-29 hrs., 29-31 hrs., 31-33 hrs., 33-35 hrs., 35-37 hrs., 37-39 hrs., 39-41 hrs., 2 days, 3 days, 4 days, 5 days, 6 days, 7 days, 8 days, 9 days, 10 days, 14 days, 16 days, 20 days, or longer than 20 days after introducing said CRISPR or after said nuclease or polynucleic acid encoding said nuclease. In some cases, the AAV vector is introduced to a cell from 15 to 18 hours after introducing a CRISPR system or a nuclease or polynucleotide encoding said nuclease. In some cases, the AAV vector is introduced to a cell 16 hours after introducing a CRISPR system or a nuclease or polynucleotide encoding said nuclease.

In some cases, at least one exogenous transgene (e.g., exogenous transgene encoding a TCR) is randomly inserted into the genome. In some cases, at least one exogenous transgene is inserted into a CISH gene and/or a TCR gene of the genome. In some cases, at least one exogenous transgene is inserted in a CISH gene of the genome. In some cases, at least one exogenous transgene is not inserted in a CISH gene of the genome. In some cases, at least one exogenous transgene is inserted in a break in a CISH gene of the genome. In some cases, the transgene (e.g., at least one transgene encoding a TCR) is inserted in a TCR gene. In some cases, at least one exogenous transgene is inserted into a CISH gene in a random and/or site specific manner. In some cases, at least one exogenous transgene is flanked by engineered sites complementary to a break in a CISH gene and/or a TCR gene. In some cases, at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 97%, or at least about 98%, or at least about 99% of the cells in a population of cells or a population of genetically modified cells or a population of genetically modified TILs, comprise at least one exogenous transgene.

In some cases, the method of treating cancer can comprise administering a therapeutically effective amount of a population of cells of the present disclosure. In some cases, a therapeutically effective amount of a population of cells can comprise a lower number of cells compared to the number of cells required to provide the same therapeutic effect produced from a corresponding unmodified or wild-type AAV vector or from a minicircle, respectively.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative cases, in which the principles of the invention are utilized, and the accompanying drawings of which:

FIG. 1 depicts an example of a method which can identify a cancer-related target sequence, for example, a Neoantigen, from a sample obtained from a cancer patient using an in vitro assay (e.g. whole-exomic sequencing). The method can further identify a TCR transgene from a first T cell that recognizes the target sequence. The cancer-related target sequence and a TCR transgene can be obtained from samples of the same patient or different patients. The method can effectively and efficiently deliver a nucleic acid comprising a TCR transgene across membrane of a second T cell. In some instances, the first and second T cells can be obtained from the same patient. In other instances, the first and second T cells can be obtained from different patients. In other instances, the first and second T cells can be obtained from different patients. The method can safely and efficiently integrate a TCR transgene into the genome of a T cell using a non-viral integration system (e.g., CRISPR, TALEN, transposon-based, ZEN, meganuclease, or Mega-TAL) to generate an engineered T cell and thus, a TCR transgene can be reliably expressed in the engineered T cell. The engineered T cell can be grown and expanded in a condition that maintains its immunologic and anti-tumor potency and can further be administered into a patient for cancer treatment.

FIG. 2 shows some exemplary transposon constructs for TCR transgene integration and TCR expression.

FIG. 3 demonstrates the in vitro transcription of mRNA and its use as a template to generate homologous recombination (HR) substrate in any type of cell (e.g., primary cells, cell lines, etc.). Upstream of the 5′ LTR region of the viral genome a T7, T3, or other transcriptional start sequence can be placed for in vitro transcription of the viral cassette. mRNAs encoding both the sense and anti-sense strand of the viral vector can be used to improve yield.

FIG. 4 demonstrates the structures of four plasmids, including Cas9 nuclease plasmid, HPRT gRNA plasmid, Amaxa EGFPmax plasmid and HPRT target vector.

FIG. 5 shows an exemplary HPRT target vector with targeting arms of 0.5 kb.

FIG. 6 demonstrates three potential TCR transgene knock-in designs targeting an exemplary gene (e.g., HPRT gene). (1) Exogenous promoter: TCR transgene (“TCR”) transcribed by exogenous promoter (“Promoter”); (2) SA in-frame transcription: TCR transgene transcribed by endogenous promoter (indicated by the arrow) via splicing; and (3) Fusion in frame translation: TCR transgene transcribed by endogenous promoter via in frame translation. All three exemplary designs can knock-out the gene function. For example, when a HPRT gene or a PD-1 gene is knocked out by insertion of a TCR transgene, a 6-thiogaunine selection can be used as the selection assay.

FIG. 7 demonstrates that Cas9+gRNA+Target plasmids co-transfection had good transfection efficiency in bulk population.

FIG. 8 demonstrates the results of the EGFP FACS analysis of CD3+ T cells.

FIG. 9 shows two types of T cell receptors.

FIG. 10 shows successful T cell transfection efficiency using two platforms.

FIG. 11 shows efficient transfection as T cell number is scaled up, e.g., as T cell number increases.

FIG. 12 shows % gene modification occurring by CRISPR gRNAs at potential target sites.

FIG. 13 demonstrates CRISPR-induced DSBs in stimulated T cells.

FIG. 14 shows optimization of RNA delivery.

FIG. 15 demonstrates double strand breaks at target sites. The gene targeting was successful in inducing double strand breaks in T cells activated with anti-CD3 and anti-CD28 prior to introduction of the targeted CRISPR-Cas system. By way of example, immune checkpoint genes PD-1, CCR5, and CTLA4 were used to validate the system.

FIG. 16 shows a representation of TCR integration at CCR5. Exemplary design of a plasmid targeting vector with 1 kb recombination arms to CCR5. The 3 kb TCR expression transgene can be inserted into a similar vector with recombination arms to a different gene in order to target other genes of interest using homologous recombination. Analysis by PCR using primers outside of the recombination arms can demonstrate successful TCR integration at a gene.

FIG. 17 depicts TCR integration at the CCR5 gene in stimulated T cells. Positive PCR results demonstrate successful homologous recombination at CCR5 gene at 72 hours post transfection.

FIG. 18 shows T death in response to plasmid DNA transfection.

FIG. 19 is schematic of the innate immune sensing pathway of cytosolic DNA present in different types of cells, including but not limited to T cells. T cells express both pathways for detecting foreign DNA. The cellular toxicity can result from activation of these pathways during genome engineering.

FIG. 20 demonstrates that the inhibitors of FIG. 19 block apoptosis and pyropoptosis.

FIG. 21 shows a schematic of representative plasmid modifications. A standard plasmid contains bacterial methylation that can trigger an innate immune sensing system. Removing bacterial methylation can reduce toxicity caused by a standard plasmid. Bacterial methylation can also be removed and mammalian methylation added so that the vector looks like “self-DNA.” A modification can also include the use of a synthetic single stranded DNA.

FIG. 22 shows a representative functional engineered TCR antigen receptor. This engineered TCR is highly reactive against MART-1 expressing melanoma tumor cell lines. The TCR α and β chains are linked with a furin cleavage site, followed by a 2A ribosomal skip peptide.

FIG. 23 A and FIG. 23 B show PD-1, CTLA-4, PD-1 and CTLA-2, or CCR5, PD-1, and CTLA-4 expression on day 6 post transfection with guide RNAs. Representative guides: PD-1 (P2, P6, P2/6), CTLA-4 (C2, C3, C2/3), or CCR5 (CC2). FIG. 23A shows the percent inhibitory receptor expression. FIG. 23B shows normalized inhibitory receptor expression to a control guide RNA.

FIG. 24 A shows CTLA-4 expression in primary human T cells after electroporation with CRISPR and CTLA-4 specific guide RNAs, guides #2 and #3, as compared to unstained and a no guide control. FIG. 24B shows PD-1 expression in primary human T cells after electroporation with CRISPR and PD-1 specific guide RNAs, guides #2 and #6, as compared to unstained and a no guide control.

FIG. 25 shows FACs results of CTLA-4 and PD-1 expression in primary human T cells after electroporation with CRISPR and multiplexed CTLA-4 and PD-1 guide RNAs.

FIG. 26 A and FIG. 26 B show percent double knock out in primary human T cells post treatment with CRISPR. FIG. 26A shows percent CTLA-4 knock out in T cells treated with CTLA-4 guides #2, #3, #2 and #3, PD-1 guide #2 and CTLA-4 guide #2, PD-1 guide #6 and CTLA-4 guide #3, as compared to Zap only, Cas9 only, and an all guide RNA control. FIG. 26B shows percent PD-1 knock out in T cells treated with PD-1 guide#2, PD-1 guide #6, PD-1 guides #2 and #6, PD-1 guide #2 and CTLA-4 guide #2, PD-1 guide #6 and CTLA-4 guide #3, as compared to Zap only, Cas9 only, and an all guide RNA control.

FIG. 27 shows T cell viability post electroporation with CRISPR and guide RNAs specific to CTLA-4, PD-1, or combinations.

FIG. 28 results of a CEL-I assay showing cutting by PD-1 guide RNAs #2, #6, #2 and #6, under conditions where only PD-1 guide RNA is introduced, PD-1 and CTLA-4 guide RNAs are introduced or CCR5, PD-1, and CLTA-4 guide RNAs, Zap only, or gRNA only controls.

FIG. 29 results of a CEL-I assay showing cutting by CTLA-4 guide RNAs #2, #3, #2 and #3, under conditions where only CLTA-4 guide RNA is introduced, PD-1 and CTLA-4 guide RNAs are introduced or CCR5, PD-1, and CLTA-4 guide RNAs, Zap only, or gRNA only controls.

FIG. 30 results of a CEL-I assay showing cutting by CCR5 guide RNA #2 in conditions where CCR5 guide RNA is introduced, CCR5 guide RNA, PD-1 guide RNA, or CTLA-4 guide RNA, as compared to Zap only, Cas 9 only, or guide RNA only controls.

FIG. 31 shows knockout of TCR alpha, as measured by CD3 FACs expression, in primary human T cells utilizing optimized CRISPR guide RNAs with 2′ 0-Methyl RNA modification at 5 micrograms and 10 micrograms.

FIG. 32 depicts a method of measuring T cell viability and phenotype post treatment with CRISPR and guide RNAs to CTLA-4. Phenotype was measured by quantifying the frequency of treated cells exhibiting a normal FSC/SSC profile normalized to frequency of electroporation alone control. Viability was also measured by exclusion of viability dye by cells within the FSC/SSC gated population. T cell phenotype is measured by CD3 and CD62L.

FIG. 33 shows method of measuring T cell viability and phenotype post treatment with CRISPR and guide RNAs to PD-1, and PD-1 and CTLA-4. Phenotype was measured by quantifying the frequency of treated cells exhibiting a normal FSC/SSC profile normalized to frequency of electroporation alone control. Viability was also measured by exclusion of viability dye by cells within the FSC/SSC gated population. T cell phenotype is measured by CD3 and CD62L.

FIG. 34 shows results of a T7E1 assay to detect CRISPR gene editing on day 4 post transfection with PD-1 or CTKA-4 guide RNA of primary human T cells and Jurkat control. NN is a no T7E1 nuclease control.

FIG. 35 shows results of a tracking of indels by decomposition (TIDE) analysis. Percent gene editing efficiency as shows to PD-1 and CTLA-4 guide RNAs.

FIG. 36 shows results of a tracking of indels by decomposition (TIDE) analysis for single guide transfections. Percent of sequences with either deletions or insertions are shown for primary human T cells transfected with PD-1 or CTLA-1 guide RNAs and CRISPR.

FIG. 37 shows PD-1 sequence deletion with dual targeting.

FIG. 38 shows sequencing results of PCR products of PD-1 sequence deletion with dual targeting. Samples 6 and 14 are shown with a fusion of the two gRNA sequences with the intervening 135 bp excised.

FIG. 39 shows dual targeting sequence deletion of CTLA-4. Deletion between the two guide RNA sequences is also present in the sequencing of dual guide targeted CTLA-4 (samples 9 and 14). A T7E1 Assay confirms the deletion by PCR.

FIG. 40 A shows viability of human T cells on day 6 post CRISPR transfection. FIG. 40B shows FACs analysis of transfection efficiency of human T cells (% pos GFP).

FIG. 41 shows FACs analysis of CTLA-4 expression in stained human T cells transfected with anti-CTLA-4 CRISPR guide RNAs. PE is anti-human CD152 (CTLA-4).

FIG. 42 A shows CTLA-4 FACs analysis of CTLA-4 positive human T cells post transfection with anti-CTLA-4 guide RNAs and CRISPR. FIG. 42B shows CTLA-4 knock out efficiency relative to a pulsed control in human T cells post transfection with anti-CTLA-4 guide RNAs and CRISPR.

FIG. 43 shows minicircle DNA containing an engineered TCR.

FIG. 44 depicts modified sgRNA for CISH, PD-1, CTLA4 and AAVS1.

FIG. 45. Depicts FACs results of PD-1 KO on day 14 post transfection with CRISPR and anti-PD-1 guide RNAs. PerCP-Cy5.5 is mouse anti-human CD279 (PD-1).

FIG. 46 A shows percent PD-1 expression post transfection with an anti-PD-1 CRISPR system. FIG. 46B shows percent PD-1 knock out efficiency as compared to Cas9 only control.

FIG. 47 shows FACs analysis of the FSC/SSC subset of human T cells transfected with CRISPR system with anti-PD-1 guide #2, anti-PD-1 guide #6, anti-PD1 guides #2 and #6, or anti-PD-1 guides #2 and #6 and anti-CTLA-4 guides #2 and #3.

FIG. 48 shows FACs analysis of human T cells on day 6 post transfection with CRISPR and anti-CTLA-4 guide RNAs. PE is mouse anti-human CD152 (CTLA-4).

FIG. 49 shows FACs analysis of human T cells and control Jurkat cells on day 1 post transfection with CRISPR and anti-PD-1 and anti-CTLA-4 guide RNAs. Viability and transfection efficiency of human T cells is shown as compared to transfected Jurkat cells.

FIG. 50 depicts quantification data from a FACs analysis of CTLA-4 stained human T cells transfected with CRISPR and anti-CTLA-4 guide RNAs. Day 6 post transfection data is shown of percent CTLA-4 expression and percent knock out.

FIG. 51 shows FACs analysis of PD-1 stained human T cells transfected with CRISPR and anti-PD-1 guide RNAs. Day 14 post transfection data is shown of PD-1 expression (anti-human CD279 PerCP-Cy5.5)

FIG. 52 shows percent PD-1 expression and percent knock out of PD-1 compared to Cas9 only control of human T cells transfected with CRISPR and anti-PD-1 guide RNAs.

FIG. 53 shows day 14 cell count and viability of transfected human T cells with CRISPR, anti-CTLA-4, and anti-PD-1 guide RNAs.

FIG. 54 shows FACs data for human T cells on day 14 post electroporation with CRISPR, and anti-PD-1 guide #2 alone, anti-PD-1 guide #2 and #6, or anti-CTLA-4 guide #3 alone. The engineered T cells were re-stimulated for 48 hours to assess expression of CTLA-4 and PD-1 and compared to control cells electroporated with no guide RNA.

FIG. 55 shows FACs data for human T cells on day 14 post electroporation with CRISPR, and anti-CTLA-4 guide #2 and #3, anti-PD-1 guide #2 and anti-CTLA-4 guide #3, or anti-PD-1 guide #2 and #6, anti-CTLA-4 guide #3 and #2. The engineered T cells were re-stimulated for 48 hours to assess expression of CTLA-4 and PD-1 and compared to control cells electroporated with no guide RNA.

FIG. 56 depicts results of a surveyor assay for CRISPR mediated gene-modification of the CISH locus in primary human T cells.

FIG. 57 A depicts a schematic of a T cell receptor (TCR). FIG. 57B shows a schematic of a chimeric antigen receptor. FIG. 57C shows a schematic of a B cell receptor (BCR).

FIG. 58. Shows that somatic mutational burden varies among tumor type. Tumor-specific neo-antigen generation and presentation is theoretically directly proportional to mutational burden.

FIG. 59 shows pseudouridine-5′-Triphosphate and 5-Methylcytidine-5-Triphosphate modifications that can be made to nucleic acid.

FIG. 60 shows TIDE and densitometry data comparison for 293T cells transfected with CRISPR and CISH gRNAs 1, 3, 4, 5 or 6.

FIG. 61 depicts duplicate experiments of densitometry analysis for 293T cells transfected with CRISPR and CISH gRNAs 1, 3, 4, 5 or 6.

FIG. 62 A shows TIDE analysis of CISH gRNA 1. FIG. 62B shows duplicate TIDE analysis of CISH gRNA 1.

FIG. 63 A shows duplicate TIDE analysis of CISH gRNA 3. FIG. 63B shows duplicate TIDE analysis of CISH gRNA 3.

FIG. 64 A shows duplicate TIDE analysis of CISH gRNA 4. FIG. 64B shows duplicate TIDE analysis of CISH gRNA 4.

FIG. 65 A shows duplicate TIDE analysis of CISH gRNA 5. FIG. 65B shows duplicate TIDE analysis of CISH gRNA 5.

FIG. 66 A shows duplicate TIDE analysis of CISH gRNA 6. FIG. 66B shows duplicate TIDE analysis of CISH gRNA 6.

FIG. 67 shows a western blot showing loss of CISH protein after CRISPR knock out in primary T cells.

FIG. 68 depicts DNA viability by cell count at 1 day post transfection with single or double-stranded DNA. M13 ss/dsDNA is 7.25 kb. pUC57 is 2.7 kb. GFP plasmid is 6.04 kb., FIG. 68B depicts DNA viability by cell count at 2 days post transfection with single or double-stranded DNA. M13 ss/dsDNA is 7.25 kb. pUC57 is 2.7 kb. GFP plasmid is 6.04 kb FIG. 68C depicts DNA viability by cell count at 3 days post transfection with single or double-stranded DNA. M13 ss/dsDNA is 7.25 kb. pUC57 is 2.7 kb. GFP plasmid is 6.04 kb.

FIG. 69 shows a mechanistic pathway that can be modulated during preparation or post preparation of engineered cells.

FIG. 70 A depicts cell count post transfection with the CRISPR system (15ug Cas9, 10 ug gRNA) on day 3. FIG. 70 B depicts cell count post transfection with the CRISPR system (15ug Cas9, 10ug gRNA) on day 7. Sample 1-non treated. Sample 2-pulse only. Sample 3-GFP mRNA. Sample 4-Cas9 pulsed only. Sample 5-5 microgram minicircle donor pulsed only. Sample 6-20 micrograms minicircle donor pulsed only. Sample 7-plasmid donor (5 micrograms). Sample 8-plasmid donor (20 micrograms). Sample 9-+guide PD1-2/+Cas9/−donor. Sample 10-+guide PD1-6/+Cas9/−donor. Sample 11-+guide CTLA4-2/+Cas9/−donor. Sample 12-+guide CTLA4-3/+Cas9/−donor. Sample 13—PD1-2/5 ug donor. Sample 14—PD1 dual/5 ug donor. Sample 15-CTLA4-3/5 ug donor. Sample 16—CTLA4 dual/5ug donor. Sample 17—PD1-2/20ug donor. Sample 18—PD1 dual/20 ug donor. Sample 19—CTLA4-3/20 ug donor. Sample 20—CTLA4 dual/20 ug donor.

FIG. 71 A shows Day 4 TIDE analysis of PD-1 gRNA 2. FIG. 71B shows Day 4 TIDE analysis of PD-1 gRNA6 with no donor nucleic acid.

FIG. 72 A shows Day 4 TIDE analysis of CTLA4 gRNA 2. FIG. 72B shows Day 4 TIDE analysis of CTLA4 gRNA3 with no donor nucleic acid.

FIG. 73 shows FACs analysis of day 7 TCR beta detection in control cells, cells electroporated with 5 micrograms of donor DNA (minicircle), or cells electroporated with 20 micrograms of donor DNA (minicircle).

FIG. 74 shows a summary of day 7 T cells electroporated with the CRISPR system and either no polynucleic acid donor (control), 5 micrograms of polynucleic acid donor (minicircle), or 20 micrograms of polynucleic acid donor (minicircle). A summary of FACs analysis of TCR positive cells is shown.

FIG. 75 shows integration of the TCR minicircle in the forward direction into the PD1 gRNA#2 cut site.

FIG. 76 A shows percentage of live cells at day 4 using a GUIDE-Seq dose test of human T cells transfected with CRISPR and PD-1 or CISH gRNAs with 5′ or 3′ modifications (or both) at increasing concentrations of a double stranded polynucleic acid donor. FIG. 76B shows efficiency of integration at the PD-1 or CISH locus of human T cells transfected with CRISPR and PD-1 or CISH specific gRNAs.

FIG. 77 shows GoTaq and PhusionFlex analysis of dsDNA integration at the PD-1 or CISH gene sites.

FIG. 78 shows day 15 FACs analysis of human T cells transfected with CRISPR and 5 micrograms or 20 micrograms of minicircle DNA encoding for an exogenous TCR.

FIG. 79 shows a summary of day 15 T cells electroporated with the CRISPR system and either no polynucleic acid donor (control), 5 micrograms of polynucleic acid donor (minicircle), or 20 micrograms of polynucleic acid donor (minicircle). A summary of FACs analysis of TCR positive cells is shown.

FIG. 80 depicts digital PCR copy number data copy number relative to RNaseP on Day 4 post transfection of CRISPR, and a minicircle encoding an mTCRb chain. A plasmid donor encoding the mTCRb chain was used as a control.

FIG. 81 A shows day 3 T cell viability with increasing dose of minicircle encoding an exogenous TCR.

FIG. 81B shows day 7 T cell viability with increasing dose of minicircle encoding an exogenous TCR.

FIG. 82A shows the optimization conditions for Lonza nucleofection of T cell double strand DNA transfection. Cell number vs concentration of a plasmid encoding GFP. FIG. 82B shows the optimization conditions for Lonza nucleofection of T cells with double strand DNA encoding a GFP protein. Percent transduction is shown vs concentration of GFP plasmid used for transfection.

FIG. 83 A depicts a pDG6-AAV helper-free packaging plasmid for AAV TCR delivery. FIG. 83B shows a schematic of a protocol for AAV transient transfection of 293 cells for virus production. Virus will be purified and stored for transduction into primary human T cells.

FIG. 84 shows a rAAV donor encoding an exogenous TCR flanked by 900 bp homology arms to an endogenous immune checkpoint (CTLA4 and PD1 are shown as exemplary examples).

FIG. 85 shows a genomic integration schematic of a rAAV homologous recombination donor encoding an exogenous TCR flanked by homology arms to the AAVS1 gene.

FIG. 86A shows homology directed repair of double stand breaks at AAVS1 with integration of the transgene, a possible recombination event that may occur using the AAVS1 system. FIG. 86B shows homology directed repair of one stand of the AAVS1 gene and non-homologous end joining indel of the complementary stand of AAVS1, a possible recombination event that may occur using the AAVS1 system. FIG. 86C shows non-homologous end joining insertion of the transgene into the AAVS1 gene site and non-homologous end joining indel at AAVS1, a possible recombination event that may occur using the AAVS1 system. FIG. 86D shows nonhomologous idels at both AAVS1 locations with random integration of the transgene into a genomic site.

FIG. 87 shows a combined CRISPR and rAAV targeting approach of introducing a transgene encoding an exogenous TCR into an immune checkpoint gene.

FIG. 88A shows results from CRISPR electroporation experiment in which caspase and TBK inhibitors were used during the electroporation of a 7.5 microgram minicircle donor encoding an exogenous TCR. Viability is plotted in comparison to concentration of inhibitor used at day 3 post transfection. FIG. 88B shows the efficiency of electroporation at day 3 post transfection. Percent positive TCR is shown vs. concentration of inhibitor used.

FIG. 89 shows FACs data of human T cells electroporated with CRISPR and minicircle DNA (7.5 microgram) encoding an exogenous TCR. Caspase and TBK inhibitors were added during the electroporation.

FIG. 90A shows the electroporation efficiency showing transgene TCR positive cells vs immune checkpoint specific guide(s) used. FACS data of human T cells electroporated with CRISPR and a minicircle DNA encoding an exogenous TCR (20 micrograms). FIG. 90B shows FACS data of the electroporation efficiency showing TCR positive cells vs. immune checkpoint specific guide(s) used. FACS data of human T cells electroporated with CRISPR and a minicircle DNA encoding an exogenous TCR (20 micrograms).

FIG. 91 shows TCR expression on day 13 post electroporation with CRISPR and a minicircle encoding an exogenous TCR at varying concentrations of minicircle.

FIG. 92A shows a cell death inhibitor study in which human T cells were pre-treated with Brefeldin A and ATM-inhibitors prior to transfection with CRISPR and minicircle DNA encoding for an exogenous TCR, the figure shows viability of T cells on day 3 post electroporation. FIG. 92B shows a cell death inhibitor study in which human T cells were pre-treated with Brefeldin A and ATM-inhibitors prior to transfection with CRISPR and minicircle DNA encoding for an exogenous TCR, the figure shows viability of T cells on day 7 post electroporation.

FIG. 93A shows a cell death inhibitor study in which human T cells were pre-treated with Brefeldin A and ATM-inhibitors prior to transfection with CRISPR and minicircle DNA encoding for an exogenous TCR, the figure shows transgene (e.g., TCR transgene or an oncogene) expression on T cells on day 3 post electroporation. FIG. 93B a cell death inhibitor study in which human T cells were pre-treated with Brefeldin A and ATM-inhibitors prior to transfection with CRISPR and minicircle DNA encoding for an exogenous TCR, the figure shows TCR expression on T cells on day 7 post electroporation.

FIG. 94 shows a splice-acceptor GFP reporter assay to rapidly detect integration of an exogenous transgene (e.g., TCR).

FIG. 95 shows a locus-specific digital PCR assay to rapidly detect integration of an exogenous transgene (e.g., TCR).

FIG. 96 shows recombinant (rAAV) donor constructs encoding for an exogenous TCR using either a PGK promoter or a splice acceptor. Each construct is flanked by 850 base pair homology arms (HA) to the AAVS1 checkpoint gene.

FIG. 97 shows the rAAV AAVS1-TCR gene targeting vector. The schematic depiction of the rAAV targeting vector used to insert the transgenic TCR expression cassette into the AAVS1 “safe-harbour” locus within the intronic region of the PPP1R12C gene. Major features are shown along with their sizes in numbers of nucleotides (bp). ITR: internal tandem repeat; PGK: phosphoglycerate kinase; mTCR: murine T-cell receptor beta; SV40 PolyA: Simian virus 40 polyadenylation signal.

FIG. 98 shows T cells electroporated with a GFP+ transgene 48 hours post stimulation with modified gRNAs. gRNAs were modified with pseudouridine, 5′moC, 5′meC, 5′moU, 5′hmC+S′moU, m6A, or 5′moC+5′meC.

FIG. 99A depicts viability of GFP expressing cells for T cells electroporated with a GFP+ transgene 48 hours post stimulation with modified gRNAs. gRNAs were modified with pseudouridine, 5′moC, 5′meC, 5′moU, 5′hmC+S′moU, m6A, or 5′moC+S′meC. FIG. 99B depicts MFI of GFP expressing cells for T cells electroporated with a GFP+ transgene 48 hours post stimulation with modified gRNAs. gRNAs were modified with pseudouridine, 5′moC, 5′meC, 5′moU, 5′hmC+S′moU, m6A, or 5′moC+S′meC.

FIG. 100A shows TIDE results of a comparison of a modified clean cap Cas9 protein. Genomic integration was measured at the CCR5 locus of T cells electroporated with unmodified Cas9 or clean cap Cas9 at 15 micrograms of Cas9 and 10 micrograms of a chemically modified gRNA. FIG. 100B shows TIDE results of a comparison of a unmodified Cas9 protein. Genomic integration was measured at the CCR5 locus of T cells electroporated with unmodified Cas9 or clean cap Cas9 at 15 micrograms of Cas9 and 10 micrograms of a chemically modified gRNA.

FIG. 101A showed viability for Jurkat cells expressing reverse transcriptase (RT) reporter RNA that were transfected using the Neon Transfection System with RT encoding plasmids and primers (see table for concentrations) and assayed for cell viability and GFP expression on Days 3 post transfection. GFP positive cells represent cells with RT activity. FIG. 101B shows reverse transcriptase activity for Jurkat cells expressing reverse transcriptase (RT) reporter RNA that were transfected using the Neon Transfection System with RT encoding plasmids and primers (see table for concentrations) and assayed for cell viability and GFP expression on Days 3 post transfection. GFP positive cells represent cells with RT activity.

FIG. 102A shows absolute cell count pre and post stimulation of human TILs of a first donor's cell count pre- and post-stimulation cultured in either RPMI media or ex vivo media. FIG. 102B shows absolute cell count pre and post stimulation of human TILs of a second donor's cell count pre- and post-stimulation cultured in RPMI media.

FIG. 103A shows cellular expansion of human tumor infiltrating lymphocytes (TILs) electroporated with a CRISPR system targeting PD-1 locus or controls cells with the addition of autologous feeders.

FIG. 103B shows cellular expansion of human tumor infiltrating lymphocytes (TILs) electroporated with a CRISPR system targeting PD-1 locus or controls cells without the addition of autologous feeders.

FIG. 104A shows human T cells electroporated with the CRISPR system alone (control); GFP plasmid (donor) alone (control); donor and CRISPR system; donor, CRISPR, and cFLP protein; donor, CRISPR, and hAd5 E1A (E1A) protein; or donor, CRISPR, and HPV18 E7 protein. FACs analysis of GFP measured at 48 hours. FIG. 104B shows human T cells electroporated with the CRISPR system alone (control); GFP plasmid (donor) alone (control); donor and CRISPR system; donor, CRISPR, and cFLP protein; donor, CRISPR, and hAd5 E1A (E1A) protein; or donor, CRISPR, and HPV18 E7 protein. FACs analysis of GFP was measured at 8 days post electroporation.

FIG. 105 shows flow cytometry analysis of T cells transfected with a recombinant AAV (rAAV) vector containing a transgene encoding for a splice acceptor GFP using the CRISPR system on day 4 post transfection with serum. Conditions shown are Cas9 and gRNA, GFP mRNA, Virapur low titre virus, Virapur low titre virus and CRISPR, SA-GFP pAAV plasmid, SA-GFP pAAV plasmid and CRISPR, AAVananced virus, or AAVanced virus and CRISPR.

FIG. 106 shows shows flow cytometry analysis of T cells transfected with a recombinant AAV (rAAV) vector containing a transgene encoding for a splice acceptor GFP using the CRISPR system on day 4 post transfection, without serum. Conditions shown are Cas9 and gRNA, GFP mRNA, Virapur low titre virus, Virapur low titre virus and CRISPR, SA-GFP pAAV plasmid, SA-GFP pAAV plasmid and CRISPR, AAVananced virus, or AAVanced virus and CRISPR.

FIG. 107A shows flow cytometry analysis of T cells transfected with a recombinant AAV (rAAV) vector containing a transgene encoding for a splice acceptor GFP using the CRISPR system on day 7 post transfection with serum. Conditions shown are SA-GFP pAAV plasmid and SA-GFP pAAV plasmid and CRISPR. FIG. 107B shows flow cytometry analysis of T cells transfected with a recombinant AAV (rAAV) vector containing a transgene encoding for a splice acceptor GFP using the CRISPR system on day 7 post transfection with serum or without serum. Conditions shown are AAVanced virus only or AAVanced virus and CRISPR.

FIG. 108 demonstrates cell viability post transfection of SA-GFP pAAV plasmid or SA-GFP pAAV plasmid and CRISPR at time of transfection (+), at 4 hours post serum removal and transfection, or at 16 hrs post serum removal and transfection.

FIG. 109 shows read out of knock in of a splice acceptor-GFP (SA-GFP) pAAV plasmid at 3-4 days under conditions of serum, serum removal at 4 hours, or serum removal at 16 hours. Control (non-transfected) cells are compared to cells transfected with SA-GFP pAAV plasmid only or SA-GFP pAAV plasmid and CRISPR.

FIG. 110 shows FACS analysis of human T cells transfected with rAAV or rAAV and CRISPR encoding an SA-GFP transgene on day 3 post transfection at concentrations of 1×10⁵ MOI, 3×10⁵ MOI, or 1×10⁶ MOI.

FIG. 111 shows FACS analysis of human T cells transfected with rAAV or rAAV and CRISPR encoding an SA-GFP transgene on day 7 post transfection at concentrations of 1×10⁵ MOI, 3×10⁵ MOI, or 1×10⁶ MOI.

FIG. 112 shows FACS analysis of human T cells transfected with rAAV or rAAV and CRISPR encoding a TCR transgene on day 3 post transfection at concentrations of 1×10⁵ MOI, 3×10⁵ MOI, or 1×10⁶ MOI.

FIG. 113 shows FACS analysis of human T cells transfected with rAAV or rAAV and CRISPR encoding a TCR transgene on day 7 post transfection at concentrations of 1×10⁵ MOI, 3×10⁵ MOI, or 1×10⁶ MOI.

FIG. 114A demonstrates FACs analysis of human T cells transfected with Cas9 and gRNA only and a SA-GFP transgene at time points of 4 hours, 6 hours, 8 hours, 12 hours, 18 hours, and 24 hours. FIG. 114B demonstrates FACs analysis of human T cells transfected with rAAV, CRISPR, and a SA-GFP transgene at time points of 4 hours, 6 hours, 8 hours, 12 hours, 18 hours, and 24 hours.

FIG. 115A shows rAAV transduction (% GFP+) as a function of time on day 4 post stimulation. FIG. 115B shows viable cell count of transfected or untransfected cells with rAAV on day 4 post stimulation at time points of 4 hours, 6 hours, 8 hours, 12 hours, 18 hours, and 24 hours.

FIG. 116 shows FACS analysis of human T cells transfected with rAAV or rAAV and CRISPR encoding an SA-GFP transgene on day 4 post transfection at concentrations of 1×10⁵ MOI, 3×10⁵ MOI, 1×10⁶ MOI, 3×10⁶ MOI, or 5×10⁶ MOI.

FIG. 117A shows GFP positive (GFP+ve) expression of human T cells transfected with an AAV vector encoding a SA-GFP transgene on day 4 post stimulation at different multiplicity of infection (MOI) levels, 1 to 5×10⁶. FIG. 117B shows viable cell number on day 4 post stimulation of human T cells transfected or non-transfected with an AAV encoding a SA-GFP transgene at MOI levels from 0 to 5×10⁶.

FIG. 118 shows FACs analysis of human T cells transfected with rAAV or rAAV and CRISPR on day 4 post stimulation. Cells were transfected at MOI levels of 1×10⁵ MOI, 3×10⁵ MOI, 1×10⁶ MOI, 3×10⁶ MOI, or 5×10⁶ MOI.

FIG. 119 shows TCR positive (TCR+ve) expression of human T cells transfected with an AAV vector encoding a TCR transgene on day 4 post stimulation at different multiplicity of infection (MOI) levels, 1 to 5×10⁶.

FIG. 120A and FIG. 120B shows A. percent expression efficiency of human T cells virally transfected with AAV encoding a SA-GFP transgene, AAV encoding a TCR transgene, CRISPR targeting CISH and a TCR transgene, or CRISPR targeting CTLA-4 and a TCR transgene. B. are FACs plots showing TCR expression on day 4 post stimulation of cells transfected with rAAV or rAAV and CRISP gRNAs targeting CISH or CTLA-4 genes.

FIG. 120A shows the percent expression efficiency of human T cells virally transfected with AAV encoding a SA-GFP transgene, AAV encoding a TCR transgene, CRISPR targeting CISH and a TCR transgene, or CRISPR targeting CTLA-4 and a TCR transgene. FIG. 120B shows FACs plots showing TCR transgene expression on day 4 post stimulation of cells transfected with rAAV or rAAV and CRISP gRNAs targeting CISH or CTLA-4 genes.

FIG. 121A depicts a FACs plot of TCR expression on human T cells on day 4 post stimulation of control non-transfected cells. FIG. 121B shows FACs plotS of TCR expression on human T cells on day 4 post stimulation of cells transfected with AAS1pAAV plasmid only, CRISPR targeting CISH and pAAV, CRISPR targeting CTLA-4 and pAAV, NHEJ minicircle vector, AAVS1pAAV and CRISPR, CRISIR targeting CISH and pAAV-CISH plasmid, CTLA-4pAAV plasmid and CRISPR, or NHEJ minicircle and CRISPR.

FIG. 122A shows the percent GFP positive (GFP+) expression of human T cells transfected with a rAAV encoding SA-GFP on day 3 post transfection at MOI from 1×10⁵ MOI, 3×10⁵ MOI, 1×10⁶ MOI or pre-transfection (control). FIG. 122B shows TCR positive expression on human T cells transfected with rAAV encoding a TCR on day 3 post transfection or pre-transfection (control) at MOI from 1×10⁵ MOI, 3×10⁵ MOI, to 1×10⁶.

FIG. 123A shows expression of an exogenous TCR on human T cells from 4 to 19 days post transfection with a rAAV virus encoding for the TCR. FIG. 123B shows expression of an SA-GFP on human T cells from 2 to 19 days post transfection with an rAAV virus encoding for SA-GFP.

FIG. 124 depicts FACs plots of human T cells transfected with rAAV or rAAV+CRISPR each rAAV encoding for a SA-GFP transgene at MOI from 1×10⁵ MOI, 3×10⁵ MOI, or 1×10⁶ on day 14 post transfection.

FIG. 125 depicts FACs plots of human T cells transfected with rAAV or rAAV+CRISPR each rAAV encoding for a TCR transgene at MOI from 1×10⁵ MOI, 3×10⁵ MOI, or 1×10⁶ on day 14 post transfection.

FIG. 126 shows FACs plots of human T cells transfected with rAAV or rAAV+CRISPR each rAAV encoding for a SA-GFP transgene at MOI from 1×10⁵ MOI, 3×10⁵ MOI, or 1×10⁶ on day 19 post transfection.

FIG. 127 shows FACs plots of human T cells transfected with rAAV or rAAV+CRISPR each rAAV encoding for a TCR transgene at MOI from 1×10⁵ MOI, 3×10⁵ MOI, or 1×10⁶ on day 19 post transfection.

FIG. 128 shows FACs plots of human T cells transfected with AAV encoding for a SA-GFP or TCR on days 3 or 4, 7, 14 or 19 post transfection. X axis shows transgene expression.

FIG. 129A shows TCR expression on human T cells transfected with rAAV encoding a TCR at MOIs from 1×10⁵ MOI, 3×10⁵ MOI, 1×10⁶, 3×10⁶ MOI, or 5×10⁶ on days 3 to 14 post stimulation. FIG. 129B shows viable cell number on day 14 post stimulation of cells transfected with rAAV encoding a TCR at MOIs from 1×10⁵ MOI, 3×10⁵ MOI, 1×10⁶, 3×10⁶ MOI, or 5×10⁶ with and without CRISPR.

FIG. 130 shows TCR expression on day 14 post stimulation of cells transfected with rAAV only or rAAV and CRISPR at MOI of 1×10⁵ MOI, 3×10⁵ MOI, 1×10⁶, 3×10⁶ MOI, or 5×10⁶.

FIG. 131 shows TCR expression of cells transfected with rAAV only or rAAV and CRISPR targeting the CISH gene and encoding a TCR from day 4 to day 14.

FIG. 132 shows TCR expression of cells transfected with rAAV only or rAAV and CRISPR targeting the CTLA-4 gene and encoding a TCR from day 4 to day 14.

FIG. 133A shows GFP FACS day 3 post stimulation data of human T cells transfected with a transgene encoding SA-GFP, the figure shows non-transfected controls or GFP mRNA transfected control cells. FIG. 133B GFP FACS day 3 post stimulation data of human T cells transfected with a transgene encoding SA-GFP, the figure shows rAAV pulsed or rAAV and CRISPR transfected cells with no viral proteins, E4orf6 only, E1b55k H373A, or E4orf6+E1b55K H373A.

FIG. 134 shows FACS analysis of human T cells transfected with rAAV encoding a TCR on day 3 post stimulation with rAAV pulsed or rAAV and CRISPR utilizing no viral proteins or E4orf6 and E1b55k H373A. The AAVS1 gene was utilized for TCR integration.

FIG. 135A shows FACS analysis of human T cells transfected with rAAV encoding a TCR on day 3 post stimulation with rAAV pulsed or rAAV and CRISPR utilizing no viral proteins or E4orf6 and E1b55k H373A. The CTLA4 gene was utilized for TCR integration. FIG. 135B shows FACs data of non-transfected controls and a mini-circle only control.

FIG. 136A shows expression data of human T cells transfected with rAAV encoding a TCR on day 3 post stimulation; the figure shows a summary of flow cytometric data of TCR expression on T cells with genomic modifications of CTLA4, PD-1, AAVS1, or CISH as compared to control cells (NT). FIG. 136B shows expression data of human T cells transfected with rAAV encoding TCR on day 3 post stimulation; the figure shows flow data of TCR expression of T cells with genomic modifications of CTLA4, PD-1, AAVS1, or CISH as compared to control cells (NT).

FIG. 137A shows expression data of human T cells transfected with rAAV encoding a TCR on day 3 and day 7 post stimulation, the figure shows a summary of flow cytometric data of TCR expression on T cells with genomic modifications of CTLA4, PD-1, AAVS1, or CISH as compared to control cells (NT) on days 3 and 7. FIG. 1037B shows expression data of human T cells transfected with rAAV encoding a TCR on day 3 and day 7 post stimulation, the figure shows flow data of TCR expression of T cells with genomic modifications of CTLA4, PD-1, AAVS1, or CISH as compared to control cells (NT) on day 7 post stimulation.

FIG. 138 schematics of rAAV donor designs.

FIG. 139 shows TCR expression on day 14 post transduction with rAAV. Cells are also modified with CRISPR to knock down PD-1 or CTLA-4. Data shows engineered cells as compared to non-transduced (NT) cells.

FIG. 140 shows PD-1 and CTLA-4 expression after TCR knock-in with rAAV. FACs data on day 17 post transfection is shown.

FIG. 141A shows percent TCR expression for CRISPR and rAAV engineered cells for multiple PBMC donors. FIG. 141 B shows single nucleotide polymorphism (SNP) data for donors 91, 92, and 93.

FIG. 142 shows SNP frequency at PD-1, AAVS1, CISH, and CTLA-4 for multiple donors.

FIG. 143 shows data from an mTOR assay for cells engineered to express a TCR and have a CISH knock out. Data summary is for day 3, 7, and 14 post electroporation.

FIG. 144 shows copy number of CISH as compared to reference control for T cells engineered to express an exogenous TCR and have a CISH knock out using CRISPR and rAAV.

FIG. 145 A shows ddPCR data for mTOR1 vs GAPDH control on days 3, 7, 14 post CISH KO. FIG. 145 B shows TCR expression on days 3, 7, 14 post CISH KO and TCR knock in via rAAV.

FIG. 146 A shows a summary of off-target (OT) analysis for the presence of Indels at PD-1. FIG. 146 B shows a summary of off-target analysis for the presence of Indels at CISH.

FIG. 147 A shows digital PCR primer and probe placement relative to the incorporated TCR. FIG. 147B shows digital PCR data showing the integrated TCR relative to a reference gene for untreated cells and CRISPR CISH KO+rAAV modified cells.

FIG. 148A shows percent TCR integration by ddPCR in CISH KO cells. FIG. 148 B shows TCR integration and protein expression on days 3, 7, and 14 post electroporation with CRISPR and transduction with rAAV.

FIG. 149 shows digital PCR data showing the integrated TCR relative to a reference gene for untreated cells and CRISPR CTLA-4 KO+rAAV modified cells.

FIG. 150 A shows percent TCR integration by ddPCR in CTLA-4 KO cells on days 3, 7, and 14. FIG. 150 B shows shows TCR integration and protein expression on days 3, 7, and 14 post electroporation with CRISPR CTLA-4 KO and transduction with rAAV encoding an exogenous TCR.

FIG. 151 shows flow cytometry data for perfect TCR expression on days 3, 7, and 14 post transfection with rAAV (small scale transfection with 2×10⁵ cells and large scale transfection with 1×10⁶ cells) and electroporation with CRISPR.

FIG. 152 shows TCR expression by FACs analysis on day 14 post transduction with rAAV on CRISPR treated cells (2×10⁵ cells). Cells were also electroporated with CRISPR and guide RNAs against CTLA-4 or PD-1.

FIG. 153 shows percent TCR expression on day 14 post transduction with rAAV and CRISPR KO at AAVS1, PD-1, CISH, or CTLA-4 for multiple PBMC donors.

FIG. 154 shows GUIDE-seq data at the CISH utilizing 8 pmol double strand (ds) or 16 pmol ds donor (ODN).

FIG. 155 A shows a vector map for a rAAV vector encoding for an exogenous TCR with homology arms to PD-1. FIG. 155 B shows shows a vector map for a rAAV vector encoding for an exogenous TCR with homology arms to PD-1 and an MND promoter.

FIG. 156 shows a comparison of a single cell PCR without the use of lysis buffer or with lysis buffer. Cells were treated with CRISPR and have a knockout at the CISH gene.

FIG. 157 A shows a schematic showing a TCR knock in. FIG. 157 B shows a western blot of cells with a rAAV TCR knock in.

FIG. 158 shows single cell PCR at the CISH locus on day 28 post transfection with CRISPR and anti-CISH guide RNA. Cells were also transduced with rAAV encoding an exogenous TCR.

FIG. 159 A shows TCR expression on day 7 post transduction with rAAV encoding an exogenous TCR. FIG. 159 B shows a western blot on day 7 post transduction with rAAV encoding an exogenous TCR.

FIG. 160 shows a schematic of HIF-1 and its involvement in metabolism.

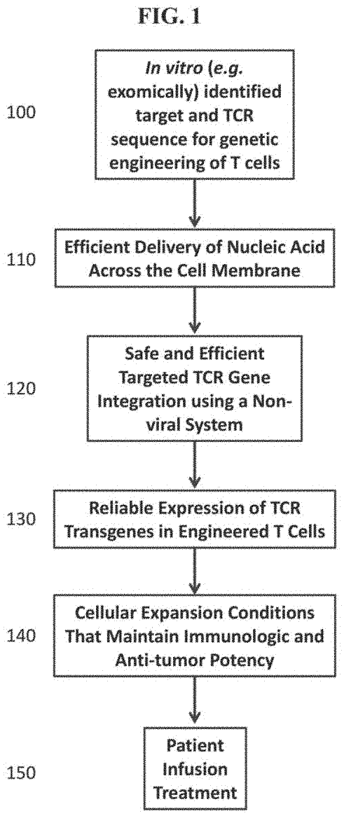

### DETAILED DESCRIPTION OF THE DISCLOSURE

The following description and examples illustrate embodiments of the present disclosure in detail. It is to be understood that the present disclosure is not limited to the particular embodiments described herein and as such can vary. Those of skill in the art will recognize that there are numerous variations and modifications of the present disclosure, which are encompassed within its scope.

### Definitions

The terms “AAV” or “recombinant AAV” or “rAAV” refer to adeno-associated virus of any of the known serotypes, including AAV-1, AAV-2, AAV-3, AAV-4, AAV-5, AAV-6, AAV-7, AAV-8, AAV-9, AAV-10, AAV-11, or AAV-12, self-complementary AAV (scAAV), rh10, or hybrid AAV, or any combination, derivative, or variant thereof. AAV is a small non-enveloped single-stranded DNA virus. They are non-pathogenic parvoviruses and may require helper viruses, such as adenovirus, herpes simplex virus, vaccinia virus, and CMV, for replication. Wild-type AAV is common in the general population, and is not associated with any known pathologies. A hybrid AAV is an AAV comprising genetic material from an AAV and from a different virus. A chimeric AAV is an AAV comprising genetic material from two or more AAV serotypes. An AAV variant is an AAV comprising one or more amino acid mutations in its capsid protein as compared to its parental AAV. AAV, as used herein, includes avian AAV, bovine AAV, canine AAV, equine AAV, primate AAV, non-primate AAV, and ovine AAV, wherein primate AAV refers to AAV that infect non-primates, and wherein non-primate AAV refers to AAV that infect non-primate animals, such as avian AAV that infects avian animals. In some cases, the wild-type AAV contains rep and cap genes, wherein the rep gene is required for viral replication and the cap gene is required for the synthesis of capsid proteins.

The terms “recombinant AAV vector” or “rAAV vector” or “AAV vector” refer to a vector derived from any of the AAV serotypes mentioned above. In some cases, an AAV vector may comprise one or more of the AAV wild-type genes deleted in whole or part, such as the rep and/or cap genes, but contains functional elements that are required for packaging and use of AAV virus for gene therapy. For example, functional inverted terminal repeats or ITR sequences that flank an open reading frame or exogenous sequences cloned in are known to be important for replication and packaging of an AAV virion, but the ITR sequences may be modified from the wild-type nucleotide sequences, including insertions, deletions, or substitutions of nucleotides, so that the AAV is suitable for use for the embodiments described herein, such as a gene therapy or gene delivery system. In some aspects, a self-complementary vector (sc) may be used, such as a self-complementary AAV vector, which may bypass the requirement for viral second-strand DNA synthesis and may lead to higher rate of expression of a transgene protein, as described in Wu, Hum Gene Ther. 2007, 18(2):171-82, incorporated by reference herein. In some aspects, AAV vectors may be generated to allow selection of an optimal serotype, promoter, and transgene. In some cases, the vector may be targeted vector or a modified vector that selectively binds or infects immune cells.

The terms “AAV virion” or “rAAV virion” refer to a virus particle comprising a capsid comprising at least one AAV capsid protein that encapsidates an AAV vector as described herein, wherein the vector may further comprise a heterologous polynucleotide sequence or a transgene in some embodiments.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

The term “activation” and its grammatical equivalents as used herein can refer to a process whereby a cell transitions from a resting state to an active state. This process can comprise a response to an antigen, migration, and/or a phenotypic or genetic change to a functionally active state. For example, the term “activation” can refer to the stepwise process of T cell activation. For example, a T cell can require at least two signals to become fully activated. The first signal can occur after engagement of a TCR by the antigen-MHC complex, and the second signal can occur by engagement of co-stimulatory molecules. Anti-CD3 can mimic the first signal and anti-CD28 can mimic the second signal in vitro.

The term “adjacent” and its grammatical equivalents as used herein can refer to right next to the object of reference. For example, the term adjacent in the context of a nucleotide sequence can mean without any nucleotides in between. For instance, polynucleotide A adjacent to polynucleotide B can mean AB without any nucleotides in between A and B.

The term “antigen” and its grammatical equivalents as used herein can refer to a molecule that contains one or more epitopes capable of being bound by one or more receptors. For example, an antigen can stimulate a host's immune system to make a cellular antigen-specific immune response when the antigen is presented, or a humoral antibody response. An antigen can also have the ability to elicit a cellular and/or humoral response by itself or when present in combination with another molecule. For example, a tumor cell antigen can be recognized by a TCR.

The term “epitope” and its grammatical equivalents as used herein can refer to a part of an antigen that can be recognized by antibodies, B cells, T cells or engineered cells. For example, an epitope can be a cancer epitope that is recognized by a TCR. Multiple epitopes within an antigen can also be recognized. The epitope can also be mutated.

The term “autologous” and its grammatical equivalents as used herein can refer to as originating from the same being. For example, a sample (e.g., cells) can be removed, processed, and given back to the same subject (e.g., patient) at a later time. An autologous process is distinguished from an allogenic process where the donor and the recipient are different subjects.

The term “barcoded to” refers to a relationship between molecules where a first molecule contains a barcode that can be used to identify a second molecule.

The term “cancer” and its grammatical equivalents as used herein can refer to a hyperproliferation of cells whose unique trait—loss of normal controls—results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

The term “cancer neo-antigen” or “neo-antigen” or “neo-epitope” and its grammatical equivalents as used herein can refer to antigens that are not encoded in a normal, non-mutated host genome. A “neo-antigen” can in some instances represent either oncogenic viral proteins or abnormal proteins that arise as a consequence of somatic mutations. For example, a neo-antigen can arise by the disruption of cellular mechanisms through the activity of viral proteins. Another example can be an exposure of a carcinogenic compound, which in some cases can lead to a somatic mutation. This somatic mutation can ultimately lead to the formation of a tumor/cancer.

The term “cytotoxicity” as used in this specification, refers to an unintended or undesirable alteration in the normal state of a cell. The normal state of a cell may refer to a state that is manifested or exists prior to the cell's exposure to a cytotoxic composition, agent and/or condition. Generally, a cell that is in a normal state is one that is in homeostasis. An unintended or undesirable alteration in the normal state of a cell can be manifested in the form of, for example, cell death (e.g., programmed cell death), a decrease in replicative potential, a decrease in cellular integrity such as membrane integrity, a decrease in metabolic activity, a decrease in developmental capability, or any of the cytotoxic effects disclosed in the present application.

The phrase “reducing cytotoxicity” or “reduce cytotoxicity” refers to a reduction in degree or frequency of unintended or undesirable alterations in the normal state of a cell upon exposure to a cytotoxic composition, agent and/or condition. The phrase can refer to reducing the degree of cytotoxicity in an individual cell that is exposed to a cytotoxic composition, agent and/or condition, or to reducing the number of cells of a population that exhibit cytotoxicity when the population of cells is exposed to a cytotoxic composition, agent and/or condition.

The term “engineered” and its grammatical equivalents as used herein can refer to one or more alterations of a nucleic acid, e.g., the nucleic acid within an organism's genome. The term “engineered” can refer to alterations, additions, and/or deletion of genes. An engineered cell can also refer to a cell with an added, deleted and/or altered gene.

The term “cell” or “engineered cell” or “genetically modified cell” and their grammatical equivalents as used herein can refer to a cell of human or non-human animal origin. The terms “engineered cell” and “genetically modified cell” are used interchangeably herein.

The term “checkpoint gene” and its grammatical equivalents as used herein can refer to any gene that is involved in an inhibitory process (e.g., feedback loop) that acts to regulate the amplitude of an immune response, for example, an immune inhibitory feedback loop that mitigates uncontrolled propagation of harmful responses (e.g., CTLA-4, and PD-1). These responses can include contributing to a molecular shield that protects against collateral tissue damage that might occur during immune responses to infections and/or maintenance of peripheral self-tolerance. Non-limiting examples of checkpoint genes can include members of the extended CD28 family of receptors and their ligands as well as genes involved in co-inhibitory pathways (e.g., CTLA-4, and PD-1). The term “checkpoint gene” can also refer to an immune checkpoint gene.

A “CRISPR,” “CRISPR system,” or “CRISPR nuclease system” and their grammatical equivalents can include a non-coding RNA molecule (e.g., guide RNA) that binds to DNA and Cas proteins (e.g., Cas9) with nuclease functionality (e.g., two nuclease domains) See, e.g., Sander, J. D., et al., “CRISPR-Cas systems for editing, regulating and targeting genomes,” Nature Biotechnology, 32:347-355 (2014); see also e.g., Hsu, P. D., et al., “Development and applications of CRISPR-Cas9 for genome engineering,” Cell 157(6):1262-1278 (2014).

The term “disrupting” and its grammatical equivalents as used herein can refer to a process of altering a gene, e.g., by cleavage, deletion, insertion, mutation, rearrangement, or any combination thereof. A disruption can result in the knockout or knockdown of protein expression. A knockout can be a complete or partial knockout. For example, a gene can be disrupted by knockout or knockdown. Disrupting a gene can partially reduce or completely suppress expression of a protein encoded by the gene. Disrupting a gene can also cause activation of a different gene, for example, a downstream gene. In some cases, the term “disrupting” can be used interchangeably with terms such as suppressing, interrupting, or engineering.

The term “function” and its grammatical equivalents as used herein can refer to the capability of operating, having, or serving an intended purpose. Functional can comprise any percent from baseline to 100% of normal function. For example, functional can comprise or comprise about 5, 10, 15, 20, 25, 30, 35, 40, 45, 50, 55, 60, 65, 70, 75, 80, 85, 90, 95, and/or 100% of normal function. In some cases, the term functional can mean over or over about 100% of normal function, for example, 125, 150, 175, 200, 250, 300% and/or above normal function.

The term “gene editing” and its grammatical equivalents as used herein can refer to genetic engineering in which one or more nucleotides are inserted, replaced, or removed from a genome. Gene editing can be performed using a nuclease (e.g., a natural-existing nuclease or an artificially engineered nuclease).

The term “mutation” and its grammatical equivalents as used herein can include the substitution, deletion, and insertion of one or more nucleotides in a polynucleotide. For example, up to 1, 2, 3, 4, 5, 6, 7, 8, 9, 10, 11, 12, 13, 14, 15, 20, 25, 30, 40, 50, or more nucleotides/amino acids in a polynucleotide (cDNA, gene) or a polypeptide sequence can be substituted, deleted, and/or inserted. A mutation can affect the coding sequence of a gene or its regulatory sequence. A mutation can also affect the structure of the genomic sequence or the structure/stability of the encoded mRNA.

The term “non-human animal” and its grammatical equivalents as used herein can include all animal species other than humans, including non-human mammals, which can be a native animal or a genetically modified non-human animal. The terms “nucleic acid,” “polynucleotide,” “polynucleic acid,” and “oligonucleotide” and their grammatical equivalents can be used interchangeably and can refer to a deoxyribonucleotide or ribonucleotide polymer, in linear or circular conformation, and in either single- or double-stranded form. For the purposes of the present disclosure, these terms should not to be construed as limiting with respect to length. The terms can also encompass analogues of natural nucleotides, as well as nucleotides that are modified in the base, sugar and/or phosphate moieties (e.g., phosphorothioate backbones). Modifications of the terms can also encompass demethylation, addition of CpG methylation, removal of bacterial methylation, and/or addition of mammalian methylation. In general, an analogue of a particular nucleotide can have the same base-pairing specificity, i.e., an analogue of A can base-pair with T.

The term “peripheral blood lymphocytes” (PBL) and its grammatical equivalents as used herein can refer to lymphocytes that circulate in the blood (e.g., peripheral blood). Peripheral blood lymphocytes can refer to lymphocytes that are not localized to organs. Peripheral blood lymphocytes can comprise T cells, NK cells, B cell, or any combinations thereof.

The term “phenotype” and its grammatical equivalents as used herein can refer to a composite of an organism's observable characteristics or traits, such as its morphology, development, biochemical or physiological properties, phenology, behavior, and products of behavior. Depending on the context, the term “phenotype” can sometimes refer to a composite of a population's observable characteristics or traits.

The term “protospacer” and its grammatical equivalents as used herein can refer to a PAM-adjacent nucleic acid sequence capable to hybridizing to a portion of a guide RNA, such as the spacer sequence or engineered targeting portion of the guide RNA. A protospacer can be a nucleotide sequence within gene, genome, or chromosome that is targeted by a guide RNA. In the native state, a protospacer is adjacent to a PAM (protospacer adjacent motif). The site of cleavage by an RNA-guided nuclease is within a protospacer sequence. For example, when a guide RNA targets a specific protospacer, the Cas protein will generate a double strand break within the protospacer sequence, thereby cleaving the protospacer. Following cleavage, disruption of the protospacer can result though non-homologous end joining (NHEJ) or homology-directed repair (HDR). Disruption of the protospacer can result in the deletion of the protospacer. Additionally or alternatively, disruption of the protospacer can result in an exogenous nucleic acid sequence being inserted into or replacing the protospacer.

The term “recipient” and their grammatical equivalents as used herein can refer to a human or non-human animal. The recipient can also be in need thereof.

The term “recombination” and its grammatical equivalents as used herein can refer to a process of exchange of genetic information between two polynucleic acids. For the purposes of this disclosure, “homologous recombination” or “HR” can refer to a specialized form of such genetic exchange that can take place, for example, during repair of double-strand breaks. This process can require nucleotide sequence homology, for example, using a donor molecule to template repair of a target molecule (e.g., a molecule that experienced the double-strand break), and is sometimes known as non-crossover gene conversion or short tract gene conversion. Such transfer can also involve mismatch correction of heteroduplex DNA that forms between the broken target and the donor, and/or synthesis-dependent strand annealing, in which the donor can be used to resynthesize genetic information that can become part of the target, and/or related processes. Such specialized HR can often result in an alteration of the sequence of the target molecule such that part or all of the sequence of the donor polynucleotide can be incorporated into the target polynucleotide. In some cases, the terms “recombination arms” and “homology arms” can be used interchangeably.

The terms “target vector” and “targeting vector” are used interchangeably herein.

The term “transgene” and its grammatical equivalents as used herein can refer to a gene or genetic material that is transferred into an organism. For example, a transgene can be a stretch or segment of DNA containing a gene that is introduced into an organism. When a transgene is transferred into an organism, the organism is then referred to as a transgenic organism. A transgene can retain its ability to produce RNA or polypeptides (e.g., proteins) in a transgenic organism. A transgene can be composed of different nucleic acids, for example RNA or DNA. A transgene may encode for an engineered T cell receptor, for example a TCR transgene. A transgene may comprise a TCR sequence. A transgene can comprise recombination arms. A transgene can comprise engineered sites.

The term “T cell” and its grammatical equivalents as used herein can refer to a T cell from any origin. For example, a T cell can be a primary T cell, e.g., an autologous T cell, a cell line, etc. The T cell can also be human or non-human.

The term “TIL” or tumor infiltrating lymphocyte and its grammatical equivalents as used herein can refer to a cell isolated from a tumor. For example, a TIL can be a cell that has migrated to a tumor. A TIL can also be a cell that has infiltrated a tumor. A TIL can be any cell found within a tumor. For example, a TIL can be a T cell, B cell, monocyte, natural killer cell, or any combination thereof. A TIL can be a mixed population of cells. A population of TILs can comprise cells of different phenotypes, cells of different degrees of differentiation, cells of different lineages, or any combination thereof.

A “therapeutic effect” may occur if there is a change in the condition being treated. The change may be positive or negative. For example, a ‘positive effect’ may correspond to an increase in the number of activated T-cells in a subject. In another example, a ‘negative effect’ may correspond to a decrease in the amount or size of a tumor in a subject. There is a “change” in the condition being treated if there is at least 10% improvement, preferably at least 25%, more preferably at least 50%, even more preferably at least 75%, and most preferably 100%. The change can be based on improvements in the severity of the treated condition in an individual, or on a difference in the frequency of improved conditions in populations of individuals with and without treatment with the therapeutic compositions with which the compositions of the present invention are administered in combination. Similarly, a method of the present disclosure may comprise administering to a subject an amount of cells that is “therapeutically effective”. The term “therapeutically effective” should be understood to have a definition corresponding to ‘having a therapeutic effect’.

The term “safe harbor” and “immune safe harbor”, and their grammatical equivalents as used herein can refer to a location within a genome that can be used for integrating exogenous nucleic acids wherein the integration does not cause any significant effect on the growth of the host cell by the addition of the nucleic acid alone. Non-limiting examples of safe harbors can include HPRT, AAVS SITE (E.G. AAVS1, AAVS2, ETC.), CCR5, or Rosa26. For example, the human parvovirus, AAV, is known to integrate preferentially into human chromosome 19 q13.3-qter, or the AAVS1 locus. Integration of a gene of interest at the AAVS1 locus can support stable expression of a transgene in various cell types. In some cases, a nuclease may be engineered to target generation of a double strand break at the AAVS1 locus to allow for integration of a transgene at the AAVS1 locus or to facilitate homologous recombination at the AAVS1 locus for integrating an exogenous nucleic acid sequence at the AAVS1 site, such as a transgene, a cell receptor, or any gene of interest as disclosed herein. In some cases, an AAV viral vector is used to deliver a transgene for integration at the AAVS1 site with or without an exogenous nuclease.

The term “sequence” and its grammatical equivalents as used herein can refer to a nucleotide sequence, which can be DNA or RNA; can be linear, circular or branched; and can be either single-stranded or double stranded. A sequence can be mutated. A sequence can be of any length, for example, between 2 and 1,000,000 or more nucleotides in length (or any integer value there between or there above), e.g., between about 100 and about 10,000 nucleotides or between about 200 and about 500 nucleotides.

The term “viral vector” refers to a gene transfer vector or a gene delivery system derived from a virus. Such vector may be constructed using recombinant techniques known in the art. In some aspects, the virus for deriving such vector is selected from adeno-associated virus (AAV), helper-dependent adenovirus, hybrid adenovirus, Epstein-Bar virus, retrovirus, lentivirus, herpes simplex virus, hemmaglutinating virus of Japan (HVJ), Moloney murine leukemia virus, poxvirus, and HIV-based virus.

### Overview

Disclosed herein are methods of producing a population of genetically modified cells. In some cases, at least one method comprises providing a population of cells from a human subject. In some cases, at least one method comprises modifying (e.g., ex vivo) at least one cell in said population of cells by introducing at least a break in at least one gene (e.g., Cytokine Inducible SH2 Containing Protein (CISH) gene and/or a T cell receptor (TCR) gene). In some cases, a break may suppress said at least one gene protein function (e.g., suppress CISH and/or TCR protein function). In some cases, a gene suppression can be partial or complete. In some cases, a break is introduced using a clustered regularly interspaced short palindromic repeats (CRISPR) system and/or a guide polynucleic acid. In some cases, a break is introduced using a CRISPR system comprising a nuclease and/or a guide polynucleic acid. In some cases, a break is introduced using a nuclease or a polypeptide comprising a nuclease and/or a guide polynucleic acid. In some cases, a guide polynucleic acid specifically binds to at least one gene (e.g., CISH and/or TCR) in at least one cell or in a plurality of cells. In some cases, an adeno-associated virus (AAV) vector is introduced to at least one cell in said population of cells. In some cases, said AAV comprises at least one exogenous transgene encoding a T cell receptor (TCR). In some cases, said AAV integrates said exogenous transgene into the genome of said at least one cell. In some cases, said AAV is introduced after, at the same time, or before a CRISPR system and/or a guide polynucleic acid and/or a nuclease or polypeptide encoding a nuclease. In some cases, at least one exogenous transgene can be integrated into the genome of at least one cell using a minicircle vector. In some cases, said at least one exogenous transgene is integrated at said break. In some cases, said at least one exogenous transgene is integrated randomly and/or site specific in said genome. In some cases, said at least one exogenous transgene is integrated at least once in said genome. In some cases, integrating said at least one exogenous transgene using an AAV vector reduces cellular toxicity compared to using a minicircle vector in a comparable cell. In some cases, said population of cells comprises at least about 90% viable cells at about 4 days after introducing said AAV vector. In some cases, cell viability is measured by fluorescence-activated cell sorting (FACS). In some cases, at least about 10% of the cells in said population of genetically modified cells expresses said at least one exogenous transgene. In some cases, said AAV vector comprises a modified AAV.

Disclosed herein are methods of treating cancer in a human subject. In one case, a method comprises administering a therapeutically effective amount of a population of ex vivo genetically modified cells to a human subject. In some cases, at least one of said ex vivo genetically modified cells comprises a genomic alteration in at least one gene (e.g., Cytokine Inducible SH2 Containing Protein (CISH) gene and/or TCR). In some cases, said genomic alteration results in suppression (e.g., partial or complete) of said at least one gene (e.g., CISH and/or TCR) protein function in said at least one ex vivo genetically modified cell. In some cases, said genomic alteration is introduced by a clustered regularly interspaced short palindromic repeats (CRISPR) system. In some cases, said at least one ex vivo genetically modified cell further comprises an exogenous transgene encoding a T cell receptor (TCR). In some cases, said exogenous transgene is introduced into the genome of said at least one genetically modified cell by an adeno-associated virus (AAV) vector. In some cases, administering a therapeutically effective amount of said population of genetically modified cells treats cancer or ameliorates at least one symptom of cancer in a human subject. In some cases, said AAV vector comprises a modified AAV.

Disclosed herein are ex vivo populations of genetically modified cells. In one case, an ex vivo population of genetically modified cells comprises an exogenous genomic alteration in at least one gene (e.g., Cytokine Inducible SH2 Containing Protein (CISH) gene and/or TCR gene). In some cases, said genomic alteration suppresses said at least one gene (e.g., CISH and/or TCR) protein function in at least one genetically modified cell. In some cases, said population further comprises an adeno-associated virus (AAV) vector. In some cases, said population comprises a minicircle vector rather than an AAV vector. In some cases, said AAV vector (or minicircle vector) comprises at least one exogenous transgene. In some cases, said exogenous transgene encodes a T cell receptor (TCR) for insertion into the genome of said at least one genetically modified cell. In some cases, said AAV vector comprises a modified AAV. In some cases, said AAV vector comprises an unmodified or wild type AAV. In some cases, a therapeutically effective amount of said population is administered to a subject to treat or ameliorate cancer. In some cases, said therapeutically effective amount of said population comprises a lower number of cells compared to the number of cells required to provide the same therapeutic effect produced from a corresponding unmodified or wild-type AAV vector or from a minicircle, respectively.

Disclosed herein are systems for introducing at least one exogenous transgene to a cell. In some cases, a system comprises a nuclease or a polynucleotide encoding said nuclease. In some cases, said system further comprises an adeno-associated virus (AAV) vector. In some cases, said nuclease or polynucleotide encoding said nuclease introduces a double strand break in at least one gene (e.g., a Cytokine Inducible SH2 Containing Protein (CISH) gene and/or TCR gene) of at least one cell. In some cases, said AAV vector introduces at least one exogenous transgene into the genome of said cell. In some cases, said at least one exogenous transgene encodes a T cell receptor (TCR). In some cases, the system comprises a minicircle vector rather than an AAV vector. In some cases, said minicircle vector introduces at least one exogenous transgene into the genome of a cell. In some cases, said system has higher efficiency of introduction of said transgene into said genome and results in lower cellular toxicity compared to a similar system comprising a minicircle and said nuclease or polynucleotide encoding said nuclease, wherein said minicircle introduces said at least one exogenous transgene into said genome. In some cases, said AAV vector comprises a modified AAV. In some cases, said AAV vector comprises an unmodified or wild type AAV.

Disclosed herein are methods of treating cancer in a human subject. In one case, a method of treating cancer comprises modifying, ex vivo, at least one gene (e.g., Cytokine Inducible SH2 Containing Protein (CISH) gene and/or a TCR gene) in a population of cells from a human subject. In some cases, said modifying comprises using a clustered regularly interspaced short palindromic repeats (CRISPR) system. In some cases, said modifying comprises using a guide polynucleic acid and/or a nuclease or a polypeptide comprising a nuclease. In some cases, said CRISPR system (or said guide polynucleic acid and/or a nuclease or a polypeptide comprising a nuclease) introduces a double strand break in said at least one gene (e.g., CISH gene and/or TCR gene) to generate a population of engineered cells. In some cases, said method further comprises introducing a cancer-responsive receptor into said population of engineered cells. In some cases, said introducing comprises using an adeno-associated viral gene delivery system to integrate at least one exogenous transgene at said double strand break, thereby generating a population of cancer-responsive cells. In some cases, said introducing comprises using a minicircle non-viral gene delivery system to integrate at least one exogenous transgene at said double strand break, thereby generating a population of cancer-responsive cells. In some cases, said adeno-associated viral gene delivery system comprises an adeno-associated virus (AAV) vector. In some cases, said method further comprises administering a therapeutically effective amount of said population of cancer-responsive cells to said subject. In some cases, said AAV vector comprises a modified AAV. In some cases, said AAV vector comprises an unmodified or wild type AAV. In some cases, a therapeutically effective amount of said population of cancer-responsive cells is administered to a subject to treat or ameliorate cancer. In some cases, said therapeutically effective amount of said population of cancer-responsive cells comprises a lower number of cells compared to the number of cells required to provide the same therapeutic effect produced from a corresponding unmodified or wild-type AAV vector or from a minicircle, respectively.

Disclosed herein are methods of making a genetically modified cell. In one case, a method comprises providing a population of host cells. In some cases, the method comprises introducing a modified adeno-associated virus (AAV) vector and a clustered regularly interspaced short palindromic repeats (CRISPR) system. In some cases, the method comprises introducing a minicircle vector and a clustered regularly interspaced short palindromic repeats (CRISPR) system. In some cases, the CRISPR system comprises a nuclease or a polynucleotide encoding said nuclease. In some cases, said nuclease introduces a break in at least one gene (Cytokine Inducible SH2 Containing Protein (CISH) gene and/or TCR gene). In some cases, said AAV vector introduces an exogenous nucleic acid. In some cases, said minicircle vector introduces an exogenous nucleic acid. In some cases, said exogenous nucleic acid is introduced at said break. In some embodiments using said AAV vector for integrating said at least one exogenous transgene reduces cellular toxicity compared to using a minicircle vector for integrating said at least one exogenous transgene in a comparable cell. In some cases, said exogenous nucleic acid is introduced at a higher efficiency compared to a comparable population of host cells to which said CRISPR system and a corresponding unmodified or wild-type AAV vector have been introduced.

Disclosed herein are methods of producing a population of genetically modified tumor infiltrating lymphocytes (TILs). In one case, a method comprises providing a population of TILs from a human subject. In some cases, the method comprises electroporating, ex vivo, said population of TILs with a clustered regularly interspaced short palindromic repeats (CRISPR) system. In some cases, said CRISPR system comprises a nuclease or a polynucleotide encoding said nuclease and at least one guide polynucleic acid (e.g., guide ribonucleic acid (gRNA)). In some cases, said CRISPR system comprises a nuclease or a polynucleotide encoding said nuclease comprising a guide ribonucleic acid (gRNA). In some cases, said gRNA comprises a sequence complementary to at least one gene (Cytokine Inducible SH2 Containing Protein (CISH) gene and/or TCR). In some cases, said at least one gRNA comprises a gRNA comprising a sequence complementary to a first gene (e.g., Cytokine Inducible SH2 Containing Protein (CISH) gene) and a gRNA comprising a sequence complementary to a second gene (e.g., T cell receptor (TCR) gene). In some cases, said nuclease or polynucleotide encoding said nuclease introduces a double strand break in said at least one gene (e.g., CISH gene and/or TCR) of at least one TIL in said population of TILs. In some cases, said nuclease or polynucleotide encoding said nuclease introduces a double strand break in said first gene (e.g., CISH gene) and/or of said second gene (e.g., TCR gene) of at least one TIL in said population of TILs. In some cases, said nuclease is Cas9 or said polynucleotide encodes Cas9. In some cases, the method further comprises introducing an adeno-associated virus (AAV) vector to said at least one TIL in said population of TILs. In some cases, said introducing comprises about 1 hour to about 4 days after the electroporation of said CRISPR system. In some cases, said AAV vector is introduced at some time later than about 1 hour after the electroporation with said CRISPR system (e.g., 10 hours after, 1 day after, 2 days after, 5 days after, 10 days after, 30 days after, one month after, two months after said electroporation with said CRISPR system, and so on). In some cases, said AAV vector is introduced before the electroporation with said CRISPR system (e.g., 30 minutes, 1 hr, 2 hr, 5 hr, 10 hr, 18 hr, 1 day, 2 days, 3 days, 5 days, 8 days, 10 days, 30 days, one month, two months before said electroporation with said CRISPR system, and so on). In some cases, said introducing integrates at least one exogenous transgene into said double strand break or into at least one of said double strand break. In some cases, said at least one exogenous transgene encodes a T cell receptor (TCR). In some cases, said AAV vector comprises a modified AAV. In some cases, said AAV vector comprises an unmodified or wild type AAV.

In some cases, any of the methods and/or any of the systems disclosed herein can further comprise a nuclease or a polypeptide encoding a nuclease. In some cases, any of the methods and/or any of the systems disclosed herein can further comprise a guide polynucleic acid. In some cases, any of the methods and/or any of the systems disclosed herein can comprise electroporation and/or nucleofection.

### Cells

Compositions and methods disclosed herein can utilize cells. Cells can be primary cells. Primary cells can be primary lymphocytes. A population of primary cells can be a population of primary lymphocytes. Cells can be recombinant cells. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. For example, any T cell lines can be used. Alternatively, the cell can be derived from a healthy donor, from a patient diagnosed with cancer, or from a patient diagnosed with an infection. In another case, the cell can be part of a mixed population of cells which present different phenotypic characteristics. A cell can also be obtained from a cell therapy bank. Disrupted cells resistant to an immunosuppressive treatment can be obtained. A desirable cell population can also be selected prior to modification. A selection can include at least one of: magnetic separation, flow cytometric selection, antibiotic selection. The one or more cells can be any blood cells, such as peripheral blood mononuclear cell (PBMC), lymphocytes, monocytes or macrophages. The one or more cells can be any immune cells such as lymphocytes, B cells, or T cells. Cells can also be obtained from whole food, apheresis, or a tumor sample of a subject. A cell can be a tumor infiltrating lymphocytes (TIL). In some cases an apheresis can be a leukapheresis. Leukapheresis can be a procedure in which blood cells are isolated from blood. During a leukapheresis, blood can be removed from a needle in an arm of a subject, circulated through a machine that divides whole blood into red cells, plasma and lymphocytes, and then the plasma and red cells are returned to the subject through a needle in the other arm. In some cases, cells are isolated after an administration of a treatment regime and cellular therapy. For example, an apheresis can be performed in sequence or concurrent with a cellular administration. In some cases, an apheresis is performed prior to and up to about 6 weeks following administration of a cellular product. In some cases, an apheresis is performed −3 weeks, −2 weeks, −1 week, 0, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or up to about 10 years after an administration of a cellular product. In some cases, cells acquired by an apheresis can undergo testing for specific lysis, cytokine release, metabolomics studies, bioenergetics studies, intracellular FACs of cytokine production, ELISA-spot assays, and lymphocyte subset analysis. In some cases, samples of cellular products or apheresis products can be cryopreserved for retrospective analysis of infused cell phenotype and function.

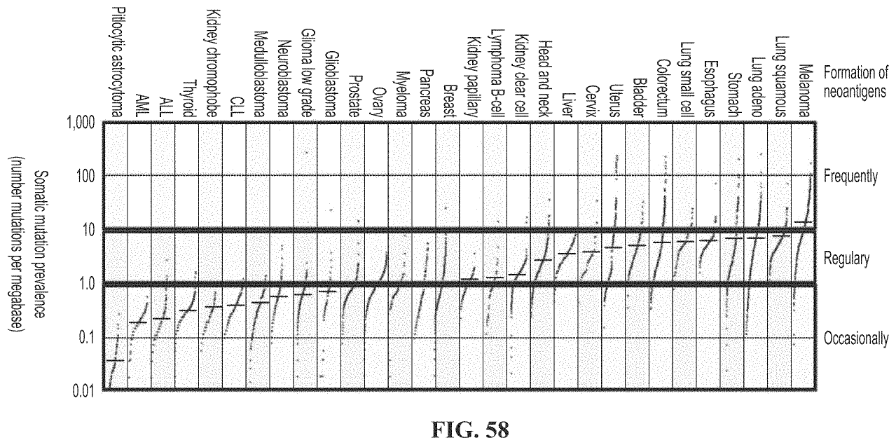

Disclosed herein are compositions and methods useful for performing an intracellular genomic transplant. Exemplary methods for genomic transplantation are described in PCT/US2016/044858, which is hereby incorporated by reference in its entirety. An intracellular genomic transplant may comprise genetically modifying cells and nucleic acids for therapeutic applications. The compositions and methods described throughout can use a nucleic acid-mediated genetic engineering process for delivering a tumor-specific TCR in a way that improves physiologic and immunologic anti-tumor potency of an engineered cell. Effective adoptive cell transfer-based immunotherapies (ACT) can be useful to treat cancer (e.g., metastatic cancer) patients. For example, autologous peripheral blood lymphocytes (PBL) can be modified using viral or non-viral methods to express a transgene such as a T Cell Receptors (TCR) that recognize unique mutations, neo-antigens, on cancer cells and can be used in the disclosed compositions and methods of an intracellular genomic transplant. A Neoantigen can be associated with tumors of high mutational burden, FIG. 58.

Cells can be genetically modified or engineered. Cells (e.g., genetically modified or engineered cells) can be grown and expanded in conditions that can improve its performance once administered to a patient. The engineered cell can be selected. For example, prior to expansion and engineering of the cells, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. For example, any T cell lines can be used. Alternatively, the cell can be derived from a healthy donor, from a patient diagnosed with cancer, or from a patient diagnosed with an infection. In another case, the cell can be part of a mixed population of cells which present different phenotypic characteristics. A cell line can also be obtained from a transformed T-cell according to the method previously described. A cell can also be obtained from a cell therapy bank. Modified cells resistant to an immunosuppressive treatment can be obtained. A desirable cell population can also be selected prior to modification. An engineered cell population can also be selected after modification.

In some cases, the engineered cell can be used in autologous transplantation. Alternatively, the engineered cell can be used in allogeneic transplantation. In some cases, the engineered cell can be administered to the same patient whose sample was used to identify the cancer-related target sequence and/or a transgene (e.g., a TCR transgene). In some cases, the engineered cell can be administered to a patient different from the patient whose sample was used to identify the cancer-related target sequence and/or a transgene (e.g., a TCR transgene). One or more homologous recombination enhancers can be introduced with cells of the present disclosure. Enhancers can facilitate homology directed repair of a double strand break. Enhancers can facilitate integration of a transgene (e.g., a TCR transgene) into a cell of the present disclosure. An enhancer can block non-homologous end joining (NHEJ) so that homology directed repair of a double strand break occurs preferentially.

One or more cytokines can be introduced with cells of the present disclosure. Cytokines can be utilized to boost cytotoxic T lymphocytes (including adoptively transferred tumor-specific cytotoxic T lymphocytes) to expand within a tumor microenvironment. In some cases, IL-2 can be used to facilitate expansion of the cells described herein. Cytokines such as IL-15 can also be employed. Other relevant cytokines in the field of immunotherapy can also be utilized, such as IL-2, IL-7, IL-12, IL-15, IL-21, or any combination thereof. In some cases, IL-2, IL-7, and IL-15 are used to culture cells of the invention.

In some cases, cells can be treated with agents to improve in vivo cellular performance, for example, S-2-hydroxyglutarate (S-2HG). Treatment with S-2HG can improve cellular proliferation and persistence in vivo when compared to untreated cells. S-2HG also can improve anti-tumor efficacy in treated cells compared to cells not treated with S-2HG. In some cases, treatment with S-2HG can result in increased expression of CD62L. In some cases, cells treated with S-2HG can express higher levels of CD127, CD44, 4-1BB, Eomes compared to untreated cells. In some cases, cells treated with S-2HG can have reduced expression of PD-1 when compared to untreated cells. Increased levels of CD127, CD44, 4-1BB, and Eomes can be from about 5% to about 700% when compared to untreated cells, for example, from about 5%, 10%, 20%, 30%, 40%, 50%, 60%, 70%, 80%, 90%, 100%, 150%, 200%, 250%, 300%, 350%, 400%, 450%, 500%, or up to a 700% increase in expression of CD127, CD44, 4-1BB, and Eomes in cells treated with S-2HG. In some cases, cells treated with S-2HG can have from about 5% to about 700% increased cellular expansion and/or proliferation when compared to untreated cells as measured by flow cytometry analysis, e.g., from about 5%, 10%, 20%, 30%, 40%, 50%, 60%, 70%, 80%, 90%, 100%, 150%, 200%, 250%, 300%, 350%, 400%, 450%, 500%, or up to 700% increased cellular expansion and/or proliferation when compared to untreated cells as measured by flow cytometry analysis.

Cells treated with S-2HG can be exposed to a concentration from about 10 μM to about 500 μM. A concentration can be from about 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, or up to 500 μM.

Cytotoxicity may generally refer to the quality of a composition, agent, and/or condition (e.g., exogenous DNA) being toxic to a cell. In some aspects, the methods of the present disclosure generally relate to reduce the cytotoxic effects of exogenous DNA introduced into one or more cells during genetic modification. In some cases, cytotoxicity, or the effects of a substance being cytotoxic to a cell, can comprise DNA cleavage, cell death, autophagy, apoptosis, nuclear condensation, cell lysis, necrosis, altered cell motility, altered cell stiffness, altered cytoplasmic protein expression, altered membrane protein expression, undesired cell differentiation, swelling, loss of membrane integrity, cessation of metabolic activity, hypoactive metabolism, hyperactive metabolism, increased reactive oxygen species, cytoplasmic shrinkage, production of pro-inflammatory cytokines (e.g., as a product of a DNA sensing pathway) or any combination thereof. Non-limiting examples of pro-inflammatory cytokines include interleukin 6 (IL-6), interferon alpha (IFNα), interferon beta (IFNβ), C—C motif ligand 4 (CCL4), C—C motif ligand 5 (CCL5), C—X—C motif ligand 10 (CXCL10), interleukin 1 beta (IL-1β), IL-18 and IL-33. In some cases, cytotoxicity may be affected by introduction of a polynucleic acid, such as a transgene or TCR. A change in cytotoxicity can be measured in any of a number of ways known in the art. In some cases, a change in cytotoxicity can be assessed based on a degree and/or frequency of occurrence of cytotoxicity-associated effects, such as cell death or undesired cell differentiation. In some cases, reduction in cytotoxicity is assessed by measuring amount of cellular toxicity using assays known in the art, which include standard laboratory techniques such as dye exclusion, detection of morphologic characteristics associated with cell viability, injury and/or death, and measurement of enzyme and/or metabolic activities associated with the cell type of interest.

In some cases, cells to undergo genomic transplant can be activated or expanded by co-culturing with tissue or cells. A cell can be an antigen presenting cell. An artificial antigen presenting cells (aAPCs) can express ligands for T cell receptor and costimulatory molecules and can activate and expand T cells for transfer, while improving their potency and function in some cases. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof.

In some cases an artificial antigen presenting cell (aAPC) may be used to activate and/or expand a cell population. In some cases, an artificial may not induce allospecificity. An aAPC may not express HLA in some cases. An aAPC may be genetically modified to stably express genes that can be used to activation and/or stimulation. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, II, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83.

An aAPC can be a bead. A spherical polystyrene bead can be coated with antibodies against CD3 and CD28 and be used for T cell activation. A bead can be of any size. In some cases, a bead can be or can be about 3 and 6 micrometers. A bead can be or can be about 4.5 micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a 4, poly(lactic-co-glycolic acid) (PLGA) microsphere, a nonspherical particle, a 5, carbon nanotube bundle, a 6, ellipsoid PLGA microparticle, a 7, nanoworms, a fluidic lipid bilayer-containing system, an 8, 2D-supported lipid bilayer (2D-SLBs), a 9, liposome, a 10, RAFTsomes/microdomain liposome, an 11, SLB particle, or any combination thereof.

In some cases, an aAPC can expand CD4 T cells. For example, an aAPC can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells.

In some cases, the use of aAPCs can be combined with exogenously introduced cytokines for cell (e.g., T cell) activation, expansion, or any combination. Cells can also be expanded in vivo, for example in the subject's blood after administration of genomically transplanted cells into a subject.

These compositions and methods for intracellular genomic transplant can provide a cancer therapy with many advantages. For example, they can provide high efficiency gene transfer, expression, increased cell survival rates, an efficient introduction of recombinogenic double strand breaks, and a process that favors the Homology Directed Repair (HDR) over Non-Homologous End Joining (NHEJ) mechanism, and efficient recovery and expansion of homologous recombinants.

### Intracellular Genomic Transplant

Intracellular genomic transplant can be method of genetically modifying cells and nucleic acids for therapeutic applications. The compositions and methods described throughout can use a nucleic acid-mediated genetic engineering process for tumor-specific TCR expression in a way that leaves the physiologic and immunologic anti-tumor potency of the T cells unperturbed. Effective adoptive cell transfer-based immunotherapies (ACT) can be useful to treat cancer (e.g., metastatic cancer) patients. For example, autologous peripheral blood lymphocytes (PBL) can be modified using non-viral methods to express T Cell Receptors (TCR) that recognize unique mutations, neo-antigens, on cancer cells and can be used in the disclosed compositions and methods of an intracellular genomic transplant.

One exemplary method of identifying a sequence of cancer-specific TCR that recognizes unique immunogenic mutations on the patient's cancer are described in PCT/US14/58796. For example, a transgene (e.g., cancer-specific TCR, or an exogenous transgene) can be inserted into the genome of a cell (e.g., T cell) using random or specific insertions. In some cases, an insertion can be a viral insertion. In some cases, an insertion can be via a non-viral insertion (e.g., with a minicircle vector). In some cases, a viral insertion of a transgene can be targeted to a particular genomic site or in other cases a viral insertion of a transgene can be a random insertion into a genomic site. In some cases, a transgene (e.g., at least one exogenous transgene, a T cell receptor (TCR)) or a nucleic acid (e.g., at least one exogenous nucleic acid) is inserted once into the genome of a cell. In some cases, a transgene (e.g., at least one exogenous transgene, a T cell receptor (TCR)) or a nucleic acid (e.g., at least one exogenous nucleic acid) is randomly inserted into a genomic locus. In some cases, a transgene (e.g., at least one exogenous transgene, a T cell receptor (TCR)) or a nucleic acid (e.g., at least one exogenous nucleic acid) is randomly inserted into more than one genomic locus. In some cases, a transgene (e.g., at least one exogenous transgene, a T cell receptor (TCR)) or a nucleic acid (e.g., at least one exogenous nucleic acid) is inserted in at least one gene (e.g., CISH and/or TCR). In some cases, a transgene (e.g., at least one exogenous transgene, a TCR) or a nucleic acid (e.g., at least one exogenous nucleic acid) is inserted at a break in a gene (e.g., CISH and/or TCR). In some cases, more than one transgene (e.g., exogenous transgene, a TCR) is inserted into the genome of a cell. In some cases, more than one transgene is inserted into one or more genomic locus. In some cases, a transgene (e.g., at least one exogenous transgene) or a nucleic acid (e.g., at least one exogenous nucleic acid) is inserted in at least one gene. In some cases, a transgene (e.g., at least one exogenous transgene) or a nucleic acid (e.g., at least one exogenous nucleic acid) is inserted in two or more genes (e.g., CISH and/or TCR). In some cases, a transgene (e.g., at least one exogenous transgene) or a nucleic acid (e.g., at least one exogenous nucleic acid) is inserted into the genome of a cell in a random and/or specific manner. In some cases, a transgene is an exogenous transgene. In some cases, a transgene (e.g., at least one exogenous transgene) is flanked by engineered sites complementary to at least a portion of a gene (e.g., CISH and/or TCR). In some cases, a transgene (e.g., at least one exogenous transgene) is flanked by engineered sites complementary to a break in a gene (e.g., CISH and/or TCR). In some cases, a transgene (e.g., at least one exogenous transgene) is not inserted in a gene (e.g., not inserted in CISH and/or TCR). In some cases, a transgene is not inserted at a break in a gene (e.g., break in CISH and/or TCR).

In some cases, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 97%, or at least about 98%, or at least about 99% of the cells in a population of genetically modified cells, or in a population of genetically modified TILs comprise at least one exogenous transgene (e.g., exogenous TCR). In some cases, any of the methods of the present disclosure can result in at least about or about 5%, or at least about or about 10%, or at least about or about 15%, or at least about or about 20%, or at least about or about 25%, or at least about or about 30%, or at least about or about 35%, or at least about or about 40%, or at least about or about 45%, or at least about or about 50%, or at least about or about 55%, or at least about or about 60%, or at least about or about 65%, or at least about or about 70%, or at least about or about 75%, or at least about or about 80%, or at least about or about 85%, or at least about or about 90%, or at least about or about 95%, or at least about or about 97%, or at least about or about 98%, or at least about or about 99% of the cells in a population of genetically modified cells or genetically modified TILS to comprise at least one exogenous transgene (e.g., a TCR). In some cases, at least about or about 3% 5%, 8%, 10%, 15%, 20%, 25%, 30%, 35%, 40%, 45%, 50%, 55%, 60%, 65%, 70%, 75%, 80%, 85%, 90%, 93%, 95%, 97%, 98%, 99%, 99.5%, or 100% of the cells in a population of genetically modified cells comprises at least one exogenous transgene (e.g., a TCR) integrated at a break in at least one gene (e.g., CISH and/or TCR). In some cases, at least one exogenous transgene is integrated at a break in one or more genes (e.g., CISH and/or TCR). In some cases, at least about or about 3% 5%, 8%, 10%, 15%, 20%, 25%, 30%, 35%, 40%, 45%, 50%, 55%, 60%, 65%, 70%, 75%, 80%, 85%, 90%, 93%, 95%, 97%, 98%, 99%, 99.5%, or 100% of the cells in a population of genetically modified cells comprises at least one exogenous transgene integrated in the genome of a cell. In some cases, at least about or about 3% 5%, 8%, 10%, 15%, 20%, 25%, 30%, 35%, 40%, 45%, 50%, 55%, 60%, 65%, 70%, 75%, 80%, 85%, 90%, 93%, 95%, 97%, 98%, 99%, 99.5%, or 100% of the cells in a population of genetically modified cells comprises at least one exogenous transgene integrated in a genomic locus (e.g., CISH and/or TCR). In some cases, the integration comprises a viral (e.g., AAV or modified AAV) or a non-viral (e.g., minicircle) system.

In some cases, the present disclosure provides a population of genetically modified cells and/or a population of tumor infiltrating lymphocytes (e.g., genetically modified TILs) and methods of producing a population of genetically modified cells (e.g., genetically modified TILs). In some cases, said population of genetically modified cells comprises at least about 30%, 35%, 40%, 45%, 50%, 55%, 60%, 65%, 70%, 75%, 80%, 85%, 90%, 95%, 97%, 98%, 99%, 99.5%, or 100% cell viability (e.g., cell viability is measured at some time after an AAV vector (or a non-viral vector (e.g., a minicircle vector)) is introduced to a population of cells and/or cell viability is measured at some time after at least one exogenous transgene is integrated into a genomic locus (e.g., CISH and/or TCR) of at least one cell). In some cases, cell viability is measured by FACS. In some cases, cell viability is measured at about, at least about, or at most about 4 hours, 6 hours, 8 hours, 10 hours, 12 hours, 18 hours, 20 hours, 24 hours, 30 hours, 36 hours, 40 hours, 48 hours, 54 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, or longer than 240 hours after a viral (e.g., AAV) or a non-viral (e.g., minicircle) vector is introduced to a cell and/or to a population of cells. In some cases, cell viability is measured at about, at least about, or at most about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 45 days, 50 days, 60 days, 70 days, 90 days, or longer than 90 days after a viral (e.g., AAV) or a non-viral (e.g., minicircle) vector is introduced to a cell and/or to a population of cells. In some cases, cell viability is measured after at least one exogenous transgene (e.g., a TCR) is integrated into a genomic locus (e.g., CISH and/or TCR) of at least one cell. In some cases, cell viability is measured at about, at least about, or at most about 4 hours, 6 hours, 8 hours, 10 hours, 12 hours, 18 hours, 20 hours, 24 hours, 30 hours, 36 hours, 40 hours, 48 hours, 54 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, longer than 240 hours, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 45 days, 50 days, 60 days, 70 days, 90 days, or longer than 90 days after at least one exogenous transgene (e.g., a TCR) is integrated into a genomic locus of at least one cell. In some cases, cell toxicity is measured after a viral or a non-viral system is introduced to a cell or to a population of cells. In some cases, cell toxicity is measured after at least one exogenous transgene (e.g., a TCR) is integrated into a genomic locus (e.g., CISH and/or TCR) of at least one cell. In some cases, cell toxicity is lower when a modified AAV vector is used than when a wild-type or unmodified AAV or when a non-viral system (e.g., minicircle vector) is introduced to a comparable cell or to a comparable population of cells. In some cases, cell toxicity is lower when an AAV vector is used than when a non-viral vector (e.g., minicircle vector) is introduced to a comparable cell or to a comparable population of cells. In some cases, cell toxicity is measured by flow cytometry. In some cases, cell toxicity is reduced by about, at least about, or at most about 1%, 2%, 3%, 4%, 5%, 6%, 7%, 8%, 9%, 10%, 12%, 15%, 17%, 18%, 19%, 20%, 25%, 30%, 35%, 40%, 45%, 50%, 55%, 60%, 65%, 70%, 75%, 80%, 82%, 85%, 88%, 90%, 92%, 95%, 97%, 98%, 99% or 100% when a modified or recombinant AAV vector is used to integrate at least one exogenous transgene (e.g., a TCR) compared to when a wild-type or unmodified AAV vector or a minicircle vector is used to integrate at least one exogenous transgene (e.g., a TCR). In some cases, cell toxicity is reduced by about, at least about, or at most about 1%, 2%, 3%, 4%, 5%, 6%, 7%, 8%, 9%, 10%, 12%, 15%, 17%, 18%, 19%, 20%, 25%, 30%, 35%, 40%, 45%, 50%, 55%, 60%, 65%, 70%, 75%, 80%, 82%, 85%, 88%, 90%, 92%, 95%, 97%, 98%, 99% or 100% when an AAV vector is used compared to when a minicircle vector or another non-viral system is used to integrate at least one exogenous transgene. In some cases, an AAV is selected from the group consisting of recombinant AAV (rAAV), modified AAV, hybrid AAV, self-complementary AAV (scAAV), and any combination thereof.

In some cases, the methods disclosed herein comprise introducing into a cell one or more nucleic acids (e.g., a first nucleic acid and/or a second nucleic acid). A person of skill in the art will appreciate that a nucleic acid may generally refer to a substance whose molecules consist of many nucleotides linked in a long chain Non-limiting examples of the nucleic acid include an artificial nucleic acid analog (e.g., a peptide nucleic acid, a morpholino oligomer, a locked nucleic acid, a glycol nucleic acid, or a threose nucleic acid), a circular nucleic acid, a DNA, a single stranded DNA, a double stranded DNA, a genomic DNA, a mini-circle DNA, a plasmid, a plasmid DNA, a viral DNA, a viral vector, a gamma-retroviral vector, a lentiviral vector, an adeno-associated viral vector, an RNA, short hairpin RNA, psiRNA and/or a hybrid or combination thereof. In some cases, a method may comprise a nucleic acid, and the nucleic acid is synthetic. In some cases, a sample may comprise a nucleic acid, and the nucleic acid may be fragmented. In some cases, a nucleic acid is a minicircle.

In some cases, a nucleic acid may comprise promoter regions, barcodes, restriction sites, cleavage sites, endonuclease recognition sites, primer binding sites, selectable markers, unique identification sequences, resistance genes, linker sequences, or any combination thereof. A nucleic acid may be generated without the use of bacteria. For example, a nucleic acid can have reduced traces of bacterial elements or completely devoid of bacterial elements. A nucleic acid when compared to a plasmid vector can have from 20%-40%, 40%-60%, 60%-80%, or 80%-100% less bacterial traces than a plasmid vector as measured by PCR. A nucleic acid when compared to a plasmid vector can have from 20%, 30%, 40%, 50%, 60%, 70%, 80%, 90%, 95%, or up to 100% less bacterial traces than a plasmid vector as measured by PCR. In some aspects, these sites may be useful for enzymatic digestion, amplification, sequencing, targeted binding, purification, providing resistance properties (e.g., antibiotic resistance), or any combination thereof. In some cases, the nucleic acid may comprise one or more restriction sites. A restriction site may generally refer to a specific peptide or nucleotide sequences at which site-specific molecules (e.g., proteases, endonucleases, or enzymes) may cut the nucleic acid. In one example, a nucleic acid may comprise one or more restriction sites, wherein cleaving the nucleic acid at the restriction site fragments the nucleic acid. In some cases, the nucleic acid may comprise at least one endonuclease recognition site.

In some cases, a nucleic acid may readily bind to another nucleic acid (e.g., the nucleic acid comprises a sticky end or nucleotide overhang). For example, the nucleic acid may comprise an overhang at a first end of the nucleic acid. Generally, a sticky end or overhang may refer to a series of unpaired nucleotides at the end of a nucleic acid. In some cases, the nucleic acid may comprise a single stranded overhang at one or more ends of the nucleic acid. In some cases, the overhang can occur on the 3′ end of the nucleic acid. In some cases, the overhang can occur on the 5′ end of the nucleic acid. The overhang can comprise any number of nucleotides. For example, the overhang can comprise 1 nucleotide, 2 nucleotides, 3 nucleotides, 4 nucleotides, or 5 or more nucleotides. In some cases, the nucleic acid may require modification prior to binding to another nucleic acid (e.g., the nucleic acid may need to be digested with an endonuclease). In some cases, modification of the nucleic acid may generate a nucleotide overhang, and the overhang can comprise any number of nucleotides. For example, the overhang can comprise 1 nucleotide, 2 nucleotides, 3 nucleotides, 4 nucleotides, or 5 or more nucleotides. In one example, the nucleic acid may comprise a restriction site, wherein digesting the nucleic acid at the restriction site with a restriction enzyme (e.g., NotI) produces a 4 nucleotide overhang. In some cases, the modifying comprises generating a blunt end at one or more ends of the nucleic acid. Generally, a blunt end may refer to a double stranded nucleic acid wherein both strands terminate in a base pair. In one example, the nucleic acid may comprise a restriction site, wherein digesting the nucleic acid at the restriction site with a restriction enzyme (e.g., BsaI) produces a blunt end.

Promoters are sequences of nucleic acid that control the binding of RNA polymerase and transcription factors, and can have a major effect on the efficiency of gene transcription, where a gene may be expressed in the cell, and/or what cell types a gene may be expressed in. Non limiting examples of promoters include a cytomegalocirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, or a combination thereof.

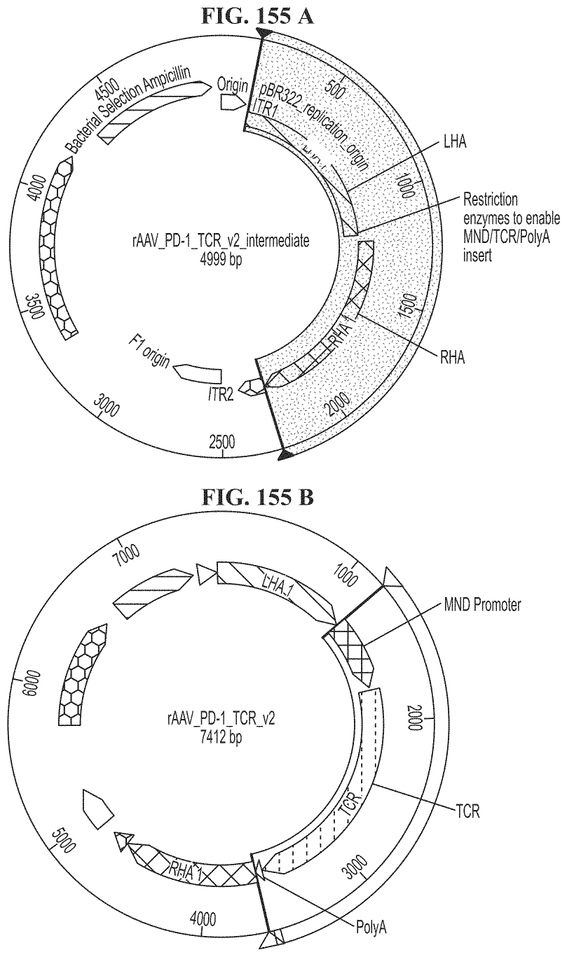

A promoter can be CMV, U6, MND, or EF1a, FIG. 155A. In some cases, a promoter can be adjacent to an exogenous TCR sequence. In some cases, an rAAV vector can further comprises a splicing acceptor. In some cases, the splicing acceptor can be adjacent to the exogenous TCR sequence. A promoter sequence can be a PKG or an MND promoter, FIG. 155B. An MND promoter can be a synthetic promoter that contains a U3 region of a modified MoMuLV LTR with a myeloproliferative sarcoma virus enhancer.

### Viral Vectors

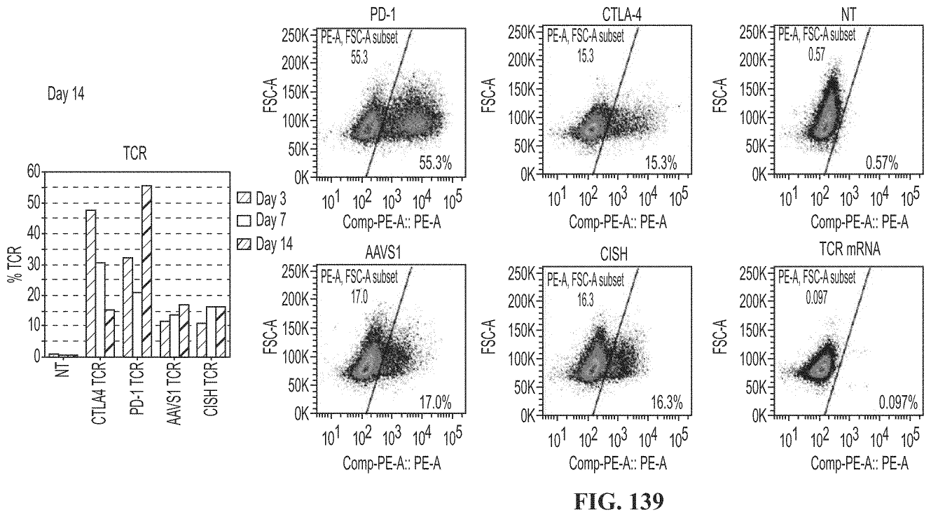

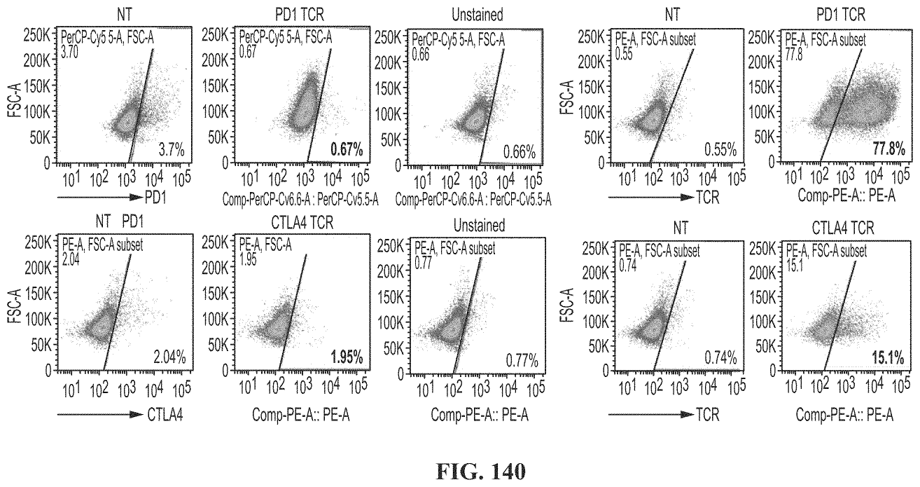

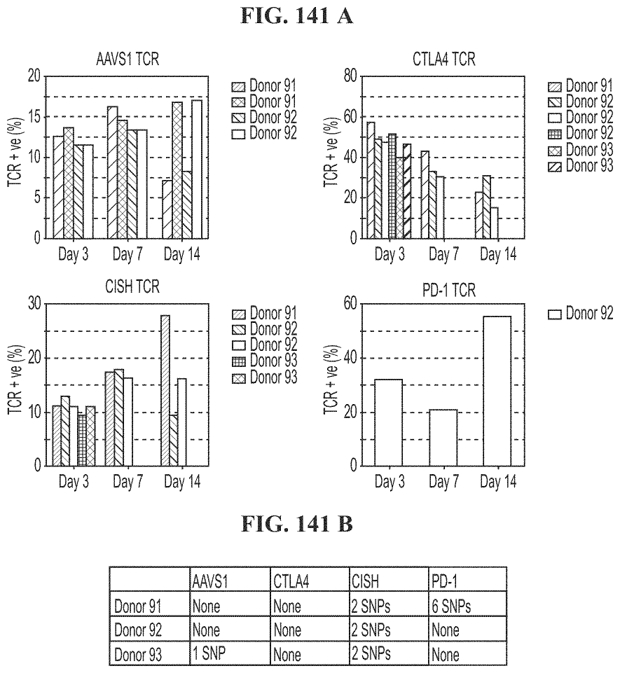

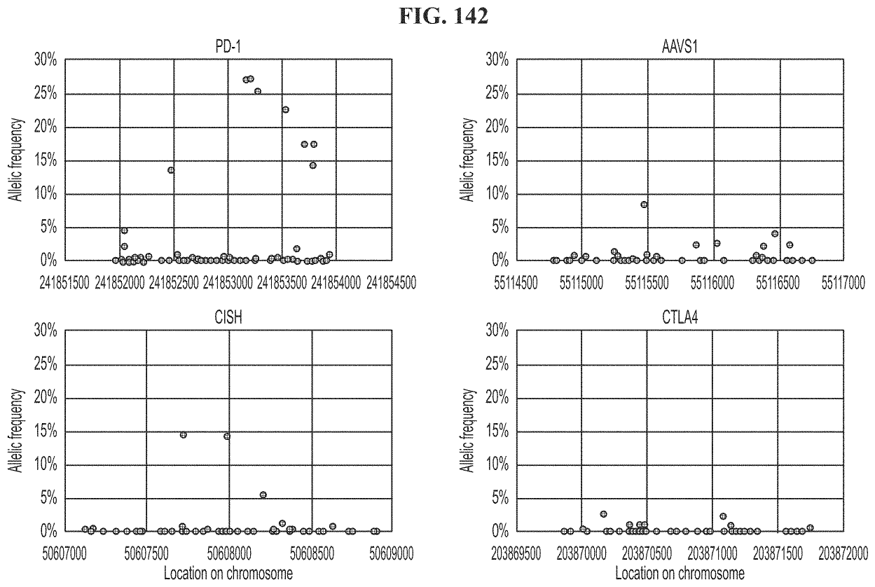

In some cases, a viral vector may be utilized to introduce a transgene into a cell. A viral vector can be, without limitation, a lentivirus, a retrovirus, or an adeno-associated virus. A viral vector may be an adeno-associated viral vector, FIG. 139 and FIG. 140. In some cases, an adeno-associated virus (AAV) vector can be a recombinant AAV (rAAV) vector, a hybrid AAV vector, a self-complementary AAV (scAAV) vector, a mutant AAV vector, and any combination thereof. In some cases, an adeno-associated virus can be used to introduce an exogenous transgene (e.g., at least one exogenous transgene). A viral vector can be isogenic in some cases. A viral vector may be integrated into a portion of a genome with known SNPs in some cases. In other cases, a viral vector may not be integrated into a portion of a genome with known SNPs. For example, a rAAV can be designed to be isogenic or homologous to a subjects own genomic DNA. In some cases, an isogenic vector can improve efficiency of homologous recombination. In some cases, a gRNA may be designed so that it does not target a region with known SNPs to improve the expression of an integrated vector transgene. The frequency of SNPs at checkpoint genes, such as PD-1, CISH, AAVS1, and CTLA-4, can be determined, FIG. 141A, FIG. 141B, and FIG. 142.

An adeno-associated virus (AAV) can be a non-pathogenic single-stranded DNA parvovirus. An AAV can have a capsid diameter of about 26 nm. A capsid diameter can also be from about 20 nm to about 50 nm in some cases. Each end of the AAV single-stranded DNA genome can contain an inverted terminal repeat (ITR), which can be the only cis-acting element required for genome replication and packaging. The genome carries two viral genes: rep and cap. The virus utilizes two promoters and alternative splicing to generate four proteins necessary for replication (Rep78, Rep 68, Rep 52 and Rep 40), while a third promoter generates the transcript for three structural viral capsid proteins, 1, 2 and 3 (VP1, VP2 and VP3), through a combination of alternate splicing and alternate translation start codons. The three capsid proteins share the same C-terminal 533 amino acids, while VP2 and VP1 contain additional N-terminal sequences of 65 and 202 amino acids, respectively. The AAV virion can contain a total of 60 copies of VP1, VP2, and VP3 at a 1:1:20 ratio, arranged in a T=1 icosahedral symmetry.

At the cellular level, AAV can undergo 5 major steps prior to achieving gene expression: 1) binding or attachment to cellular surface receptors, 2) endocytosis, 3) trafficking to the nucleus, 4) uncoating of the virus to release the genome and 5) conversion of the genome from single-stranded to double-stranded DNA as a template for transcription in the nucleus. The cumulative efficiency with which rAAV can successfully execute each individual step can determine the overall transduction efficiency. Rate limiting steps in rAAV transduction can include the absence or low abundance of required cellular surface receptors for viral attachment and internalization, inefficient endosomal escape leading to lysosomal degradation, and slow conversion of single-stranded to double-stranded DNA template. Therefore, vectors with modifications to the genome and/or the capsids can be designed to facilitate more efficient or more specific transduction or cells or tissues for gene therapy.

In some cases, a viral capsid may be modified. A modification can include modifying a combination of capsid components. For example, a mosaic capsid AAV is a virion that can be composed of a mixture of viral capsid proteins from different serotypes. The capsid proteins can be provided by complementation with separate plasmids that are mixed at various ratios. During viral assembly, the different serotypes capsid proteins can be mixed in each virion, at subunit ratios stoichiometrically reflecting the ratios of the complementing plasmids. A mosaic capsid can confer increased binding efficacy to certain cell types or improved performance as compared to an unmodified capsid.

In some cases, a chimeric capsid AAV can be generated. A chimeric capsid can have an insertion of a foreign protein sequence, either from another wild-type (wt) AAV sequence or an unrelated protein, into the open reading frame of the capsid gene. Chimeric modifications can include the use of naturally existing serotypes as templates, which can involve AAV capsid sequences lacking a certain function being co-transfected with DNA sequences from another capsid. Homologous recombination occurs at crossover points leading to capsids with new features and unique properties. In other cases, the use of epitope coding sequences fused to either the N or C termini of the capsid coding sequences to attempt to expose new peptides on the surface of the viral capsid without affecting gene function. In some cases, the use of epitope sequences inserted into specific positions in the capsid coding sequence, but using a different approach of tagging the epitope into the coding sequences itself can be performed. A chimeric capsid can also include the use of an epitope identified from a peptide library inserted into a specific position in the capsid coding sequence. The use of gene library to screen can be performed. A screen can catch insertions that do not function as intended can can subsequently be deleted and a screen. Chimeric capsids in rAAV vectors can expand the range of cell types that can be transfected and can increase the efficiency of transduction. Increased transduction can be from about a 10% increase to about a 300% increase as compared to a transduction using an unmodified capsid. A chimeric capsid can contain a degenerate, recombined, shuffled or otherwise modified Cap protein. For example targeted insertion of receptor-specific ligands or single-chain antibodies at the N-terminus of VP proteins can be performed. An insertion of a lymphocyte antibody or target into an AAV can be performed to improve binding and infection of a T cell.

In some cases, a chimeric AAV can have a modification in at least one AAV capsid protein (e.g., a modification in the VP1, VP2, and/or VP3 capsid protein). In some cases, an AAV vector comprises a modification in at least one of the VP1, VP2, and VP3 capsid gene sequences. In some cases, at least one capsid gene may be deleted from an AAV. In some cases, an AAV vector may comprise a deletion of one or more capsid gene sequences. In some cases, an AAV vector can have at least one amino acid substitution, deletion, and/or insertion in at least one of the VP1, VP2, and VP3 capsid gene sequences.

In some cases, virions having chimeric capsids (e.g., capsids containing a degenerate or otherwise modified Cap protein) can be made. To further alter the capsids of such virions, e.g., to enhance or modify the binding affinity for a specific cell type, such as a lymphocyte, additional mutations can be introduced into the capsid of the virion. For example, suitable chimeric capsids may have ligand insertion mutations for facilitating viral targeting to specific cell types. The construction and characterization of AAV capsid mutants including insertion mutants, alanine screening mutants, and epitope tag mutants is described in Wu et al., J. Virol. 74:8635-45, 2000. Methods of making AAV capsid mutants are known, and include site-directed mutagenesis (Wu et al., J. Virol. 72:5919-5926); molecular breeding, nucleic acid, exon, and DNA family shuffling (Soong et al., Nat. Genet. 25:436-439, 2000; Coco et al., Nature Biotech. 2001; 19:354; and U.S. Pat. Nos. 5,837,458; 5,811,238; and 6,180,406; Kolkman and Stemmer, Nat. Biotech. 19:423-428, 2001; Fisch et al., Proceedings of the National Academy of Sciences 93:7761-7766, 1996; Christians et al., Nat. Biotech. 17:259-264, 1999); ligand insertions (Girod et al. Nat. Med. 9:1052-1056, 1999); cassette mutagenesis (Rueda et al. Virology 263:89-99, 1999; Boyer et al., J. Virol. 66:1031-1039, 1992); and the insertion of short random oligonucleotide sequences.

In some cases, a transcapsidation can be performed. Transcapsidation can be a process that involves the packaging of the ITR of one serotype of AAV into the capsid of a different serotype. In another case, adsorption of receptor ligands to an AAV capsid surface can be performed and can be the addition of foreign peptides to the surface of an AAV capsid. In some cases, this can confer the ability to specifically target cells that no AAV serotype currently has a tropism towards, and this can greatly expand the uses of AAV as a gene therapy tool.

In some cases, an rAAV vector can be modified. For example, an rAAV vector can comprise a modification such as an insertion, deletion, chemical alteration, or synthetic modification. In some cases, a single nucleotide is inserted into an rAAV vector. In other cases, multiple nucleotides are inserted into a vector. Nucleotides that can be inserted can range from about 1 nucleotide to about 5 kb. Nucleotides that can be inserted can encode for a functional protein. A nucleotide that can be inserted can be endogenous or exogenous to a subject receiving a vector. For example, a human cell can receive an rAAV vector that can contain at least a portion of a murine genome, such as a portion of a TCR. In some cases, a modification such as an insertion or deletion of an rAAV vector can comprise a protein coding region or a non-coding region of a vector. In some cases, a modification may improve activity of a vector when introduced into a cell. For example, a modification can improve expression of protein coding regions of a vector when introduced into a human cell.

In some cases, the present disclosure provides construction of helper vectors that provide AAV Rep and Cap proteins for producing stocks of virions composed of an rAAV vector (e.g., a vector encoding an exogenous receptor sequence) and a chimeric capsid (e.g., a capsid containing a degenerate, recombined, shuffled or otherwise modified Cap protein). In some cases, a modification can involve the production of AAV cap nucleic acids that are modified, e.g., cap nucleic acids that contain portions of sequences derived from more than one AAV serotype (e.g., AAV serotypes 1-8). Such chimeric nucleic acids can be produced by a number of mutagenesis techniques. A method for generating chimeric cap genes can involve the use of degenerate oligonucleotides in an in vitro DNA amplification reaction. A protocol for incorporating degenerate mutations (e.g., polymorphisms from different AAV serotypes) into a nucleic acid sequence is described in Coco et al. (Nature Biotechnology 20:1246-1250, 2002. In this method, known as degenerate homoduplex recombination, “top-strand” oligonucleotides are constructed that contain polymorphisms (degeneracies) from genes within a gene family. Complementary degeneracies are engineered into multiple bridging “scaffold” oligonucleotides. A single sequence of annealing, gap-filling, and ligation steps results in the production of a library of nucleic acids capturing every possible permutation of the parental polymorphisms. Any portion of a capsid gene may be mutated using methods such as degenerate homoduplex recombination. Particular capsid gene sequences, however, are preferred. For example, critical residues responsible for binding of an AAV2 capsid to its cell surface receptor heparan sulfate proteoglycan (HSPG) have been mapped. Arginine residues at positions 585 and 588 appear to be critical for binding, as non-conservative mutations within these residues eliminate binding to heparin-agarose. Computer modeling of the AAV2 and AAV4 atomic structures identified seven hypervariable regions that overlap arginine residues 585 and 588, and that are exposed to the surface of the capsid. These hypervariable regions are thought to be exposed as surface loops on the capsid that mediate receptor binding. Therefore, these loops can be used as targets for mutagenesis in methods of producing chimeric virions with tropisms different from wt virions. In some cases, a modification can be of an AAV serotype 6 capsid.

Another mutagenesis technique that can be used in methods of the present disclosure is DNA shuffling. DNA or gene shuffling involves the creation of random fragments of members of a gene family and their recombination to yield many new combinations. To shuffle AAV capsid genes, several parameters can be considered, including: involvement of the three capsid proteins VP1, VP2, and VP3 and different degrees of homologies between 8 serotypes. To increase the likelihood of obtaining a viable rcAAV vector with a cell- or tissue-specific tropism, for example, a shuffling protocol yielding a high diversity and large number of permutations is preferred. An example of a DNA shuffling protocol for the generation of chimeric rcAAV is random chimeragenesis on transient templates (RACHITT), Coco et al., Nat. Biotech. 19:354-358, 2001. The RACHITT method can be used to recombine two PCR fragments derived from AAV genomes of two different serotypes (e.g., AAV 5d AAV6). For example, conservative regions of the cap gene, segments that are 85% identical, spanning approximately 1 kbp and including initiating codons for all three genes (VP1, VP2, and VP3) can be shuffled using a RATCHITT or other DNA shuffling protocol, including in vivo shuffling protocols (U.S. Pat. No. 5,093,257; Volkov et al., NAR 27:e18, 1999; and Wang P. L., Dis. Markers 16:3-13, 2000). A resulting combinatorial chimeric library can be cloned into a suitable AAV TR-containing vector to replace the respective fragment of the WT AAV genome. Random clones can be sequenced and aligned with parent genomes using AlignX application of Vector NTI 7 Suite Software. From the sequencing and alignment, the number of recombination crossovers per 1 Kbp gene can be determined. Alternatively, the variable domain of AAV genomes can be shuffled using methods of the present disclosure. For example, mutations can be generated within two amino acid clusters (amino acids 509-522 and 561-591) of AAV that likely form a particle surface loop in VP3. To shuffle this low homology domain, recombination protocols can be utilized that are independent of parent's homology (Ostermeier et al., Nat. Biotechnol. 17:1205-1209, 1999; Lutz et al., Proceedings of the National Academy of Sciences 98:11248-11253, 2001; and Lutz et al., NAR 29:E16, 2001) or a RACHITT protocol modified to anneal and recombine DNA fragments of low homology.

In some cases, a targeted mutation of S/T/K residues on an AAV capsid can be performed. Following cellular internalization of AAV by receptor-mediated endocytosis, it can travel through the cytosol, undergoing acidification in the endosomes before getting released. Post endosomal escape, AAV undergoes nuclear trafficking, where uncoating of the viral capsid takes place resulting in release of its genome and induction of gene expression. S/T/K residues are potential sites for phosphorylation and subsequent poly-ubiquitination which serves as a cue for proteasomal degradation of capsid proteins. This can prevent trafficking of the vectors into the nucleus to express its transgene, an exogenous TCR, leading to low gene expression. Also, the proteasomally degraded capsid fragments can be presented by the MHC-Class I molecules on the cell surface for CD8 T-cell recognition. This leads to immune response thus destroying the transduced cells and further reducing persistent transgene expression. Point mutations, S/T to A and K to R, can prevent/reduce phosphorylation sites on the capsid. This can lead to reduced ubiquitination and proteosomal degradation allowing more number of intact vectors to enter nucleus and express the transgene. Preventing/lowering the overall capsid degradation also reduces antigen presentation to T cells resulting in lower host immune response against the vectors.

In some aspects, an AAV vector comprising a nucleotide sequence of interest flanked by AAV ITRs can be constructed by directly inserting heterologous sequences into an AAV vector. These constructs can be designed using techniques well known in the art. See, e.g., Carter B., Adeno-associated virus vectors, Curr. Opin. Biotechnol., 3:533-539 (1992); and Kotin R M, Prospects for the use of adeno-associated virus as a vector for human gene therapy, Hum Gene Ther 5:793-801 (1994).

In some cases, an AAV expression vector comprises a heterologous nucleic acid sequence of interest, such as a transgene with a therapeutic effect. A rAAV virion can be constructed using methods that are known in the art. See, e.g., Koerber et al. (2009) Mol. Ther. 17:2088; Koerber et al. (2008) Mol Ther. 16:1703-1709; U.S. Pat. Nos. 7,439,065 and 6,491,907. For example, exogenous or heterologous sequence(s) can be inserted into an AAV genome wherein its major AAV open reading frames have been excised therefrom. Other portions of the AAV genome can also be deleted, which certain portions of the ITRs remain intact to support replication and packaging functions. Such constructs can be designed using techniques well known in the art. See, e.g., U.S. Pat. Nos. 5,173,414 and 5,139,941; Lebkowski et al. (1988) Molec. Cell. Biol. 8:3988-3996.

The present application provides methods and materials for producing recombinant AAVs that can express one or more proteins of interest in a cell. As described herein, the methods and materials disclosed herein allow for high production or production of the proteins of interest at levels that would achieve a therapeutic effect in vivo. An example of a protein of interest is an exogenous receptor. An exogenous receptor can be a TCR.

In general, rAAV virions or viral particles, or an AAV expression vector is introduced into a suitable host cell using known techniques, such as by transfection. Transfection techniques are known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Suitable transfection methods include calcium phosphate co-precipitation, direct micro-injection, electroporation, liposome mediated gene transfer, and nucleic acid delivery using high-velocity microprojectiles, which are known in the art.

In some cases, methods for producing a recombinant AAV include providing a packaging cell line with a viral construct comprising a 5′ inverted terminal repeat (ITR) of AAV and a 3′ AAV ITR, such as described herein, helper functions for generating a productive AAV infection, and AAV cap genes; and recovering a recombinant AAV from the supernatant of the packaging cell line. Various types of cells can be used as the packaging cell line. For example, packaging cell lines that can be used include, but are not limited to, HEK 293 cells, HeLa cells, and Vero cells to name a few. In some cases, supernatant of the packaging cell line is treated by PEG precipitation for concentrating the virus. In other cases, a centrifugation step can be used to concentrate a virus. For example a column can be used to concentration a virus during a centrifugation. In some cases, a precipitation occurs at no more than about 4° C. (for example about 3° C., about 2° C., about 1° C., or about 1° C.) for at least about 2 hours, at least about 3 hours, at least about 4 hours, at least about 6 hours, at least about 9 hours, at least about 12 hours, or at least about 24 hours. In some cases, the recombinant AAV is isolated from the PEG-precipitated supernatant by low-speed centrifugation followed by CsCl gradient. The low-speed centrifugation can be to can be about 4000 rpm, about 4500 rpm, about 5000 rpm, or about 6000 rpm for about 20 minutes, about 30 minutes, about 40 minutes, about 50 minutes or about 60 minutes. In some cases, recombinant AAV is isolated from the PEG-precipitated supernatant by centrifugation at about 5000 rpm for about 30 minutes followed by CsCl gradient

In some cases, helper functions are provided by one or more helper plasmids or helper viruses comprising adenoviral helper genes. Non-limiting examples of the adenoviral helper genes include E1A, E1B, E2A, E4 and VA, which can provide helper functions to AAV packaging. In some cases, an AAV cap gene can be present in a plasmid. A plasmid can further comprise an AAV rep gene.

Serology can be defined as the inability of an antibody that is reactive to the viral capsid proteins of one serotype in neutralizing those of another serotype. In some cases, a cap gene and/or rep gene from any AAV serotype (including, but not limited to, AAV1, AAV2, AAV3, AAV4, AAVS, AAV6, AAV7, AAVS, AAVS, AAV10, AAV11, AAV12, and any variant or derivative thereof) can be used herein to produce the recombinant AAV disclosed herein to express one or more proteins of interest. An adeno-associated virus can be AAVS or AAV6 or a variant thereof. In some cases, an AAV cap gene can encode a capsid from serotype 1, serotype 2, serotype 3, serotype 4, serotype 5, serotype 6, serotype 7, serotype 8, serotype 9, serotype 10, serotype 11, serotype 12, or a variant thereof. In some cases, a packaging cell line can be transfected with the helper plasmid or helper virus, the viral construct and the plasmid encoding the AAV cap genes; and the recombinant AAV virus can be collected at various time points after co-transfection. For example, the recombinant AAV virus can be collected at about 12 hours, about 24 hours, about 36 hours, about 48 hours, about 72 hours, about 96 hours, about 120 hours, or a time between any of these two time points after the co-transfection.

Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US Publication No. 20110201088, helper vectors pHELP (Applied Viromics). A skilled artisan will appreciate that any helper virus or helper plasmid of AAV that can provide adequate helper function to AAV can be used herein. The recombinant AAV viruses disclosed herein can also be produced using any convention methods known in the art suitable for producing infectious recombinant AAV. In some instances, a recombinant AAV can be produced by using a cell line that stably expresses some of the necessary components for AAV particle production. For example, a plasmid (or multiple plasmids) comprising AAV rep and cap genes, and a selectable marker, such as a neomycin resistance gene, can be integrated into the genome of a cell (the packaging cells). The packaging cell line can then be co-infected with a helper virus (e.g., adenovirus providing the helper functions) and the viral vector comprising the 5′ and 3′ AAV ITR and the nucleotide sequence encoding the protein(s) of interest. In another non-limiting example, adenovirus or baculovirus rather than plasmids can be used to introduce rep and cap genes into packaging cells. As yet another non-limiting example, both the viral vector containing the 5′ and 3′ AAV ITRs and the rep-cap genes can be stably integrated into the DNA of producer cells, and the helper functions can be provided by a wild-type adenovirus to produce the recombinant AAV.

Suitable host cells that can be used to produce rAAV virions or viral particles include yeast cells, insect cells, microorganisms, and mammalian cells. Various stable human cell lines can be used, including, but not limited to, 293 cells. Host cells can be engineered to provide helper functions in order to replicate and encapsidate nucleotide sequences flanked by AAV ITRs to produce viral particles or AAV virions. AAV helper functions can be provided by AAV-derived coding sequences that are expressed in host cells to provide AAV gene products in trans for AAV replication and packaging. AAV virus can be made replication competent or replication deficient. In general, a replication-deficient AAV virus lacks one or more AAV packaging genes. Cells may be contacted with viral vectors, viral particles, or virus as described herein in vitro, ex vivo, or in vivo. In some cases, cells that are contacted in vitro can be derived from established cell lines or primary cells derived from a subject, either modified ex vivo for return to the subject, or allowed to grow in culture in vitro. In some aspects, a virus is used to deliver a viral vector into primary cells ex vivo to modify the cells, such as introducing an exogenous nucleic acid sequence, a transgene, or an engineered cell receptor in an immune cell, or a T cell in particular, followed by expansion, selection, or limited number of passages in culture before such modified cells are returned back to the subject. In some aspects, such modified cells are used in cell-based therapy to treat a disease or condition, including cancer. In some cases, a primary cell can be a primary lymphocyte. In some cases, a population of primary cells can be a population of primary lymphocytes. In some cases, a primary cell is a tumor infiltrating lymphocytes (TIL). In some cases, a population of primary cells is a population of TILs.

In some cases, the recombinant AAV is not a self-complementary AAV (scAAV). Any conventional methods suitable for purifying AAV can be used in the embodiments described herein to purify the recombinant AAV. For example, the recombinant can be isolated and purified from packaging cells and/or the supernatant of the packaging cells. In some cases, the AAV can be purified by separation method using a CsCl gradient. Also, US Patent Publication No. 20020136710 describes another non-limiting example of method for purifying AAV, in which AAV was isolated and purified from a sample using a solid support that includes a matrix to which an artificial receptor or receptor-like molecule that mediates AAV attachment is immobilized.

In some cases, a population of cells can be transduced with a viral vector, an AAV, modified AAV, or rAAV for example. A transduction with a virus can occur before a genomic disruption with a CRISPR system, after a genomic disruption with a CRISPR system, or at the same time as a genomic disruption with a CRISPR system. For example, a genomic disruption with a CRISPR system may facilitate integration of an exogenous polynucleic acid into a portion of a genome. In some cases, a CRISPR system may be used to introduce a double strand break in a portion of a genome comprising a gene, such as an immune checkpoint gene or a safe harbor loci. In some cases, a CRISPR system can be used to introduce a break in at least one gene (e.g., CISH and/or TCR). A double strand break can be repaired by introducing an exogenous receptor sequence delivered to a cell by a viral vector, an AAV or modified AAV or rAAV in some cases. In some cases, a double strand break can be repaired by integrating an exogenous transgene (e.g., a TCR) in said break. An AAV or modified AAV or rAAV can comprise a polynucleic acid with recombination arms to a portion of a gene disrupted by a CRISPR system. In some cases, a CRISPR system comprises a guide polynucleic acid. In some cases, a guide polynucleic acid is a guide ribonucleic acid (gRNA) and/or a guide deoxyribonucleic acid (gDNA). For example, a CRISPR system may introduce a double strand break at a CISH and/or TCR gene. A CISH and/or TCR gene can then be repaired by introduction of a transgene (e.g., transgene encoding an exogenous TCR), wherein a transgene can be flanked by recombination arms with regions complementary to a portion of a genome previously disrupted by a CRISPR system. A population of cells comprising a genomic disruption and a viral introduction can be transduced. A transduced population of cells can be from about 5% to about 100%. For example, a population of cells can be transduced from about 5%, 10%, 20%, 30%, 40%, 50%, 60%, 70%, 80%, 90%, or up to about 100%.

In some cases, a virus (e.g., AAV or modified AAV) and/or a viral vector (e.g., AAV vector or modified AAV vector), and/or a non-viral vector (e.g., minicircle vector) is introduced to a cell or to a population of cells at about, from about, at least about, or at most about 1-3 hrs., 3-6 hrs., 6-9 hrs., 9-12 hrs., 12-15 hrs., 15-18 hrs., 18-21 hrs., 21-23 hrs., 23-26 hrs., 26-29 hrs., 29-31 hrs., 31-33 hrs., 33-35 hrs., 35-37 hrs., 37-39 hrs., 39-41 hrs., 2 days, 3 days, 4 days, 5 days, 6 days, 7 days, 8 days, 9 days, 10 days, 14 days, 16 days, 20 days, or longer than 20 days after a CRISPR system or after a nuclease or a polynucleotide encoding a nuclease or after a guide polynucleic acid is introduced to said cell or to said population of cells. In some cases, a viral vector comprises at least one exogenous transgene (e.g., an AAV vector comprises at least one exogenous transgene). In some cases, a non-viral vector comprises at least one exogenous transgene (e.g., a minicircle vector comprises at least one exogenous transgene). In some cases, an AAV vector (e.g., a modified AAV vector) comprises at least one exogenous nucleic acid. In some cases, an AAV vector (e.g., a modified AAV vector) is introduced to at least one cell in a population of cells to integrate at least one exogenous nucleic acid into a genomic locus of at least one cell.

In some cases, the nucleic acid may comprise a barcode or a barcode sequence. A barcode or barcode sequence relates to a natural or synthetic nucleic acid sequence comprised by a polynucleotide allowing for unambiguous identification of the polynucleotide and other sequences comprised by the polynucleotide having said barcode sequence. For example, a nucleic acid comprising a barcode can allow for identification of the encoded transgene. A barcode sequence can comprise a sequence of at least 3, 4, 5, 6, 7, 8, 9, 10, 11, 12, 13, 14, 15, 16, 17, 18, 19, 20, 25, 30, 40, 45, or 50 or more consecutive nucleotides. A nucleic acid can comprise two or more barcode sequences or compliments thereof. A barcode sequence can comprise a randomly assembled sequence of nucleotides. A barcode sequence can be a degenerate sequence. A barcode sequence can be a known sequence. A barcode sequence can be a predefined sequence.

In some cases, the methods disclosed herein may comprise a nucleic acid (e.g., a first nucleic acid and/or a second nucleic acid). In some cases, the nucleic acid may encode a transgene. Generally, a transgene may refer to a linear polymer comprising multiple nucleotide subunits. A transgene may comprise any number of nucleotides. In some cases, a transgene may comprise less than about 100 nucleotides. In some cases, a transgene may comprise at least about 100 nucleotides. In some cases, a transgene may comprise at least about 200 nucleotides. In some cases, a transgene may comprise at least about 300 nucleotides. In some cases, a transgene may comprise at least about 400 nucleotides. In some cases, a transgene may comprise at least about 500 nucleotides. In some cases, a transgene may comprise at least about 1000 nucleotides. In some cases, a transgene may comprise at least about 5000 nucleotides. In some cases, a transgene may comprise at least about 10,000 nucleotides. In some cases, a transgene may comprise at least about 20,000 nucleotides. In some cases, a transgene may comprise at least about 30,000 nucleotides. In some cases, a transgene may comprise at least about 40,000 nucleotides. In some cases, a transgene may comprise at least about 50,000 nucleotides. In some cases, a transgene may comprise between about 500 and about 5000 nucleotides. In some cases, a transgene may comprise between about 5000 and about 10,000 nucleotides. In any of the cases disclosed herein, the transgene may comprise DNA, RNA, or a hybrid of DNA and RNA. In some cases, the transgene may be single stranded. In some cases, the transgene may be double stranded.

a. Random Insertion

One or more transgenes of the methods described herein can be inserted randomly into the genome of a cell. These transgenes can be functional if inserted anywhere in a genome. For instance, a transgene can encode its own promoter or can be inserted into a position where it is under the control of an endogenous promoter. Alternatively, a transgene can be inserted into a gene, such as an intron of a gene, an exon of a gene, a promoter, or a non-coding region.

A nucleic acid, e.g., RNA, encoding a transgene sequences can be randomly inserted into a chromosome of a cell. A random integration can result from any method of introducing a nucleic acid, e.g., RNA, into a cell. For example, the method can be, but is not limited to, electroporation, sonoporation, use of a gene gun, lipotransfection, calcium phosphate transfection, use of dendrimers, microinjection, and use of viral vectors including adenoviral, AAV, and retroviral vectors, and/or group II ribozymes.

A RNA encoding a transgene can also be designed to include a reporter gene so that the presence of a transgene or its expression product can be detected via activation of the reporter gene. Any reporter gene can be used, such as those disclosed above. By selecting in cell culture those cells in which a reporter gene has been activated, cells can be selected that contain a transgene.

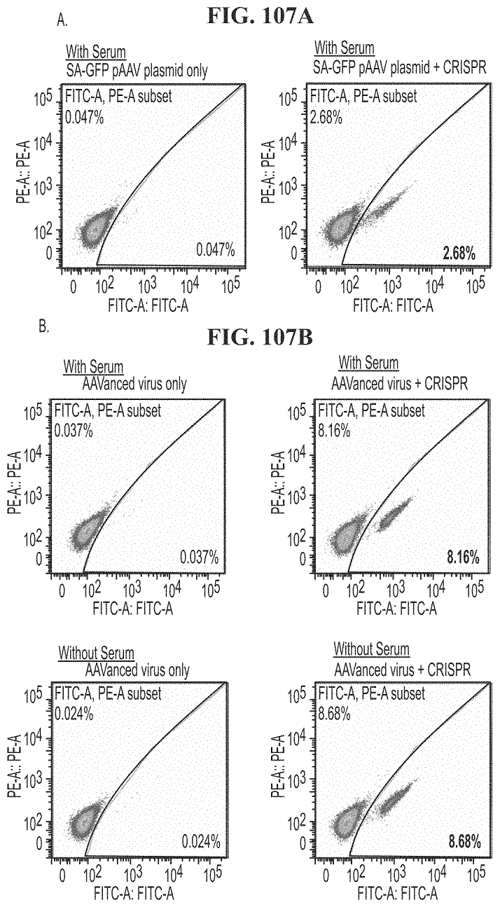

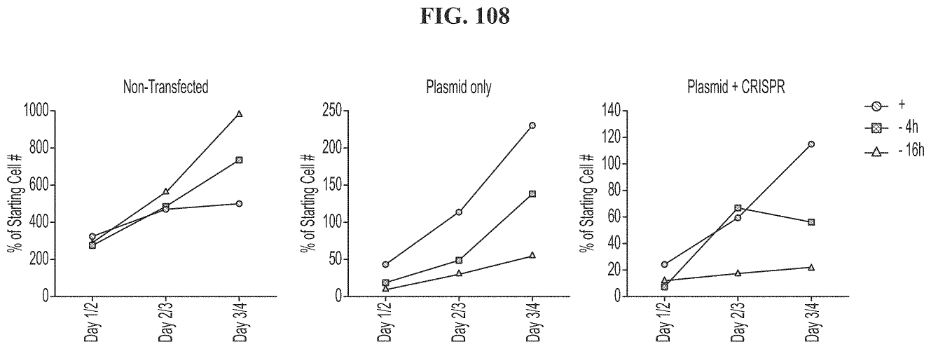

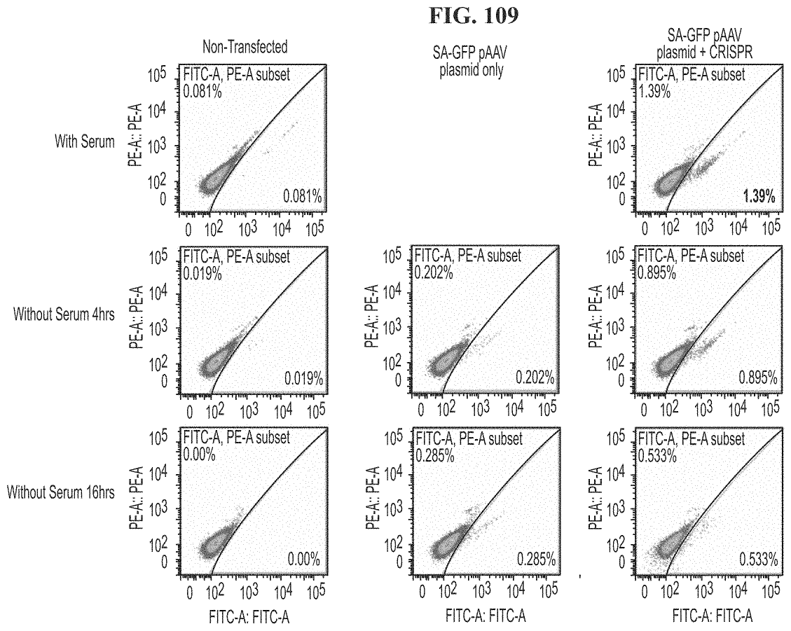

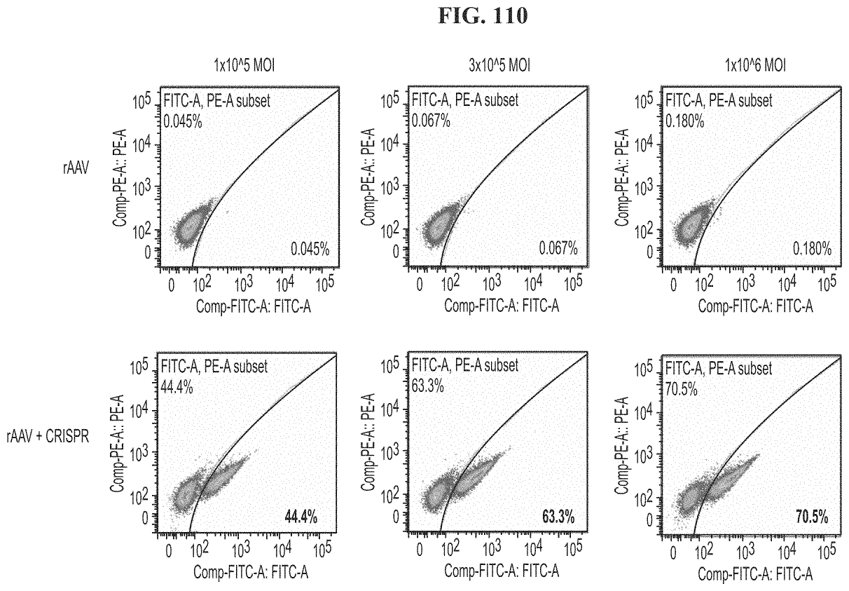

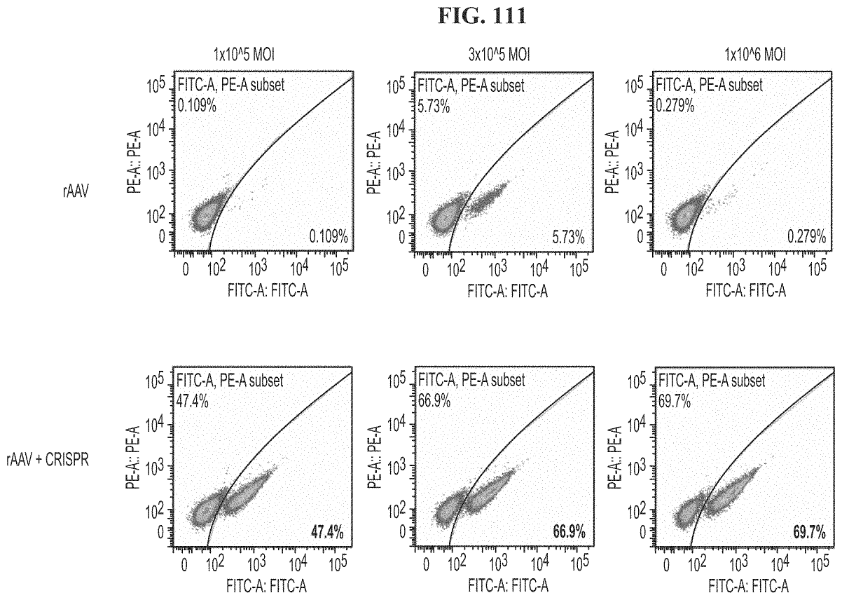

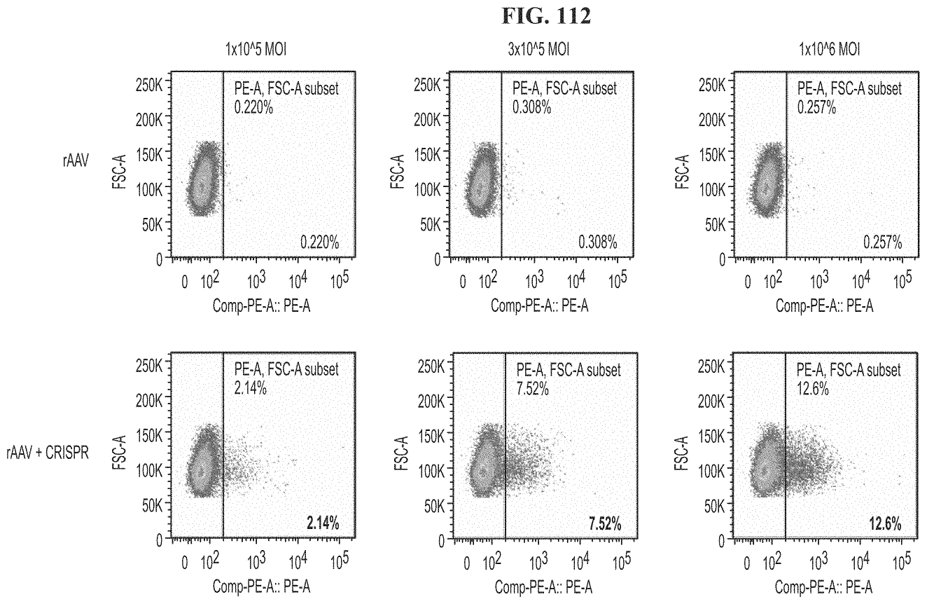

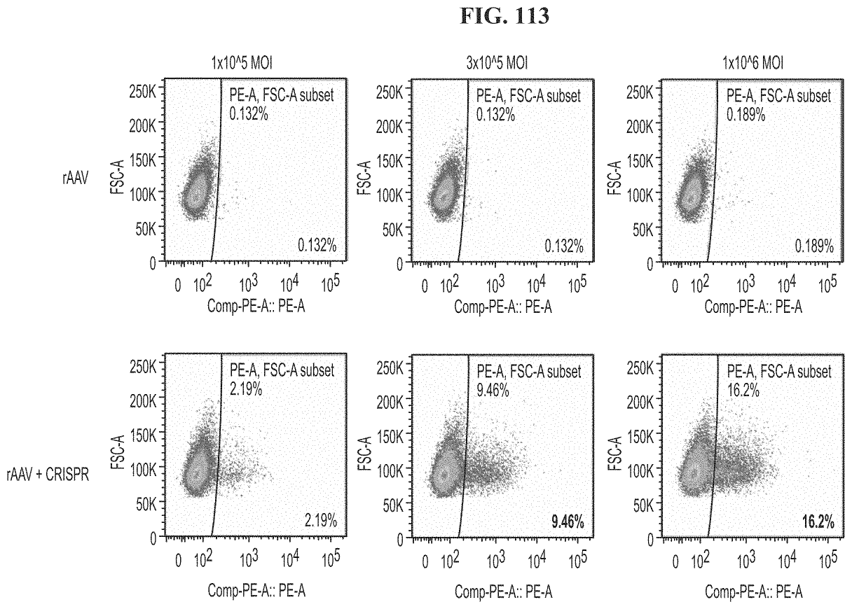

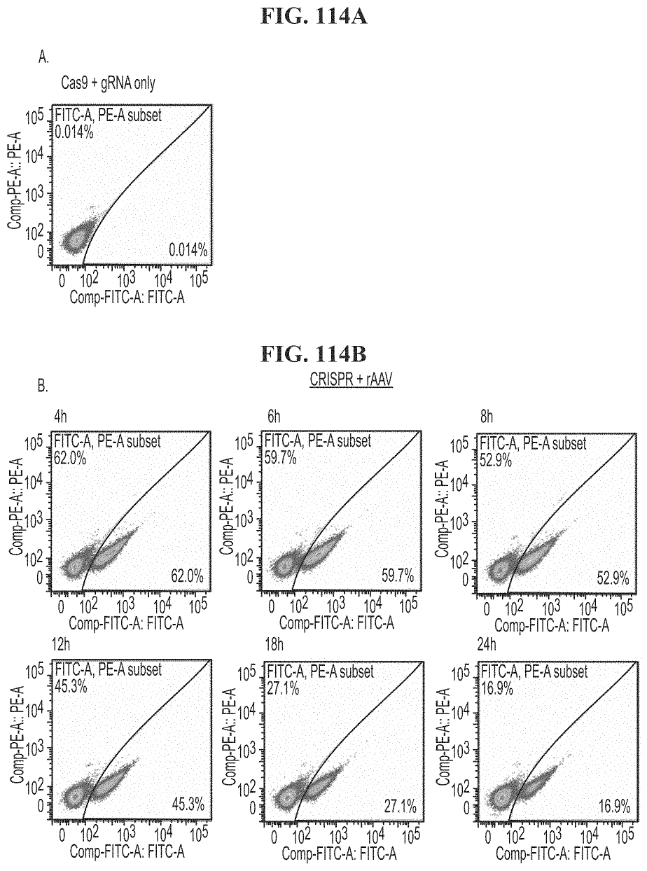

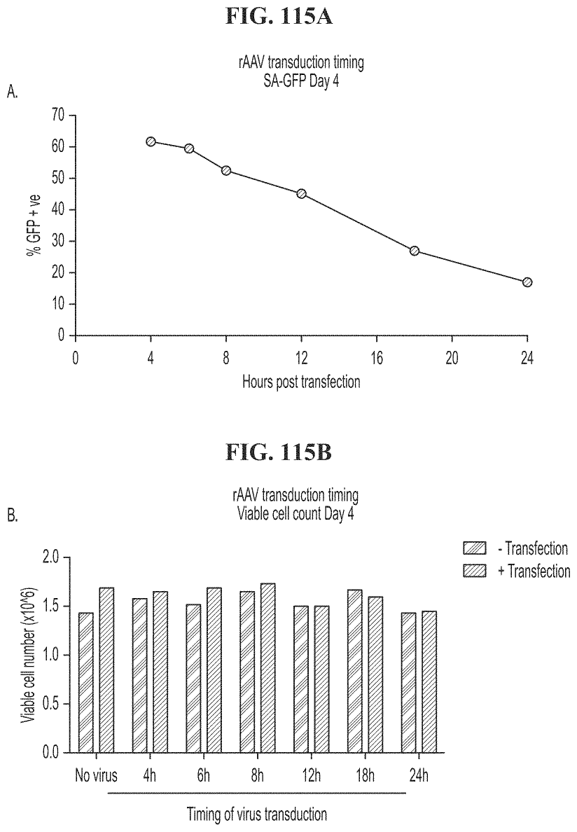

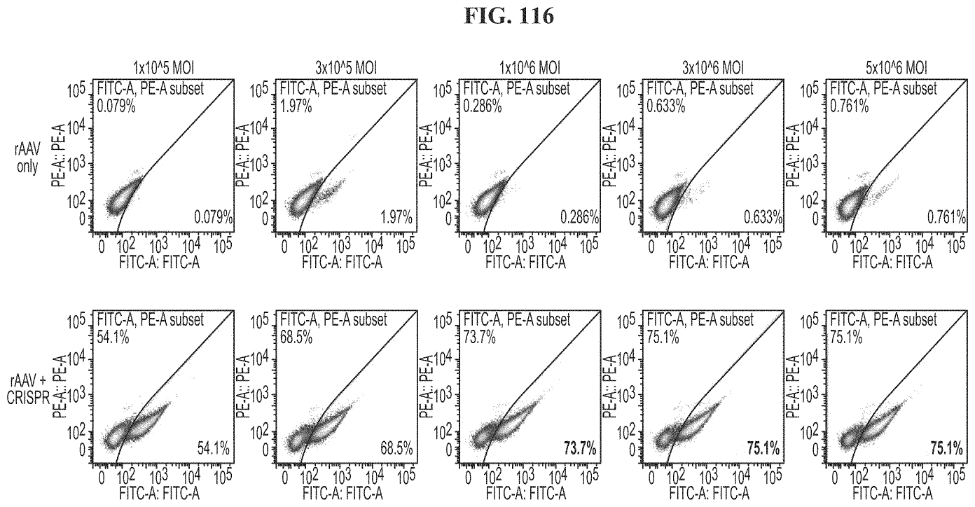

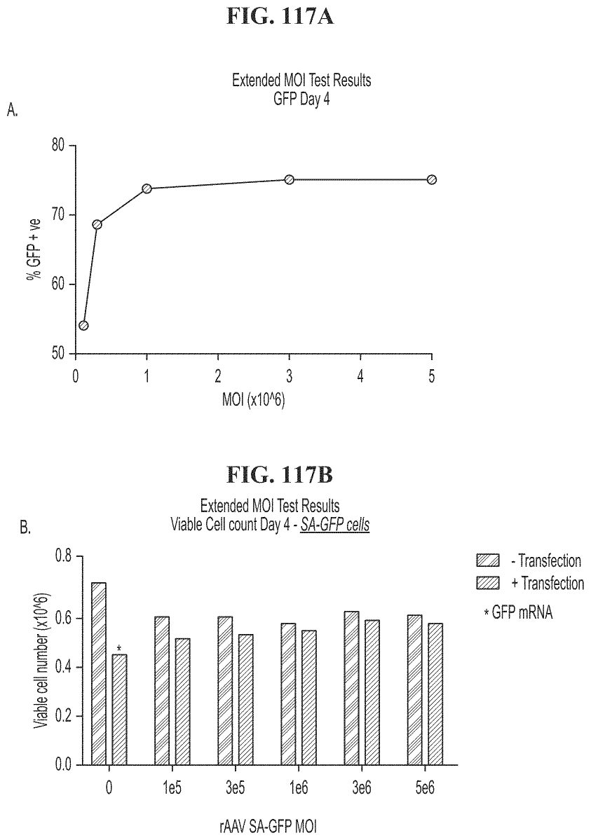

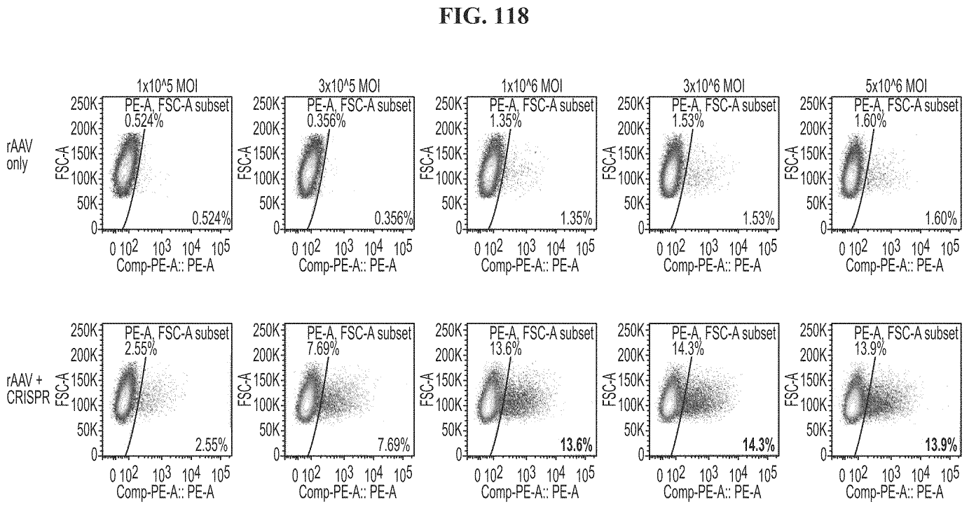

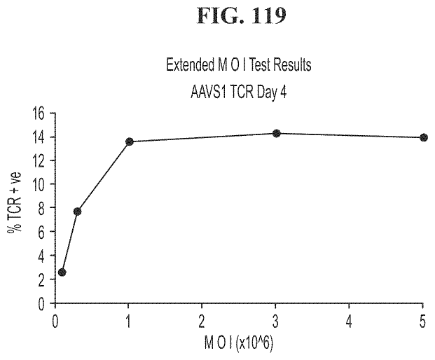

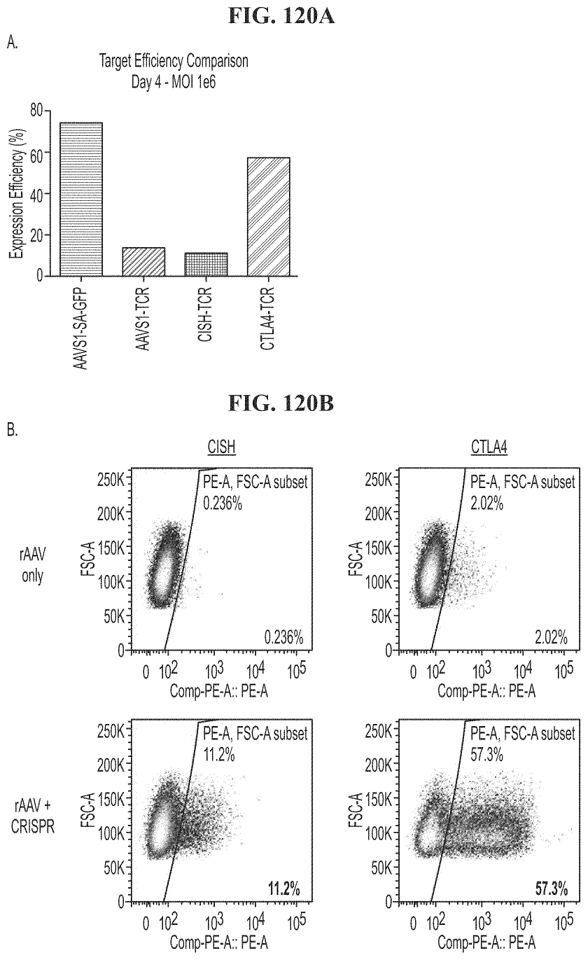

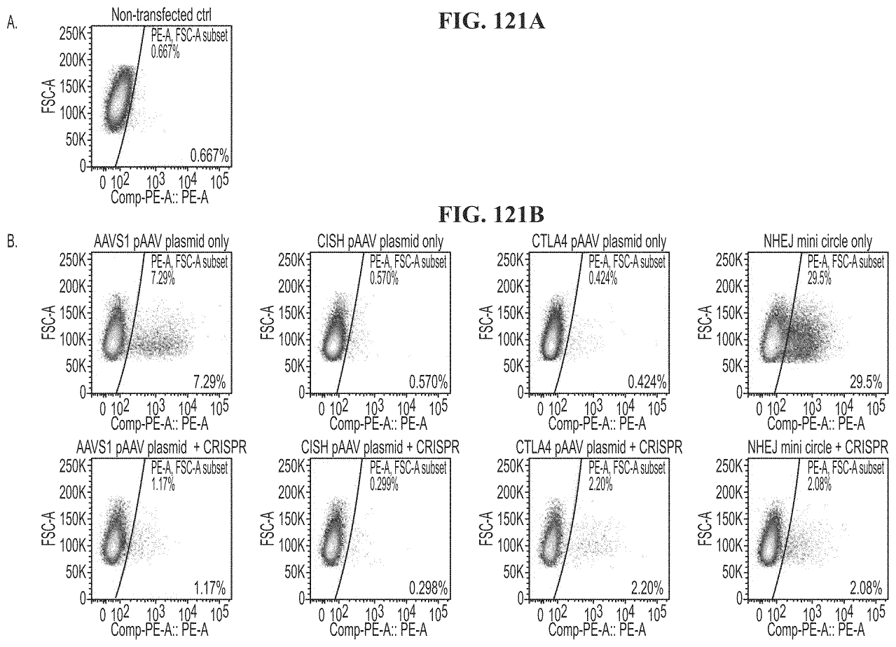

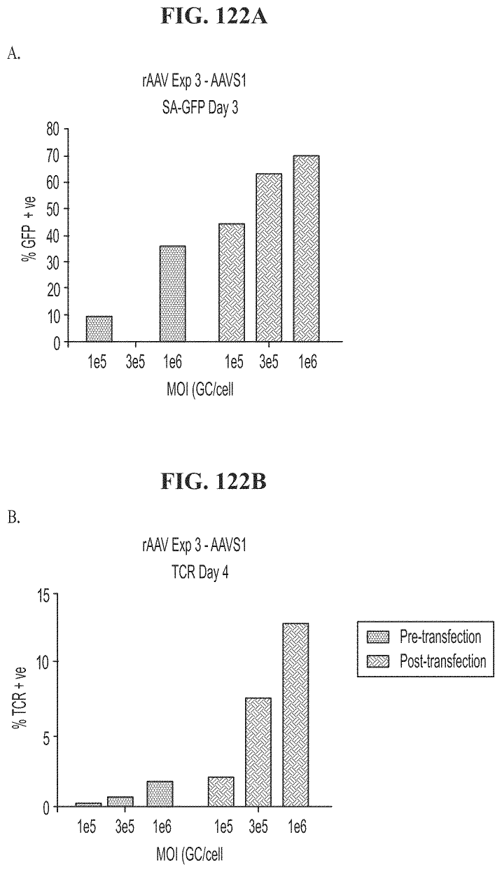

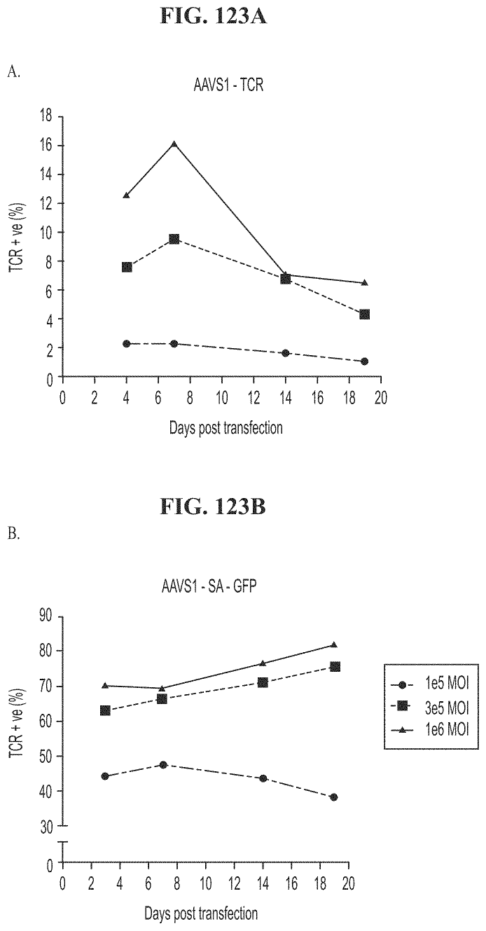

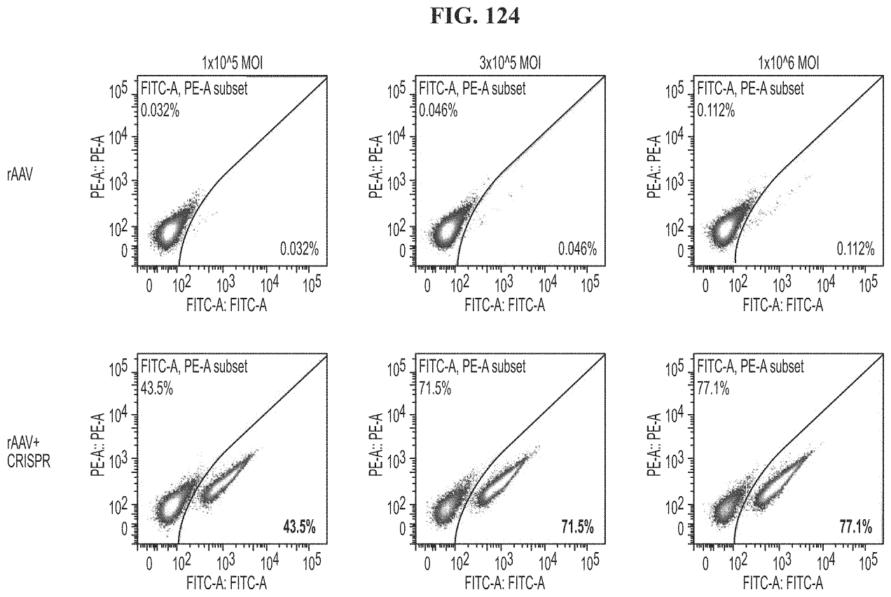

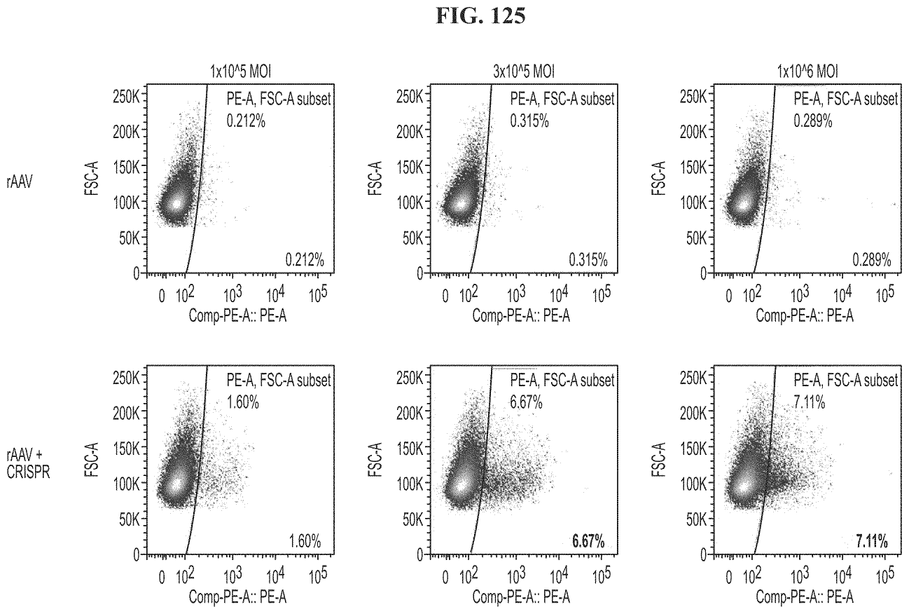

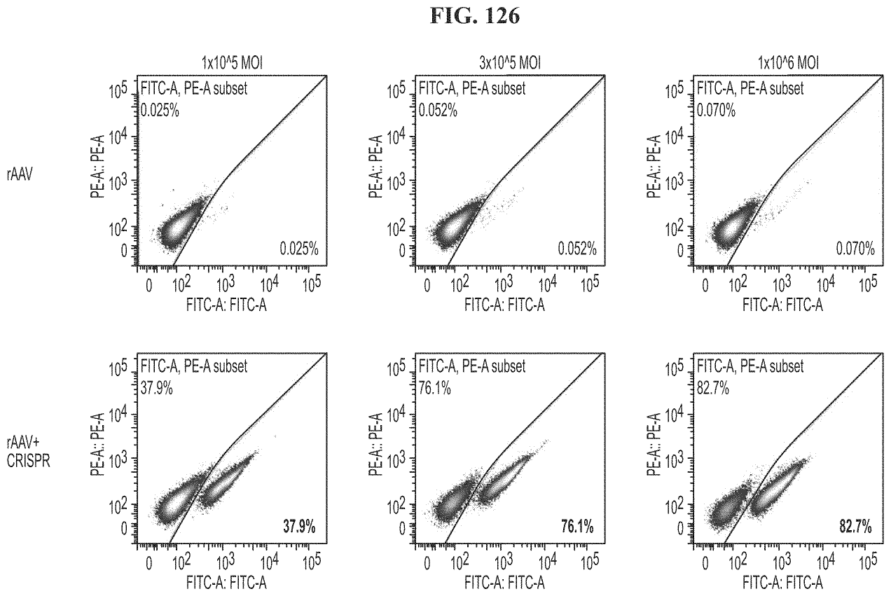

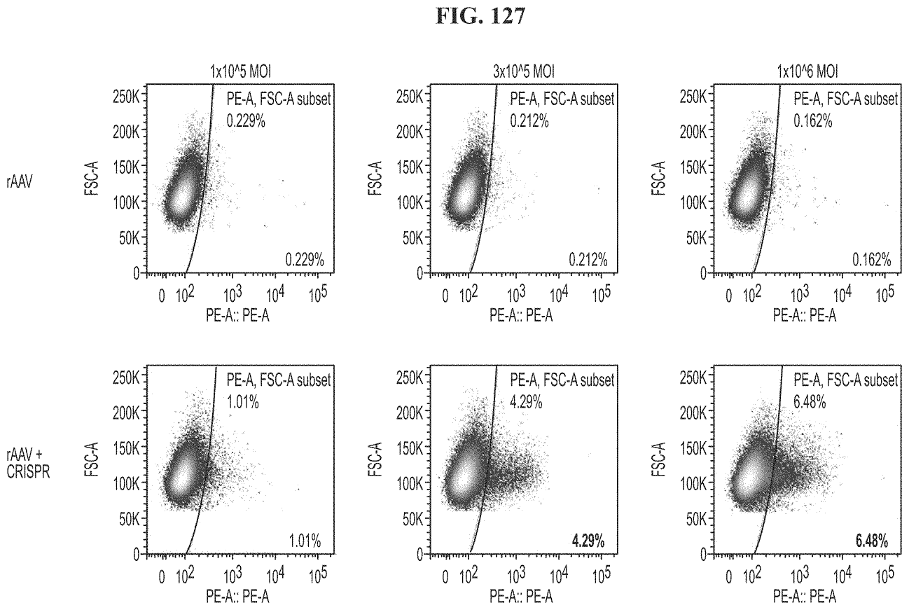

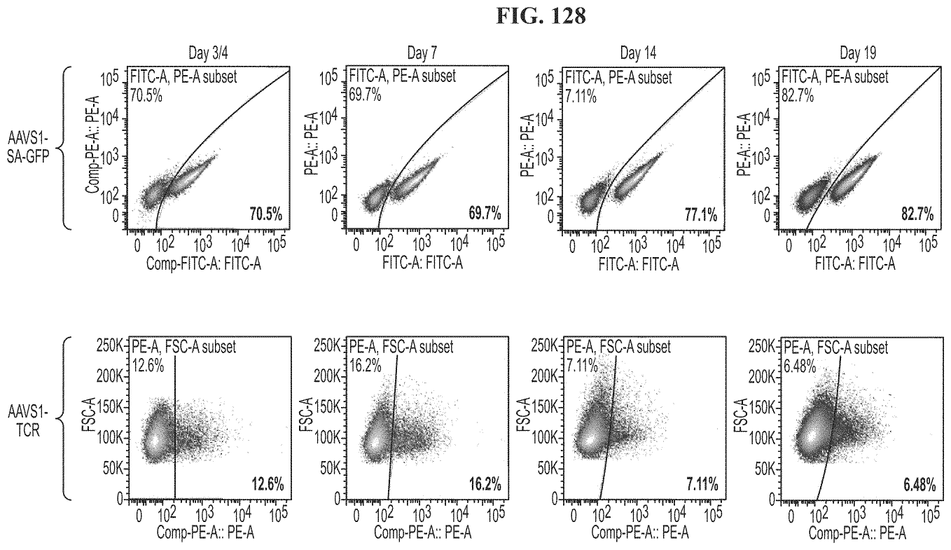

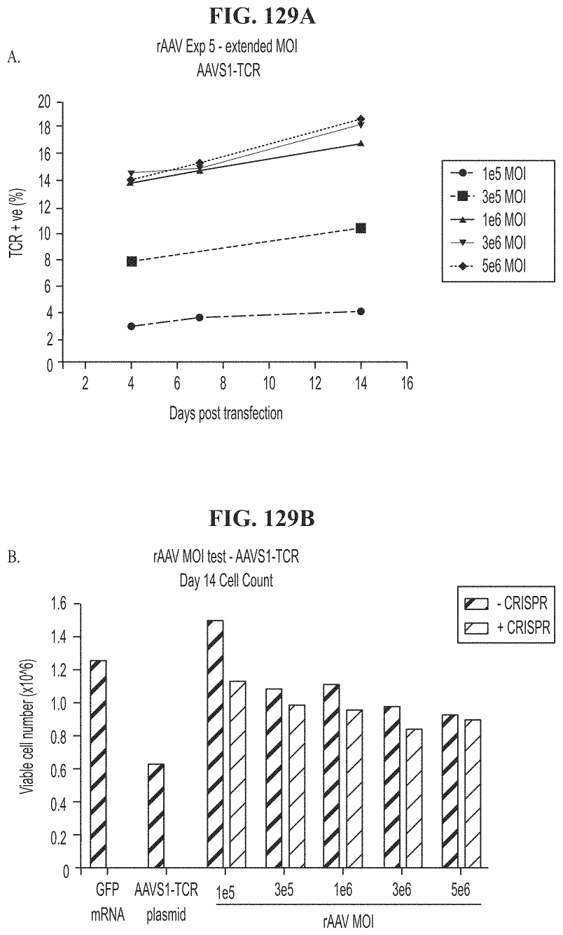

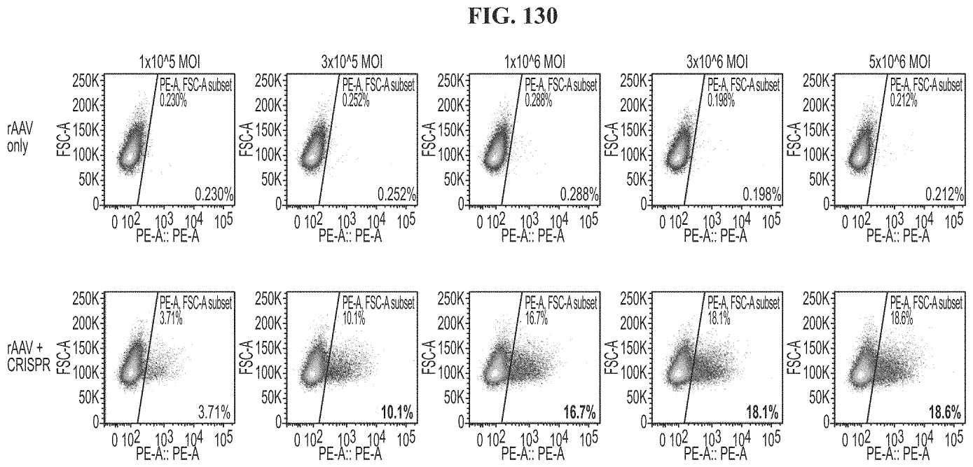

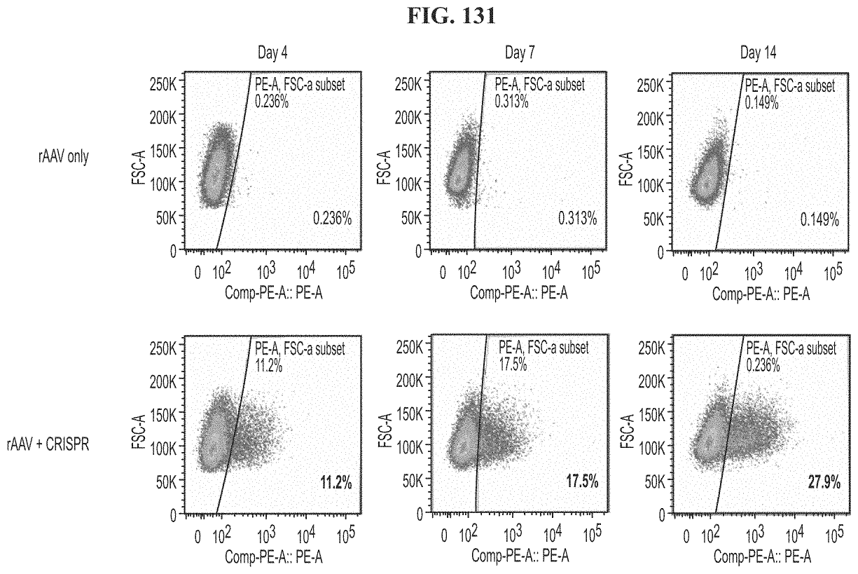

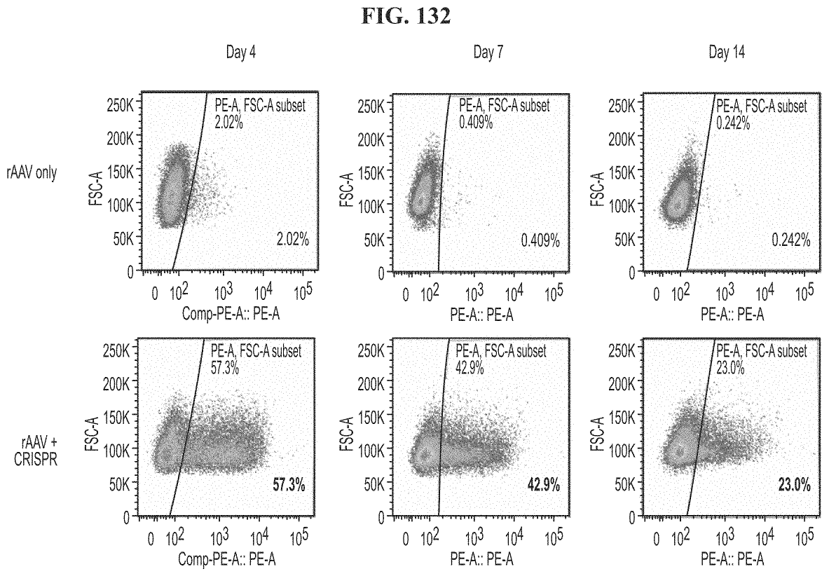

A transgene to be inserted can be flanked by engineered sites analogous to a targeted double strand break site in the genome to excise the transgene from a polynucleic acid so it can be inserted at the double strand break region. A transgene can be virally introduced in some cases. For example, an AAV virus can be utilized to infect a cell with a transgene. Flow cytometry can be utilized to measure expression of an integrated transgene by an AAV virus, FIG. 107A, FIG. 107B, and FIG. 128. Integration of a transgene by an AAV virus may not induce cellular toxicity, FIG. 108. In some cases, cellular viability as measured by flow cytometry of a cellular population engineered utilizing an AAV virus can be from about 30% to 100% viable. Cellular viability as measured by flow cytometry of an engineered cellular population can be from about 30%, 40%, 50%, 60%, 70%, 80%, 90%, to about 100%. In some cases, a rAAV virus can introduce a transgene into the genome of a cell, FIG. 109, FIG. 130, FIG. 131, and FIG. 132. An integrated transgene can be expressed by an engineered cell from immediately after genomic introduction to the duration of the life of an engineered cell. For example, an integrated transgene can be measured from about 0.1 min after introduction into a genome of a cell up, 1 hour to 5 hours, 5 hours to 10 hours, 10 hours to 20 hours, 20 hours to 1 day, 1 day to 3 days, 3 days to 5 days, 5 days to 15 days, 15 days to 30 days, 30 days to 50 days, 50 days to 100 days, or up to 1000 days after the initial introduction of a transgene into a cell. Expression of a transgene can be detected from 3 days, FIG. 110, and FIG. 112. Expression of a transgene can be detected from 7 days, FIG. 111, FIG. 113. Expression of a transgene can be detected from about 4 hours, 6 hours, 8 hours, 12 hours, 18 hours, to about 24 hours after introduction of a transgene into a genome of a cell, FIG. 114A, FIG. 114B, FIG. 115A, and FIG. 115B. In some cases, viral titer can influence the percent of transgene expression, FIG. 116, FIG. 117A, FIG. 117B, FIG. 118, FIG. 119A, FIG. 120A, FIG. 120B, FIG. 121A, FIG. 121B, FIG. 122A, FIG. 122B, FIG. 123A, FIG. 123B, FIG. 124, FIG. 125, FIG. 126, FIG. 127, FIG. 129A, FIG. 129B, FIG. 130A, FIG. 130B,

In some cases, a viral vector, such as an AAV viral vector, containing a gene of interest or a transgene as described herein may be inserted randomly into a genome of a cell following transfection of the cell by a viral particle containing the viral vector. Such random sites for insertion include genomic sites with a double strand break. Some viruses, such as retrovirus, comprise factors, such as integrase, that can result in random insertions of the viral vector.

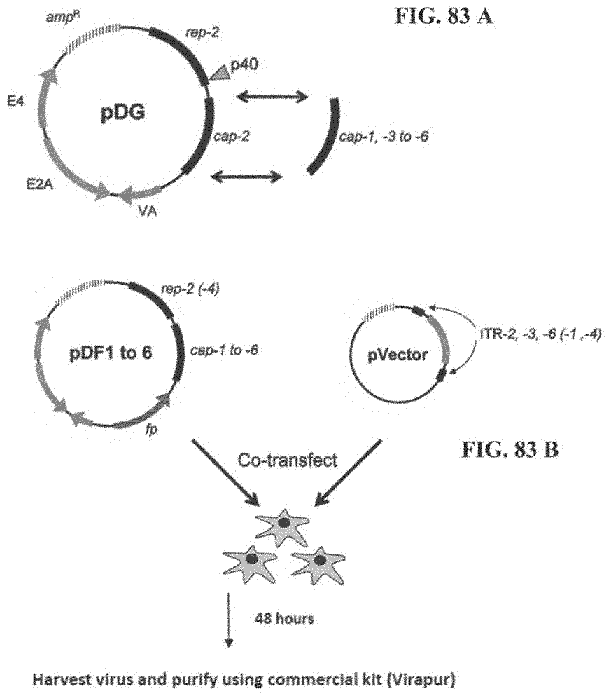

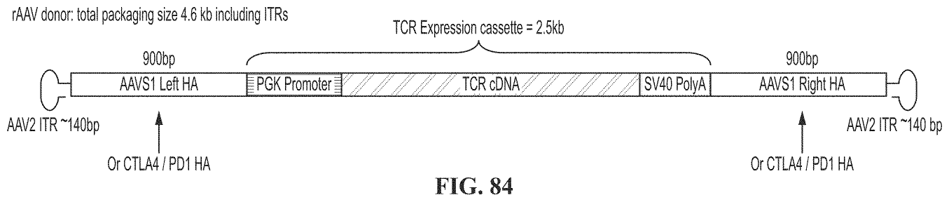

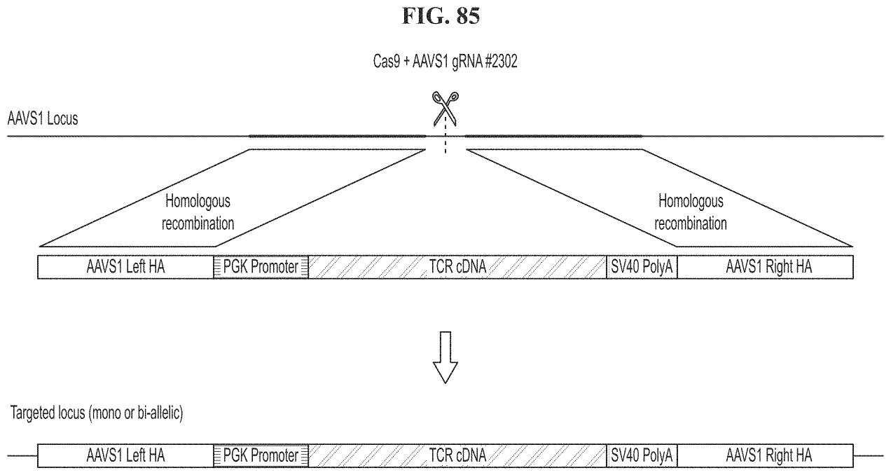

In some cases, a modified or engineered AAV virus can be used to introduce a transgene to a cell, FIG. 83 A. and FIG. 83 B. A modified or wildtype AAV can comprise homology arms to at least one genomic location, FIG. 84 to FIG. 86 D.

A RNA encoding a transgene can be introduced into a cell via electroporation. RNA can also be introduced into a cell via lipofection, infection, or transformation. Electroporation and/or lipofection can be used to transfect primary cells. Electroporation and/or lipofection can be used to transfect primary hematopoietic cells. In some cases, RNA can be reverse transcribed within a cell into DNA. A DNA substrate can then be used in a homologous recombination reaction. A DNA can also be introduced into a cell genome without the use of homologous recombination. In some cases, a DNA can be flanked by engineered sites that are complementary to the targeted double strand break region in a genome. In some cases, a DNA can be excised from a polynucleic acid so it can be inserted at a double strand break region without homologous recombination.

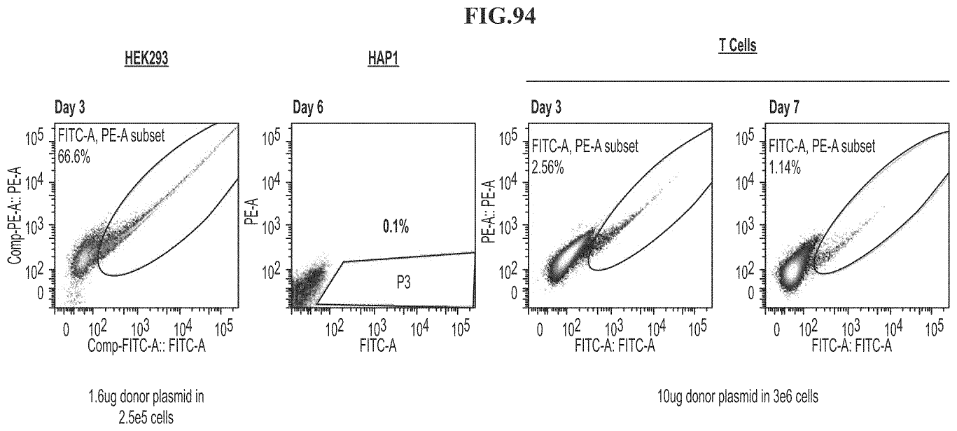

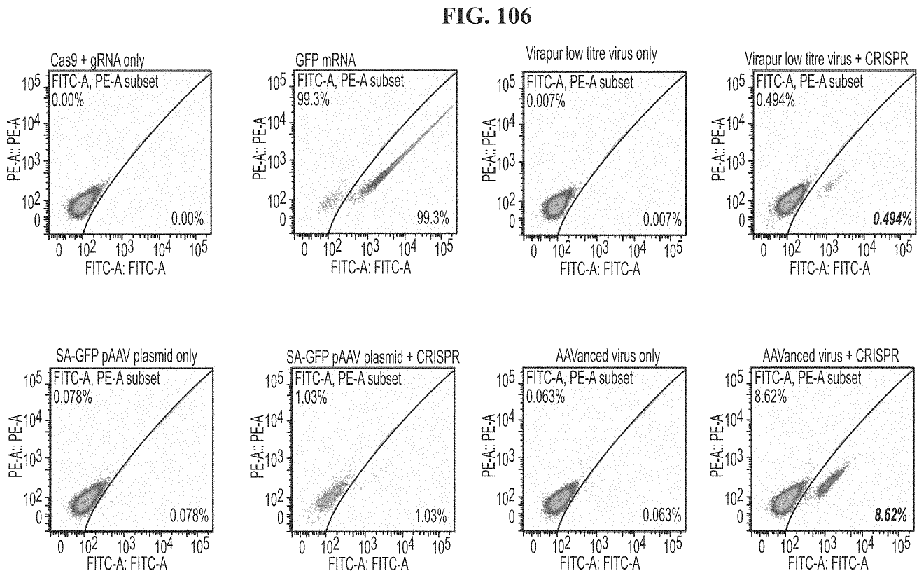

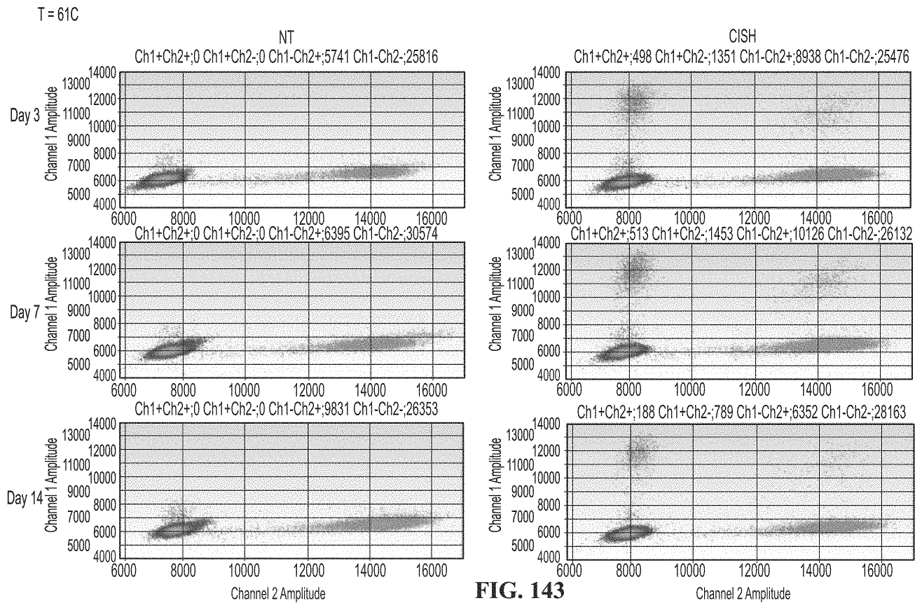

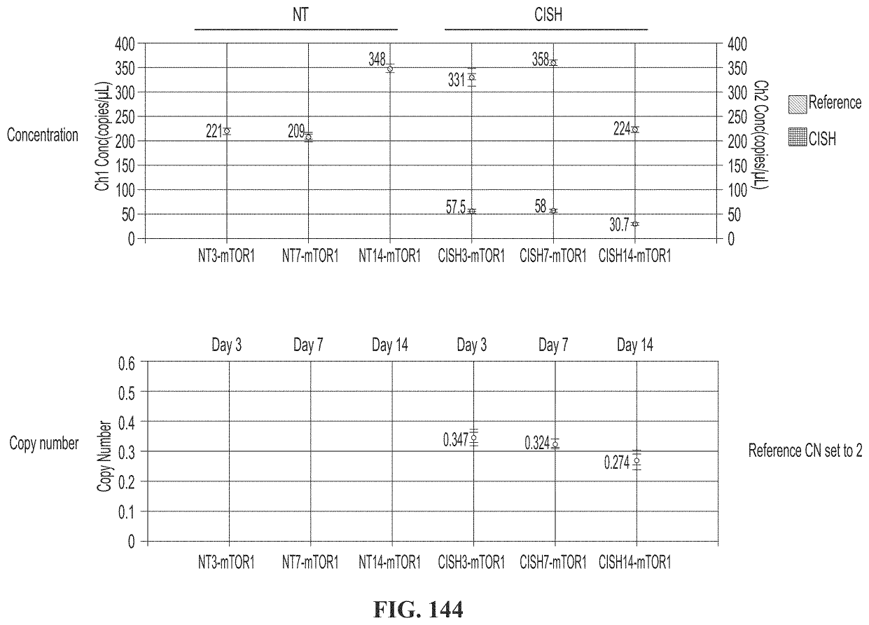

Expression of a transgene can be verified by an expression assay, for example, qPCR or by measuring levels of RNA. Expression level can be indicative also of copy number, FIG. 143 and FIG. 144. For example, if expression levels are extremely high, this can indicate that more than one copy of a transgene was integrated in a genome. Alternatively, high expression can indicate that a transgene was integrated in a highly transcribed area, for example, near a highly expressed promoter. Expression can also be verified by measuring protein levels, such as through Western blotting. In some cases, a splice acceptor assay can be used with a reporter system to measure transgene integration, FIG. 94. In some cases, a splice acceptor assay can be used with a reporter system to measure transgene integration when a transgene is introduced to a genome using an AAV system, FIG. 106.

b. Site Specific Insertion

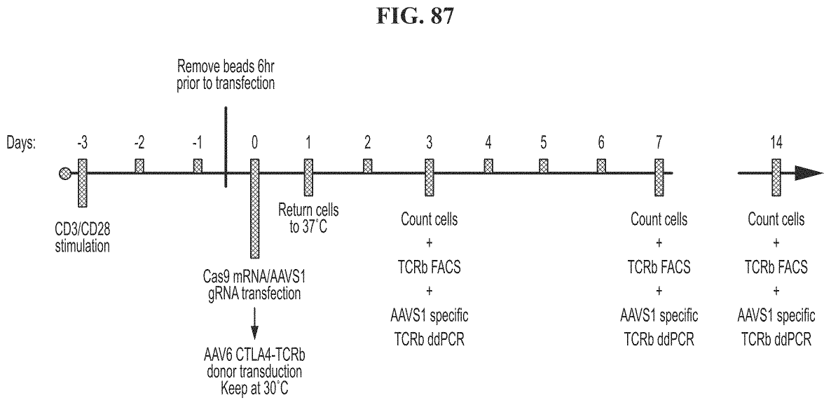

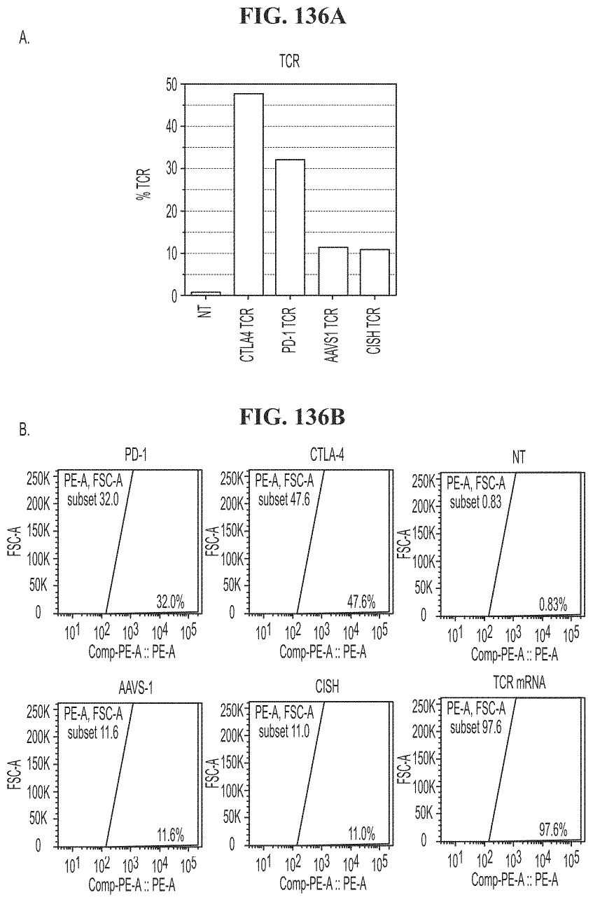

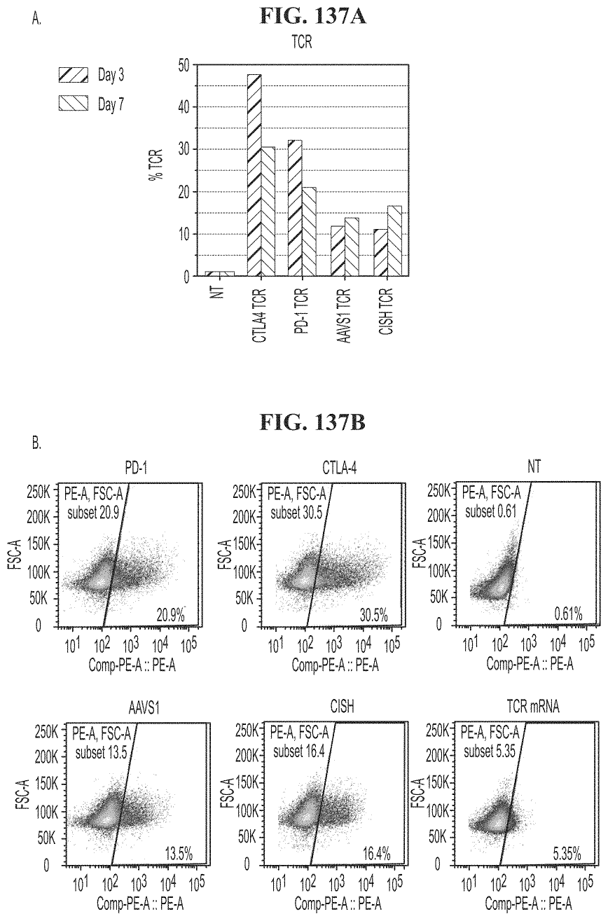

Inserting one or more transgenes in any of the methods disclosed herein can be site-specific. For example, one or more transgenes can be inserted adjacent to or near a promoter. In another example, one or more transgenes can be inserted adjacent to, near, or within an exon of a gene (e.g., CISH gene and/or TCR gene). Such insertions can be used to knock-in a transgene (e.g., cancer-specific TCR transgene) while simultaneously disrupting another gene (e.g., CISH gene and/or TCR). In another example, one or more transgenes can be inserted adjacent to, near, or within an intron of a gene. A transgene can be introduced by an AAV viral vector and integrate into a targeted genomic location, FIG. 87. In some cases, a rAAV vector can be utilized to direct insertion of a transgene into a certain location. For example in some cases, a transgene can be integrated into at least a portion of a TCR, CTLA4, PD-1, AAVS1, TCR, or CISH gene by a rAAV or an AAV vector, FIG. 136A, FIG. 136B, FIG. 137A, and FIG. 137B.

Modification of a targeted locus of a cell can be produced by introducing DNA into cells, where the DNA has homology to the target locus. DNA can include a marker gene, allowing for selection of cells comprising the integrated construct. Complementary DNA in a target vector can recombine with a chromosomal DNA at a target locus. A marker gene can be flanked by complementary DNA sequences, a 3′ recombination arm, and a 5′ recombination arm. Multiple loci within a cell can be targeted. For example, transgenes with recombination arms specific to 1 or more target loci can be introduced at once such that multiple genomic modifications occur in a single step.

In some cases, recombination arms or homology arms to a particular genomic site can be from about 0.2 kb to about 5 kb in length. Recombination arms can be from about 0.2 kb, 0.4 kb 0.6 kb, 0.8 kb, 1.0 kb, 1.2 kb, 1.4 kb, 1.6 kb, 1.8 kb, 2.0kb, 2.2 kb, 2.4 kb, 2.6 kb, 2.8 kb, 3.0 kb, 3.2 kb, 3.4 kb, 3.6 kb, 3.8 kb, 4.0 kb, 4.2 kb, 4.4 kb, 4.6kb, 4.8 kb, to about 5.0kb in length.

A variety of enzymes can catalyze insertion of foreign DNA into a host genome. For example, site-specific recombinases can be clustered into two protein families with distinct biochemical properties, namely tyrosine recombinases (in which DNA is covalently attached to a tyrosine residue) and serine recombinases (where covalent attachment occurs at a serine residue). In some cases, recombinases can comprise Cre, fC31 integrase (a serine recombinase derived from Streptomyces phage fC31), or bacteriophage derived site-specific recombinases (including Flp, lambda integrase, bacteriophage HK022 recombinase, bacteriophage R4 integrase and phage TP901-1 integrase).

Expression control sequences can also be used in constructs. For example, an expression control sequence can comprise a constitutive promoter, which is expressed in a wide variety of cell types. Tissue-specific promoters can also be used and can be used to direct expression to specific cell lineages.

Site specific gene editing can be achieved using non-viral gene editing such as CRISPR, TALEN (see U.S. patent Ser. No. 14/193,037), transposon-based, ZEN, meganuclease, or Mega-TAL, or Transposon-based system. For example, PiggyBac (see Moriarty, B. S., et al., “Modular assembly of transposon integratable multigene vectors using RecWay assembly,” Nucleic Acids Research (8):e92 (2013) or sleeping beauty (see Aronovich, E. L, et al., “The Sleeping Beauty transposon system: a non-viral vector for gene therapy,” Hum. Mol. Genet., 20(R1): R14-R20. (2011) transposon systems can be used.

Site specific gene editing can also be achieved without homologous recombination. An exogenous polynucleic acid can be introduced into a cell genome without the use of homologous recombination. In some cases, a transgene can be flanked by engineered sites that are complementary to a targeted double strand break region in a genome. A transgene can be excised from a polynucleic acid so it can be inserted at a double strand break region without homologous recombination.

In some cases, where genomic integration of a transgene is desired, an exogenous or an engineered nuclease can be introduced to a cell in addition to a plasmid, a linear or circular polynucleotide, a viral or a non-viral vector comprising a transgene to facilitate integration of the transgene at a site where the nuclease cleaves the genomic DNA. Integration of the transgene into the cell's genome allows stable expression of the transgene over time. In some aspects, a viral vector can be used to introduce a promoter that is operably linked to the transgene. In other cases, a viral vector may not comprise a promoter, which requires insertion of the transgene at a target locus that comprises an endogenous promoter for expressing the inserted transgene.

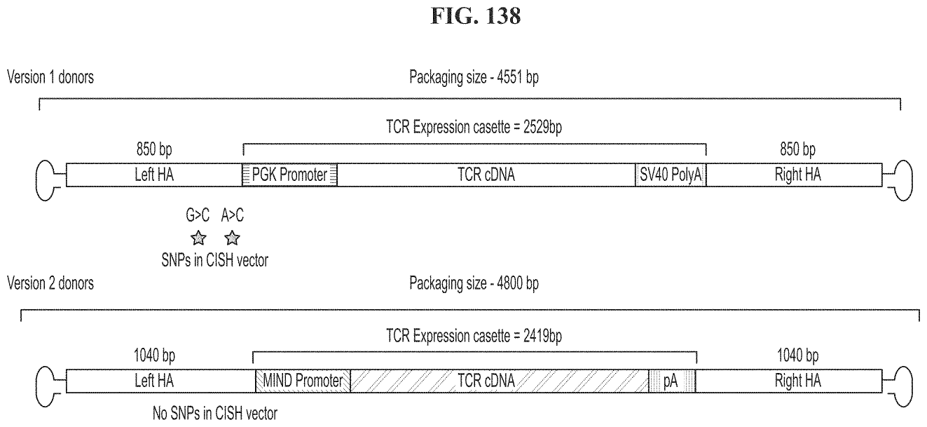

In some cases, a viral vector, FIG. 138, comprises homology arms that direct integration of a transgene into a target genomic locus, such as CISH and/or TCR and/or a safe harbor site. In some cases, a first nuclease is engineered to cleave at a specific genomic site to suppress (e.g., partial or complete suppression of a gene (e.g., CISH and/or TCR)) or disable a deleterious gene, such as an oncogene, a checkpoint inhibitor gene, or a gene that is implicated in a disease or condition, such as cancer. After a double strand break is generated at such genomic locus by the nuclease, a non-viral or a viral vector (e.g., an AAV viral vector) may be introduced to allow integration of a transgene or any exogenous nucleic acid sequence with a therapeutic effect at the site of DNA cleavage or site of the double strand break generated by the nuclease. Alternatively, the transgene may be inserted at a different genomic site using methods known in the art, such as site directed insertion via homologous recombination, using homology arms comprising sequences complementary to the desired site of insertion, such as the CISH and/or TCR or a safe harbor locus. In some cases, a second nuclease may be provided to facilitate site specific insertion of a transgene at a different locus than the site of DNA cleavage by the first nuclease. In some cases, an AAV virus or an AAV viral vector can be used as a delivery system for introducing the transgene, such as a T cell receptor. Homology arms on a rAAV donor can be from 500 base pairs to 2000 base pairs. For example, homology arms on a rAAV donor can be from 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, or up to 2000 bp long. Homology arm length can be 850 bp. In other cases, homology arm length can be 1040 bp. In some cases, homology arms are extended to allow for accurate integration of a donor. In other cases, homology arms are extended to improve integration of a donor. In some cases, in order to increase the length of homology arms without compromising the size of the donor polynucleic acid, an alternate part of the donor polynucleic acid can be eliminated. In some cases, a poly A tail may be reduced to allow for increased homology arm length.

c. Transgenes or a Nucleic Acid Sequence of Interest

Transgenes can be useful for expressing, e.g., overexpressing, endogenous genes at higher levels than without a transgenes. Additionally, transgenes can be used to express exogenous genes at a level greater than background, i.e., a cell that has not been transfected with a transgenes. Transgenes can also encompass other types of genes, for example, a dominant negative gene.

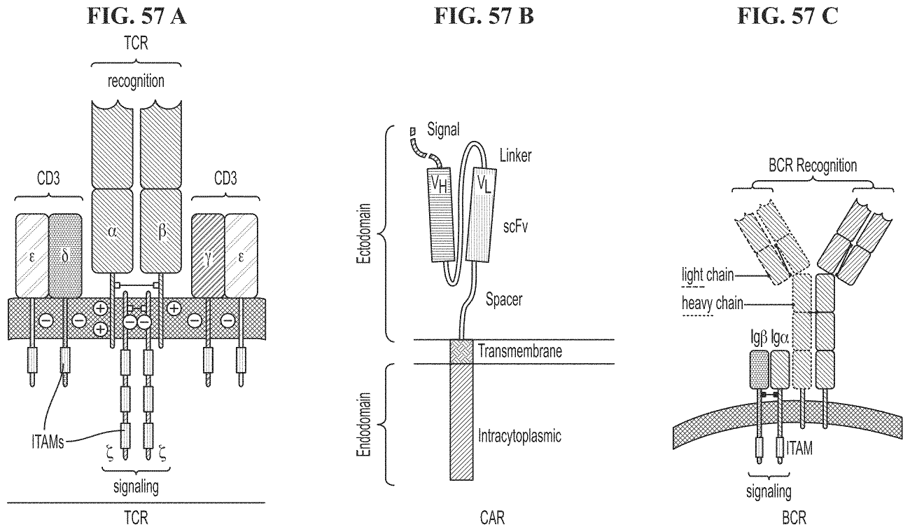

Transgenes can be placed into an organism, cell, tissue, or organ, in a manner which produces a product of a transgene. A polynucleic acid can comprise a transgene. A polynucleic acid can encode an exogenous receptor, FIG. 57 A, FIG. 57 B, and FIG. 57 C. For example, disclosed herein is a polynucleic acid comprising at least one exogenous T cell receptor (TCR) sequence flanked by at least two recombination arms having a sequence complementary to polynucleotides within a genomic sequence that is adenosine A2a receptor, CD276, V-set domain containing T cell activation inhibitor 1, B and T lymphocyte associated, cytotoxic T-lymphocyte-associated protein 4, indoleamine 2,3-dioxygenase 1, killer cell immunoglobulin-like receptor, three domains, long cytoplasmic tail, 1, lymphocyte-activation gene 3, programmed cell death 1, hepatitis A virus cellular receptor 2, V-domain immunoglobulin suppressor of T-cell activation, or natural killer cell receptor 2B4. One or more transgenes can be in combination with one or more disruptions.

In some cases, a transgene (e.g., at least one exogenous transgene) or a nucleic acid (e.g., at least one exogenous nucleic acid) can be integrated into a genomic locus and/or at a break in a gene (e.g., CISH and/or TCR) using non-viral integration or viral integration methods. In some cases, viral integration comprises AAV (e.g., AAV vector or modified AAV vector or recombinant AAV vector). In some cases, an AAV vector comprises at least one exogenous transgene. In some cases, cell viability is measured after an AAV vector comprising at least one exogenous transgene (e.g., at least one exogenous transgene) is introduced to a cell or to a population of cells. In some cases, cell viability is measured after a transgene is integrated into a genomic locus of at least one cell in a population of cells (e.g., by viral or non-viral methods). In some cases, cell viability is measured by fluorescence-activated cell sorting (FACS). In some cases cell viability is measured after a viral or a non-viral vector comprising at least one exogenous transgene is introduced to a cell or to a population of cells. In some cases, at least about, or at most about, or about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.8%, or 100% of the cells in a population of cells are viable after a viral vector (e.g., AAV vector comprising at least one exogenous transgene) or a non-viral vector (e.g., minicircle vector comprising at least one exogenous transgene) is introduced to a cell or to a population of cells. In some cases, cell viability is measured at about, at least about, or at most about 4 hours, 6 hours, 8 hours, 10 hours, 12 hours, 18 hours, 20 hours, 24 hours, 30 hours, 36 hours, 40 hours, 48 hours, 54 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, or longer than 240 hours after a viral (e.g., AAV) or a non-viral (e.g., minicircle) vector is introduced to a cell and/or to a population of cells. In some cases, cell viability is measured at about, at least about, or at most about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 45 days, 50 days, 60 days, 70 days, 90 days, or longer than 90 days after a viral (e.g., AAV) or a non-viral (e.g., minicircle) vector is introduced to a cell and/or to a population of cells. In some cases, cell viability is measured after at least one exogenous transgene is introduced to at least once cell in a population of cells. In some cases, a viral vector or a non-viral vector comprises at least one exogenous transgene. In some cases, cell viability and/or cell toxicity is improved when at least one exogenous transgene is integrated to a cell and/or to a population of cells using viral methods (e.g., AAV vector) compared to when non-viral methods are used (e.g., minicircle vector). In some cases, cell toxicity is measured by flow cytometry. In some cases, cell toxicity is measured after a viral or a non-viral vector comprising at least one exogenous transgene is introduced to a cell or to a population of cells. In some cases, cell toxicity is reduced by at least about, or at most about, or about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.8%, or 100% when a viral vector (e.g., AAV vector comprising at least one exogenous transgene) is introduced to a cell or to a population of cells compared to when a non-viral vector is introduced (e.g., a minicircle comprising at least one exogenous transgene). In some cases, cellular toxicity is measured at about, at least about, or at most about 4 hours, 6 hours, 8 hours, 12 hours, 18 hours, 24 hours, 30 hours, 36 hours, 42 hours, 48 hours, 54 hours, 60 hours, 66 hours, 72 hours, 78 hours, 84 hours, 90 hours, 96 hours, 102 hours, 108 hours, 114 hours, 120 hours, 126 hours, 132 hours, 138 hours, 144 hours, 150 hours, 156 hours, 168 hours, 180 hours, 192 hours, 204 hours, 216 hours, 228 hours, 240 hours, or longer than 240 hours after a viral vector or a non-viral vector is introduced to a cell or to a population of cells (e.g., post introduction of an AAV vector comprising at least one exogenous transgene or post introduction of a minicircle vector comprising at least one exogenous transgene to a cell or to a population of cells). In some cases, cellular toxicity is measured at about, at least about, or at most about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 45 days, 50 days, 60 days, 70 days, 90 days, or longer than 90 days after a viral vector or a non-viral vector is introduced to a cell or to a population of cells (e.g., post introduction of an AAV vector comprising at least one exogenous transgene or post introduction of a minicircle vector comprising at least one exogenous transgene to a cell or to a population of cells). In some cases, cellular toxicity is measured after at least one exogenous transgene is integrated in at least one cell in a population of cells.

In some cases, a transgene can be inserted into the genome of a cell (e.g., T cell) using random or site specific insertions. In some cases, an insertion can be via a viral insertion. In some cases, a viral insertion of a transgene can be targeted to a particular genomic site or in other cases a viral insertion of a transgene can be a random insertion into a genomic site. In some cases, a transgene is inserted once into the genome of a cell. In some cases, a transgene is randomly inserted into a locus in the genome. In some cases, a transgene is randomly inserted into more than one locus in the genome. In some cases, a transgene is inserted in a gene (e.g., CISH and/or TCR). In some cases, a transgene is inserted at a break in a gene (e.g., CISH and/or TCR). In some cases, more than one transgene is inserted into the genome of a cell. In some cases, more than one transgene is inserted into one or more locus in the genome. In some cases, a transgene is inserted in at least one gene. In some cases, a transgene is inserted in two or more genes (e.g., CISH and/or TCR). In some cases, a transgene or at least one transgene is inserted into a genome of a cell in a random and/or specific manner. In some cases, a transgene is an exogenous transgene. In some cases, a transgene is flanked by engineered sites complementary to at least a portion of a gene (e.g., CISH and/or TCR). In some cases, a transgene is flanked by engineered sites complementary to a break in a gene (e.g., CISH and/or TCR). In some cases, a transgene is not inserted in a gene (e.g., not inserted in a CISH and/or TCR gene). In some cases, a transgene is not inserted at a break in a gene (e.g., break in CISH and/or TCR). In some cases, a transgene is flanked by engineered sites complementary to a break in a genomic locus.

### T Cell Receptor (TCR)

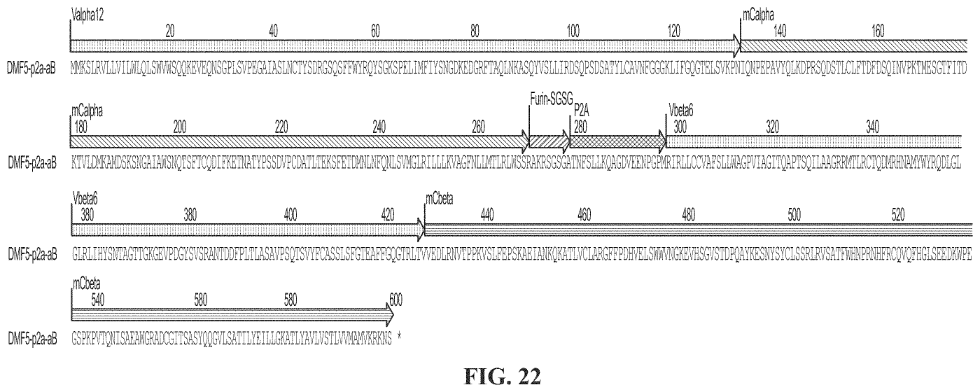

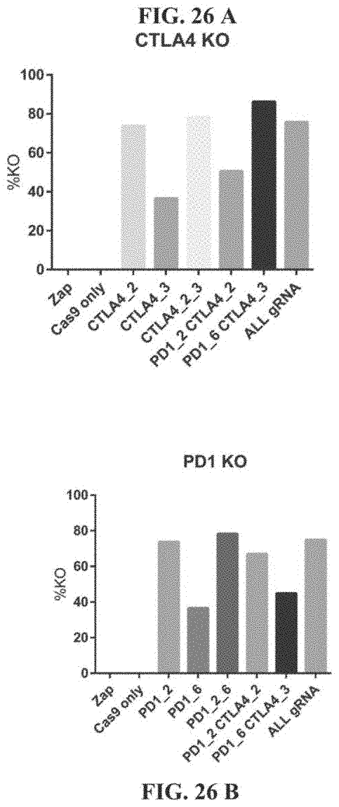

A T cell can comprise one or more transgenes. One or more transgenes can express a TCR alpha, beta, gamma, and/or delta chain protein recognizing and binding to at least one epitope (e.g., cancer epitope) on an antigen or bind to a mutated epitope on an antigen. A TCR can bind to a cancer neo-antigen. A TCR can be a functional TCR as shown in FIG. 22 and FIG. 26. A TCR can comprise only one of the alpha chain or beta chain sequences as defined herein (e.g., in combination with a further alpha chain or beta chain, respectively) or may comprise both chains A TCR can comprise only one of the gamma chain or delta chain sequences as defined herein (e.g., in combination with a further gamma chain or delta chain, respectively) or may comprise both chains. A functional TCR maintains at least substantial biological activity in the fusion protein. In the case of the alpha and/or beta chain of a TCR, this can mean that both chains remain able to form a T cell receptor (either with a non-modified alpha and/or beta chain or with another fusion protein alpha and/or beta chain) which exerts its biological function, in particular binding to the specific peptide-MHC complex of a TCR, and/or functional signal transduction upon peptide activation. In the case of the gamma and/or delta chain of a TCR, this can mean that both chains remain able to form a T cell receptor (either with a non-modified gamma and/or delta chain or with another fusion protein gamma and/or delta chain) which exerts its biological function, in particular binding to the specific peptide-MHC complex of a TCR, and/or functional signal transduction upon peptide activation. A T cell can also comprise one or more TCRs. A T cell can also comprise a single TCRs specific to more than one target.

A TCR can be identified using a variety of methods. In some cases a TCR can be identified using whole-exomic sequencing. For example, a TCR can target an ErbB2 interacting protein (ERBB2IP) antigen containing an E805G mutation identified by whole-exomic sequencing. Alternatively, a TCR can be identified from autologous, allogenic, or xenogeneic repertoires. Autologous and allogeneic identification can entail a multistep process. In both autologous and allogeneic identification, dendritic cells (DCs) can be generated from CD14-selected monocytes and, after maturation, pulsed or transfected with a specific peptide. Peptide-pulsed DCs can be used to stimulate autologous or allogeneic T cells. Single-cell peptide-specific T cell clones can be isolated from these peptide-pulsed T cell lines by limiting dilution. TCRs of interest can be identified and isolated. a and β chains of a TCR of interest can be cloned, codon optimized, and encoded into a vector or transgene. Portions of a TCR can be replaced. For example, constant regions of a human TCR can be replaced with the corresponding murine regions. Replacement of human constant regions with corresponding murine regions can be performed to increase TCR stability. A TCR can also be identified with high or supraphysiologic avidity ex vivo.

To generate a successful tumor-specific TCR, an appropriate target sequence should be identified. The sequence may be found by isolation of a rare tumor-reactive T cell or, where this is not possible, alternative technologies can be employed to generate highly active anti-tumor T-cell antigens. One approach can entail immunizing transgenic mice that express the human leukocyte antigen (HLA) system with human tumor proteins to generate T cells expressing TCRs against human antigens (see e.g., Stanislawski et al., Circumventing tolerance to a human MDM2-derived tumor antigen by TCR gene transfer, Nature Immunology 2, 962-970 (2001)). An alternative approach can be allogeneic TCR gene transfer, in which tumor-specific T cells are isolated from a patient experiencing tumor remission and reactive TCR sequences can be transferred to T cells from another patient who shares the disease but may be non-responsive (de Witte, M. A., et al., Targeting self-antigens through allogeneic TCR gene transfer, Blood 108, 870-877(2006)). Finally, in vitro technologies can be employed to alter a sequence of a TCR, enhancing their tumor-killing activity by increasing the strength of the interaction (avidity) of a weakly reactive tumor-specific TCR with target antigen (Schmid, D. A., et al., Evidence for a TCR affinity threshold delimiting maximal CD8 T cell function. J. Immunol. 184, 4936-4946 (2010)). Alternatively, a TCR can be identified using whole-exomic sequencing.

The present functional TCR fusion protein can be directed against an MHC-presented epitope. The MHC can be a class I molecule, for example HLA-A. The MHC can be a class II molecule. The present functional TCR fusion protein can also have a peptide-based or peptide-guided function in order to target an antigen. The present functional TCR can be linked, for example, the present functional TCR can be linked with a 2A sequence. The present functional TCR can also be linked with furin-V5-SGSGF2A as shown in FIG. 26. The present functional TCR can also contain mammalian components. For example, the present functional TCR can contain mouse constant regions. The present functional TCR can also in some cases contain human constant regions. The peptide-guided function can in principle be achieved by introducing peptide sequences into a TCR and by targeting tumors with these peptide sequences. These peptides may be derived from phage display or synthetic peptide library (see e.g., Arap, W., et al., “Cancer Treatment by Targeted Drug Delivery to Tumor Vasculature in a Mouse Model,” Science, 279, 377-380 (1998); Scott, C. P., et al., “Structural requirements for the biosynthesis of backbone cyclic peptide libraries,” 8: 801-815 (2001)). Among others, peptides specific for breast, prostate and colon carcinomas as well as those specific for neo-vasculatures were already successfully isolated and may be used in the present disclosure (Samoylova, T. I., et al., “Peptide Phage Display: Opportunities for Development of Personalized Anti-Cancer Strategies,” Anti-Cancer Agents in Medicinal Chemistry, 6(1): 9-17(9) (2006)). The present functional TCR fusion protein can be directed against a mutated cancer epitope or mutated cancer antigen.

Transgenes that can be used and are specifically contemplated can include those genes that exhibit a certain identity and/or homology to genes disclosed herein, for example, a TCR gene. Therefore, it is contemplated that if a gene exhibits at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology (at the nucleic acid or protein level), it can be used as a transgene. It is also contemplated that a gene that exhibits at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity (at the nucleic acid or protein level) can be used as a transgene. In some cases, the transgene can be functional.

Transgene can be incorporated into a cell. For example, a transgene can be incorporated into an organism's germ line. When inserted into a cell, a transgene can be either a complementary DNA (cDNA) segment, which is a copy of messenger RNA (mRNA), or a gene itself residing in its original region of genomic DNA (with or without introns). A transgene of protein X can refer to a transgene comprising a nucleotide sequence encoding protein X. As used herein, in some cases, a transgene encoding protein X can be a transgene encoding 100% or about 100% of the amino acid sequence of protein X. In other cases, a transgene encoding protein X can be a transgene encoding at least or at least about 99%, 98%, 97%, 96%, 95%, 94%, 93%, 92%, 91%, 90%, 85%, 80%, 75%, 70%, 65%, 60%, 55%, 50%, 40%, 30%, 20%, 10%, 5%, or 1% of the amino acid sequence of protein X. Expression of a transgene can ultimately result in a functional protein, e.g., a partially, fully, or overly functional protein. As discussed above, if a partial sequence is expressed, the ultimate result can be a nonfunctional protein or a dominant negative protein. A nonfunctional protein or dominant negative protein can also compete with a functional (endogenous or exogenous) protein. A transgene can also encode RNA (e.g., mRNA, shRNA, siRNA, or microRNA). In some cases, where a transgene encodes for an mRNA, this can in turn be translated into a polypeptide (e.g., a protein). Therefore, it is contemplated that a transgene can encode for protein. A transgene can, in some instances, encode a protein or a portion of a protein. Additionally, a protein can have one or more mutations (e.g., deletion, insertion, amino acid replacement, or rearrangement) compared to a wild-type polypeptide. A protein can be a natural polypeptide or an artificial polypeptide (e.g., a recombinant polypeptide). A transgene can encode a fusion protein formed by two or more polypeptides. A T cell can comprise or can comprise about 1, 2, 3, 4, 5, 6, 7, 8, 9, 10, 11, 12, 13, 14, 15, 16, 17, 18, 19, 20, or more transgenes. For example, a T cell can comprise one or more transgene comprising a TCR gene.

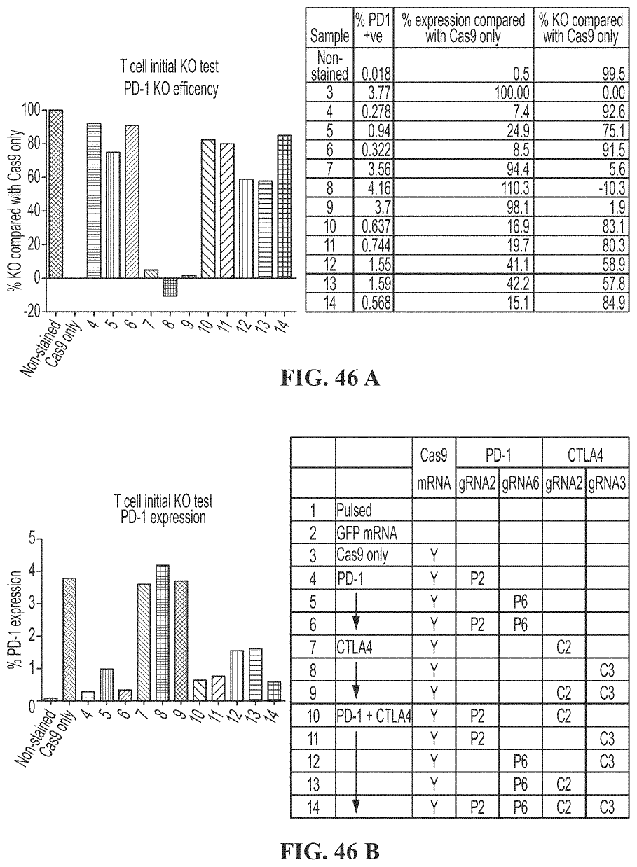

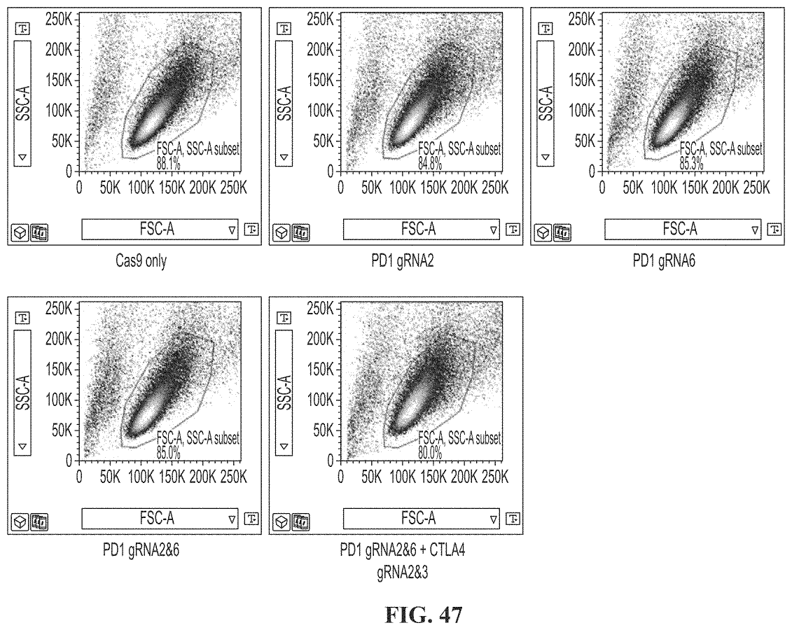

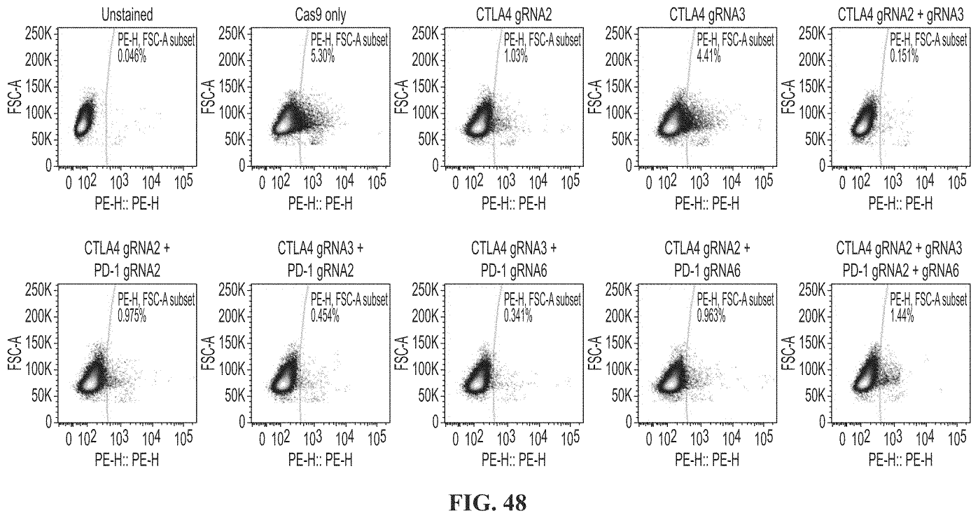

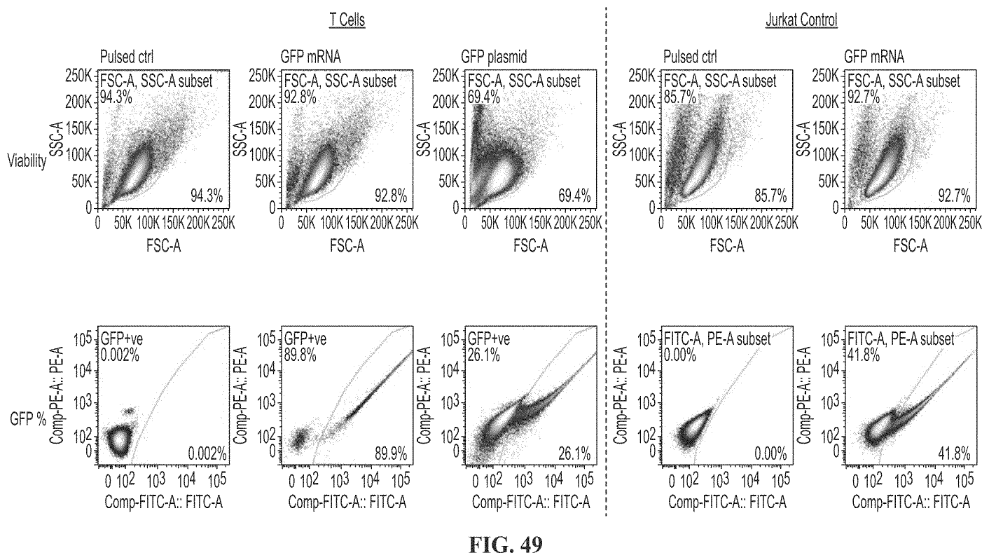

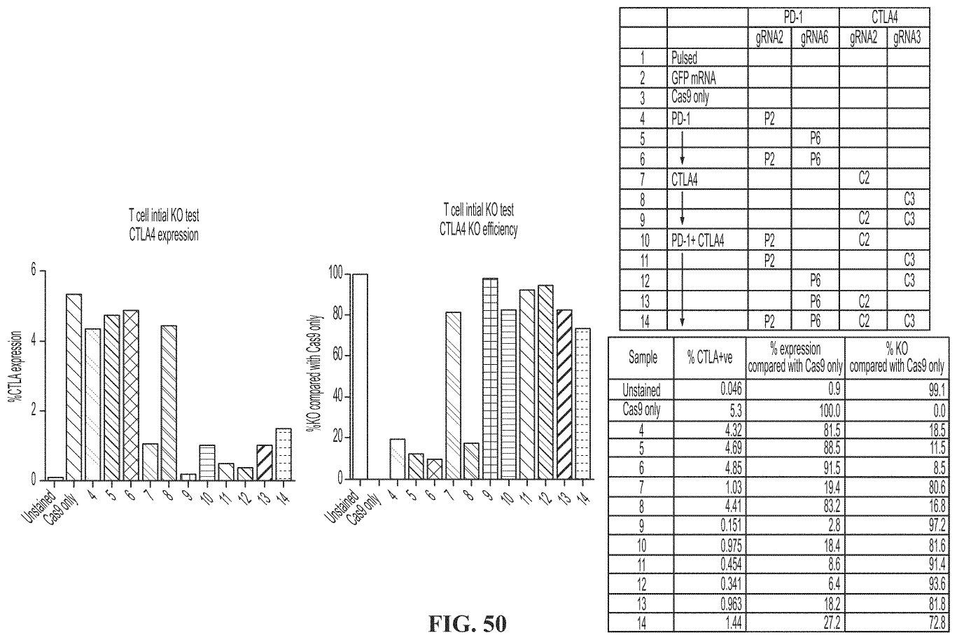

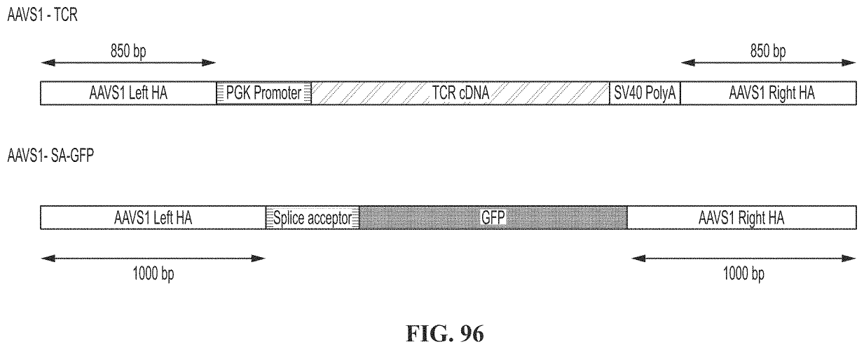

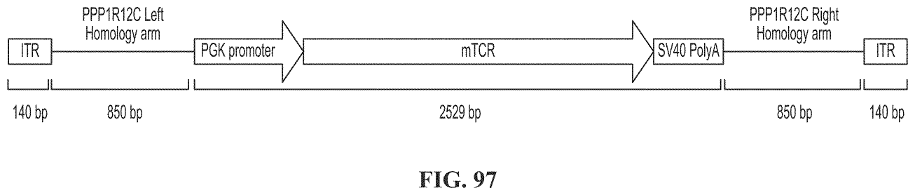

A transgene (e.g., TCR gene) can be inserted in a safe harbor locus. A safe harbor can comprise a genomic location where a transgene can integrate and function without perturbing endogenous activity. For example, one or more transgenes can be inserted into any one of HPRT, AAVS SITE (E.G. AAVS1, AAVS2, ETC.), CCR5, hROSA26, and/or any combination thereof. A transgene (e.g., TCR gene) can also be inserted in an endogenous immune checkpoint gene. An endogenous immune checkpoint gene can be stimulatory checkpoint gene or an inhibitory checkpoint gene. A transgene (e.g., TCR gene) can also be inserted in a stimulatory checkpoint gene such as CD27, CD40, CD122, OX40, GITR, CD137, CD28, or ICOS Immune checkpoint gene locations are provided using the Genome Reference Consortium Human Build 38 patch release 2 (GRCh38.p2) assembly. A transgene (e.g., TCR gene) can also be inserted in an endogenous inhibitory checkpoint gene such as A2AR, B7-H3, B7-H4, BTLA, CTLA-4, IDO, KIR, LAG3, PD-1, TIM-3, VISTA, TCR, or CISH. For example, one or more transgene can be inserted into any one of CD27, CD40, CD122, OX40, GITR, CD137, CD28, ICOS, A2AR, B7-H3, B7-H4, BTLA, CTLA-4, IDO, KIR, LAG3, PD-1, TIM-3, VISTA, HPRT, AAVS SITE (E.G. AAVS1, AAVS2, ETC.), PHD1, PHD2, PHD3, CCR5, TCR, CISH, PPP1R12C, and/or any combination thereof. A transgene can be inserted in an endogenous TCR gene. A transgene can be inserted within a coding genomic region. A transgene can also be inserted within a noncoding genomic region. A transgene can be inserted into a genome without homologous recombination. Insertion of a transgene can comprise a step of an intracellular genomic transplant. A transgene can be inserted at a PD-1 gene, FIG. 46 A and FIG. 46 B. In some cases, more than one guide can target an immune checkpoint, FIG. 47. In other cases, a transgene can be integrated at a CTLA-4 gene, FIG. 48 and FIG. 50. In other cases, a transgene can be integrated at a CTLA-4 gene and a PD-1 gene, FIG. 49. A transgene can also be integrated into a safe harbor such as AAVS1, FIG. 96 and FIG. 97. A transgene can be inserted at a CISH gene. A transgene can be inserted at a TCR gene. A transgene can be inserted into an AAV integration site. An AAV integration site can be a safe harbor in some cases. Alternative AAV integration sites may exist, such as AAVS2 on chromosome 5 or AAVS3 on chromosome 3. Additional AAV integration sites such as AAVS 2, AAVS3, AAVS4, AAVS5, AAVS6, AAVS7, AAVS8, and the like are also considered to be possible integration sites for an exogenous receptor, such as a TCR. As used herein, AAVS can refer to AAVS1 as well as related adeno-associated virus (AAVS) integration sites.

A chimeric antigen receptor can be comprised of an extracellular antigen recognition domain, a transmembrane domain, and a signaling region that controls T cell activation. The extracellular antigen recognition domain can be derived from a murine, a humanized or fully human monoclonal antibody. Specifically, the extracellular antigen recognition domain is comprised of the variable regions of the heavy and light chains of a monoclonal antibody that is cloned in the form of single-chain variable fragments (scFv) and joined through a hinge and a transmembrane domain to an intracellular signaling molecule of the T-cell receptor (TCR) complex and at least one co-stimulatory molecule. In some cases a co-stimulatory domain is not used.

A CAR of the present disclosure can be present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, and an NK cell. When present in the plasma membrane of a eukaryotic cell, a CAR can be active in the presence of its binding target. A target can be expressed on a membrane. A target can also be soluble (e.g., not bound to a cell). A target can be present on the surface of a cell such as a target cell. A target can be presented on a solid surface such as a lipid bilayer; and the like. A target can be soluble, such as a soluble antigen. A target can be an antigen. An antigen can be present on the surface of a cell such as a target cell. An antigen can be presented on a solid surface such as a lipid bilayer; and the like. In some cases, a target can be an epitope of an antigen. In some cases a target can be a cancer neo-antigen.

Some recent advances have focused on identifying tumor-specific mutations that in some cases trigger an antitumor T cell response. For example, these endogenous mutations can be identified using a whole-exomic-sequencing approach. Tran E, et al., “Cancer immunotherapy based on mutation-specific CD4+ T cells in a patient with epithelial cancer,” Science 344: 641-644 (2014). Therefore, a CAR can be comprised of a scFv targeting a tumor-specific neo-antigen.

A method can identify a cancer-related target sequence from a sample obtained from a cancer patient using an in vitro assay (e.g. whole-exomic sequencing). A method can further identify a TCR transgene from a first T cell that recognizes the target sequence. A cancer-related target sequence and a TCR transgene can be obtained from samples of the same patient or different patients. A cancer-related target sequence can be encoded on a CAR transgene to render a CAR specific to a target sequence. A method can effectively deliver a nucleic acid comprising a CAR transgene across a membrane of a T cell. In some instances, the first and second T cells can be obtained from the same patient. In other instances, the first and second T cells can be obtained from different patients. In other instances, the first and second T cells can be obtained from different patients. The method can safely and efficiently integrate a CAR transgene into the genome of a T cell using a non-viral integration or a viral integration system to generate an engineered T cell and thus, a CAR transgene can be reliably expressed in the engineered T cell

A T cell can comprise one or more disrupted genes and one or more transgenes. For example, one or more genes whose expression is disrupted can comprise any one of CD27, CD40, CD122, OX40, GITR, CD137, CD28, ICOS, A2AR, B7-H3, B7-H4, BTLA, CTLA-4, IDO, KIR, LAG3, PD-1, TIM-3, PHD1, PHD2, PHD3, VISTA, TCR, CISH, PPP1R12C, TCR and/or any combination thereof. For example, solely to illustrate various combinations, one or more genes whose expression is disrupted can comprise PD-1 and one or more transgenes comprise TCR. For example, solely to illustrate various combinations, one or more genes whose expression is disrupted can comprise CISH and one or more transgenes comprise TCR. For example, solely to illustrate various combinations, one or more genes whose expression is disrupted can comprise TCR and one or more transgenes comprise TCR. In another example, one or more genes whose expression is disrupted can also comprise CTLA-4, and one or more transgenes comprise TCR. A disruption can result in a reduction of copy number of genomic transcript of a disrupted gene or portion thereof. For example, a gene that can be disrupted may have reduced transcript quantities compared to the same gene in an undisrupted cell. A disruption can result in disruption results in less than 145 copies/μL, 140 copies/μL, 135 copies/μL, 130 copies/μL, 125 copies/μL, 120 copies/μL, 115 copies/μL, 110 copies/μL, 105 copies/μL, 100 copies/μL, 95 copies/μL, 190 copies/μL, 185 copies/μL, 80 copies/μL, 75 copies/μL, 70 copies/μL, 65 copies/μL, 60 copies/μL, 55 copies/μL, 50 copies/μL, 45 copies/μL, 40 copies/μL, 35 copies/μL, 30 copies/μL, 25 copies/μL, 20 copies/μL, 15 copies/μL, 10 copies/μL, 5 copies/μL, 1 copies/μL, or 0.05 copies/μL. A disruption can result in less than 100 copies/μL in some cases.

A T cell can comprise one or more suppressed genes and one or more transgenes. For example, one or more genes whose expression is suppressed can comprise any one of CD27, CD40, CD122, OX40, GITR, CD137, CD28, ICOS, A2AR, B7-H3, B7-H4, BTLA, CTLA-4, IDO, KIR, LAG3, PD-1, TIM-3, PHD1, PHD2, PHD3, VISTA, CISH, PPP1R12C, TCR and/or any combination thereof. For example, solely to illustrate various combinations, one or more genes whose expression is suppressed can comprise PD-1 and one or more transgenes comprise TCR. For example, solely to illustrate various combinations, one or more genes whose expression is suppressed can comprise CISH and one or more transgenes comprise TCR. For example, solely to illustrate various combinations, one or more genes whose expression is suppressed can comprise TCR and one or more transgenes comprise TCR. In another example, one or more genes whose expression is suppressed can also comprise CTLA-4, and one or more transgenes comprise TCR.

A T cell can also comprise or can comprise about 1, 2, 3, 4, 5, 6, 7, 8, 9, 10, 11, 12, 13, 14, 15, 16, 17, 18, 19, 20, or more dominant negative transgenes. Expression of a dominant negative transgenes can suppress expression and/or function of a wild type counterpart of the dominant negative transgene. Thus, for example, a T cell comprising a dominant negative transgene X can have similar phenotypes compared to a different T cell comprising an X gene whose expression is suppressed. One or more dominant negative transgenes can be dominant negative CD27, dominant negative CD40, dominant negative CD122, dominant negative OX40, dominant negative GITR, dominant negative CD137, dominant negative CD28, dominant negative ICOS, dominant negative A2AR, dominant negative B7-H3, dominant negative B7-H4, dominant negative BTLA, dominant negative CTLA-4, dominant negative IDO, dominant negative KIR, dominant negative LAG3, dominant negative PD-1, dominant negative TIM-3, dominant negative VISTA, dominant negative PHD1, dominant negative PHD2, dominant negative PHD3, dominant negative CISH, dominant negative TCR, dominant negative CCR5, dominant negative HPRT, dominant negative AAVS SITE (e.g. AAVS1, AAVS2, ETC.), dominant negative PPP1R12C, or any combination thereof.

Also provided is a T cell comprising one or more transgenes that encodes one or more nucleic acids that can suppress genetic expression, e.g., can knockdown a gene. RNAs that suppress genetic expression can comprise, but are not limited to, shRNA, siRNA, RNAi, and microRNA. For example, siRNA, RNAi, and/or microRNA can be delivered to a T cell to suppress genetic expression. Further, a T cell can comprise one or more transgene encoding shRNAs. shRNA can be specific to a particular gene. For example, a shRNA can be specific to any gene described in the application, including but not limited to, CD27, CD40, CD122, OX40, GITR, CD137, CD28, ICOS, A2AR, B7-H3, B7-H4, BTLA, CTLA-4, IDO, KIR, LAG3, PD-1, TIM-3, VISTA, HPRT, AAVS SITE (E.G. AAVS1, AAVS2, ETC.), PHD1, PHD2, PHD3, CCR5, TCR, CISH, PPP1R12C, and/or any combination thereof.

One or more transgenes can be from different species. For example, one or more transgenes can comprise a human gene, a mouse gene, a rat gene, a pig gene, a bovine gene, a dog gene, a cat gene, a monkey gene, a chimpanzee gene, or any combination thereof. For example, a transgene can be from a human, having a human genetic sequence. One or more transgenes can comprise human genes. In some cases, one or more transgenes are not adenoviral genes.

A transgene can be inserted into a genome of a T cell in a random or site-specific manner, as described above. For example, a transgene can be inserted to a random locus in a genome of a T cell. These transgenes can be functional, e.g., fully functional if inserted anywhere in a genome. For instance, a transgene can encode its own promoter or can be inserted into a position where it is under the control of an endogenous promoter. Alternatively, a transgene can be inserted into a gene, such as an intron of a gene or an exon of a gene, a promoter, or a non-coding region. A transgene can be inserted such that the insertion disrupts a gene, e.g., an endogenous checkpoint. A transgene insertion can comprise an endogenous checkpoint region. A transgene insertion can be guided by recombination arms that can flank a transgene.

Sometimes, more than one copy of a transgene can be inserted into more than a random locus in a genome. For example, multiple copies can be inserted into a random locus in a genome. This can lead to increased overall expression than if a transgene was randomly inserted once. Alternatively, a copy of a transgene can be inserted into a gene, and another copy of a transgene can be inserted into a different gene. A transgene can be targeted so that it could be inserted to a specific locus in a genome of a T cell.

Expression of a transgene can be controlled by one or more promoters. A promoter can be a ubiquitous, constitutive (unregulated promoter that allows for continual transcription of an associated gene), tissue-specific promoter or an inducible promoter. Expression of a transgene that is inserted adjacent to or near a promoter can be regulated. For example, a transgene can be inserted near or next to a ubiquitous promoter. Some ubiquitous promoters can be a CAGGS promoter, an hCMV promoter, a PGK promoter, an SV40 promoter, or a ROSA26 promoter.

A promoter can be endogenous or exogenous. For example, one or more transgenes can be inserted adjacent or near to an endogenous or exogenous ROSA26 promoter. Further, a promoter can be specific to a T cell. For example, one or more transgenes can be inserted adjacent or near to a porcine ROSA26 promoter.

Tissue specific promoter or cell-specific promoters can be used to control the location of expression. For example, one or more transgenes can be inserted adjacent or near to a tissue-specific promoter. Tissue-specific promoters can be a FABP promoter, an Lck promoter, a CamKII promoter, a CD19 promoter, a Keratin promoter, an Albumin promoter, an aP2 promoter, an insulin promoter, an MCK promoter, a MyHC promoter, a WAP promoter, or a Col2A promoter.

Tissue specific promoter or cell-specific promoters can be used to control the location of expression. For example, one or more transgenes can be inserted adjacent or near to a tissue-specific promoter. Tissue-specific promoters can be a FABP promoter, an Lck promoter, a CamKII promoter, a CD19 promoter, a Keratin promoter, an Albumin promoter, an aP2 promoter, an insulin promoter, an MCK promoter, a MyHC promoter, a WAP promoter, or a Col2A promoter.

Inducible promoters can be used as well. These inducible promoters can be turned on and off when desired, by adding or removing an inducing agent. It is contemplated that an inducible promoter can be, but is not limited to, a Lac, tac, trc, trp, araBAD, phoA, recA, proU, cst-1, tetA, cadA, nar, PL, cspA, T7, VHB, Mx, and/or Trex.

A cell can be engineered to knock out endogenous genes. Endogenous genes that can be knocked out can comprise immune checkpoint genes. An immune checkpoint gene can be stimulatory checkpoint gene or an inhibitory checkpoint gene Immune checkpoint gene locations can be provided using the Genome Reference Consortium Human Build 38 patch release 2 (GRCh38.p2) assembly.

A gene to be knocked out can be selected using a database. In some cases, certain endogenous genes are more amendable to genomic engineering. A database can comprise epigenetically permissive target sites. A database can be ENCODE (encyclopedia of DNA Elements) (http://www.genome.gov/10005107) in some cases. A databased can identify regions with open chromatin that can be more permissive to genomic engineering.

A T cell can comprise one or more disrupted genes. For example, one or more genes whose expression is disrupted can comprise any one of adenosine A2a receptor (ADORA), CD276, V-set domain containing T cell activation inhibitor 1 (VTCN1), B and T lymphocyte associated (BTLA), cytotoxic T-lymphocyte-associated protein 4 (CTLA4), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), programmed cell death 1 (PD-1), hepatitis A virus cellular receptor 2 (HAVCR2), V-domain immunoglobulin suppressor of T-cell activation (VISTA), natural killer cell receptor 2B4 (CD244), cytokine inducible SH2-containing protein (CISH), hypoxanthine phosphoribosyltransferase 1 (HPRT), adeno-associated virus integration site (AAVS SITE (E.G. AAVS1, AAVS2, ETC.)), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), guanylate cyclase 1, soluble, beta 3 (GUCY1B3), cytokine inducible SH2-containing protein (CISH), prolyl hydroxylase domain (PHD1, PHD2, PHD3) family of proteins, TCR, or any combination thereof. In some cases an endogenous TCR can also be knocked out. For example, solely to illustrate various combinations, one or more genes whose expression is disrupted can comprise PD-1, CLTA-4, TCR, and CISH.

A T cell can comprise one or more suppressed genes. For example, one or more genes whose expression is suppressed can comprise any one of adenosine A2a receptor (ADORA), CD276, V-set domain containing T cell activation inhibitor 1 (VTCN1), B and T lymphocyte associated (BTLA), cytotoxic T-lymphocyte-associated protein 4 (CTLA4), indoleamine 2,3-dioxygenase 1 (IDO1), TCR, killer cell immunoglobulin-like receptor, three domains, long cytoplasmic tail, 1 (KIR3DL1), lymphocyte-activation gene 3 (LAG3), programmed cell death 1 (PD-1), hepatitis A virus cellular receptor 2 (HAVCR2), V-domain immunoglobulin suppressor of T-cell activation (VISTA), natural killer cell receptor 2B4 (CD244), cytokine inducible SH2-containing protein (CISH), hypoxanthine phosphoribosyltransferase 1 (HPRT), adeno-associated virus integration site (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), guanylate cyclase 1, soluble, beta 3 (GUCY1B3), prolyl hydroxylase domain (PHD1, PHD2, PHD3) family of proteins, cytokine inducible SH2-containing protein (CISH), or any combination thereof. For example, solely to illustrate various combinations, one or more genes whose expression is suppressed can comprise PD-1, CLTA-4, TCR, and/or CISH.

d. Cancer Target

An engineered cell can target an antigen. An engineered cell can also target an epitope. An antigen can be a tumor cell antigen. An epitope can be a tumor cell epitope. Such a tumor cell epitope may be derived from a wide variety of tumor antigens such as antigens from tumors resulting from mutations (neo antigens or neo epitopes), shared tumor specific antigens, differentiation antigens, and antigens overexpressed in tumors. Those antigens, for example, may be derived from alpha-actinin-4, ARTC1, BCR-ABL fusion protein (b3a2), B-RAF, CASP-5, CASP-8, beta-catenin, Cdc27, CDK4, CDKN2A, COA-1, dek-can fusion protein, EFTUD2, Elongation factor 2, ETV6-AML1 fusion protein, FLT3-ITD, FN1, GPNMB, LDLR-fucosyltransferase fusion protein, HLA-A2d, HLA-A11d, hsp70-2, KIAAO205, MART2, ME1, MUM-1f, MUM-2, MUM-3, neo-PAP, Myosin class I, NFYC, OGT, OS-9, p53, pml-RARalpha fusion protein, PRDX5, PTPRK, K-ras, N-ras, RBAF600, SIRT2, SNRPD1, SYT-SSX1- or -SSX2 fusion protein, TGF-betaRII, triosephosphate isomerase, BAGE-1, GAGE-1, 2, 8, Gage 3, 4, 5, 6, 7, GnTVf, HERV-K-MEL, KK-LC-1, KM-HN-1, LAGE-1, MAGE-A1, MAGE-A2, MAGE-A3, MAGE-A4, MAGE-A6, MAGE-A9, MAGE-A10, MAGE-Al2, MAGE-C2, mucink, NA-88, NY-ESO-1/LAGE-2, SAGE, Sp17, SSX-2, SSX-4, TAG-1, TAG-2, TRAG-3, TRP2-INT2g, XAGE-1b, CEA, gp100/Pme117, Kallikrein 4, mammaglobin-A, Melan-A/MART-1, NY-BR-1, OA1, PSA, RAB38/NY-MEL-1, TRP-1/gp75, TRP-2, tyrosinase, adipophilin, AIM-2, ALDH1A1, BCLX (L), BCMA, BING-4, CPSF, cyclin D1, DKK1, ENAH (hMena), EP-CAM, EphA3, EZH2, FGF5, G250/MN/CAIX, HER-2/neu, IL13Ralpha2, intestinal carboxyl esterase, alpha fetoprotein, M-CSFT, MCSP, mdm-2, MMP-2, MUC1, p53, PBF, PRAME, PSMA, RAGE-1, RGSS, RNF43, RU2AS, secernin 1, SOX10, STEAP1, survivin, Telomerase, VEGF, and/or WT1, just to name a few. Tumor-associated antigens may be antigens not normally expressed by the host; they can be mutated, truncated, misfolded, or otherwise abnormal manifestations of molecules normally expressed by the host; they can be identical to molecules normally expressed but expressed at abnormally high levels; or they can be expressed in a context or environment that is abnormal. Tumor-associated antigens may be, for example, proteins or protein fragments, complex carbohydrates, gangliosides, haptens, nucleic acids, other biological molecules or any combinations thereof.

In some cases, a target is a neo antigen or neo epitope. For example, a neo antigen can be an E805G mutation in ERBB2IP. Neo antigen and neo epitopes can be identified by whole-exome sequencing in some cases. A neo antigen and neo epitope target can be expressed by a gastrointestinal cancer cell in some cases. A neo antigen and neo epitope can be expressed on an epithelial carcinoma.

e. Other Targets

An epitope can be a stromal epitope. Such an epitope can be on the stroma of the tumor microenvironment. The antigen can be a stromal antigen. Such an antigen can be on the stroma of the tumor microenvironment. Those antigens and those epitopes, for example, can be present on tumor endothelial cells, tumor vasculature, tumor fibroblasts, tumor pericytes, tumor stroma, and/or tumor mesenchymal cells, just to name a few. Those antigens, for example, can comprise CD34, MCSP, FAP, CD31, PCNA, CD117, CD40, MMP4, and/or Tenascin.

f. Disruption of Genes

The insertion of transgene can be done with or without the disruption of a gene. A transgene can be inserted adjacent to, near, or within a gene such as CD27, CD40, CD122, OX40, GITR, CD137, CD28, ICOS, A2AR, B7-H3, B7-H4, BTLA, CTLA-4, IDO, KIR, LAG3, PD-1, TIM-3, VISTA, HPRT, AAVS SITE (E.G. AAVS1, AAVS2, ETC.), CCR5, PPP1R12C, TCR, or CISH to reduce or eliminate the activity or expression of the gene. For example, a cancer-specific TCR transgene can be inserted adjacent to, near, or within a gene (e.g., CISH and/or TCR) to reduce or eliminate the activity or expression of the gene. The insertion of a transgene can be done at an endogenous TCR gene.

The disruption of genes can be of any particular gene. It is contemplated that genetic homologues (e.g., any mammalian version of the gene) of the genes within this applications are covered. For example, genes that are disrupted can exhibit a certain identity and/or homology to genes disclosed herein, e.g., CD27, CD40, CD122, OX40, GITR, CD137, CD28, ICOS, A2AR, B7-H3, B7-H4, BTLA, CTLA-4, IDO, KIR, LAG3, PD-1, TIM-3, VISTA, HPRT, CCR5, AAVS SITE (E.G. AAVS1, AAVS2, ETC.), PPP1R12C, TCR, and/or CISH. Therefore, it is contemplated that a gene that exhibits or exhibits about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology (at the nucleic acid or protein level) can be disrupted. It is also contemplated that a gene that exhibits or exhibits about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity (at the nucleic acid or protein level) can be disrupted. Some genetic homologues are known in the art, however, in some cases, homologues are unknown. However, homologous genes between mammals can be found by comparing nucleic acid (DNA or RNA) sequences or protein sequences using publically available databases such as NCBI BLAST.

A gene that can be disrupted can be a member of a family of genes. For example, a gene that can be disrupted can improve therapeutic potential of cancer immunotherapy. In some instances, a gene can be CISH. A CISH gene can be a member of a cytokine-induced STAT inhibitor (CIS), also known as suppressor of cytokine signaling (SOCS) or STAT-induced STAT inhibitor (SSI), protein family (see e.g., Palmer et al., Cish actively silences TCR signaling in CD8+ T cells to maintain tumor tolerance, The Journal of Experimental Medicine 202(12), 2095-2113 (2015)). A gene can be part of a SOCS family of proteins that can form part of a classical negative feedback system that can regulate cytokine signal transduction. A gene to be disrupted can be CISH. CISH can be involved in negative regulation of cytokines that signal through the JAK-STATS pathway such as erythropoietin, prolactin or interleukin 3 (IL-3) receptor. A gene can inhibit STATS trans-activation by suppressing its tyrosine phosphorylation. CISH family members are known to be cytokine-inducible negative regulators of cytokine signaling. Expression of a gene can be induced by IL2, IL3, GM-CSF or EPO in hematopoietic cells. Proteasome-mediated degradation of a gene protein can be involved in the inactivation of an erythropoietin receptor. In some cases, a gene to be targeted can be expressed in tumor-specific T cells. A gene to be targeted can increase infiltration of an engineered cell into antigen-relevant tumors when disrupted. In some cases, a gene to be targeted can be CISH.

A gene that can be disrupted can be involved in attenuating TCR signaling, functional avidity, or immunity to cancer. In some cases, a gene to be disrupted is upregulated when a TCR is stimulated. A gene can be involved in inhibiting cellular expansion, functional avidity, or cytokine polyfunctionality. A gene can be involved in negatively regulating cellular cytokine production. For example, a gene can be involved in inhibiting production of effector cytokines, IFN-gamma and/or TNF for example. A gene can also be involved in inhibiting expression of supportive cytokines such as IL-2 after TCR stimulation. Such a gene can be CISH.

Gene suppression can also be done in a number of ways. For example, gene expression can be suppressed by knock out, altering a promoter of a gene, and/or by administering interfering RNAs. This can be done at an organism level or at a tissue, organ, and/or cellular level. If one or more genes are knocked down in a cell, tissue, and/or organ, the one or more genes can be suppressed by administrating RNA interfering reagents, e.g., siRNA, shRNA, or microRNA. For example, a nucleic acid which can express shRNA can be stably transfected into a cell to knockdown expression. Furthermore, a nucleic acid which can express shRNA can be inserted into the genome of a T cell, thus knocking down a gene within the T cell.

Disruption methods can also comprise overexpressing a dominant negative protein. This method can result in overall decreased function of a functional wild-type gene. Additionally, expressing a dominant negative gene can result in a phenotype that is similar to that of a knockout and/or knockdown.

Sometimes a stop codon can be inserted or created (e.g., by nucleotide replacement), in one or more genes, which can result in a nonfunctional transcript or protein (sometimes referred to as knockout). For example, if a stop codon is created within the middle of one or more genes, the resulting transcription and/or protein can be truncated, and can be nonfunctional. However, in some cases, truncation can lead to an active (a partially or overly active) protein. If a protein is overly active, this can result in a dominant negative protein.

This dominant negative protein can be expressed in a nucleic acid within the control of any promoter. For example, a promoter can be a ubiquitous promoter. A promoter can also be an inducible promoter, tissue specific promoter, cell specific promoter, and/or developmental specific promoter.

The nucleic acid that codes for a dominant negative protein can then be inserted into a cell. Any method can be used. For example, stable transfection can be used. Additionally, a nucleic acid that codes for a dominant negative protein can be inserted into a genome of a T cell.

One or more genes in a T cell can be knocked out or disrupted using any method. For example, knocking out one or more genes can comprise deleting one or more genes from a genome of a T cell. Knocking out can also comprise removing all or a part of a gene sequence from a T cell. It is also contemplated that knocking out can comprise replacing all or a part of a gene in a genome of a T cell with one or more nucleotides. Knocking out one or more genes can also comprise inserting a sequence in one or more genes thereby disrupting expression of the one or more genes. For example, inserting a sequence can generate a stop codon in the middle of one or more genes. Inserting a sequence can also shift the open reading frame of one or more genes.

Knockout can be done in any cell, organ, and/or tissue, e.g., in a T cell, hematopoietic stem cell, in the bone marrow, and/or the thymus. For example, knockout can be whole body knockout, e.g., expression of one or more genes is suppressed in all cells of a human. Knockout can also be specific to one or more cells, tissues, and/or organs of a human. This can be achieved by conditional knockout, where expression of one or more genes is selectively suppressed in one or more organs, tissues or types of cells. Conditional knockout can be performed by a Cre-lox system, wherein cre is expressed under the control of a cell, tissue, and/or organ specific promoter. For example, one or more genes can be knocked out (or expression can be suppressed) in one or more tissues, or organs, where the one or more tissues or organs can include brain, lung, liver, heart, spleen, pancreas, small intestine, large intestine, skeletal muscle, smooth muscle, skin, bones, adipose tissues, hairs, thyroid, trachea, gall bladder, kidney, ureter, bladder, aorta, vein, esophagus, diaphragm, stomach, rectum, adrenal glands, bronchi, ears, eyes, retina, genitals, hypothalamus, larynx, nose, tongue, spinal cord, or ureters, uterus, ovary, testis, and/or any combination thereof. One or more genes can also be knocked out (or expression can be suppressed) in one types of cells, where one or more types of cells include trichocytes, keratinocytes, gonadotropes, corticotropes, thyrotropes, somatotropes, lactotrophs, chromaffin cells, parafollicular cells, glomus cells melanocytes, nevus cells, merkel cells, odontoblasts, cementoblasts corneal keratocytes, retina muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, Enterochromaffin-like cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells, pancreatic E cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, and/or any combination thereof.

In some cases, the methods of the present disclosure may comprise obtaining one or more cells from a subject. A cell may generally refer to any biological structure comprising cytoplasm, proteins, nucleic acids, and/or organelles enclosed within a membrane. In some cases, a cell may be a mammalian cell. In some cases, a cell may refer to an immune cell. Non-limiting examples of a cell can include a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof.

In some cases, the cell may be an ILC, and the ILC is a group 1 ILC, a group 2 ILC, or a group 3 ILC. Group 1 ILCs may generally be described as cells controlled by the T-bet transcription factor, secreting type-1 cytokines such as IFN-gamma and TNF-alpha in response to intracellular pathogens. Group 2 ILCs may generally be described as cells relying on the GATA-3 and ROR-alpha transcription factors, producing type-2 cytokines in response to extracellular parasite infections. Group 3 ILCs may generally be described as cells controlled by the ROR-gamma t transcription factor, and produce IL-17 and/or IL-22.

In some cases, the cell may be a cell that is positive or negative for a given factor. In some cases, a cell may be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin− cell, a CD25+, a CD25− cell, a CD27+, a CD27− cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor G1positive cell, a lectin-like receptor G1 negative cell, or an differentiated or de-differentiated cell thereof. The examples of factors expressed by cells is not intended to be limiting, and a person having skill in the art will appreciate that a cell may be positive or negative for any factor known in the art. In some cases, a cell may be positive for two or more factors. For example, a cell may be CD4+ and CD8+. In some cases, a cell may be negative for two or more factors. For example, a cell may be CD25−, CD44−, and CD69−. In some cases, a cell may be positive for one or more factors, and negative for one or more factors. For example, a cell may be CD4+ and CD8−. The selected cells can then be infused into a subject. In some cases, the cells may be selected for having or not having one or more given factors (e.g., cells may be separated based on the presence or absence of one or more factors). Separation efficiency can affect the viability of cells, and the efficiency with which a transgene may be integrated into the genome of a cell and/or expressed. In some cases, the selected cells can also be expanded in vitro. The selected cells can be expanded in vitro prior to infusion. It should be understood that cells used in any of the methods disclosed herein may be a mixture (e.g., two or more different cells) of any of the cells disclosed herein. For example, a method of the present disclosure may comprise cells, and the cells are a mixture of CD4+ cells and CD8+ cells. In another example, a method of the present disclosure may comprise cells, and the cells are a mixture of CD4+ cells and naïve cells.

Naïve cells retain several properties that may be particularly useful for the methods disclosed herein. For example, naïve cells are readily capable of in vitro expansion and T-cell receptor transgene expression, they exhibit fewer markers of terminal differentiation (a quality which may be associated with greater efficacy after cell infusion), and retain longer telomeres, suggestive of greater proliferative potential (Hinrichs, C. S., et al., “Human effector CD8+ T cells derived from naïve rather than memory subsets possess superior traits for adoptive immunotherapy,” Blood, 117(3):808-14 (2011)). The methods disclosed herein may comprise selection or negative selection of markers specific for naïve cells. In some cases, the cell may be a naïve cell. A naïve cell may generally refer to any cell that has not been exposed to an antigen. Any cell in the present disclosure may be a naïve cell. In one example, a cell may be a naïve T cell. A naïve T cell may generally be described a cell that has differentiated in bone marrow, and successfully undergone the positive and negative processes of central selection in the thymus, and/or may be characterized by the expression or absence of specific markers (e.g., surface expression of L-selectin, the absence of the activation markers CD25, CD44 or CD69, and the absence of memory CD45RO isoform).

In some cases, cells may comprise cell lines (e.g., immortalized cell lines). Non-limiting examples of cell lines include human BC-1 cells, human BJAB cells, human IM-9 cells, human Jiyoye cells, human K-562 cells, human LCL cells, mouse MPC-11 cells, human Raji cells, human Ramos cells, mouse Ramos cells, human RPMI8226 cells, human RS4-11 cells, human SKW6.4 cells, human Dendritic cells, mouse P815 cells, mouse RBL-2H3 cells, human HL-60 cells, human NAMALWA cells, human Macrophage cells, mouse RAW 264.7 cells, human KG-1 cells, mouse M1 cells, human PBMC cells, mouse BW5147 (T200-A)5.2 cells, human CCRF-CEM cells, mouse EL4 cells, human Jurkat cells, human SCID.adh cells, human U-937 cells or any combination of cells thereof.

Stem cells can give rise to a variety of somatic cells and thus have in principle the potential to serve as an endless supply of therapeutic cells of virtually any type. The re-programmability of stem cells also allows for additional engineering to enhance the therapeutic value of the reprogrammed cell. In any of the methods of the present disclosure, one or more cells may be derived from a stem cell. Non-limiting examples of stem cells include embryonic stem cells, adult stem cells, tissue-specific stem cells, neural stem cells, allogenic stem cells, totipotent stem cells, multipotent stem cells, pluripotent stem cells, induced pluripotent stem cells, hematopoietic stem cells, epidermal stem cells, umbilical cord stem cells, epithelial stem cells, or adipose-derived stem cells. In one example, a cell may be hematopoietic stem cell-derived lymphoid progenitor cells. In another example, a cell may be embryonic stem cell-derived T cell. In yet another example, a cell may be an induced pluripotent stem cell (iPSC)-derived T cell.

Conditional knockouts can be inducible, for example, by using tetracycline inducible promoters, development specific promoters. This can allow for eliminating or suppressing expression of a gene/protein at any time or at a specific time. For example, with the case of a tetracycline inducible promoter, tetracycline can be given to a T cell any time after birth. A cre/lox system can also be under the control of a developmental specific promoter. For example, some promoters are turned on after birth, or even after the onset of puberty. These promoters can be used to control cre expression, and therefore can be used in developmental specific knockouts.

It is also contemplated that any combinations of knockout technology can be combined. For example, tissue specific knockout or cell specific knockout can be combined with inducible technology, creating a tissue specific or cell specific, inducible knockout. Furthermore, other systems such developmental specific promoter, can be used in combination with tissues specific promoters, and/or inducible knockouts.

Knocking out technology can also comprise gene editing. For example, gene editing can be performed using a nuclease, including CRISPR associated proteins (Cas proteins, e.g., Cas9), Zinc finger nuclease (ZFN), Transcription Activator-Like Effector Nuclease (TALEN), and meganucleases. Nucleases can be naturally existing nucleases, genetically modified, and/or recombinant. Gene editing can also be performed using a transposon-based system (e.g. PiggyBac, Sleeping beauty). For example, gene editing can be performed using a transposase.

In some cases, a nuclease or a polypeptide encoding a nuclease introduces a break into at least one gene (e.g., CISH and/or TCR). In some cases, a nuclease or a polypeptide encoding a nuclease comprises and/or results in an inactivation or reduced expression of at least one gene (e.g., CISH and/or TCR). In some cases, a gene is selected from the group consisting of CISH, TCR, adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), T-cell receptor alpha locus (TRA), T cell receptor beta locus (TRB), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), and any combinations or derivatives thereof.

### CRISPR System

Methods described herein can take advantage of a CRISPR system. There are at least five types of CRISPR systems which all incorporate RNAs and Cas proteins. Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

The general mechanism and recent advances of CRISPR system is discussed in Cong, L. et al., “Multiplex genome engineering using CRISPR systems,” Science, 339(6121): 819-823 (2013); Fu, Y. et al., “High-frequency off-target mutagenesis induced by CRISPR-Cas nucleases in human cells,” Nature Biotechnology, 31, 822-826 (2013); Chu, V T et al. “Increasing the efficiency of homology-directed repair for CRISPR-Cas9-induced precise gene editing in mammalian cells,” Nature Biotechnology 33, 543-548 (2015); Shmakov, S. et al., “Discovery and functional characterization of diverse Class 2 CRISPR-Cas systems,” Molecular Cell, 60, 1-13 (2015); Makarova, K S et al., “An updated evolutionary classification of CRISPR-Cas systems,”, Nature Reviews Microbiology, 13, 1-15 (2015). Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). For example, an engineered cell can be generated using a CRISPR system, e.g., a type II CRISPR system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence.

A CRISPR system can be introduced to a cell or to a population of cells using any means. In some cases, a CRISPR system may be introduced by electroporation or nucleofection. Electroporation can be performed for example, using the Neon® Transfection System (ThermoFisher Scientific) or the AMAXA® Nucleofector (AMAXA® Biosystems) can also be used for delivery of nucleic acids into a cell. Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some cases, the electroporation pulse voltage, the electroporation pulse width, number of pulses, cell density, and tip type may be adjusted to optimize transfection efficiency and/or cell viability.

a. Cas Protein

A vector can be operably linked to an enzyme-coding sequence encoding a CRISPR enzyme, such as a Cas protein (CRISPR-associated protein). In some cases, a nuclease or a polypeptide encoding a nuclease is from a CRISPR system (e.g., CRISPR enzyme). Non-limiting examples of Cas proteins can include Cas1, Cas1B, Cas2, Cas3, Cas4, Cas5, Cas6, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof. In some cases, a catalytically dead Cas protein can be used (e.g., catalytically dead Cas9 (dCas9)). An unmodified CRISPR enzyme can have DNA cleavage activity, such as Cas9. In some cases, a nuclease is Cas9. In some cases, a polypeptide encodes Cas9. In some cases, a nuclease or a polypeptide encoding a nuclease is catalytically dead. In some cases, a nuclease is a catalytically dead Cas9 (dCas9). In some cases, a polypeptide encodes a catalytically dead Cas9 (dCas9). A CRISPR enzyme can direct cleavage of one or both strands at a target sequence, such as within a target sequence and/or within a complement of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. A vector that encodes a CRISPR enzyme that is mutated with respect to a corresponding wild-type enzyme such that the mutated CRISPR enzyme lacks the ability to cleave one or both strands of a target polynucleotide containing a target sequence can be used. A Cas protein can be a high fidelity Cas protein such as Cas9HiFi.

A vector that encodes a CRISPR enzyme comprising one or more nuclear localization sequences (NLSs), such as more than or more than about 1, 2, 3, 4, 5, 6, 7, 8, 9, 10, NLSs can be used. For example, a CRISPR enzyme can comprise more than or more than about 1, 2, 3, 4, 5, 6, 7, 8, 9, 10, NLSs at or near the amino-terminus, more than or more than about 1, 2, 3, 4, 5, 6, 7, 8, 9, 10, NLSs at or near the carboxyl-terminus, or any combination of these (e.g., one or more NLS at the amino-terminus and one or more NLS at the carboxyl terminus). When more than one NLS is present, each can be selected independently of others, such that a single NLS can be present in more than one copy and/or in combination with one or more other NLSs present in one or more copies.

Cas9 can refer to a polypeptide with at least or at least about 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most or at most about 50%, 60%, 70%, 80%, 90%, 100% sequence identity and/or sequence similarity to a wild type exemplary Cas9 polypeptide (e.g., from S. pyogenes). Cas9 can refer to the wild type or a modified form of the Cas9 protein that can comprise an amino acid change such as a deletion, insertion, substitution, variant, mutation, fusion, chimera, or any combination thereof.

A polynucleotide encoding a nuclease or an endonuclease (e.g., a Cas protein such as Cas9) can be codon optimized for expression in particular cells, such as eukaryotic cells. This type of optimization can entail the mutation of foreign-derived (e.g., recombinant) DNA to mimic the codon preferences of the intended host organism or cell while encoding the same protein.

CRISPR enzymes used in the methods can comprise NLSs. The NLS can be located anywhere within the polypeptide chain, e.g., near the N- or C-terminus. For example, the NLS can be within or within about 1, 2, 3, 4, 5, 10, 15, 20, 25, 30, 40, 50 amino acids along a polypeptide chain from the N- or C-terminus. Sometimes the NLS can be within or within about 50 amino acids or more, e.g., 100, 200, 300, 400, 500, 600, 700, 800, 900, or 1000 amino acids from the N- or C-terminus.

A nuclease or an endonuclease can comprise an amino acid sequence having at least or at least about 50%, 60%, 70%, 75%, 80%, 85%, 90%, 95%, 99%, or 100%, amino acid sequence identity to the nuclease domain of a wild type exemplary site-directed polypeptide (e.g., Cas9 from S. pyogenes).

While S. pyogenes Cas9 (SpCas9), Table 11, is commonly used as a CRISPR endonuclease for genome engineering, it may not be the best endonuclease for every target excision site. For example, the PAM sequence for SpCas9 (5′ NGG 3′) is abundant throughout the human genome, but a NGG sequence may not be positioned correctly to target a desired gene for modification. In some cases, a different endonuclease may be used to target certain genomic targets. In some cases, synthetic SpCas9-derived variants with non-NGG PAM sequences may be used. Additionally, other Cas9 orthologues from various species have been identified and these “non-SpCas9s” bind a variety of PAM sequences that could also be useful for the present disclosure. For example, the relatively large size of SpCas9 (approximately 4kb coding sequence) means that plasmids carrying the SpCas9 cDNA may not be efficiently expressed in a cell. Conversely, the coding sequence for Staphylococcus aureus Cas9 (SaCas9) is approximately 1 kilo base shorter than SpCas9, possibly allowing it to be efficiently expressed in a cell. Similar to SpCas9, the SaCas9 endonuclease is capable of modifying target genes in mammalian cells in vitro and in mice in vivo.

Alternatives to S. pyogenes Cas9 may include RNA-guided endonucleases from the Cpf1 family that display cleavage activity in mammalian cells. Unlike Cas9 nucleases, the result of Cpf1-mediated DNA cleavage is a double-strand break with a short 3′ overhang. Cpf1's staggered cleavage pattern may open up the possibility of directional gene transfer, analogous to traditional restriction enzyme cloning, which may increase the efficiency of gene editing. Like the Cas9 variants and orthologues described above, Cpf1 may also expand the number of sites that can be targeted by CRISPR to AT-rich regions or AT-rich genomes that lack the NGG PAM sites favored by SpCas9.

Any functional concentration of Cas protein can be introduced to a cell. For example, 15 micrograms of Cas mRNA can be introduced to a cell. In other cases, a Cas mRNA can be introduced from 0.5 micrograms to 100 micrograms. A Cas mRNA can be introduced from 0.5, 5, 10, 15, 20, 25, 30, 35, 40, 45, 50, 55, 60, 65, 70, 75, 80, 85, 90, 95, or 100 micrograms.

In some cases, a dual nickase approach may be used to introduce a double stranded break or a genomic break. Cas proteins can be mutated at known amino acids within either nuclease domains, thereby deleting activity of one nuclease domain and generating a nickase Cas protein capable of generating a single strand break. A nickase along with two distinct guide RNAs targeting opposite strands may be utilized to generate a double strand break (DSB) within a target site (often referred to as a “double nick” or “dual nickase” CRISPR system). This approach can increase target specificity because it is unlikely that two off-target nicks will be generated within close enough proximity to cause a DSB.

b. Guiding Polynucleic Acid (e.g., gRNA or gDNA)

A guiding polynucleic acid (or a guide polynucleic acid) can be DNA or RNA. A guiding polynucleic acid can be single stranded or double stranded. In some cases, a guiding polynucleic acid can contain regions of single stranded areas and double stranded areas. A guiding polynucleic acid can also form secondary structures. In some cases, a guiding polynucleic acid can contain internucleotide linkages that can be phosphorothioates. Any number of phosphorothioates can exist. For example from 1 to about 100 phosphorothioates can exist in a guiding polynucleic acid sequence. In some cases, from 1 to 10 phosphorothioates are present. In some cases, 0, 1, 2, 3, 4, 5, 6, 7, 8, 9, 10, 11, 12, 13, 14, 15, 16, 17, 18, 19, or 20 phosphorothioates exist in a guiding polynucleic acid sequence.

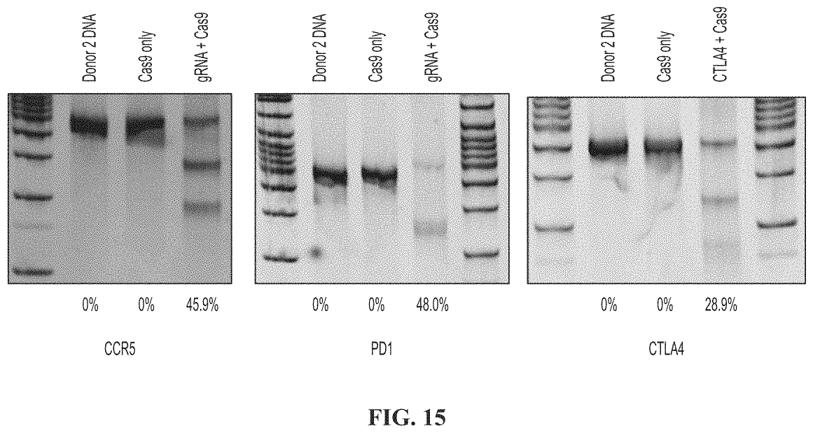

As used herein, the term “guide RNA (gRNA)”, and its grammatical equivalents can refer to an RNA which can be specific for a target DNA and can form a complex with a nuclease such as a Cas protein. A guide RNA can comprise a guide sequence, or spacer sequence, that specifies a target site and guides an RNA/Cas complex to a specified target DNA for cleavage. For example, FIG. 15 demonstrates that guide RNA can target a CRISPR complex to three genes and perform a targeted double strand break. Site-specific cleavage of a target DNA occurs at locations determined by both 1) base-pairing complementarity between a guide RNA and a target DNA (also called a protospacer) and 2) a short motif in a target DNA referred to as a protospacer adjacent motif (PAM). Similarly, a guide RNA (“gDNA”) can be specific for a target DNA and can form a complex with a nuclease to direct its nucleic acid-cleaving activity.

A method disclosed herein can also comprise introducing into a cell or embryo or to a population of cells at least one guide polynucleic acid (e.g., guide DNA, or guide RNA) or nucleic acid (e.g., DNA encoding at least one guide RNA)). A guide RNA can interact with a RNA-guided endonuclease or nuclease to direct the endonuclease or nuclease to a specific target site, at which site the 5′ end of the guide RNA base pairs with a specific protospacer sequence in a chromosomal sequence. In some cases, a guide polynucleic acid can be gRNA and/or gDNA. In some cases, a guide polynucleic acid can have a complementary sequence to at least one gene (e.g., CISH and/or TCR). In some cases, a CRISPR system comprises a guide polynucleic acid. In some cases, a CRISPR system comprises a guide polynucleic acid and/or a nuclease or a polypeptide encoding a nuclease. In some cases, the methods or the systems of the present disclosure further comprises a guide polynucleic acid and/or a nuclease or a polypeptide encoding a nuclease. In some cases, a guide polynucleic acid is introduced at the same time, before, or after a nuclease or a polypeptide encoding a nuclease is introduced to a cell or to a population of cells. In some cases, a guide polynucleic acid is introduced at the same time, before, or after a viral (e.g., AAV) vector or a non-viral (e.g., minicircle) vector is introduced to a cell or to a population of cells (e.g., a guide polynucleic acid is introduced at the same time, before, or after an AAV vector comprising at least one exogenous transgene is introduced to a cell or to a population of cells).

A guide RNA can comprise two RNAs, e.g., CRISPR RNA (crRNA) and transactivating crRNA (tracrRNA). A guide RNA can sometimes comprise a single-guide RNA (sgRNA) formed by fusion of a portion (e.g., a functional portion) of crRNA and tracrRNA. A guide RNA can also be a dual RNA comprising a crRNA and a tracrRNA. A guide RNA can comprise a crRNA and lack a tracrRNA. Furthermore, a crRNA can hybridize with a target DNA or protospacer sequence.

As discussed above, a guide RNA can be an expression product. For example, a DNA that encodes a guide RNA can be a vector comprising a sequence coding for the guide RNA. A guide RNA can be transferred into a cell or organism by transfecting the cell or organism with an isolated guide RNA or plasmid DNA comprising a sequence coding for the guide RNA and a promoter. A guide RNA can also be transferred into a cell or organism in other way, such as using virus-mediated gene delivery.

A guide RNA can be isolated. For example, a guide RNA can be transfected in the form of an isolated RNA into a cell or organism. A guide RNA can be prepared by in vitro transcription using any in vitro transcription system. A guide RNA can be transferred to a cell in the form of isolated RNA rather than in the form of plasmid comprising encoding sequence for a guide RNA.

A guide RNA can comprise a DNA-targeting segment and a protein binding segment. A DNA-targeting segment (or DNA-targeting sequence, or spacer sequence) comprises a nucleotide sequence that can be complementary to a specific sequence within a target DNA (e.g., a protospacer). A protein-binding segment (or protein-binding sequence) can interact with a site-directed modifying polypeptide, e.g. an RNA-guided endonuclease such as a Cas protein. By “segment” it is meant a segment/section/region of a molecule, e.g., a contiguous stretch of nucleotides in RNA. A segment can also mean a region/section of a complex such that a segment may comprise regions of more than one molecule. For example, in some cases a protein-binding segment of a DNA-targeting RNA is one RNA molecule and the protein-binding segment therefore comprises a region of that RNA molecule. In other cases, the protein-binding segment of a DNA-targeting RNA comprises two separate molecules that are hybridized along a region of complementarity.

A guide RNA can comprise two separate RNA molecules or a single RNA molecule. An exemplary single molecule guide RNA comprises both a DNA-targeting segment and a protein-binding segment.

An exemplary two-molecule DNA-targeting RNA can comprise a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A first RNA molecule can be a crRNA-like molecule (targeter-RNA), that can comprise a DNA-targeting segment (e.g., spacer) and a stretch of nucleotides that can form one half of a double-stranded RNA (dsRNA) duplex comprising the protein-binding segment of a guide RNA. A second RNA molecule can be a corresponding tracrRNA-like molecule (activator-RNA) that can comprise a stretch of nucleotides that can form the other half of a dsRNA duplex of a protein-binding segment of a guide RNA. In other words, a stretch of nucleotides of a crRNA-like molecule can be complementary to and can hybridize with a stretch of nucleotides of a tracrRNA-like molecule to form a dsRNA duplex of a protein-binding domain of a guide RNA. As such, each crRNA-like molecule can be said to have a corresponding tracrRNA-like molecule. A crRNA-like molecule additionally can provide a single stranded DNA-targeting segment, or spacer sequence. Thus, a crRNA-like and a tracrRNA-like molecule (as a corresponding pair) can hybridize to form a guide RNA. A subject two-molecule guide RNA can comprise any corresponding crRNA and tracrRNA pair.

A DNA-targeting segment or spacer sequence of a guide RNA can be complementary to sequence at a target site in a chromosomal sequence, e.g., protospacer sequence) such that the DNA-targeting segment of the guide RNA can base pair with the target site or protospacer. In some cases, a DNA-targeting segment of a guide RNA can comprise from or from about 10 nucleotides to from or from about 25 nucleotides or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more than 25 nucleotides in length. Sometimes, a first region of a guide RNA can be or can be about 19, 20, or 21 nucleotides in length.

A guide RNA can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30 or more nucleotides. A target nucleic acid can be at most or at most about 5, 10, 15, 16, 17, 18, 19, 20, 21, 22, 23, 24, 25, 30 or more nucleotides. A target nucleic acid sequence can be or can be about 20 bases immediately 5′ of the first nucleotide of the PAM. A guide RNA can target the nucleic acid sequence. In some cases, a guiding polynucleic acid, such as a guide RNA, can bind a genomic region from about 1 basepair to about 20 basepairs away from a PAM. A guide can bind a genomic region from about 1, 2, 3, 4, 5 6, 7, 8, 9, 10, 11, 12, 13, 14, 15, 16, 17, 18, 19, or up to about 20 base pairs away from a PAM.

A guide nucleic acid, for example, a guide RNA, can refer to a nucleic acid that can hybridize to another nucleic acid, for example, the target nucleic acid or protospacer in a genome of a cell. A guide nucleic acid can be RNA. A guide nucleic acid can be DNA. The guide nucleic acid can be programmed or designed to bind to a sequence of nucleic acid site-specifically. A guide nucleic acid can comprise a polynucleotide chain and can be called a single guide nucleic acid. A guide nucleic acid can comprise two polynucleotide chains and can be called a double guide nucleic acid.

A guide nucleic acid can comprise one or more modifications to provide a nucleic acid with a new or enhanced feature. A guide nucleic acid can comprise a nucleic acid affinity tag. A guide nucleic acid can comprise synthetic nucleotide, synthetic nucleotide analog, nucleotide derivatives, and/or modified nucleotides.

A guide nucleic acid can comprise a nucleotide sequence (e.g., a spacer), for example, at or near the 5′ end or 3′ end, that can hybridize to a sequence in a target nucleic acid (e.g., a protospacer). A spacer of a guide nucleic acid can interact with a target nucleic acid in a sequence-specific manner via hybridization (i. e., base pairing). A spacer sequence can hybridize to a target nucleic acid that is located 5′ or 3′ of a protospacer adjacent motif (PAM). The length of a spacer sequence can be at least or at least about 5, 10, 15, 16, 17, 18, 19, 20, 21, 22, 23, 24, 25, 30 or more nucleotides. The length of a spacer sequence can be at most or at most about 5, 10, 15, 16, 17, 18, 19, 20, 21, 22, 23, 24, 25, 30 or more nucleotides.

A guide RNA can also comprise a dsRNA duplex region that forms a secondary structure. For example, a secondary structure formed by a guide RNA can comprise a stem (or hairpin) and a loop. A length of a loop and a stem can vary. For example, a loop can range from about 3 to about 10 nucleotides in length, and a stem can range from about 6 to about 20 base pairs in length. A stem can comprise one or more bulges of 1 to about 10 nucleotides. The overall length of a second region can range from about 16 to about 60 nucleotides in length. For example, a loop can be or can be about 4 nucleotides in length and a stem can be or can be about 12 base pairs. A dsRNA duplex region can comprise a protein-binding segment that can form a complex with an RNA-binding protein, such as a RNA-guided endonuclease, e.g. Cas protein. 1003891A guide RNA can also comprise a tail region at the 5′ or 3′ end that can be essentially single-stranded. For example, a tail region is sometimes not complementarity to any chromosomal sequence in a cell of interest and is sometimes not complementarity to the rest of a guide RNA. Further, the length of a tail region can vary. A tail region can be more than or more than about 4 nucleotides in length. For example, the length of a tail region can range from or from about 5 to from or from about 60 nucleotides in length.

A guide RNA can be introduced into a cell or embryo as an RNA molecule. For example, a RNA molecule can be transcribed in vitro and/or can be chemically synthesized. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III).

A DNA molecule encoding a guide RNA can also be linear. A DNA molecule encoding a guide RNA can also be circular.

A DNA sequence encoding a guide RNA can also be part of a vector. Some examples of vectors can include plasmid vectors, phagemids, cosmids, artificial/mini-chromosomes, transposons, and viral vectors. For example, a DNA encoding a RNA-guided endonuclease is present in a plasmid vector. Other non-limiting examples of suitable plasmid vectors include pUC, pBR322, pET, pBluescript, and variants thereof. Further, a vector can comprise additional expression control sequences (e g., enhancer sequences, Kozak sequences, polyadenylation sequences, transcriptional termination sequences, etc.), selectable marker sequences (e.g., antibiotic resistance genes), origins of replication, and the like.

When both a RNA-guided endonuclease and a guide RNA are introduced into a cell as DNA molecules, each can be part of a separate molecule (e.g., one vector containing fusion protein coding sequence and a second vector containing guide RNA coding sequence) or both can be part of a same molecule (e.g., one vector containing coding (and regulatory) sequence for both a fusion protein and a guide RNA).

A Cas protein, such as a Cas9 protein or any derivative thereof, can be pre-complexed with a guide RNA to form a ribonucleoprotein (RNP) complex. The RNP complex can be introduced into primary immune cells. Introduction of the RNP complex can be timed. The cell can be synchronized with other cells at GI, S, and/or M phases of the cell cycle. The RNP complex can be delivered at a cell phase such that HDR is enhanced. The RNP complex can facilitate homology directed repair.

A guide RNA can also be modified. The modifications can comprise chemical alterations, synthetic modifications, nucleotide additions, and/or nucleotide subtractions. The modifications can also enhance CRISPR genome engineering. A modification can alter chirality of a gRNA. In some cases, chirality may be uniform or stereopure after a modification. A guide RNA can be synthesized. The synthesized guide RNA can enhance CRISPR genome engineering. A guide RNA can also be truncated. Truncation can be used to reduce undesired off-target mutagenesis. The truncation can comprise any number of nucleotide deletions. For example, the truncation can comprise 1, 2, 3, 4, 5, 10, 15, 20, 25, 30, 40, 50 or more nucleotides. A guide RNA can comprise a region of target complementarity of any length. For example, a region of target complementarity can be less than 20 nucleotides in length. A region of target complementarity can be more than 20 nucleotides in length. A region of target complementarity can target from about 5 bp to about 20 bp directly adjacent to a PAM sequence. A region of target complementarity can target about 13 bp directly adjacent to a PAM sequence.

In some cases, a GUIDE-Seq analysis can be performed to determine the specificity of engineered guide RNAs. The general mechanism and protocol of GUIDE-Seq profiling of off-target cleavage by CRISPR system nucleases is discussed in Tsai, S. et al., “GUIDE-Seq enables genome-wide profiling of off-target cleavage by CRISPR system nucleases,” Nature, 33: 187-197 (2015).

A gRNA can be introduced at any functional concentration. For example, a gRNA can be introduced to a cell at 10 micrograms. In other cases, a gRNA can be introduced from 0.5 micrograms to 100 micrograms. A gRNA can be introduced from 0.5, 5, 10, 15, 20, 25, 30, 35, 40, 45, 50, 55, 60, 65, 70, 75, 80, 85, 90, 95, or 100 micrograms.

In some cases, a method can comprise a nuclease or an endonuclease selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof or modified versions thereof. A Cas protein can be Cas9. In some cases, a method can further comprise at least one guide RNA (gRNA). A gRNA can comprise at least one modification. An exogenous TCR can bind a cancer neo-antigen.

Disclosed herein is a method of making an engineered cell comprising: introducing at least one polynucleic acid encoding at least one exogenous T cell receptor (TCR) receptor sequence; introducing at least one guide RNA (gRNA) comprising at least one modification; and introducing at least one endonuclease; wherein the gRNA comprises at least one sequence complementary to at least one endogenous genome. In some cases, a modification is on a 5′ end, a 3′ end, from a 5′ end to a 3′ end, a single base modification, a 2′-ribose modification, or any combination thereof. A modification can be selected from a group consisting of base substitutions, insertions, deletions, chemical modifications, physical modifications, stabilization, purification, and any combination thereof.

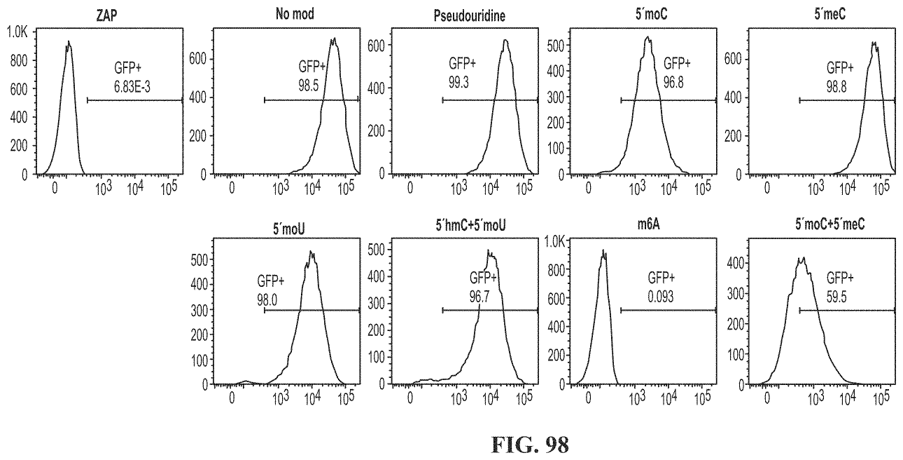

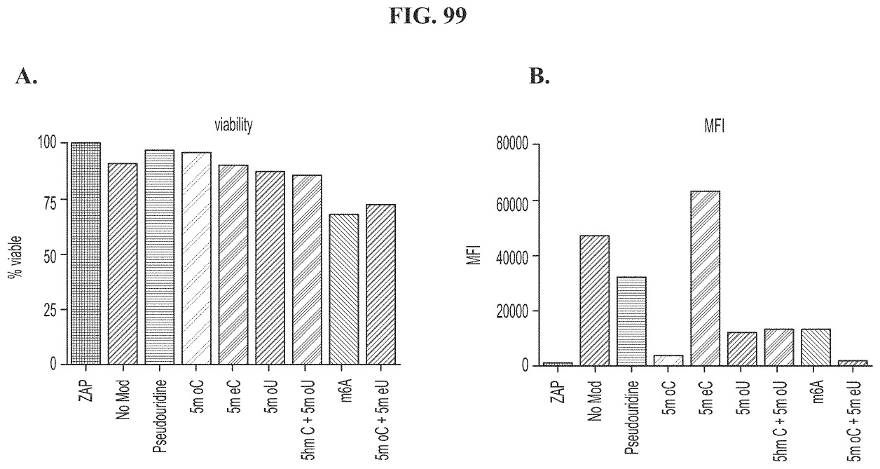

In some cases, a modification is a chemical modification. A modification can be selected from 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′ deoxyribonucleoside analog pyrimidine, ribonucleoside analog, 2′-0-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof. A modification can be a pseudouride modification as shown in FIG. 98. In some cases, a modification may not affect viability, FIG. 99 A and FIG. 99B.

In some cases, a modification is a 2-O-methyl 3 phosphorothioate addition. A 2-O-methyl 3 phosphorothioate addition can be performed from 1 base to 150 bases. A 2-O-methyl 3 phosphorothioate addition can be performed from 1 base to 4 bases. A 2-O-methyl 3 phosphorothioate addition can be performed on 2 bases. A 2-O-methyl 3 phosphorothioate addition can be performed on 4 bases. A modification can also be a truncation. A truncation can be a 5 base truncation.

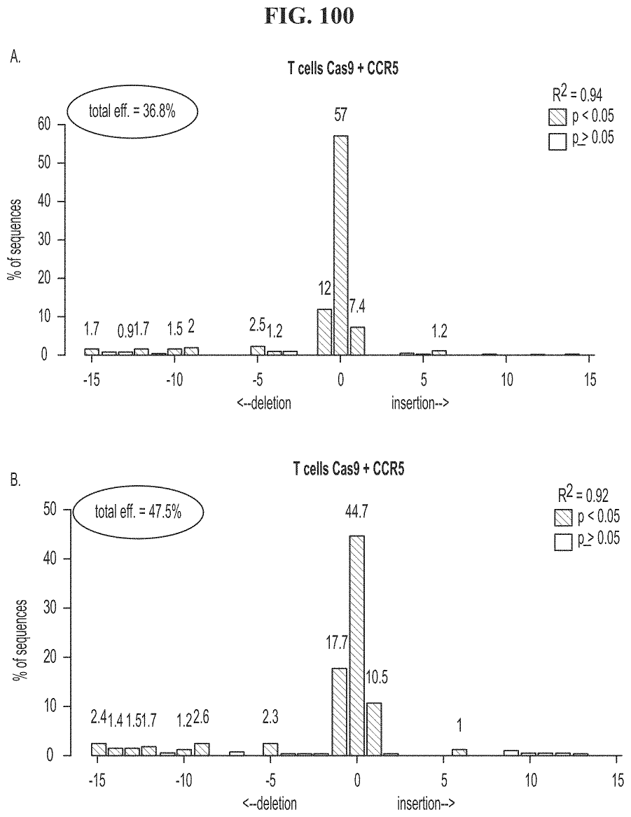

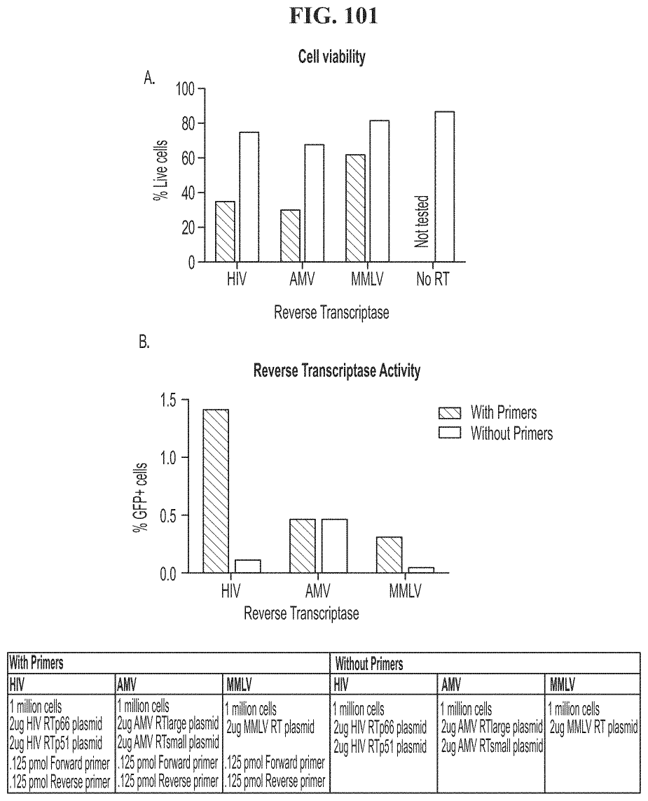

In some cases, a 5 base truncation can prevent a Cas protein from performing a cut. An endonuclease or a nuclease or a polypeptide encoding a nuclease can be selected from the group consisting of a CRISPR system, TALEN, Zinc Finger, transposon-based, ZEN, meganuclease, Mega-TAL, and any combination thereof. In some cases, an endonuclease or a nuclease or a polypeptide encoding a nuclease can be from a CRISPR system. An endonuclease or a nuclease or a polypeptide encoding a nuclease can be a Cas or a polypeptide encoding a Cas. In some cases, an endonuclease or a nuclease or a polypeptide encoding a nuclease can be selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof or modified versions thereof. A modified version of a Cas can be a clean Cas, as shown in FIGS. 100 A and B. A Cas protein can be Cas9. A Cas9 can create a double strand break in said at least one endogenous genome. In some cases, an endonuclease or a nuclease or a polypeptide encoding a nuclease can be Cas9 or a polypeptide encoding Cas9. In some cases, an endonuclease or a nuclease or a polypeptide encoding a nuclease can be catalytically dead. In some cases, an endonuclease or a nuclease or a polypeptide encoding a nuclease can be a catalytically dead Cas9 or a polypeptide encoding a catalytically dead Cas9. In some cases, an endogenous genome comprises at least one gene. A gene can be CISH, TCR, TRA, TRB, or a combination thereof. In some cases, a double strand break can be repaired using homology directed repair (HR), non-homologous end joining (NHEJ), microhomology-mediated end joining (MMEJ), or any combination or derivative thereof. A TCR can be integrated into a double strand break.

c. Transgene

Insertion of a transgene (e.g., exogenous sequence) can be used, for example, for expression of a polypeptide, correction of a mutant gene or for increased expression of a wild-type gene. A transgene is typically not identical to the genomic sequence where it is placed. A donor transgene can contain a non-homologous sequence flanked by two regions of homology to allow for efficient HDR at the location of interest. Additionally, transgene sequences can comprise a vector molecule containing sequences that are not homologous to the region of interest in cellular chromatin. A transgene can contain several, discontinuous regions of homology to cellular chromatin. For example, for targeted insertion of sequences not normally present in a region of interest, a sequence can be present in a donor nucleic acid molecule and flanked by regions of homology to sequence in the region of interest.

A transgene polynucleic acid can be DNA or RNA, single-stranded or double-stranded and can be introduced into a cell in linear or circular form. A transgene sequence(s) can be contained within a DNA mini-circle, which may be introduced into the cell in circular or linear form. If introduced in linear form, the ends of a transgene sequence can be protected (e.g., from exonucleolytic degradation) by any method. For example, one or more dideoxynucleotide residues can be added to the 3′ terminus of a linear molecule and/or self-complementary oligonucleotides can be ligated to one or both ends. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A transgene can be flanked by recombination arms. In some instances, recombination arms can comprise complementary regions that target a transgene to a desired integration site. A transgene can also be integrated into a genomic region such that the insertion disrupts an endogenous gene. A transgene can be integrated by any method, e.g., non-recombination end joining and/or recombination directed repair. A transgene can also be integrated during a recombination event where a double strand break is repaired. A transgene can also be integrated with the use of a homologous recombination enhancer. For example, an enhancer can block non-homologous end joining so that homology directed repair is performed to repair a double strand break.

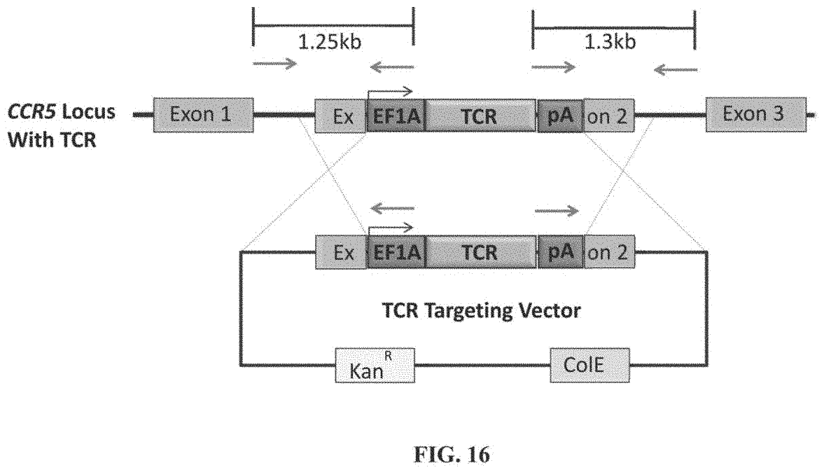

A transgene can be flanked by recombination arms where the degree of homology between the arm and its complementary sequence is sufficient to allow homologous recombination between the two. For example, the degree of homology between the arm and its complementary sequence can be 50% or greater. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, a representative transgene with recombination arms to CCR5 is shown in FIG. 16. Any other gene, e.g., the genes described herein, can be used to generate a recombination arm.

A transgene can be flanked by engineered sites that are complementary to the targeted double strand break region in a genome. In some cases, engineered sites are not recombination arms. Engineered sites can have homology to a double strand break region. Engineered sites can have homology to a gene. Engineered sites can have homology to a coding genomic region. Engineered sites can have homology to a non-coding genomic region. In some cases, a transgene can be excised from a polynucleic acid so it can be inserted at a double strand break region without homologous recombination. A transgene can integrate into a double strand break without homologous recombination.

A polynucleotide can be introduced into a cell as part of a vector molecule having additional sequences such as, for example, replication origins, promoters and genes encoding antibiotic resistance. Moreover, transgene polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)). A virus that can deliver a transgene can be an AAV virus.

A transgene is generally inserted so that its expression is driven by the endogenous promoter at the integration site, namely the promoter that drives expression of the endogenous gene into which a transgene is inserted (e.g., AAVS SITE (E.G. AAVS1, AAVS2, ETC.), CCR5, HPRT). A transgene may comprise a promoter and/or enhancer, for example a constitutive promoter or an inducible or tissue/cell specific promoter. A minicircle vector can encode a transgene.

Targeted insertion of non-coding nucleic acid sequence may also be achieved. Sequences encoding antisense RNAs, RNAi, shRNAs and micro RNAs (miRNAs) may also be used for targeted insertions.

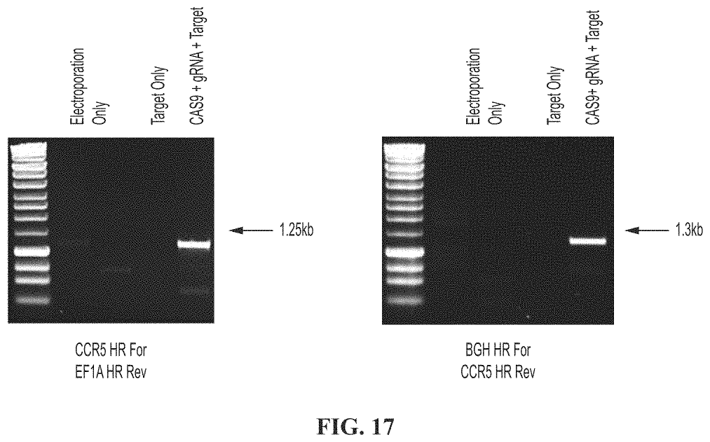

A transgene may be inserted into an endogenous gene such that all, some or none of the endogenous gene is expressed. For example, a transgene as described herein can be inserted into an endogenous locus such that some (N-terminal and/or C-terminal to a transgene) or none of the endogenous sequences are expressed, for example as a fusion with a transgene. In other cases, a transgene (e.g., with or without additional coding sequences such as for the endogenous gene) is integrated into any endogenous locus, for example a safe-harbor locus. For example, a TCR transgene can be inserted into an endogenous TCR gene. For example, FIG. 17, shows that a transgene can be inserted into an endogenous CCR5 gene. A transgene can be inserted into any gene, e.g., the genes as described herein.

When endogenous sequences (endogenous or part of a transgene) are expressed with a transgene, the endogenous sequences can be full-length sequences (wild-type or mutant) or partial sequences. The endogenous sequences can be functional. Non-limiting examples of the function of these full length or partial sequences include increasing the serum half-life of the polypeptide expressed by a transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In some cases, the exogenous sequence (e.g., transgene) comprises a fusion of a protein of interest and, as its fusion partner, an extracellular domain of a membrane protein, causing the fusion protein to be located on the surface of the cell. In some instances, a transgene encodes a TCR wherein a TCR encoding sequence is inserted into a safe harbor such that a TCR is expressed. In some instances, a TCR encoding sequence is inserted into a CISH and/or TCRlocus. In other cases, a TCR is delivered to the cell in a lentivirus for random insertion while the CISH and/or TCRspecific nucleases can be supplied as mRNAs. In some instances, a TCR is delivered via a viral vector system such as a retrovirus, AAV or adenovirus along with mRNA encoding nucleases specific for a safe harbor (e.g. AAVS site (e.g. AAVS1, AAVS2, etc.), CCR5, albumin or HPRT). The cells can also be treated with mRNAs encoding PD1 and/or CTLA-4 specific nucleases. In some cases, the polynucleotide encoding a TCR is supplied via a viral delivery system together with mRNA encoding HPRT specific nucleases and PD 1- or CTLA-4 specific nucleases. Cells comprising an integrated TCR-encoding nucleotide at the HPRT locus can be selected for using 6-thioguanine, a guanine analog that can result in cell arrest and/or initiate apoptosis in cells with an intact HPRT gene. TCRs that can be used with the methods and compositions of the present disclosure include all types of these chimeric proteins, including first, second and third generation designs. TCRs comprising specificity domains derived from antibodies can be particularly useful, although specificity domains derived from receptors, ligands and engineered polypeptides can be also envisioned by the present disclosure. The intercellular signaling domains can be derived from TCR chains such as zeta and other members of the CD3 complex such as the γ and E chains. In some cases, a TCRs may comprise additional co-stimulatory domains such as the intercellular domains from CD28, CD137 (also known as 4-1BB) or CD134. In still further cases, two types of co-stimulator domains may be used simultaneously (e.g., CD3 zeta used with CD28+CD137).

In some cases, the engineered cell can be a stem memory T_{SCM} cell comprised of CD45RO (−), CCR7 (+), CD45RA (+), CD62L+(L-selectin), CD27+, CD28+ and IL-7Rα+, stem memory cells can also express CD95, IL-2Rβ, CXCR3, and LFA-1, and show numerous functional attributes distinctive of stem memory cells. Engineered cells can also be central memory T_{CM} cells comprising L-selectin and CCR7, where the central memory cells can secrete, for example, IL-2, but not IFNγ or IL-4. Engineered cells can also be effector memory TEM cells comprising L-selectin or CCR7 and produce, for example, effector cytokines such as IFNγ and IL-4. In some cases a population of cells can be introduced to a subject. For example, a population of cells can be a combination of T cells and NK cells. In other cases, a population can be a combination of naïve cells and effector cells.

### Delivery of Homologous Recombination HR Enhancer

In some cases, a homologous recombination HR enhancer can be used to suppress non-homologous end joining (NHEJ). Non-homologous end joining can result in the loss of nucleotides at the end of double stranded breaks; non-homologous end joining can also result in frameshift. Therefore, homology-directed repair can be a more attractive mechanism to use when knocking in genes. To suppress non-homologous end-joining, a HR enhancer can be delivered. In some cases, more than one HR enhancer can be delivered. A HR enhancer can inhibit proteins involved in non-homologous end-joining, for example, KU70, KU80, and/or DNA Ligase IV. In some cases a Ligase IV inhibitor, such as Scr7, can be delivered. In some cases the HR enhancer can be L755507. In some cases, a different Ligase IV inhibitor can be used. In some cases, a HR enhancer can be an adenovirus 4 protein, for example, E1B55K and/or E4orf6. In some cases a chemical inhibitor can be used.

Non-homologous end joining molecules such as KU70, KU80, and/or DNA Ligase IV can be suppressed by using a variety of methods. For example, non-homologous end-joining molecules such as KU70, KU80, and/or DNA Ligase IV can be suppressed by gene silencing. For example, non-homologous end joining molecules KU70, KU80, and/or DNA Ligase IV can be suppressed by gene silencing during transcription or translation of factors. Non-homologous end joining molecules KU70, KU80, and/or DNA Ligase IV can also be suppressed by degradation of factors. Non-homologous end joining molecules KU70, KU80, and/or DNA Ligase IV can be also be inhibited. Inhibitors of KU70, KU80, and/or DNA Ligase IV can comprise E1B55K and/or E4orf6. Non-homologous end joining molecules KU70, KU80, and/or DNA Ligase IV can also be inhibited by sequestration. Gene expression can be suppressed by knock out, altering a promoter of a gene, and/or by administering interfering RNAs directed at the factors.

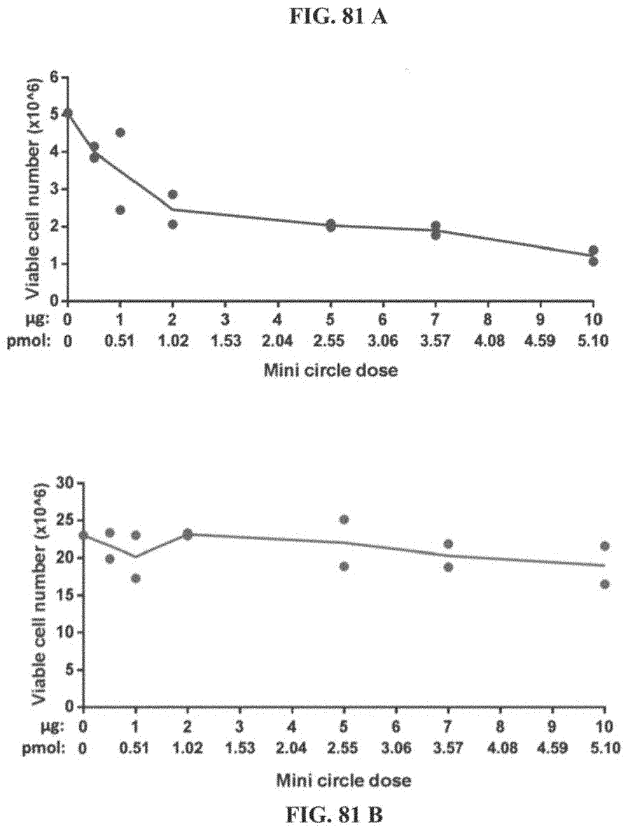

A HR enhancer that suppresses non-homologous end joining can be delivered with plasmid DNA. Sometimes, the plasmid can be a double stranded DNA molecule. The plasmid molecule can also be single stranded DNA. The plasmid can also carry at least one gene. The plasmid can also carry more than one gene. At least one plasmid can also be used. More than one plasmid can also be used. A HR enhancer that suppresses non-homologous end joining can be delivered with plasmid DNA in conjunction with CRISPR-Cas, primers, and/or a modifier compound. A modifier compound can reduce cellular toxicity of plasmid DNA and improve cellular viability. An HR enhancer and a modifier compound can be introduced to a cell before genomic engineering. The HR enhancer can be a small molecule. In some cases, the HR enhancer can be delivered to a T cell suspension. An HR enhancer can improve viability of cells transfected with double strand DNA. In some cases, introduction of double strand DNA can be toxic, FIG. 81 A. and FIG. 81 B.

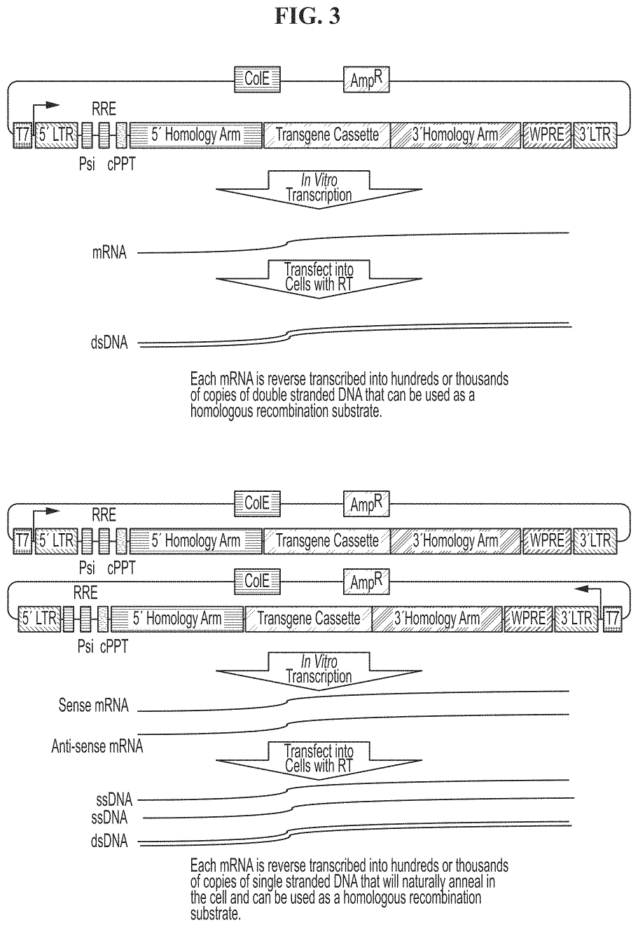

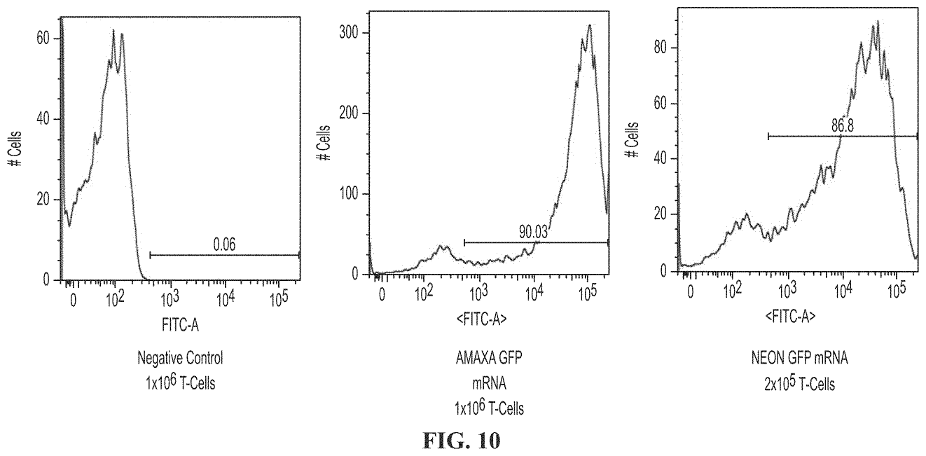

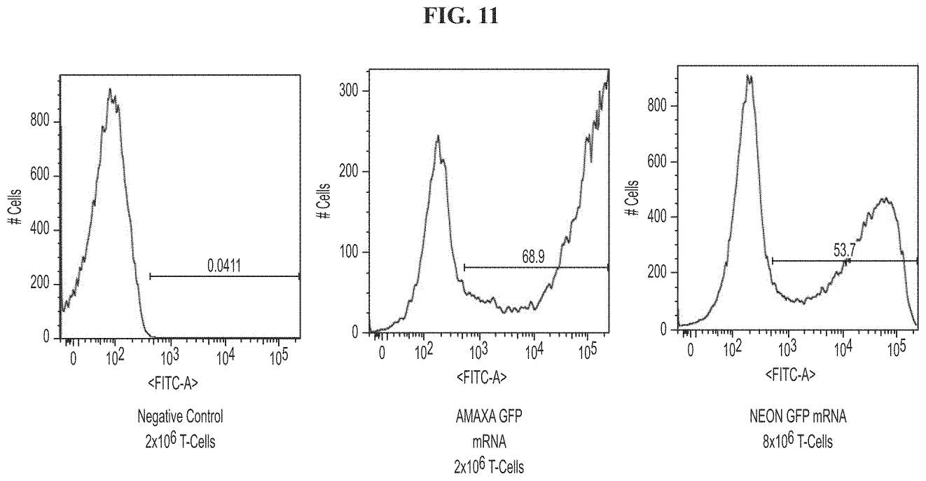

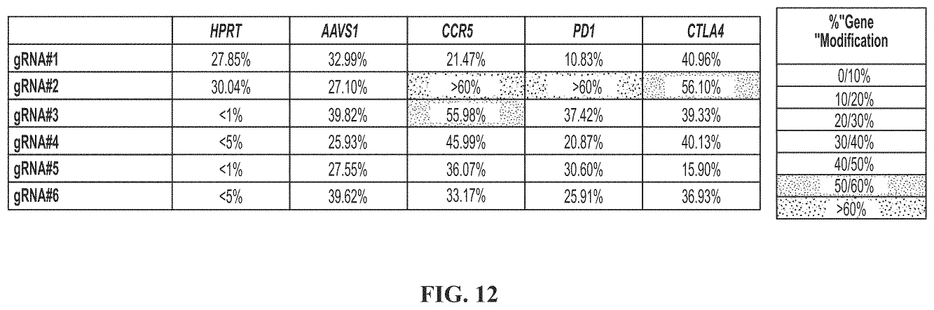

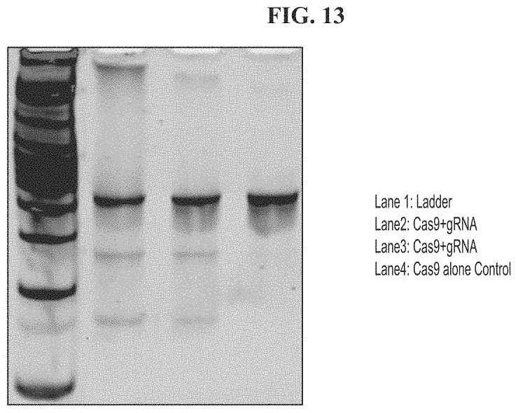

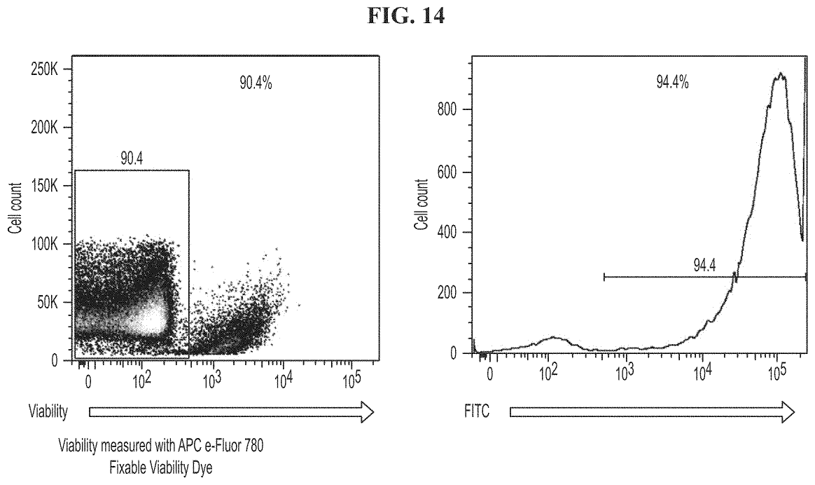

A HR enhancer that suppresses non-homologous end joining can be delivered with an HR substrate to be integrated. A substrate can be a polynucleic acid. A polynucleic acid can comprise a TCR transgene. A polynucleic acid can be delivered as mRNA (see FIG. 10 and FIG. 14). A polynucleic acid can comprise recombination arms to an endogenous region of the genome for integration of a TCR transgene. A polynucleic acid can be a vector. A vector can be inserted into another vector (e.g., viral vector) in either the sense or anti-sense orientation. Upstream of the 5′ LTR region of the viral genome a T7, T3, or other transcriptional start sequence can be placed for in vitro transcription of the viral cassette (see FIG. 3). This vector cassette can be then used as a template for in vitro transcription of mRNA. For example, when this mRNA is delivered to any cell with its cognate reverse transcription enzyme, delivered also as mRNA or protein, then the single stranded mRNA cassette can be used as a template to generate hundreds to thousands of copies in the form of double stranded DNA (dsDNA) that can be used as a HR substrate for the desired homologous recombination event to integrate a transgene cassette at an intended target site in the genome. This method can circumvent the need for delivery of toxic plasmid DNA for CRISPR mediated homologous recombination. Additionally, as each mRNA template can be made into hundreds or thousands of copies of dsDNA, the amount of homologous recombination template available within the cell can be very high. The high amount of homologous recombination template can drive the desired homologous recombination event. Further, the mRNA can also generate single stranded DNA. Single stranded DNA can also be used as a template for homologous recombination, for example with recombinant AAV (rAAV) gene targeting. mRNA can be reverse transcribed into a DNA homologous recombination HR enhancer in situ. This strategy can avoid the toxic delivery of plasmid DNA. Additionally, mRNA can amplify the homologous recombination substrate to a higher level than plasmid DNA and/or can improve the efficiency of homologous recombination.

A HR enhancer that suppresses non-homologous end joining can be delivered as a chemical inhibitor. For example, a HR enhancer can act by interfering with Ligase IV-DNA binding. A HR enhancer can also activate the intrinsic apoptotic pathway. A HR enhancer can also be a peptide mimetic of a Ligase IV inhibitor. A HR enhancer can also be co-expressed with the Cas9 system. A HR enhancer can also be co-expressed with viral proteins, such as E1B55K and/or E4orf6. A HR enhancer can also be SCR7, L755507, or any derivative thereof. A HR enhancer can be delivered with a compound that reduces toxicity of exogenous DNA insertion.

In the event that only robust reverse transcription of the single stranded DNA occurs in a cell, mRNAs encoding both the sense and anti-sense strand of the viral vector can be introduced (see FIG. 3). In this case, both mRNA strands can be reverse transcribed within the cell and/or naturally anneal to generate dsDNA.

The HR enhancer can be delivered to primary cells. A homologous recombination HR enhancer can be delivered by any suitable means. A homologous recombination HR enhancer can also be delivered as an mRNA. A homologous recombination HR enhancer can also be delivered as plasmid DNA. A homologous recombination HR enhancer can also be delivered to immune cells in conjunction with CRISPR-Cas. A homologous recombination HR enhancer can also be delivered to immune cells in conjunction with CRISPR-Cas, a polynucleic acid comprising a TCR sequence, and/or a compound that reduces toxicity of exogenous DNA insertion.

A homologous recombination HR enhancer can be delivered to any cells, e.g., to immune cells. For instance, a homologous recombination HR enhancer can be delivered to a primary immune cell. A homologous recombination HR enhancer can also be delivered to a T cell, including but not limited to T cell lines and to a primary T cell. A homologous recombination HR enhancer can also be delivered to a CD4+ cell, a CD8+ cell, and/or a tumor infiltrating cell (TIL). A homologous recombination HR enhancer can also be delivered to immune cells in conjunction with CRISPR-Cas.

In some cases, a homologous recombination HR enhancer can be used to suppress non-homologous end-joining. In some cases, a homologous recombination HR enhancer can be used to promote homologous directed repair. In some cases, a homologous recombination HR enhancer can be used to promote homologous directed repair after a CRISPR-Cas double stranded break. In some cases, a homologous recombination HR enhancer can be used to promote homologous directed repair after a CRISPR-Cas double stranded break and the knock-in and knock-out of one of more genes. The genes that are knocked-in can be a TCR. The genes that are knocked-out can also be any number of endogenous checkpoint genes. For example, the endogenous checkpoint gene can be selected from the group consisting of A2AR, B7-H3, B7-H4, BTLA, CTLA-4, IDO, KIR, LAG3, PD-1, TIM-3, VISTA, AAVS SITE (E.G. AAVS1, AAVS2, ETC.), CCR5, HPRT, PPP1R12C, TCR, and/or CISH. In some cases, the gene can be CISH. In some cases, the gene can be TCR. In some cases, the gene can be an endogenous TCT. In some cases, the gene can comprise a coding region. In some cases, the gene can comprise a non-coding region.

Increase in HR efficiency with an HR enhancer can be or can be about 10%, 20%, 30%, 40%, 50%, 60%, 70%, 80%, 90%, or 100%.

Decrease in NHEJ with an HR enhancer can be or can be about 10%, 20%, 30%, 40%, 50%, 60%, 70%, 80%, 90%, or 100%.

### Low Toxicity Engineering of Cells

Cellular toxicity to exogenous polynucleic acids can be mitigated to improve the engineering of cell, including T cells. For example, cellular toxicity can be reduced by altering a cellular response to polynucleic acid.

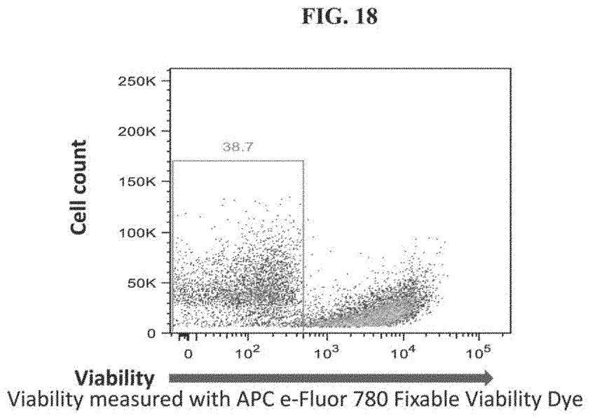

A polynucleic acid can contact a cell. The polynucleic acids can then be introduced into a cell. In some cases, a polynucleic acid is utilized to alter a genome of a cell. After insertion of the polynucleic acid, the cell can die. For example, insertion of a polynucleic acid can cause apoptosis of a cell as shown in FIG. 18. Toxicity induced by a polynucleic acid can be reduced by using a modifier compound.

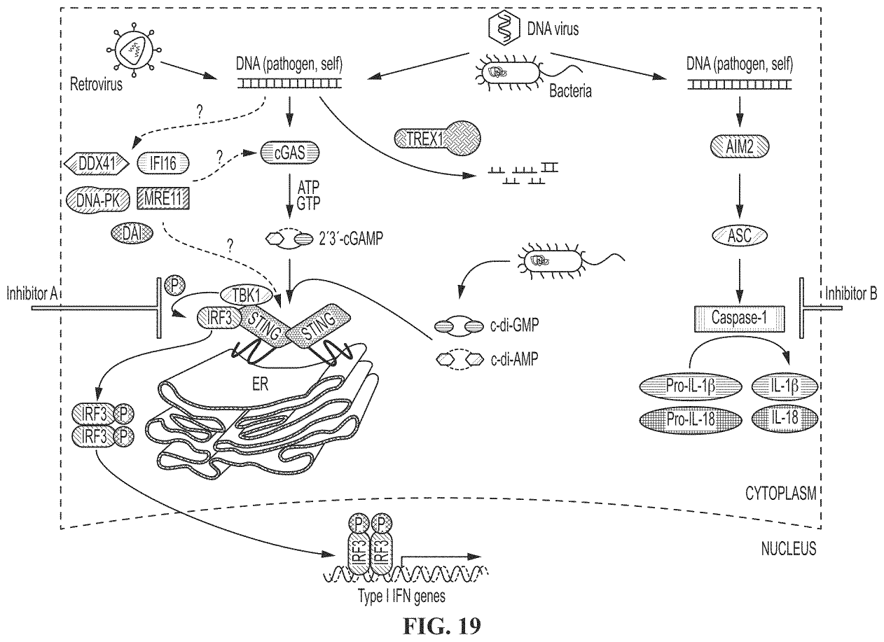

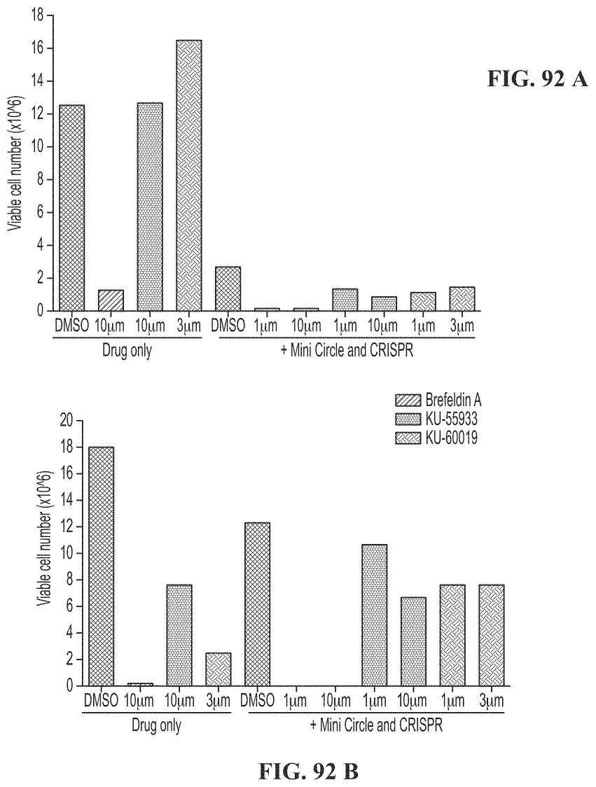

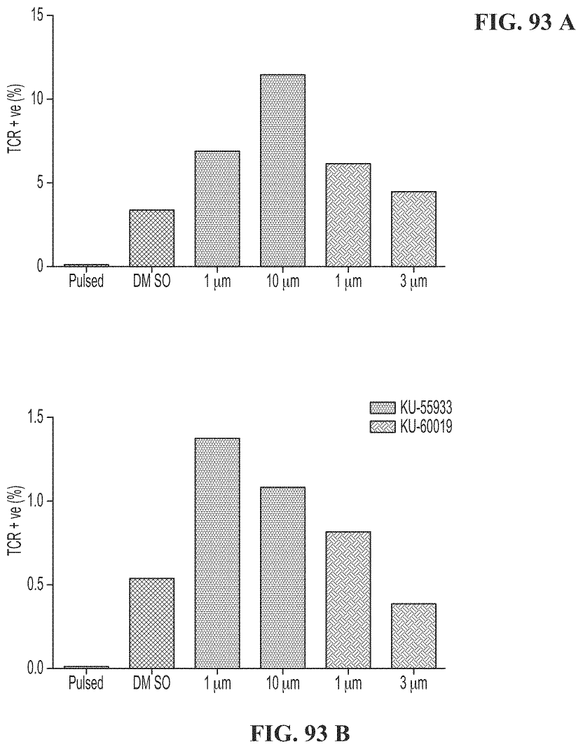

For example, a modifier compound can disrupt an immune sensing response of a cell. A modifier compound can also reduce cellular apoptosis and pyropoptosis. Depending on the situation, a modifier compound can be an activator or an inhibitor. The modifier compound can act on any component of the pathways shown in FIG. 19. For example, the modifier compound can act on Caspase-1, TBK1, IRF3, STING, DDX41, DNA-PK, DAI, IFI16, MRE11, cGAS, 2′3′-cGAMP, TREX1, AIM2, ASC, or any combination thereof. A modifier can be a TBK1 modifier. A modifier can be a caspase-1 modifier. A modifier compound can also act on the innate signaling system, thus, it can be an innate signaling modifier. In some cases, exogenous nucleic acids can be toxic to cells. A method that inhibits an innate immune sensing response of cells can improve cell viability of engineered cellular products. A modifying compound can be brefeldin A and or an inhibitor of an ATM pathway, FIG. 92A, FIG. 92B, FIG. 93A and FIG. 93B.

Reducing toxicity to exogenous polynucleic acids can be performed by contacting a compound and a cell. In some cases, a cell can be pre-treated with a compound prior to contact with a polynucleic acid. In some cases, a compound and a polynucleic acid are simultaneously introduced (e.g., concurrently introduced) to a cell. A modifying compound can be comprised within a polynucleic acid. In some cases, a polynucleic acid comprises a modifying compound. In some cases, a compound can be introduced as a cocktail comprising a polynucleic acid, an HR enhancer, and/or CRISPR-Cas. The compositions and methods as disclosed herein can provide an efficient and low toxicity method by which cell therapy, e.g., a cancer specific cellular therapy, can be produced.

A compound that can be used in the methods and/or systems and/or compositions described herein, can have one or more of the following characteristics and can have one or more of the function described herein. Despite its one or more functions, a compound described herein can decrease toxicity of exogenous polynucleotides. For example, a compound can modulate a pathway that results in toxicity from exogenously introduced polynucleic acid. In some cases, a polynucleic acid can be DNA. A polynucleic acid can also be RNA. A polynucleic acid can be single strand. A polynucleic acid can also be double strand. A polynucleic acid can be a vector. A polynucleic acid can also be a naked polynucleic acid. A polynucleic acid can encode for a protein. A polynucleic acid can also have any number of modifications. A polynucleic acid modification can be demethylation, addition of CpG methylation, removal of bacterial methylation, and/or addition of mammalian methylation. A polynucleic acid can also be introduced to a cell as a reagent cocktail comprising additional polynucleic acids, any number of HR enhancers, and/or CRISPR-Cas. A polynucleic acid can also comprise a transgene. A polynucleic acid can comprise a transgene that as a TCR sequence.

A compound can also modulate a pathway involved in initiating toxicity to exogenous DNA. A pathway can contain any number of factors. For example, a factor can comprise DNA-dependent activator of IFN regulatory factors (DAI), IFN inducible protein 16 (IFI16), DEAD box polypeptide 41 (DDX41), absent in melanoma 2 (AIM2), DNA-dependent protein kinase, cyclic guanosine monophosphate-adenosine monophosphate synthase (cGAS), stimulator of IFN genes (STING), TANK-binding kinase (TBK1), interleukin-1β (IL-1β), MRE11, meiotic recombination 11, Trex1, cysteine protease with aspartate specificity (Caspase-1), three prime repair exonuclease, DNA-dependent activator of IRFs (DAI), IFI16, DDX41, DNA-dependent protein kinase (DNA-PK), meiotic recombination 11 homolog A (MRE11), and IFN regulatory factor (IRF) 3 and 7, and/or any derivative thereof.

In some cases, a DNA sensing pathway may generally refer to any cellular signaling pathway that comprises one or more proteins (e.g., DNA sensing proteins) involved in the detection of intracellular nucleic acids, and in some instances, exogenous nucleic acids. In some cases, a DNA sensing pathway may comprise stimulator of interferon (STING). In some cases, a DNA sensing pathway may comprise the DNA-dependent activator of IFN-regulatory factor (DAI). Non-limiting examples of a DNA sensing protein include three prime repair exonuclease 1 (TREX1), DEAD-box helicase 41 (DDX41), DNA-dependent activator of IFN-regulatory factor (DAI), Z-DNA-binding protein 1 (ZBP1), interferon gamma inducible protein 16 (IFI16), leucine rich repeat (In FLII) interacting protein 1 (LRRFIP1), DEAH-box helicase 9 (DHX9), DEAH-box helicase 36 (DHX36), Lupus Ku autoantigen protein p70 (Ku70), X-ray repair complementing defective repair in chinese hamster cells 6 (XRCC6), stimulator of interferon gene (STING), transmembrane protein 173 (TMEM173), tripartite motif containing 32 (TRIM32), tripartite motif containing 56 (TRIM56), β-catenin (CTNNB1), myeloid differentiation primary response 88 (MyD88), absent in melanoma 2 (AIM2), apoptosis-associated speck-like protein containing a CARD (ASC), pro-caspase-1 (pro-CASP1), caspase-1 (CASP1), pro-interleukin 1 beta (pro-IL-1β), pro-interleukin 18 (pro-IL-18), interleukin 1 beta (IL-1β), interleukin 18 (IL-18), interferon regulatory factor 1 (IRF1), interferon regulatory Factor 3 (IRF3), interferon regulatory factor 7 (IRF7), interferon-stimulated response element 7 (ISRE7), interferon-stimulated response element 1/7 (ISRE1/7), nuclear factor kappa B (NF-κB), RNA polymerase III (RNA Pol III), melanoma differentiation-associated protein 5 (MDA-5), Laboratory of Genetics and Physiology 2 (LGP2), retinoic acid-inducible gene 1 (RIG-I), mitochondrial antiviral-signaling protein (IPS-1), TNF receptor associated factor 3 (TRAF3), TRAF family member associated NFKB activator (TANK), nucleosome assembly protein 1 (NAP1), TANK binding kinase 1 (TBK1), autophagy related 9A (Atg9a), tumor necrosis factor alpha (TNF-α), interferon lambda-1 (IFNλ1), cyclic GMP-AMP Synthase (cGAS), AMP, GMP, cyclic GMP-AMP (cGAMP), a phosphorylated form of a protein thereof, or any combination or derivative thereof. In one example of a DNA sensing pathway, DAI activates the IRF and NF-κB transcription factors, leading to production of type I interferon and other cytokines. In another example of a DNA sensing pathway, upon sensing exogenous intracellular DNA, AIM2 triggers the assembly of the inflammasome, culminating in interleukin maturation and pyroptosis. In yet another example of a DNA sensing pathway, RNA PolIII may convert exogenous DNA into RNA for recognition by the RNA sensor RIG-I.

In some aspects, the methods of the present disclosure comprise introducing into one or more cells a nucleic acid comprising a first transgene encoding at least one anti-DNA sensing protein.

An anti-DNA sensing protein may generally refer to any protein that alters the activity or expression level of a protein corresponding to a DNA sensing pathway (e.g., a DNA sensing protein). In some cases, an anti-DNA sensing protein may degrade (e.g., reduce overall protein level) of one or more DNA sensing proteins. In some cases, an anti-DNA sensing protein may fully inhibit one or more DNA sensing proteins. In some cases, an anti-DNA sensing protein may partially inhibit one or more DNA sensing proteins. In some cases, an anti-DNA sensing protein may inhibit the activity of at least one DNA sensing protein by at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, or at least about 5%. In some cases, an anti-DNA sensing protein may decrease the amount of at least one DNA sensing protein by at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, or at least about 5%.

Cell viability may be increased by introducing viral proteins during a genomic engineering procedure, which can inhibit the cells ability to detect exogenous DNA. In some cases, an anti-DNA sensing protein may promote the translation (e.g., increase overall protein level) of one or more DNA sensing proteins. In some cases, an anti-DNA sensing protein may protect or increase the activity of one or more DNA sensing proteins. In some cases, an anti-DNA sensing protein may increase the activity of at least one DNA sensing protein by at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, or at least about 5%. In some cases, an anti-DNA sensing protein may increase the amount of at least one DNA sensing protein by at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, or at least about 5%. In some cases, an anti-DNA sensing inhibitor may be a competitive inhibitor or activator of one or more DNA sensing proteins. In some cases, an anti-DNA sensing protein may be a non-competitive inhibitor or activator of a DNA sensing protein.

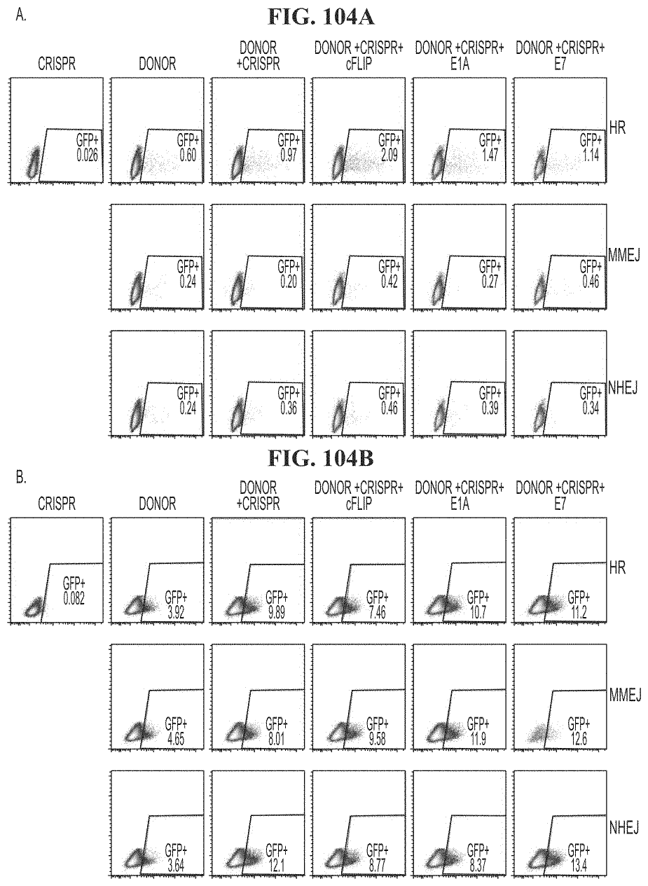

In some cases of the present disclosure, an anti-DNA sensing protein may also be a DNA sensing protein (e.g., TREX1). Non-limiting examples of anti-DNA sensing proteins include cellular FLICE-inhibitory protein (c-FLiP), Human cytomegalovirus tegument protein (HCMV pUL83), dengue virus specific NS2B-NS3 (DENV NS2B-NS3), Protein E7-Human papillomavirus type 18 (HPV18 E7), hAd5 E1A, Herpes simplex virus immediate-early protein ICP0 (HSV1 ICP0), Vaccinia virus B13 (VACV B13), Vaccinia virus C16 (VACV C16), three prime repair exonuclease 1 (TREX1), human coronavirus NL63 (HCoV-NL63), severe acute respiratory syndrome coronavirus (SARS-CoV), hepatitis B virus DNA polymerase (HBV Pol), porcine epidemic diarrhea virus (PEDV), adenosine deaminase (ADAR1), E3L, p202, a phosphorylated form of a protein thereof, and any combination or derivative thereof. In some cases, HCMV pUL83 may disrupt a DNA sensing pathway by inhibiting activation of the STING-TBK1-IRF3 pathway by interacting with the pyrin domain on IFI16 (e.g., nuclear IFI16) and blocking its oligomerization and subsequent downstream activation. In some cases, DENV Ns2B-NS3 may disrupt a DNA sensing pathway by degrading STING. In some cases, HPV18 E7 may disrupt a DNA sensing pathway by blocking the cGAS/STING pathway signaling by binding to STING. In some cases, hAd5 E1A may disrupt a DNA sensing pathway by blocking the cGAS/STING pathway signaling by binding to STING. For example, FIG. 104 A and FIG. 104B show cells transfected with a CRISPR system, an exogenous polynucleic acid, and a hAd5 E1A or HPV18 E7 protein. In some cases, HSV1 ICP0 may disrupt a DNA sensing pathway by degradation of IFI16 and/or delaying recruitment of IFI16 to the viral genome. In some cases, VACV B13 may disrupt a DNA sensing pathway by blocking Caspase 1-dependant inflammasome activation and Caspase 8-dependent extrinsic apoptosis. In some cases, VACV C16 may disrupt a DNA sensing pathway by blocking innate immune responses to DNA, leading to decreased cytokine expression.

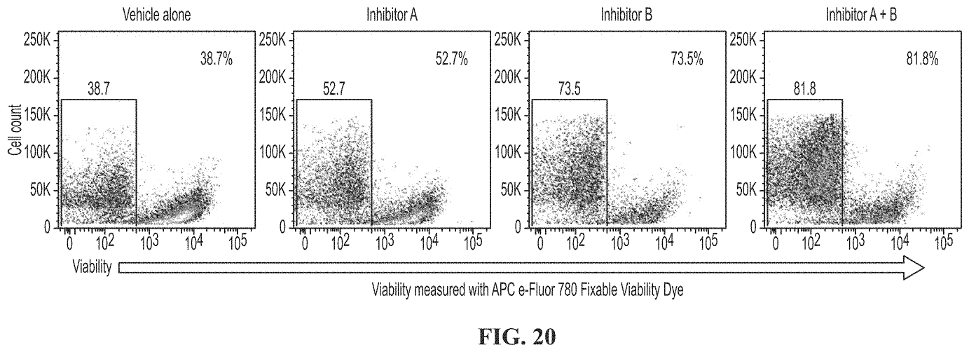

A compound can be an inhibitor. A compound can also be an activator. A compound can be combined with a second compound. A compound can also be combined with at least one compound. In some cases, one or more compounds can behave synergistically. For example, one or more compounds can reduce cellular toxicity when introduced to a cell at once as shown in FIG. 20.

A compound can be Pan Caspase Inhibitor Z-VAD-FMK and/or Z-VAD-FMK. A compound can be a derivative of any number of known compounds that modulate a pathway involved in initiating toxicity to exogenous DNA. A compound can also be modified. A compound can be modified by any number of means, for example, a modification to a compound can comprise deuteration, lipidization, glycosylation, alkylation, PEGylation, oxidation, phosphorylation, sulfation, amidation, biotinylation, citrullination, isomerization, ubiquitylation, protonation, small molecule conjugations, reduction, dephosphorylation, nitrosylation, and/or proteolysis. A modification can also be post-translational. A modification can be pre-translation. A modification can occur at distinct amino acid side chains or peptide linkages and can be mediated by enzymatic activity.

A modification can occur at any step in the synthesis of a compound. For example, in proteins, many compounds are modified shortly after translation is ongoing or completed to mediate proper compound folding or stability or to direct the nascent compound to distinct cellular compartments. Other modifications occur after folding and localization are completed to activate or inactivate catalytic activity or to otherwise influence the biological activity of the compound. Compounds can also be covalently linked to tags that target a compound for degradation. Besides single modifications, compounds are often modified through a combination of post-translational cleavage and the addition of functional groups through a step-wise mechanism of compound maturation or activation.

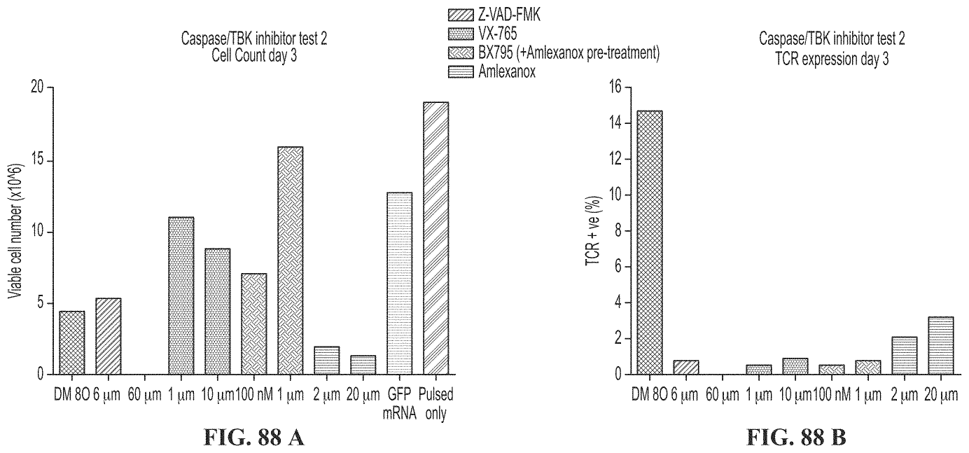

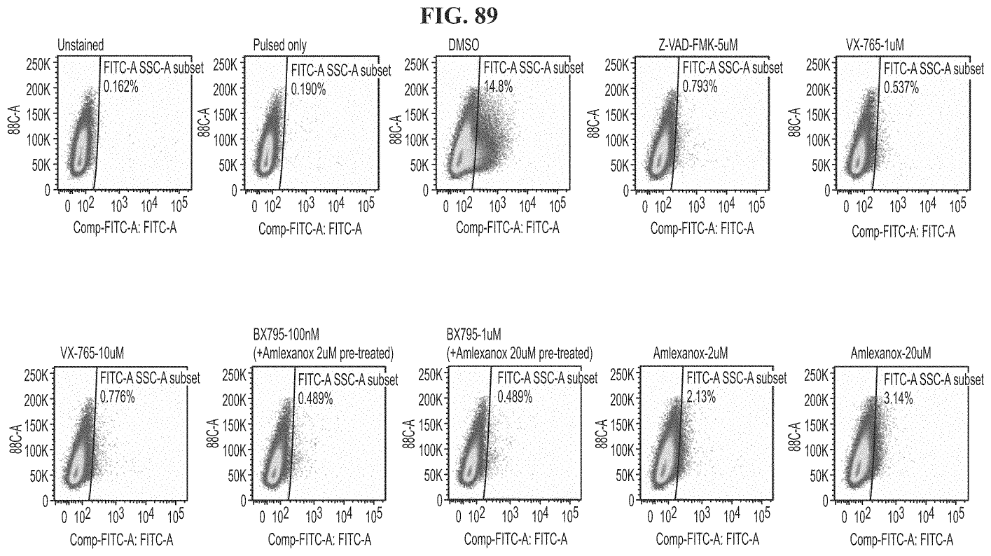

A compound can reduce production of type I interferons (IFNs), for example, IFN-α, and/or IFN-β. A compound can also reduce production of proinflammatory cytokines such as tumor necrosis factor-α (TNF-α) and/or interleukin-1β (IL-1β). A compound can also modulate induction of antiviral genes through the modulation of the Janus kinase (JAK)-signal transducer and activator of transcription (STAT) pathway. A compound can also modulate transcription factors nuclear factor κ-light-chain enhancer of activated B cells (NF-κB), and the IFN regulatory factors IRF3 and IRF7. A compound can also modulate activation of NF-κB, for example modifying phosphorylation of IκB by the IκB kinase (IKK) complex. A compound can also modulate phosphorylation or prevent phosphorylation of IκB. A compound can also modulate activation of IRF3 and/or IRF7. For example, a compound can modulate activation of IRF3 and/or IRF7. A compound can activate TBK1 and/or IKKε. A compound can also inhibit TBK1 and/or IKKε. A compound can prevent formation of an enhanceosome complex comprised of IRF3, IRF7, NF-κB and other transcription factors to turn on the transcription of type I IFN genes. A modifying compound can be a TBK1 compound and at least one additional compound, FIG. 88 A and FIG. 88. B. In some cases, a TBK1 compound and a Caspase inhibitor compound can be used to reduce toxicity of double strand DNA, FIG. 89.

A compound can prevent cellular apoptosis and/or pyropoptosis. A compound can also prevent activation of an inflammasome. An inflammasome can be an intracellular multiprotein complex that mediates the activation of the proteolytic enzyme caspase-1 and the maturation of IL-1β. A compound can also modulate AIM2 (absent in melanoma 2). For example, a compound can prevent AIM2 from associating with the adaptor protein ASC (apoptosis-associated speck-like protein containing a CARD). A compound can also modulate a homotypic PYD:PYD interaction. A compound can also modulate a homotypic CARD: CARD interaction. A compound can modulate Caspase-1. For example, a compound can inhibit a process whereby Caspase-1 converts the inactive precursors of IL-1β and IL-18 into mature cytokines.

A compound can be a component of a platform to generate a GMP compatible cellular therapy. A compound can used to improve cellular therapy. A compound can be used as a reagent. A compound can be combined as a combination therapy. A compound can be utilized ex vivo. A compound can be used for immunotherapy. A compound can be a part of a process that generates a T cell therapy for a patient in need, thereof.

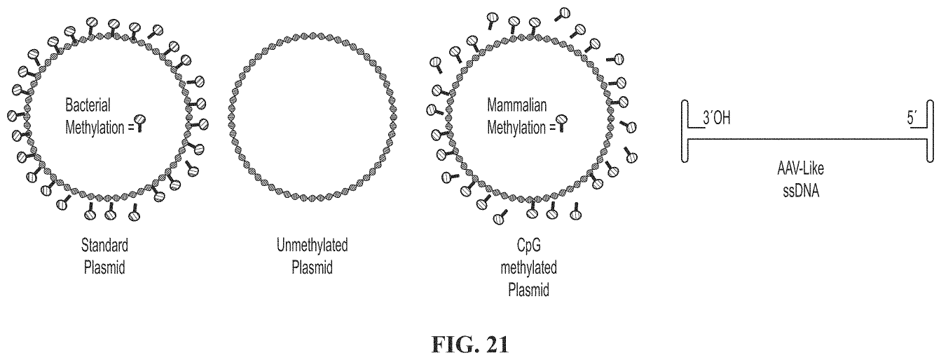

In some cases, a compound is not used to reduce toxicity. In some cases, a polynucleic acid can be modified to also reduce toxicity. For example, a polynucleic acid can be modified to reduce detection of a polynucleic acid, e.g., an exogenous polynucleic acid. A polynucleic acid can also be modified to reduce cellular toxicity. For example, a polynucleic acid can be modified by one or more of the methods depicted in FIG. 21. A polynucleic acid can also be modified in vitro or in vivo.

A compound or modifier compound can reduce cellular toxicity of plasmid DNA by or by about 10%, 20%, 30%, 40%, 50%, 60%, 70%, 80%, 90%, or 100%. A modifier compound can improve cellular viability by or by about 10%, 20%, 30%, 40%, 50%, 60%, 70%, 80%, 90%, or 100%.

Unmethylated polynucleic acid can also reduce toxicity. For example, an unmethylated polynucleic acid comprising at least one engineered antigen receptor flanked by at least two recombination arms complementary to at least one genomic region can be used to reduce cellular toxicity. The polynucleic acid can also be naked polynucleic acids. The polynucleic acids can also have mammalian methylation, which in some cases will reduce toxicity as well. In some cases, a polynucleic acid can also be modified so that bacterial methylation is removed and mammalian methylation is introduced. Any of the modifications described herein can apply to any of the polynucleic acids as described herein.

Polynucleic acid modifications can comprise demethylation, addition of CpG methylation, removal of bacterial methylation, and/or addition of mammalian methylation. A modification can be converting a double strand polynucleic acid into a single strand polynucleic acid. A single strand polynucleic acid can also be converted into a double strand polynucleic acid.

A polynucleic acid can be methylated (e.g. Human methylation) to reduce cellular toxicity. The modified polynucleic acid can comprise a TCR sequence or chimeric antigen receptor (CAR). The polynucleic acid can also comprise an engineered extracellular receptor.

Mammalian methylated polynucleic acid comprising at least one engineered antigen receptor can be used to reduce cellular toxicity. A polynucleic acid can be modified to comprise mammalian methylation. A polynucleic acid can be methylated with mammalian methylation so that it is not recognized as foreign by a cell.

Polynucleic acid modifications can also be performed as part of a culturing process. Demethylated polynucleic acid can be produced with genomically modified bacterial cultures that do not introduce bacterial methylation. These polynucleic acids can later be modified to contain mammalian methylation, e.g., human methylation.

Toxicity can also be reduced by introducing viral proteins during a genomic engineering procedure. For example, viral proteins can be used to block DNA sensing and reduce toxicity of a donor nucleic acid encoding for an exogenous TCR or CRISPR system. An evasion strategy employed by a virus to block DNA sensing can be sequestration or modification of a viral nucleic acid; interference with specific post-translational modifications of PRRs or their adaptor proteins; degradation or cleavage of pattern recognition receptors (PRRs) or their adaptor proteins; sequestration or relocalization of PRRs, or any combination thereof. In some cases, a viral protein may be introduced that can block DNA sensing by any of the evasion strategies employed by a virus.

In some cases, a viral protein can be or can be derived from a virus such as Human cytomegalovirus (HCMV), Dengue virus (DENV), Human Papillomavirus Virus (HPV), Herpes Simplex Virus type 1 (HSV1), Vaccinia Virus (VACV), Human coronaviruses (HCoVs), Severe acute respiratory syndrome (SARS) corona virus (SARS-Cov), Hepatitis B virus, Porcine epidemic diarrhea virus, or any combination thereof.

An introduced viral protein can prevent RIG-I-like receptors (RLRs) from accessing viral RNA by inducing formation of specific replication compartments that can be confined by cellular membranes, or in other cases to replicate on organelles, such as an endoplasmic reticulum, a Golgi apparatus, mitochondria, or any combination thereof. For example, a virus of the present disclosure can have modifications that prevent detection or hinder the activation of RLRs. In other cases, an RLR signaling pathway can be inhibited. For example, a Lys63-linked ubiquitylation of RIG-I can be inhibited or blocked to prevent activation of RIG-I signaling. In other cases, a viral protein can target a cellular E3 ubiquitin ligase that can be responsible for ubiquitylation of RIG-I. A viral protein can also remove a ubiquitylation of RIG-I. Furthermore, viruses can inhibit a ubiquitylation (e.g., Lys63-linked) of RIG-I independent of protein-protein interactions, by modulating the abundance of cellular microRNAs or through RNA-protein interactions.

In some cases, to prevent activation of RIG-I, viral proteins can process a 5′-triphosphate moiety in the viral RNA, or viral nucleases can digest free double-stranded RNA (dsRNA). Furthermore, viral proteins, can bind to viral RNA to inhibit the recognition of pathogen-associated molecular patterns (PAMPs) by RIG-I. Some viral proteins can manipulate specific post-translational modifications of RIG-I and/or MDA5, thereby blocking their signaling abilities. For example, viruses can prevent the Lys63-linked ubiquitylation of RIG-I by encoding viral deubiquitylating enzymes (DUBs). In other cases, a viral protein can antagonize a cellular E3 ubiquitin ligase, tripartite motif protein 25 (TRIM25) and/or Riplet, thereby also inhibiting RIG-I ubiquitylation and thus its activation. Furthermore, in other cases a viral protein can bind to TRIM25 to block sustained RIG-I signaling. To suppress the activation of MDA5, a viral protein can prevent a PP1α-mediated or PP1γ-mediated dephosphorylation of MDA5, keeping it in its phosphorylated inactive state. For example, a Middle East respiratory syndrome coronavirus (MERS-CoV) can target protein kinase R activator (PACT) to antagonize RIG-I. An NS3 protein from DENV virus can target the trafficking factor 14-3-3E to prevent translocation of RIG-I to MAVS at the mitochondria. In some cases, a viral protein can cleave RIG-I, MDA5 and/or MAVS. Other viral proteins can be introduced to subvert cellular degradation pathways to inhibit RLR-MAVS-dependent signaling. For example, an X protein from hepatitis B virus (HBV) and the 9b protein from severe acute respiratory syndrome (SARS)-associated coronavirus (SARS-CoV) can promote the ubiquitylation and degradation of MAVS.

In some cases, an introduced viral protein can allow for immune evasion of cGAS, IFI16, STING, or any combination thereof. For example, to prevent activation of cyclic GMP-AMP synthase (cGAS), a viral protein can use the cellular 3′-repair exonuclease 1 (TREX1) to degrade excess reverse transcribed viral DNA. In addition, the a viral capsid can recruit host-encoded factors, such as cyclophilin A (CYPA), which can prevent the sensing of reverse transcribed DNA by cGAS. Furthermore, an introduced viral protein can bind to both viral DNA and cGAS to inhibit the activity of cGAS. In other cases, to antagonize the activation of stimulator of interferon (IFN) genes (STING), the polymerase (Pol) of hepatitis B virus (HBV) and the papain-like proteases (PLPs) of human coronavirus NL63 (HCoV-NL63), severe acute respiratory syndrome (SARS)-associated coronavirus (SARS-CoV) for example, can prevent or remove the Lys63-linked ubiquitylation of STING. An introduced viral protein can also bind to STING and inhibit its activation or cleave STING to inactivate it. In some cases, IFI16 can be inactivated. For example, a viral protein can target IFI16 for proteasomal degradation or bind to IFI16 to prevent its oligomerization and thus its activation.

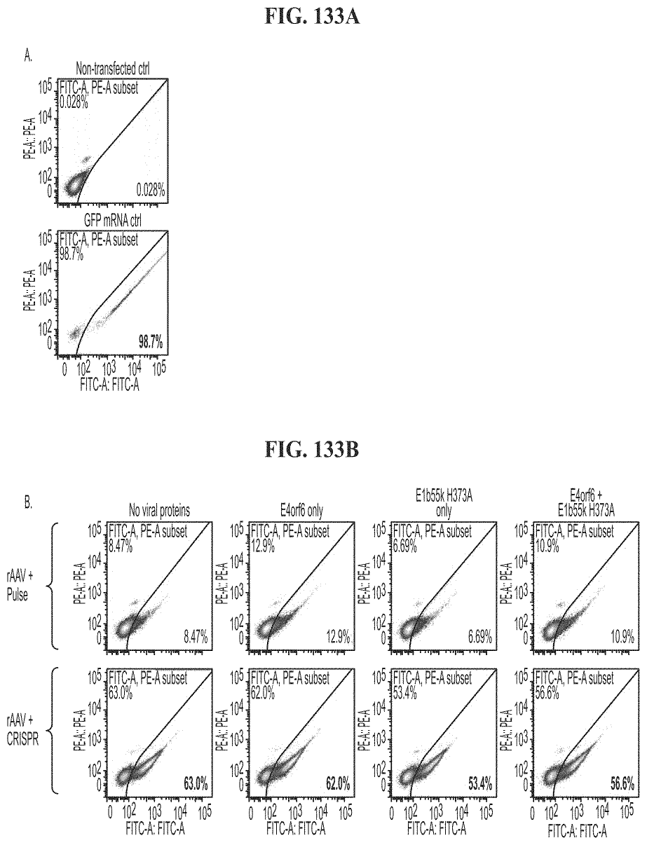

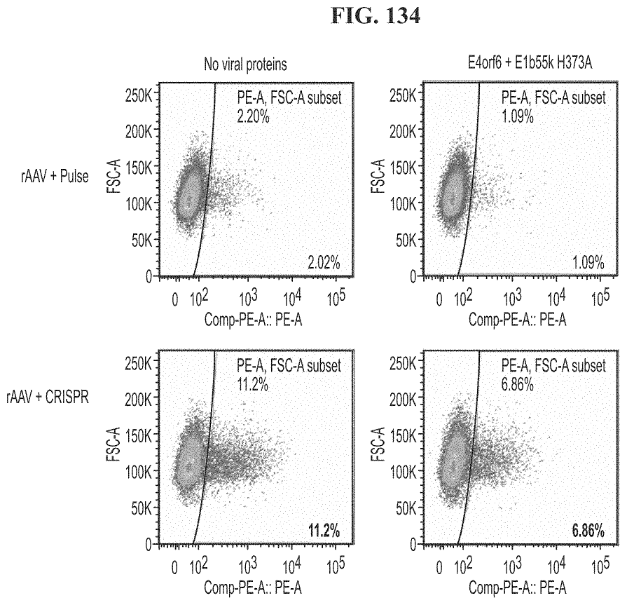

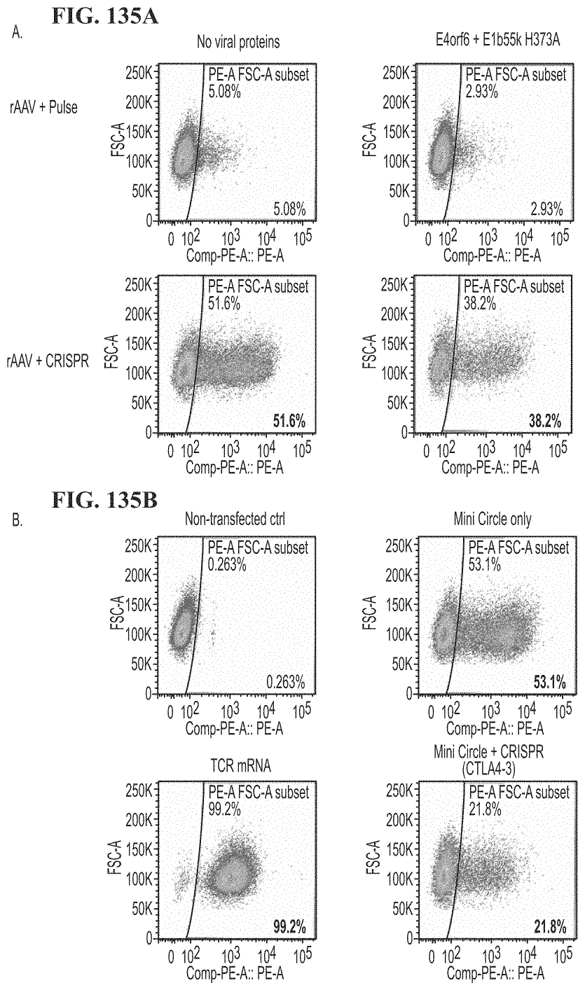

For example, a viral protein to be introduced can be or can be derived from: HCMV pUL83, DENV NS2B-NS3, HPV18 E7, hAd5 E1A, HSV1 ICP0, VACV B13, VACV C16, TREX1, HCoV-NL63, SARS-Cov, HBV Pol PEDV, or any combination thereof. A viral protein can be adenoviral. Adenoviral proteins can be adenovirus 4 E1B55K, E4orf6 protein. A viral protein can be a B13 vaccine virus protein. Viral proteins that are introduced can inhibit cytosolic DNA recognition, sensing, or a combination. In some cases, a viral protein can be utilized to recapitulate conditions of viral integration biology when engineering a cell. A viral protein can be introduced to a cell during transgene integration or genomic modification, utilizing CRISPR, FIG. 133A, FIG. 133B, FIG. 134, FIG. 135A and FIG. 135B.

In some cases, a RIP pathway can be inhibited. In other cases, a cellular FLICE (FADD-like IL-1beta-converting enzyme)-inhibitory protein (c-FLIP) pathway can be introduced to a cell. c-FLIP can be expressed as long (c-FLIPL), short (c-FLIPS), and c-FLIPR splice variants in human cells. c-FLIP can be expressed as a splice variant. c-FLIP can also be known as Casper, iFLICE, FLAME-1, CASH, CLARP, MRIT, or usurpin. c-FLIP can bind to FADD and/or caspase-8 or -10 and TRAIL receptor 5 (DR5). This interaction in turn prevents Death-Inducing Signaling Complex (DISC) formation and subsequent activation of the caspase cascade. c-FLIPL and c-FLIPS are also known to have multifunctional roles in various signaling pathways, as well as activating and/or upregulating several cytoprotective and pro-survival signaling proteins including Akt, ERK, and NF-κB. In some cases, c-FLIP can be introduced to a cell to increase viability.

In other cases, STING can be inhibited. In some cases, a caspase pathway is inhibited. A DNA sensing pathway can be a cytokine-based inflammatory pathway and/or an interferon alpha expressing pathway. In some cases, a multimodal approach is taken where at least one DNA sensing pathway inhibitor is introduced to a cell. In some cases, an inhibitor of DNA sensing can reduce cell death and allow for improved integration of an exogenous TCR transgene. A multimodal approach can be a STING and Caspase inhibitor in combination with a TBK inhibitor.

To enhance HDR, enabling the insertion of precise genetic modifications, we suppressed the NHEJ key molecules KU70, KU80 or DNA ligase IV by gene silencing, the ligase IV inhibitor SCR7 or the coexpression of adenovirus 4 E1B55K and E4orf6 proteins.

An introduced viral protein can reduce cellular toxicity of plasmid DNA by or by about 10%, 20%, 30%, 40%, 50%, 60%, 70%, 80%, 90%, or 100%. A viral protein can improve cellular viability by or by about 10%, 20%, 30%, 40%, 50%, 60%, 70%, 80%, 90%, or 100%.

In some cases, gRNA can be used to reduce toxicity. For example, a gRNA can be engineered to bind within a filler region of a vector. A vector can be a minicircle DNA vector. In some cases, a minicircle vector can be used in conjunction with a viral protein. In other cases, a minicircle vector can be used in conjunction with a viral protein and at least one additional toxicity reducing agent. In some cases, by reducing toxicity associated with exogenous DNA, such as double strand DNA, genomic disruptions can be performed more efficiently.

In some cases, an enzyme can be used to reduce DNA toxicity. For example, an enzyme such as DpnI can be utilized to remove methylated targets on a DNA vector or transgene. A vector or transgene can be pre-treated with DpnI prior to electroporation. Type IIM restriction endonucleases, such as DpnI, are able to recognize and cut methylated DNA. In some cases, a minicircle DNA is treated with DpnI. Naturally occurring restriction endonucleases are categorized into four groups (Types I, II III, and IV). In some cases, a restriction endonuclease, such as DpnI or a CRISPR system endonuclease is utilized to prepare engineered cells.

Disclosed herein, is a method of making an engineered cell comprising: introducing at least one engineered adenoviral protein or functional portion thereof; introducing at least one polynucleic acid encoding at least one exogenous receptor sequence; and genomically disrupting at least one genome with at least one endonuclease or portion thereof. In some cases, an adenoviral protein or function portion thereof is E1B55K, E4orf6, Scr7, L755507, NS2B3, HPV18 E7, hAd5 E1A, or a combination thereof. An adenoviral protein can be selected from a serotype 1 to 57. In some cases, an adenoviral protein serotype is serotype 5.

In some cases, an engineered adenoviral protein or portion thereof has at least one modification. A modification can be a substitution, insertion, deletion, or modification of a sequence of said adenoviral protein. A modification can be an insertion. An insertion can be an AGIPA insertion. In some cases, a modification is a substitution. A substitution can be a H to A at amino acid position 373 of a protein sequence. A polynucleic acid can be DNA or RNA. A polynucleic acid can be DNA. DNA can be minicircle DNA. In some cases, an exogenous receptor sequence can be selected from the group consisting of a sequence of a T cell receptor (TCR), a B cell receptor (BCR), a chimeric antigen receptor (CAR), and any portion or derivative thereof. An exogenous receptor sequence can be a TCR sequence. An endonuclease can be selected from the group consisting of CRISPR, TALEN, transposon-based, ZEN, meganuclease, Mega-TAL, and any portion or derivative thereof. An endonuclease can be CRISPR. CRISPR can comprise at least one Cas protein. A Cas protein can be selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof or modified versions thereof. A Cas protein can be Cas9.

In some cases, CRISPR creates a double strand break in a genome. A genome can comprise at least one gene. In some cases, an exogenous receptor sequence is introduced into at least one gene. An introduction can disrupt at least one gene. A gene can be CISH, TCR, TRA, TRB, or a combination thereof. A cell can be human. A human cell can be immune. An immune cell can be CD3+, CD4+, CD8+ or any combination thereof. A method can further comprise expanding a cell.

Disclosed herein, is a method of making an engineered cell comprising: virally introducing at least one polynucleic acid encoding at least one exogenous T cell receptor (TCR) sequence; and genomically disrupting at least one gene with at least one endonuclease or functional portion thereof. In some cases, a virus can be selected from retrovirus, lentivirus, adenovirus, adeno-associated virus, or any derivative thereof. A virus can be an adeno-associated virus (AAV). An AAV can be serotype 5. An AAV can be serotype 6. An AAV can comprise at least one modification. A modification can be a chemical modification. A polynucleic acid can be DNA, RNA, or any modification thereof. A polynucleic acid can be DNA. In some cases, DNA is minicircle DNA. In some cases, a polynucleic acid can further comprise at least one homology arm flanking a TCR sequence. A homology arm can comprise a complementary sequence at least one gene. A gene can be an endogenous gene. An endogenous gene can be a checkpoint gene.

In some cases, a method or a system according to any embodiment of the present disclosure can further comprise at least one toxicity reducing agent. In some cases, an AAV vector can be used in conjunction with at least one additional toxicity reducing agent. In other cases, a minicircle vector can be used in conjunction with at least one additional toxicity reducing agent. A toxicity reducing agent can be a viral protein or an inhibitor of the cytosolic DNA sensing pathway. A viral protein can be E1B55K, E4orf6, Scr7, L755507, NS2B3, HPV18 E7, hAd5 E1A, or a combination thereof. A method can further comprise expansion of cells. In some cases, an inhibitor of the cytosolic DNA sensing pathway can be used can be cellular FLICE (FADD-like IL-1β-converting enzyme)-inhibitory protein (c-FLIP).

Cell viability and/or the efficiency of integration of a transgene into a genome of one or more cells may be measured using any method known in the art. In some cases, cell viability and/or efficiency of integration may be measured using trypan blue exclusion, terminal deoxynucleotidyl transferase dUTP nick end labeling (TUNEL), the presence or absence of given cell-surface markers (e.g., CD4 or CD8), telomere length, fluorescence-activated cell sorting (FACS), real-time PCR, or droplet digital PCR. For example, FACS may be used to detect the efficiency of integration of a transgene following electroporation. In another example, apoptosis of may be measured using TUNEL. In some cases, toxicity can occur by genomic manipulation of cells, D. R. Sen et al., Science 10.1126/science.aae0491 (2016). Toxicity may result in cellular exhaustion that can affect cellular cytotoxicity against a tumor target. In some cases, an exhausted T cell may occupy a differentiation state distinct from a functional memory T cell. In some cases, identifying an altered cellular state and methods of reverting it to a baseline can be described by methods herein. For example, mapping state-specific enhancers in exhausted T cells can enable improved genomic editing for adoptive T cell therapy. In some cases, genomic editing to make T cells resistant to exhaustion may improve adoptive T cell therapy. In some cases, exhausted T cells may have an altered chromatic landscape when compared to functional memory T cells. An altered chromatin landscape may include epigenetic changes.

### Delivery of Vector into Cell Membrane

The nucleases and transcription factors, polynucleotides encoding same, and/or any transgene polynucleotides and compositions comprising the proteins and/or polynucleotides described herein can be delivered to a target cell by any suitable means.

Suitable cells can include but are not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces. In some cases, the cell line is a CHO-K1, MDCK or HEK293 cell line. In some cases, a cell or a population of cells is a primary cell or a population of primary cells. In some cases, a primary cell or a population of primary cells is a primary lymphocyte or a population of primary lymphocytes. In some cases, suitable primary cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem cell or a non-pluripotent stem cell. In some cases, the cell can be any immune cells including any T-cell such as tumor infiltrating cells (TILs), such as CD3+ T-cells, CD4+ T-cells, CD8+ T-cells, or any other type of T-cell. The T cell can also include memory T cells, memory stem T cells, or effector T cells. The T cells can also be selected from a bulk population, for example, selecting T cells from whole blood. The T cells can also be expanded from a bulk population. The T cells can also be skewed towards particular populations and phenotypes. For example, the T cells can be skewed to phenotypically comprise, CD45RO(−), CCR7(+), CD45RA(+), CD62L(+), CD27(+), CD28(+) and/or IL-7Rα(+). Suitable cells can be selected that comprise one of more markers selected from a list comprising: CD45RO(−), CCR7(+), CD45RA(+), CD62L(+), CD27(+), CD28(+) and/or IL-7Rα(+). Suitable cells also include stem cells such as, by way of example, embryonic stem cells, induced pluripotent stem cells, hematopoietic stem cells, neuronal stem cells and mesenchymal stem cells. Suitable cells can comprise any number of primary cells, such as human cells, non-human cells, and/or mouse cells. Suitable cells can be progenitor cells. Suitable cells can be derived from the subject to be treated (e.g., patient). Suitable cells can be derived from a human donor. Suitable cells can be stem memory T_{SCM} cells comprised of CD45RO (−), CCR7(+), CD45RA (+), CD62L+(L-selectin), CD27+, CD28+ and IL-7Rα+, stem memory cells can also express CD95, IL-2Rβ, CXCR3, and LFA-1, and show numerous functional attributes distinctive of stem memory cells. Suitable cells can be central memory T_{C}M cells comprising L-selectin and CCR7, central memory cells can secrete, for example, IL-2, but not IFNγ or IL-4. Suitable cells can also be effector memory T_{EM} cells comprising L-selectin or CCR7 and produce, for example, effector cytokines such as IFNγ and IL-4. In some cases, a primary cell can be a primary lymphocyte. In some cases, a population of primary cells can be a population of lymphocytes.

A method of attaining suitable cells can comprise selecting cells. In some cases, a cell can comprise a marker that can be selected for the cell. For example, such marker can comprise GFP, a resistance gene, a cell surface marker, an endogenous tag. Cells can be selected using any endogenous marker. Suitable cells can be selected using any technology. Such technology can comprise flow cytometry and/or magnetic columns. The selected cells can then be infused into a subject. The selected cells can also be expanded to large numbers. The selected cells can be expanded prior to infusion.

The transcription factors and nucleases as described herein can be delivered using vectors, for example containing sequences encoding one or more of the proteins. Transgenes encoding polynucleotides can be similarly delivered. Any vector systems can be used including, but not limited to, plasmid vectors, retroviral vectors, lentiviral vectors, adenovirus vectors, poxvirus vectors; herpesvirus vectors and adeno-associated virus vectors, etc. Furthermore, any of these vectors can comprise one or more transcription factor, nuclease, and/or transgene. Thus, when one or more CRISPR, TALEN, transposon-based, ZEN, meganuclease, or Mega-TAL molecules and/or transgenes are introduced into the cell, CRISPR, TALEN, transposon-based, ZEN, meganuclease, or Mega-TAL molecules and/or transgenes can be carried on the same vector or on different vectors. When multiple vectors are used, each vector can comprise a sequence encoding one or multiple CRISPR, TALEN, transposon-based, ZEN, meganuclease, or Mega-TAL molecules and/or transgenes.

Conventional viral and non-viral based gene transfer methods can be used to introduce nucleic acids encoding engineered CRISPR, TALEN, transposon-based, ZEN, meganuclease, or Mega-TAL molecules and/or transgenes in cells (e.g., mammalian cells) and target tissues. Such methods can also be used to administer nucleic acids encoding CRISPR, TALEN, transposon-based, ZEN, meganuclease, or Mega-TAL molecules and/or transgenes to cells in vitro. In some examples, nucleic acids encoding CRISPR, TALEN, transposon-based, ZEN, meganuclease, or Mega-TAL molecules and/or transgenes can be administered for in vivo or ex vivo immunotherapy uses. Non-viral vector delivery systems can include DNA plasmids, naked nucleic acid, and nucleic acid complexed with a delivery vehicle such as a liposome or poloxamer. Viral vector delivery systems can include DNA and RNA viruses, which have either episomal or integrated genomes after delivery to the cell.

Methods of viral or non-viral delivery of nucleic acids include electroporation, lipofection, nucleofection, gold nanoparticle delivery, microinjection, biolistics, virosomes, liposomes, immunoliposomes, polycation or lipid: nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by AMARA® Biosystems (Cologne, Germany), Life Technologies (Frederick, Md.), MAXCYTE, Inc. (Rockville, Md.), BTX Molecular Delivery Systems (Holliston, Mass.) and Copernicus Therapeutics Inc. (see for example U.S. Pat. No. 6,008,336). Lipofection reagents are sold commercially (e.g., TRANSFECTAM® and LIPOFECTIN®). Delivery can be to cells (ex vivo administration) or target tissues (in vivo administration). Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis.

Vectors including viral and non-viral vectors containing nucleic acids encoding engineered CRISPR, TALEN, transposon-based, ZEN, meganuclease, or Mega-TAL molecules, transposon and/or transgenes can also be administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application and electroporation. More than one route can be used to administer a particular composition. Pharmaceutically acceptable carriers are determined in part by the particular composition being administered, as well as by the particular method used to administer the composition.

In some cases, a vector encoding for an exogenous TCR can be shuttled to a cellular nuclease. For example, a vector can contain a nuclear localization sequence (NLS). A vector can also be shuttled by a protein or protein complex. In some cases, Cas9 can be used as a means to shuttle a minicircle vector. Cas can comprise a NLS. In some cases, a vector can be pre-complexed with a Cas protein prior to electroporation. A Cas protein that can be used for shuttling can be a nuclease-deficient Cas9 (dCas9) protein. A Cas protein that can be used for shuttling can be a nuclease-competent Cas9. In some cases, Cas protein can be pre-mixed with a guide RNA and a plasmid encoding an exogenous TCR.

Certain aspects disclosed herein can utilize vectors. For example, vectors that can be used include, but not limited to, Bacterial: pBs, pQE-9 (Qiagen), phagescript, PsiX174, pBluescript SK, pBsKS, pNH8a, pNH16a, pNH18a, pNH46a (Stratagene); pTrc99A, pKK223-3, pKK233-3, pDR54O, pRIT5 (Pharmacia). Eukaryotic: pWL-neo, pSv2cat, pOG44, pXT1, pSG (Stratagene) pSVK3, pBPv, pMSG, pSVL (Pharmiacia). Also, any other plasmids and vectors can be used as long as they are replicable and viable in a selected host. Any vector and those commercially available (and variants or derivatives thereof) can be engineered to include one or more recombination sites for use in the methods. Such vectors can be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, PerkinElmer, Pharmingen, and Research Genetics. Other vectors of interest include eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3 pREP4, pCEP4, and pEBVHis (Invitrogen, Corp.), and variants or derivatives thereof. Other vectors include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSYSPORT1 (Invitrogen) and variants or derivatives thereof. Additional vectors of interest can also include pTrxFus, pThioHis, pLEX, pTrcHis, pTrcHis2, pRSET, pBlueBa-cHis2, pcDNA3.1/His, pcDNA3.1(−)/Myc-His, pSecTag, pEBVHis, pPIC9K, pPIC3.5K, pA081S, pPICZ, pPICZA, pPICZB, pPICZC, pGAPZA, pGAPZB, pGAPZC, pBlue-Bac4.5, pBlueBacHis2, pMelBac, pSinRep5, pSinHis, pIND, pIND (SP1), pVgRXR, pcDNA2.1, pYES2, pZEr01.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; X ExCell, X gal, pTrc99A, pKK223-3, pGEX-1X T, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4-abc(+), pOCUS-2, pTAg, pET-32L1C, pET-30LIC, pBAC-2 cp LIC, pBACgus-2 cp LIC, pT7Blue-2 LIC, pT7Blue-2, X SCREEN-1, X BlueSTAR, pET-3abcd, pET-7abc, pET9abcd, pET11 abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd (+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFPN, pEGFP-C,

pEBFP, pGFPuv, pGFP, p6×His-GFP, pSEAP2-Basic, pSEAP2-Contral, pSEAP2-Promoter, pSEAP2-Enhancer, p I3 gal-Basic, p13gal-Control, p I3 gal-Promoter, p I3 gal-Enhancer, pCMV, pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, 2Xgt10, Xgt11, pWE15, and X TriplEx from Clontech; Lambda ZAP II, pBK-CMV, pBK-RSV, pBluescript II KS+/−, pBluescript II SK+/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS+/−, pBC KS+/−, pBC SK+/−, Phag-escript, pCAL-n-EK, pCAL-n, pCAL-c, pCAL-kc, pET-3abcd, pET-llabcd, pSPUTK, pESP-1, pCMVLacI, pOPRSVI/MCS, pOPI3 CAT, pXT1, pSG5, pPbac, pMbac, pMClneo, pMClneo Poly A, pOG44, p0G45, pFRTI3GAL, pNE0I3GAL, pRS403, pRS404, pRS405, pRS406, pRS413, pRS414, pRS415, and pRS416 from Stratagene, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp, and variants or derivatives thereof.

These vectors can be used to express a gene, e.g., a transgene, or portion of a gene of interest. A gene of portion or a gene can be inserted by using any method. For example; a method can be a restriction enzyme-based technique.

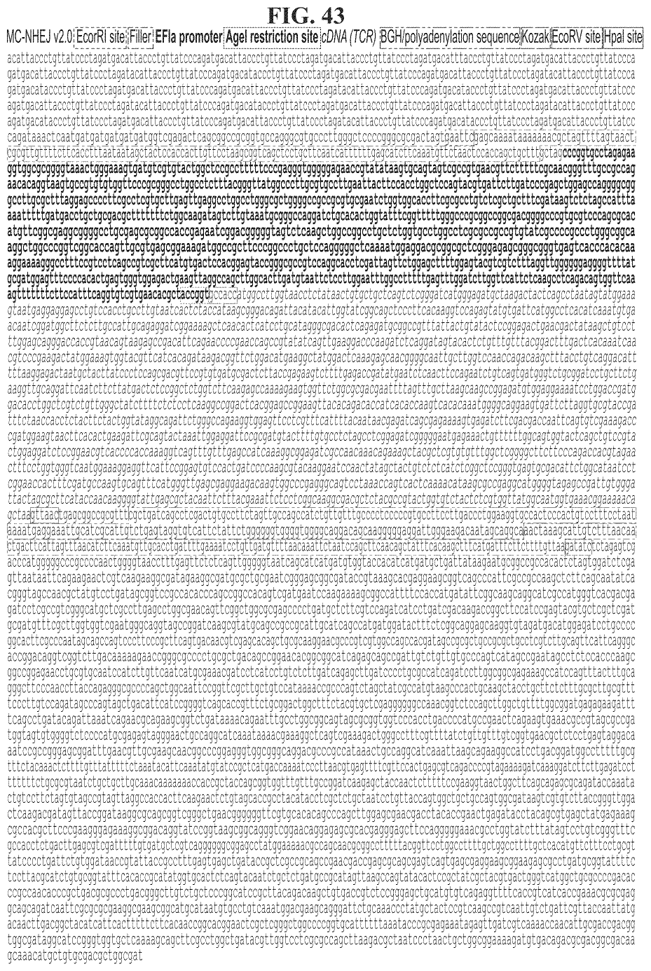

Vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, T cells, bone marrow aspirates, tissue biopsy), followed by reimplantation of the cells into a patient, usually after selection for cells which have incorporated the vector. Prior to or after selection, the cells can be expanded. A vector can be a minicircle vector, FIG. 43.

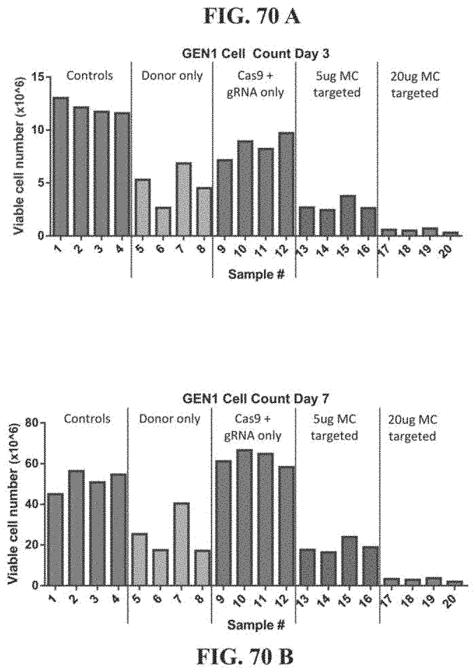

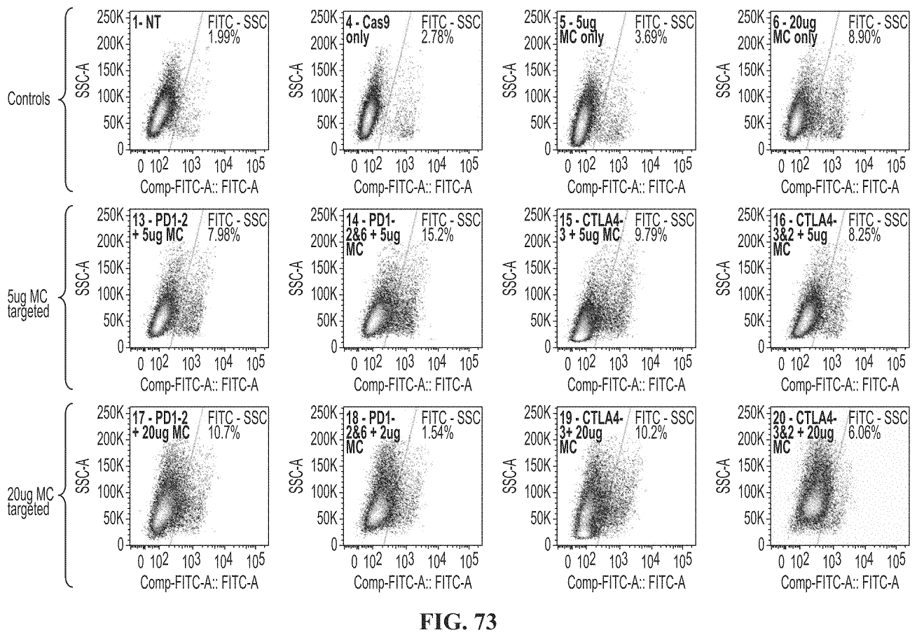

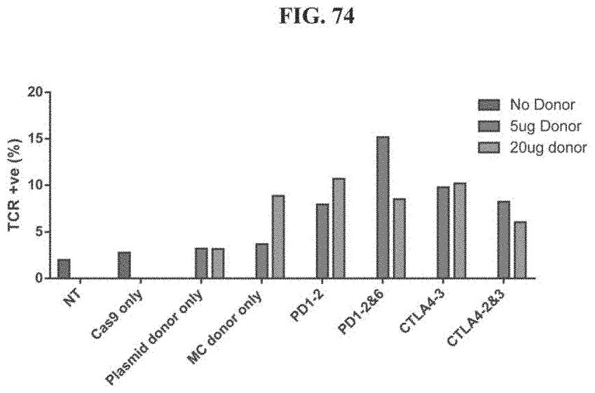

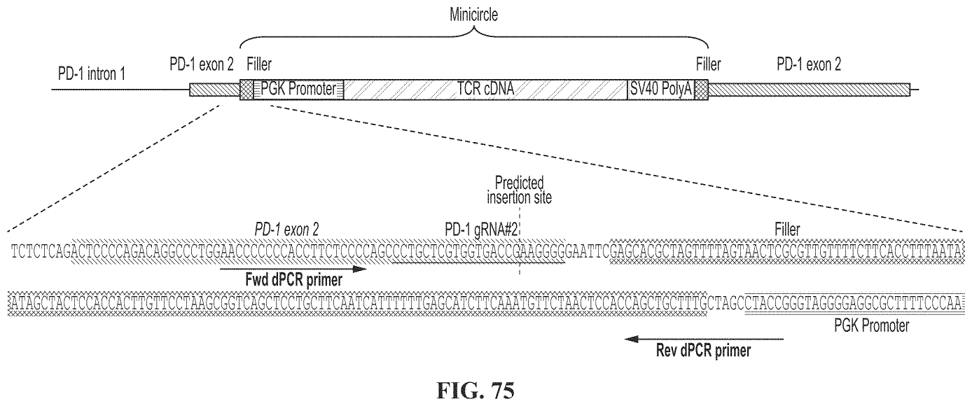

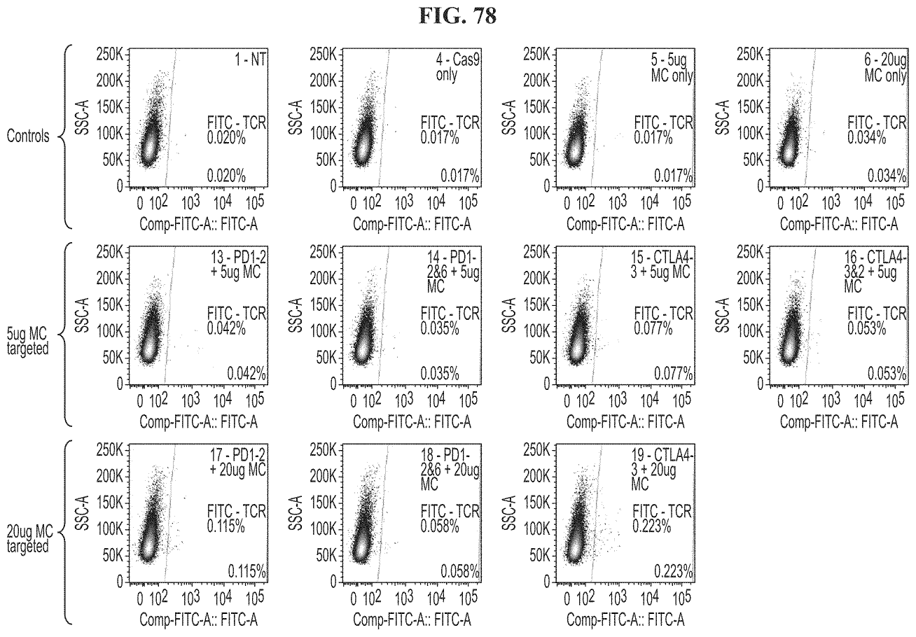

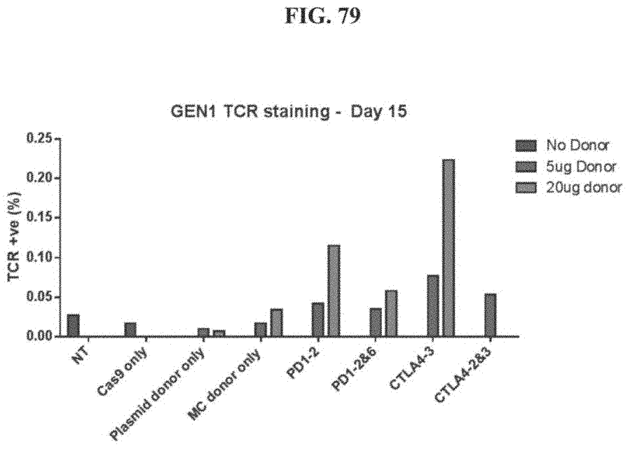

A cell can be transfected with a minicircle vector and a CRISPR system. In some cases, a minicircle vector is introduced to a cell or to a population of cells at the same time, before, or after a CRISPR system and/or a nuclease or a polypeptide encoding a nuclease is introduced to a cell or to a population of cells. A minicircle vector concentration can be from 0.5 nanograms to 50 micrograms. In some cases, the amount of nucleic acid (e.g., ssDNA, dsDNA, RNA) that may be introduced into the cell by electroporation may be varied to optimize transfection efficiency and/or cell viability. In some cases, less than about 100 picograms of nucleic acid may be added to each cell sample (e.g., one or more cells being electroporated). In some cases, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid may be added to each cell sample (e.g., one or more cells being electroporated). For example, 1 microgram of dsDNA may be added to each cell sample for electroporation. In some cases, the amount of nucleic acid (e.g., dsDNA) required for optimal transfection efficiency and/or cell viability may be specific to the cell type. In some cases, the amount of nucleic acid (e.g., dsDNA) used for each sample may directly correspond to the transfection efficiency and/or cell viability. For example, a range of concentrations of minicircle transfections are shown in FIG. 70 A, FIG. 70 B, and FIG. 73. A representative flow cytometry experiment depicting a summary of efficiency of integration of a minicircle vector transfected at a 5 and 20 microgram concentration is shown in FIG. 74, FIG. 78, and FIG. 79. A transgene encoded by a minicircle vector can integrate into a cellular genome. In some cases, integration of a transgene encoded by a minicircle vector is in the forward direction, FIG. 75. In other cases, integration of a transgene encoded by a minicircle vector is in the reverse direction. In some cases, a non-viral system (e.g., minicircle) is introduced to a cell or to a population of cells at about, from about, at least about, or at most about 1-3 hrs., 3-6 hrs., 6-9 hrs., 9-12 hrs., 12-15 hrs., 15-18 hrs., 18-21 hrs., 21-23 hrs., 23-26 hrs., 26-29 hrs., 29-31 hrs., 31-33 hrs., 33-35 hrs., 35-37 hrs., 37-39 hrs., 39-41 hrs., 2 days, 3 days, 4 days, 5 days, 6 days, 7 days, 8 days, 9 days, 10 days, 14 days, 16 days, 20 days, or longer than 20 days after a CRISPR system or after a nuclease or a polynucleic acid encoding a nuclease is introduced to said cell or to said population of cells

The transfection efficiency of cells with any of the nucleic acid delivery platforms described herein, for example, nucleofection or electroporation, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

Electroporation using, for example, the Neon® Transfection System (ThermoFisher Scientific) or the AMARA® Nucleofector (AMARA® Biosystems) can also be used for delivery of nucleic acids into a cell. Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some cases, the electroporation pulse voltage, the electroporation pulse width, number of pulses, cell density, and tip type may be adjusted to optimize transfection efficiency and/or cell viability.

In some cases, electroporation pulse voltage may be varied to optimize transfection efficiency and/or cell viability. In some cases, the electroporation voltage may be less than about 500 volts. In some cases, the electroporation voltage may be at least about 500 volts, at least about 600 volts, at least about 700 volts, at least about 800 volts, at least about 900 volts, at least about 1000 volts, at least about 1100 volts, at least about 1200 volts, at least about 1300 volts, at least about 1400 volts, at least about 1500 volts, at least about 1600 volts, at least about 1700 volts, at least about 1800 volts, at least about 1900 volts, at least about 2000 volts, at least about 2100 volts, at least about 2200 volts, at least about 2300 volts, at least about 2400 volts, at least about 2500 volts, at least about 2600 volts, at least about 2700 volts, at least about 2800 volts, at least about 2900 volts, or at least about 3000 volts. In some cases, the electroporation pulse voltage required for optimal transfection efficiency and/or cell viability may be specific to the cell type. For example, an electroporation voltage of 1900 volts may optimal (e.g., provide the highest viability and/or transfection efficiency) for macrophage cells. In another example, an electroporation voltage of about 1350 volts may optimal (e.g., provide the highest viability and/or transfection efficiency) for Jurkat cells or primary human cells such as T cells. In some cases, a range of electroporation voltages may be optimal for a given cell type. For example, an electroporation voltage between about 1000 volts and about 1300 volts may optimal (e.g., provide the highest viability and/or transfection efficiency) for human 578T cells. In some cases, a primary cell can be a primary lymphocyte. In some cases, a population of primary cells can be a population of lymphocytes.

In some cases, electroporation pulse width may be varied to optimize transfection efficiency and/or cell viability. In some cases, the electroporation pulse width may be less than about 5 milliseconds. In some cases, the electroporation width may be at least about 5 milliseconds, at least about 6 milliseconds, at least about 7 milliseconds, at least about 8 milliseconds, at least about 9 milliseconds, at least about 10 milliseconds, at least about 11 milliseconds, at least about 12 milliseconds, at least about 13 milliseconds, at least about 14 milliseconds, at least about 15 milliseconds, at least about 16 milliseconds, at least about 17 milliseconds, at least about 18 milliseconds, at least about 19 milliseconds, at least about 20 milliseconds, at least about 21 milliseconds, at least about 22 milliseconds, at least about 23 milliseconds, at least about 24 milliseconds, at least about 25 milliseconds, at least about 26 milliseconds, at least about 27 milliseconds, at least about 28 milliseconds, at least about 29 milliseconds, at least about 30 milliseconds, at least about 31 milliseconds, at least about 32 milliseconds, at least about 33 milliseconds, at least about 34 milliseconds, at least about 35 milliseconds, at least about 36 milliseconds, at least about 37 milliseconds, at least about 38 milliseconds, at least about 39 milliseconds, at least about 40 milliseconds, at least about 41 milliseconds, at least about 42 milliseconds, at least about 43 milliseconds, at least about 44 milliseconds, at least about 45 milliseconds, at least about 46 milliseconds, at least about 47 milliseconds, at least about 48 milliseconds, at least about 49 milliseconds, or at least about 50 milliseconds. In some cases, the electroporation pulse width required for optimal transfection efficiency and/or cell viability may be specific to the cell type. For example, an electroporation pulse width of 30 milliseconds may optimal (e.g., provide the highest viability and/or transfection efficiency) for macrophage cells. In another example, an electroporation width of about 10 milliseconds may optimal (e.g., provide the highest viability and/or transfection efficiency) for Jurkat cells. In some cases, a range of electroporation widths may be optimal for a given cell type. For example, an electroporation width between about 20 milliseconds and about 30 milliseconds may optimal (e.g., provide the highest viability and/or transfection efficiency) for human 578T cells.

In some cases, the number of electroporation pulses may be varied to optimize transfection efficiency and/or cell viability. In some cases, electroporation may comprise a single pulse. In some cases, electroporation may comprise more than one pulse. In some cases, electroporation may comprise 2 pulses, 3 pulses, 4 pulses, 5 pulses 6 pulses, 7 pulses, 8 pulses, 9 pulses, or 10 or more pulses. In some cases, the number of electroporation pulses required for optimal transfection efficiency and/or cell viability may be specific to the cell type. For example, electroporation with a single pulse may be optimal (e.g., provide the highest viability and/or transfection efficiency) for macrophage cells. In another example, electroporation with a 3 pulses may be optimal (e.g., provide the highest viability and/or transfection efficiency) for primary cells. In some cases, a range of electroporation widths may be optimal for a given cell type. For example, electroporation with between about 1 to about 3 pulses may be optimal (e.g., provide the highest viability and/or transfection efficiency) for human cells.

In some cases, the starting cell density for electroporation may be varied to optimize transfection efficiency and/or cell viability. In some cases, the starting cell density for electroporation may be less than about 1×10⁵ cells. In some cases, the starting cell density for electroporation may be at least about 1×10⁵ cells, at least about 2×10⁵ cells, at least about 3×10⁵ cells, at least about 4×10⁵ cells, at least about 5×10⁵ cells, at least about 6×10⁵ cells, at least about 7×10⁵ cells, at least about 8×10⁵ cells, at least about 9×10⁵ cells, at least about 1×10⁶ cells, at least about 1.5×10⁶ cells, at least about 2×10⁶ cells, at least about 2.5×10⁶ cells, at least about 3×10⁶ cells, at least about 3.5×10⁶ cells, at least about 4×10⁶ cells, at least about 4.5×10⁶ cells, at least about 5×10⁶ cells, at least about 5.5×10⁶ cells, at least about 6×10⁶ cells, at least about 6.5×10⁶ cells, at least about 7×10⁶ cells, at least about 7.5×10⁶ cells, at least about 8×10⁶ cells, at least about 8.5×10⁶ cells, at least about 9×10⁶ cells, at least about 9.5×10⁶ cells, at least about 1×10⁷ cells, at least about 1.2×10⁷ cells, at least about 1.4×10⁷ cells, at least about 1.6×10⁷ cells, at least about 1.8×10⁷ cells, at least about 2×10⁷ cells, at least about 2.2×10⁷ cells, at least about 2.4×10⁷ cells, at least about 2.6×10⁷ cells, at least about 2.8×10⁷ cells, at least about 3×10⁷ cells, at least about 3.2×10⁷ cells, at least about 3.4×10⁷ cells, at least about 3.6×10⁷ cells, at least about 3.8×10⁷ cells, at least about 4×10⁷ cells, at least about 4.2×10⁷ cells, at least about 4.4×10⁷ cells, at least about 4.6×10⁷ cells, at least about 4.8×10⁷ cells, or at least about 5×10⁷ cells. In some cases, the starting cell density for electroporation required for optimal transfection efficiency and/or cell viability may be specific to the cell type. For example, a starting cell density for electroporation of 1.5×10⁶ cells may optimal (e.g., provide the highest viability and/or transfection efficiency) for macrophage cells. In another example, a starting cell density for electroporation of 5×10⁶ cells may optimal (e.g., provide the highest viability and/or transfection efficiency) for human cells. In some cases, a range of starting cell densities for electroporation may be optimal for a given cell type. For example, a starting cell density for electroporation between of 5.6×10⁶ and 5×10⁷ cells may optimal (e.g., provide the highest viability and/or transfection efficiency) for human cells such as T cells.

The efficiency of integration of a nucleic acid sequence encoding an exogenous TCR into a genome of a cell with, for example, a CRISPR system, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

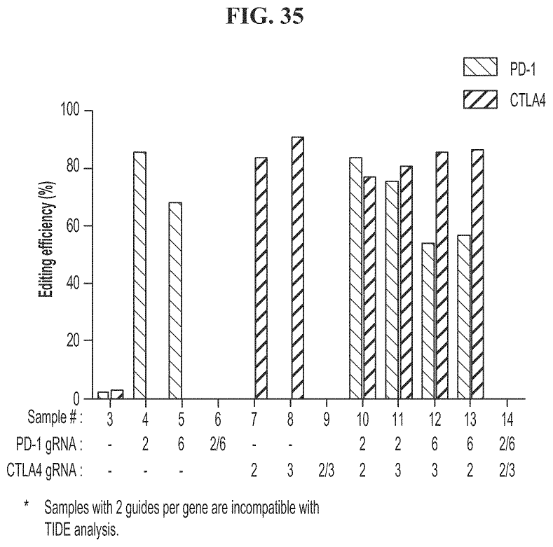

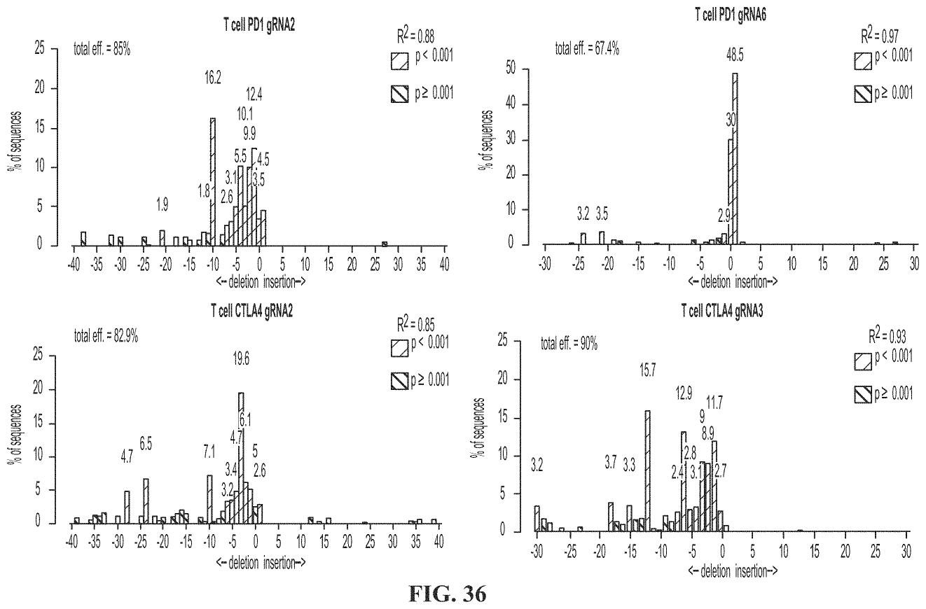

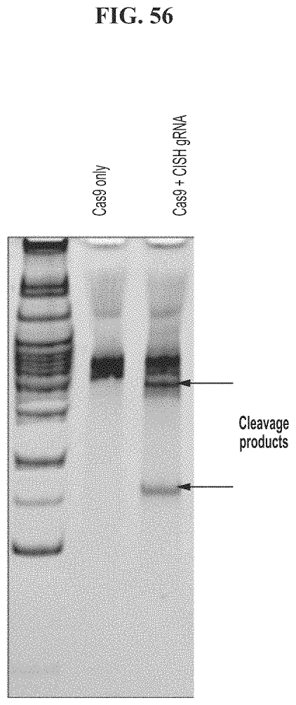

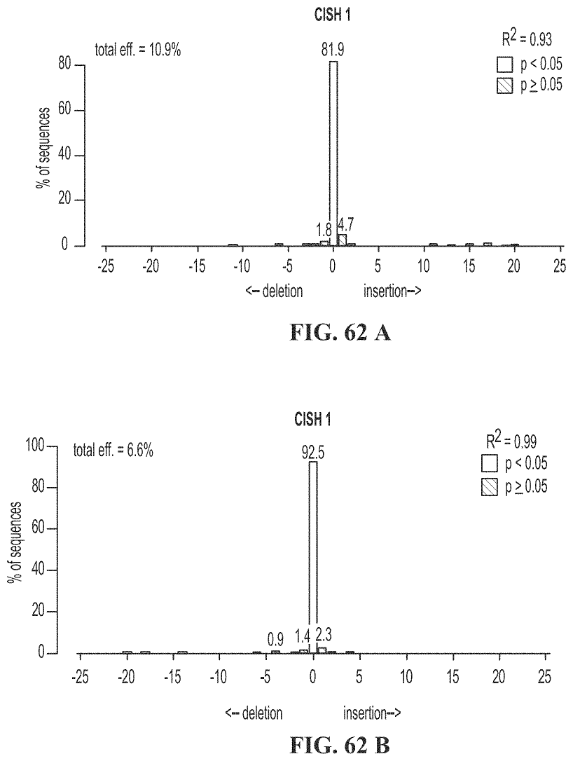

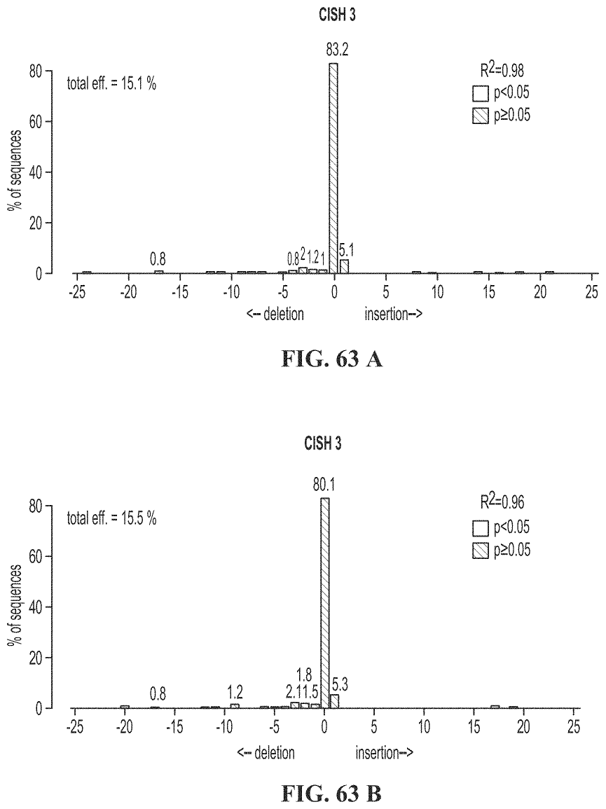

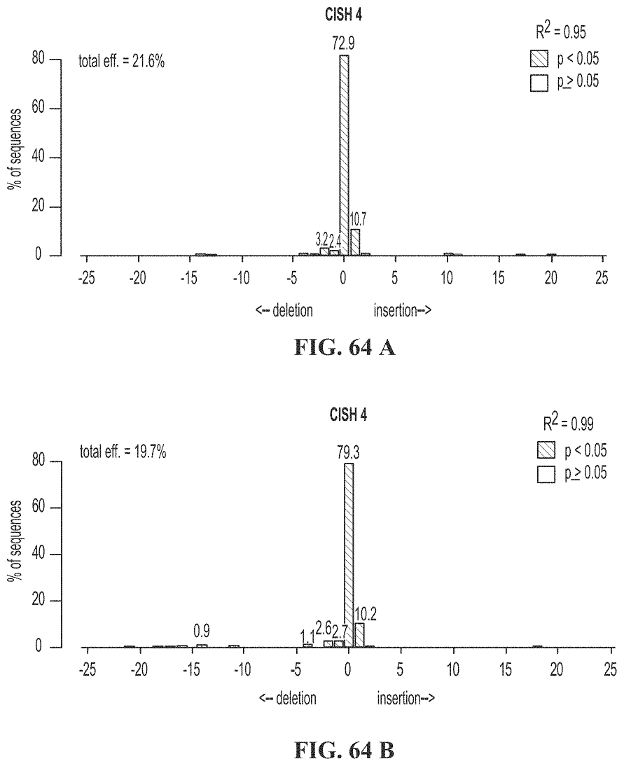

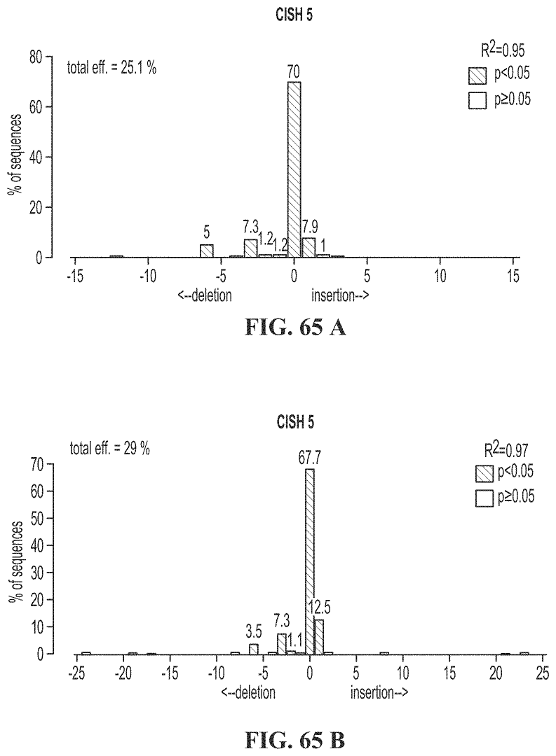

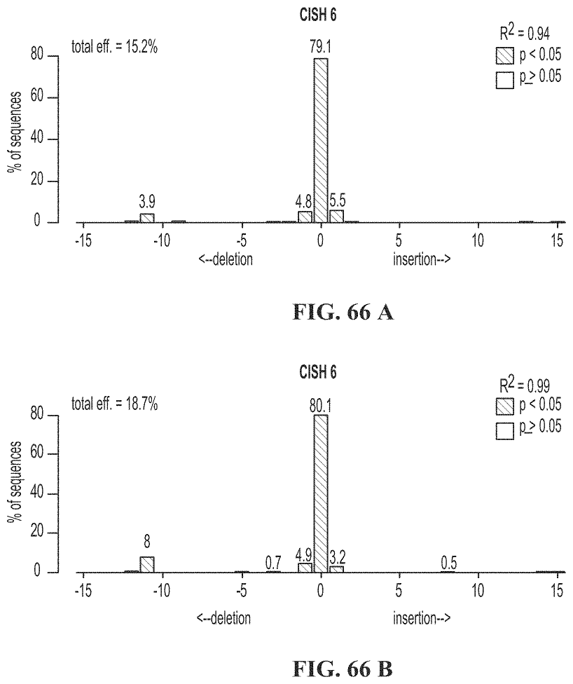

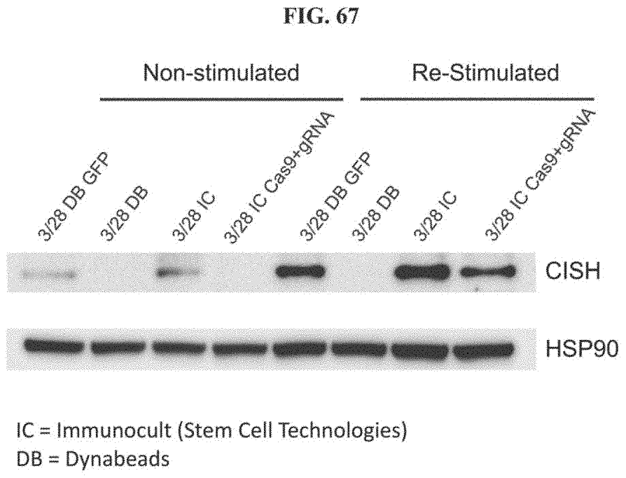

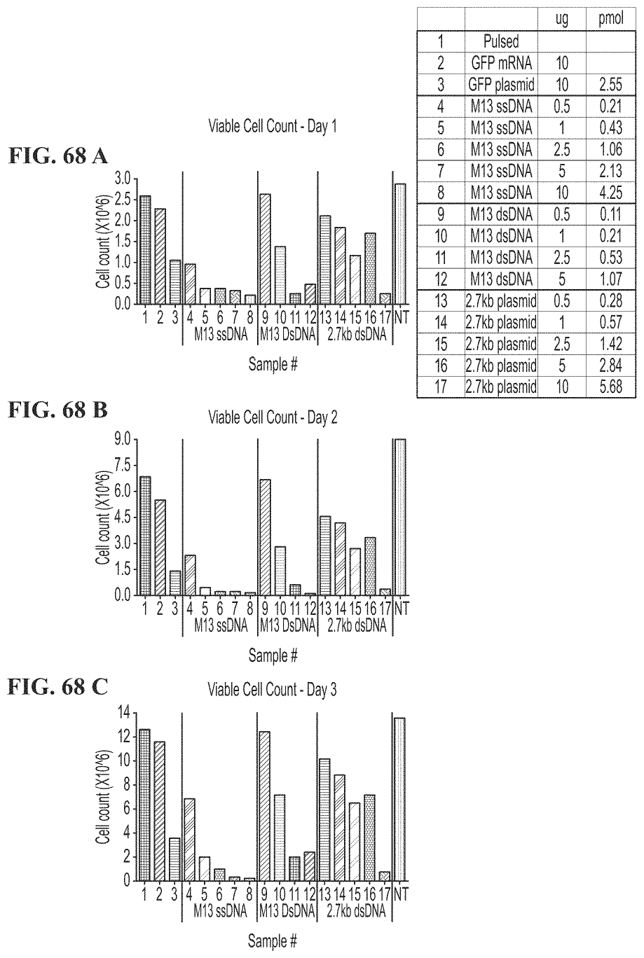

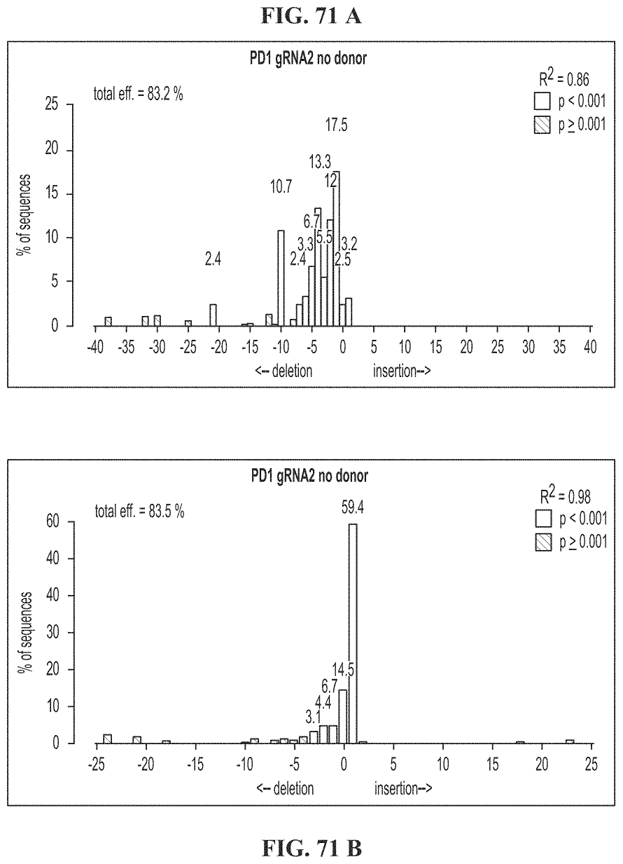

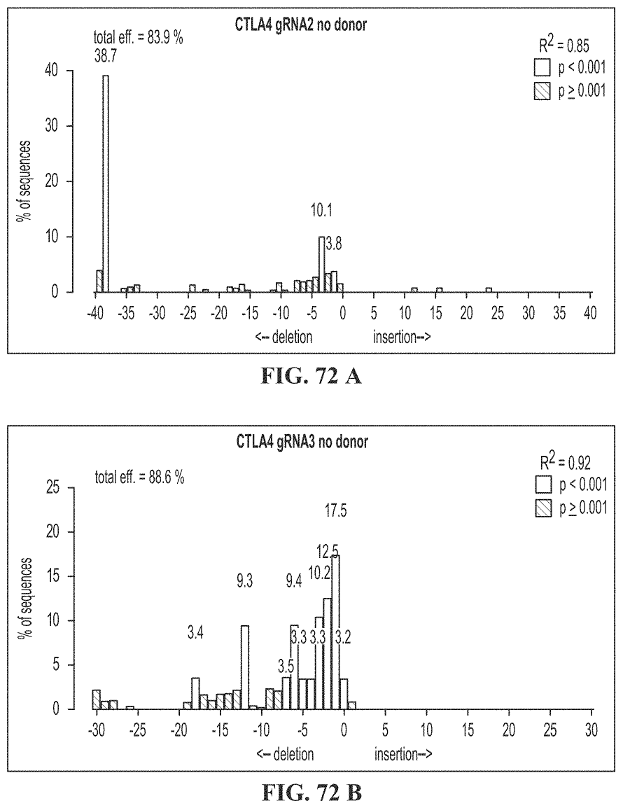

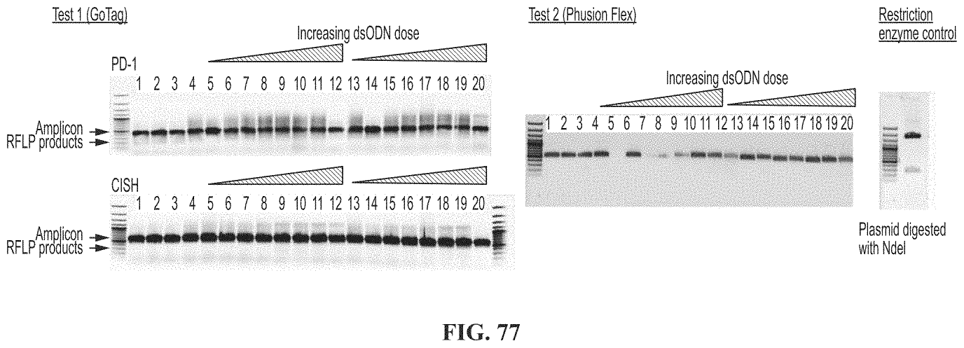

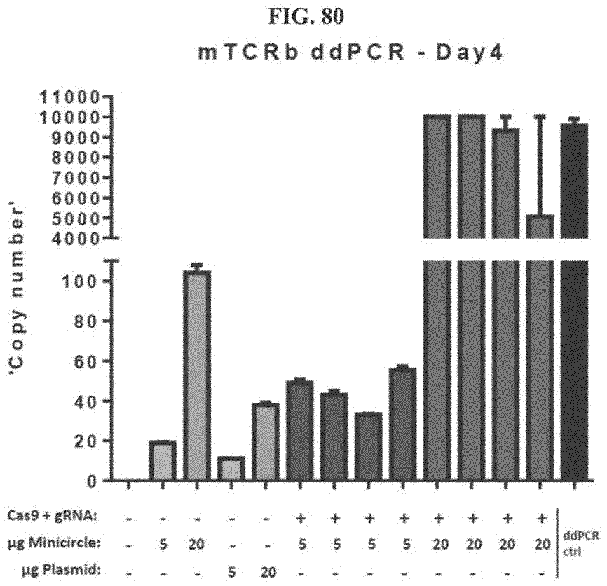

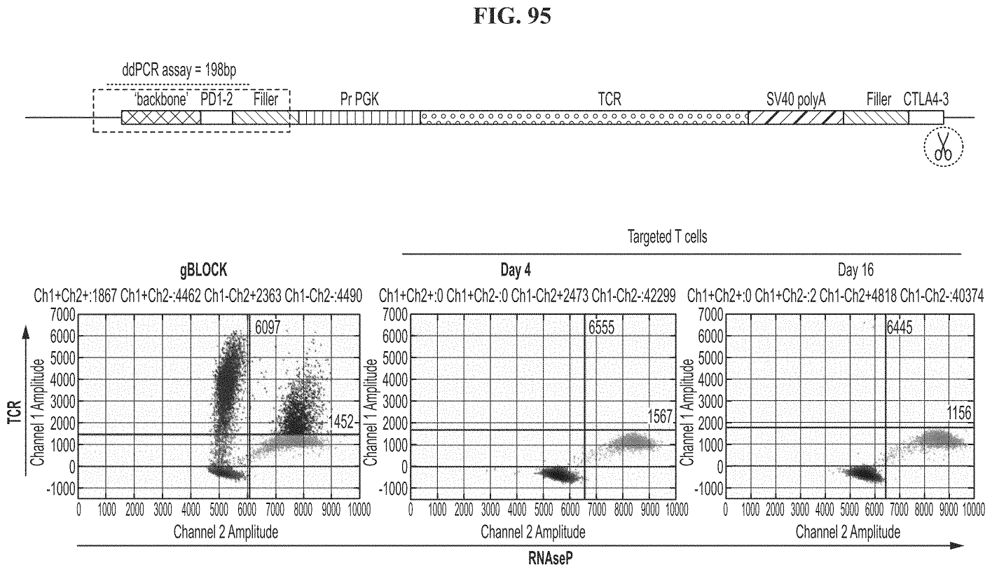

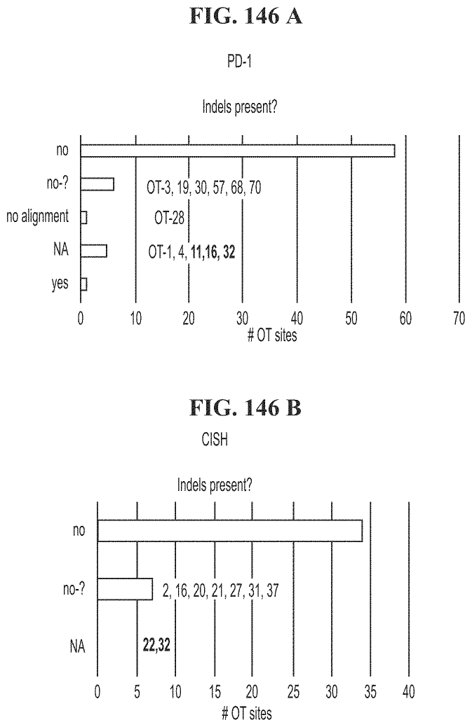

Integration of an exogenous polynucleic acid, such as a TCR, can be measured using any technique. For example, integration can be measured by flow cytometry, surveyor nuclease assay (FIG. 56), tracking of indels by decomposition (TIDE), FIG. 71 and FIG. 72, junction PCR, or any combination thereof. A representative TIDE analysis is shown for percent gene editing efficiency as show for PD-1 and CTLA-4 guide RNAs, FIG. 35 and FIG. 36. A representative TIDE analysis for CISH guide RNAs is shown from FIG. 62 to FIGS. 67 A and B. In other cases, transgene integration can be measured by PCR, FIG. 77, FIG. 80, and FIG. 95. A TIDE analysis can also be performed on cells engineered to express an exogenous TCR by rAAV transduction followed by CRISPR knock out of an endogenous checkpoint gene, FIG. 146A and FIG. 146B.

Ex vivo cell transfection can also be used for diagnostics, research, or for gene therapy (e.g., via re-infusion of the transfected cells into the host organism). In some cases, cells are isolated from the subject organism, transfected with a nucleic acid (e.g., gene or cDNA), and re-infused back into the subject organism (e.g., patient).

The amount of cells that are necessary to be therapeutically effective in a patient may vary depending on the viability of the cells, and the efficiency with which the cells have been genetically modified (e.g., the efficiency with which a transgene has been integrated into one or more cells). In some cases, the product (e.g., multiplication) of the viability of cells post genetic modification and the efficiency of integration of a transgene may correspond to the therapeutic aliquot of cells available for administration to a subject. In some cases, an increase in the viability of cells post genetic modification may correspond to a decrease in the amount of cells that are necessary for administration to be therapeutically effective in a patient. In some cases, an increase in the efficiency with which a transgene has been integrated into one or more cells may correspond to a decrease in the amount of cells that are necessary for administration to be therapeutically effective in a patient. In some cases, determining an amount of cells that are necessary to be therapeutically effective may comprise determining a function corresponding to a change in the viability of cells over time. In some cases, determining an amount of cells that are necessary to be therapeutically effective may comprise determining a function corresponding to a change in the efficiency with which a transgene may be integrated into one or more cells with respect to time dependent variables (e.g., cell culture time, electroporation time, cell stimulation time).

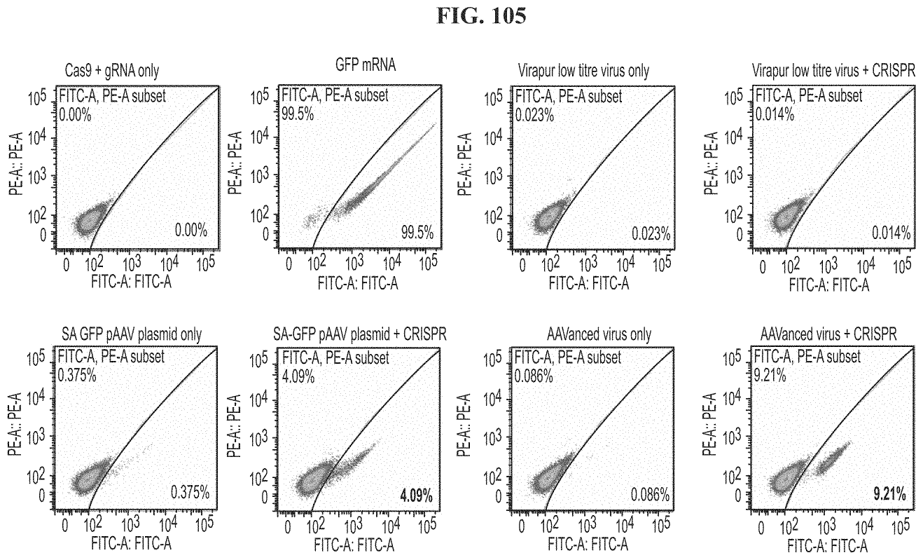

As described herein, viral particles, such as rAAV, can be used to deliver a viral vector comprising a gene of interest or a transgene into a cell ex vivo or in vivo, FIG. 105. In some cases, the viral vector as disclosed herein may be measured as pfu (plaque forming units). In some cases, the pfu of recombinant virus or viral vector of the compositions and methods of the disclosure may be about 10⁸ to about 5×10¹⁰ pfu. In some cases, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some cases, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some aspects, the viral vector of the disclosure may be measured as vector genomes. In some cases, recombinant viruses of this disclosure are 1×10¹⁰ to 3×10¹² vector genomes, or 1×10⁹ to 3×10¹³ vector genomes, or 1×10⁸ to 3×10¹⁴ vector genomes, or at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes, or are 1×10⁸ to 3×10¹⁴ vector genomes, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes.

In some cases, the viral vector (e.g., AAV or modified AAV) of the disclosure can be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵ to 1×10⁷. In some cases, the MOI may be 1×10⁴ to 1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹¹, 1×10₁₃, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, an AAV and/or modified AAV vector is introduced at a multiplicity of infection (MOI) from about 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶ 4×10⁶, 5×10⁶, 6×10⁶, 7×10⁶, 8×10⁶, 9×10⁶, 1×10⁷, 2×10⁷, 3×10⁷, or up to about 9×10⁹ genome copies/virus particles per cell.

In some aspects, a non-viral vector or nucleic acid may be delivered without the use of a virus and may be measured according to the quantity of nucleic acid. Generally, any suitable amount of nucleic acid can be used with the compositions and methods of this disclosure. In some cases, nucleic acid may be at least about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g. In some cases, nucleic acid may be at most about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

In some cases, a viral (AAV or modified AAV) or non-viral vector is introduced to a cell or to a population of cells. In some cases, cell toxicity is measured after a viral vector or a non-viral vector is introduced to a cell or to a population of cells. In some cases, cell toxicity is lower when a modified AAV is used than when a wild-type AAV or a non-viral vector (e.g., minicircle) is introduced to a comparable cell or to a comparable population of cells. In some cases, cell toxicity is measured by flow cytometry. In some cases, cell toxicity is reduced by about, at least about, or at most about 1%, 2%, 3%, 4%, 5%, 6%, 7%, 8%, 9%, 10%, 12%, 15%, 17%, 18%, 19%, 20%, 25%, 30%, 35%, 40%, 45%, 50%, 55%, 60%, 65%, 70%, 75%, 80%, 82%, 85%, 88%, 90%, 92%, 95%, 97%, 98%, 99% or 100% when a modified AAV is used compared to a wild-type or unmodified AAV or a minicircle. In some cases, cell toxicity is reduced by about, at least about, or at most about 1%, 2%, 3%, 4%, 5%, 6%, 7%, 8%, 9%, 10%, 12%, 15%, 17%, 18%, 19%, 20%, 25%, 30%, 35%, 40%, 45%, 50%, 55%, 60%, 65%, 70%, 75%, 80%, 82%, 85%, 88%, 90%, 92%, 95%, 97%, 98%, 99% or 100% when an AAV vector is used compared to when a minicircle vector or a non-viral vector is used.

a. Functional Transplant

Cells (e.g., engineered cells or engineered primary T cells) before, after, and/or during transplantation can be functional. For example, transplanted cells can be functional for at least or at least about 1, 2, 3, 4, 5, 6, 7, 8, 9, 10, 11, 12, 13, 14, 15, 16, 17, 18, 19, 20, 21, 22, 23, 24, 25, 6, 27, 28, 29, 30, 40, 50, 60, 70, 80, 90, or 100 days after transplantation. Transplanted cells can be functional for at least or at least about 1, 2, 3, 4, 5, 6, 7, 8, 9, 10, 11, or 12 months after transplantation. Transplanted cells can be functional for at least or at least about 1, 2, 3, 4, 5, 6, 7, 8, 9, 10, 15, 20, 25, or 30 years after transplantation. In some cases, transplanted cells can be functional for up to the lifetime of a recipient.

Further, transplanted cells can function at 100% of its normal intended operation. Transplanted cells can also function 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of its normal intended operation.

Transplanted cells can also function over 100% of its normal intended operation. For example, transplanted cells can function 110, 120, 130, 140, 150, 160, 170, 180, 190, 200, 250, 300, 400, 500, 600, 700, 800, 900, 1000 or more % of its normal intended operation.

### Pharmaceutical Compositions and Formulations

The compositions described throughout can be formulation into a pharmaceutical medicament and be used to treat a human or mammal, in need thereof, diagnosed with a disease, e.g., cancer. These medicaments can be co-administered with one or more T cells (e.g., engineered T cells) to a human or mammal, together with one or more chemotherapeutic agent or chemotherapeutic compound.

A “chemotherapeutic agent” or “chemotherapeutic compound” and their grammatical equivalents as used herein, can be a chemical compound useful in the treatment of cancer. The chemotherapeutic cancer agents that can be used in combination with the disclosed T cell include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein includes antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

The disclosed T cell herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed T cell include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one case, the anti-cancer drug is 5-fluorouracil, taxol, or leucovorin.

In some cases, for example, in the compositions, formulations and methods of treating cancer, the unit dosage of the composition or formulation administered can be 5, 10, 15, 20, 25, 30, 35, 40, 45, 50, 55, 60, 65, 70, 75, 80, 85, 90, 95 or 100 mg. In some cases, the total amount of the composition or formulation administered can be 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 40, 50, 60, 70, 80, 90, or 100 g.

In some cases, the present disclosure provides a pharmaceutical composition comprising a T cell can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes.

For example, cells can be administered to a patient in conjunction with (e.g., before, simultaneously, or following) any number of relevant treatment modalities, including but not limited to treatment with agents such as antiviral therapy, cidofovir and interleukin-2, or Cytarabine (also known as ARA-C). In some cases, the engineered cells can be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. The engineered cell composition can also be administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, the engineered cell compositions of the present disclosure can be administered following B-cell ablative therapy such as agents that react with CD20, e.g., Rituxan. For example, subjects can undergo standard treatment with high dose chemotherapy followed by peripheral blood stem cell transplantation. In certain cases, following the transplant, subjects can receive an infusion of the engineered cells, e.g., expanded engineered cells, of the present disclosure. Additionally, expanded engineered cells can be administered before or following surgery. The engineered cells obtained by any one of the methods described herein can be used in a particular aspect of the present disclosure for treating patients in need thereof against Host versus Graft (HvG) rejection and Graft versus Host Disease (GvHD). Therefore, a method of treating patients in need thereof against Host versus Graft (HvG) rejection and Graft versus Host Disease (GvHD) comprising treating a patient by administering to a patient an effective amount of engineered cells comprising inactivated TCR alpha and/or TCR beta genes is contemplated.

### Method of Use

Cells can be extracted from a human as described herein. Cells can be genetically altered ex vivo and used accordingly. These cells can be used for cell-based therapies. These cells can be used to treat disease in a recipient (e.g., a human). For example, these cells can be used to treat cancer.

Described herein is a method of treating a disease (e.g., cancer) in a recipient comprising transplanting to the recipient one or more cells (including organs and/or tissues) comprising engineered cells. Cells prepared by intracellular genomic transplant can be used to treat cancer.

Described herein is a method of treating a disease (e.g., cancer) in a recipient comprising transplanting to the recipient one or more cells (including organs and/or tissues) comprising engineered cells. In some cases 5×10¹⁰ cells will be administered to a patient. In other cases, 5×10¹¹ cells will be administered to a patient.

In some cases, about 5×10¹⁰ cells are administered to a subject. In some cases, about 5×10¹⁰ cells represent the median amount of cells administered to a subject. In some cases, about 5×10¹⁰ cells are necessary to affect a therapeutic response in a subject. In some cases, at least about at least about 1×10⁷ cells, at least about 2×10⁷ cells, at least about 3×10⁷ cells, at least about 4×10⁷ cells, at least about 5×10⁷ cells, at least about 6×10⁷ cells, at least about 6×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 6×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 6×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 6×10¹⁰ cells, at least about 8×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 6×10¹¹ cells, at least about 6×10¹¹ cells, at least about 8×10¹¹ cells, at least about 9×10¹¹ cells, or at least about 1×10¹² cells. For example, about 5×10¹⁰ cells may be administered to a subject. In another example, starting with 3×10⁶ cells, the cells may be expanded to about 5×10¹⁰ cells and administered to a subject. In some cases, cells are expanded to sufficient numbers for therapy. For example, 5×10⁷ cells can undergo rapid expansion to generate sufficient numbers for therapeutic use. In some cases, sufficient numbers for therapeutic use can be 5×10¹⁰. Any number of cells can be infused for therapeutic use. For example, a patient may be infused with a number of cells between 1×10⁶ to 5×10′² inclusive. A patient may be infused with as many cells that can be generated for them. In some cases, cells that are infused into a patient are not all engineered. For example, at least 90% of cells that are infused into a patient can be engineered. In other instances, at least 40% of cells that are infused into a patient can be engineered.

In some cases, a method of the present disclosure comprises calculating and/or administering to a subject an amount of engineered cells necessary to affect a therapeutic response in the subject. In some cases, calculating the amount of engineered cells necessary to affect a therapeutic response comprises the viability of the cells and/or the efficiency with which a transgene has been integrated into the genome of a cell. In some cases, in order to affect a therapeutic response in a subject, the cells administered to the subject may be viable cells. In some cases, in order to effect a therapeutic response in a subject, at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10% of the cells are viable cells. In some cases, in order to affect a therapeutic response in a subject, the cells administered to a subject may be cells that have had one or more transgenes successfully integrated into the genome of the cell. In some cases, in order to effect a therapeutic response in a subject, at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10% of the cells have had one or more transgenes successfully integrated into the genome of the cell.

The method disclosed herein can be used for treating or preventing disease including, but not limited to, cancer, cardiovascular diseases, lung diseases, liver diseases, skin diseases, or neurological diseases.

Transplanting can be by any type of transplanting. Sites can include, but not limited to, liver subcapsular space, splenic subcapsular space, renal subcapsular space, omentum, gastric or intestinal submucosa, vascular segment of small intestine, venous sac, testis, brain, spleen, or cornea. For example, transplanting can be subcapsular transplanting. Transplanting can also be intramuscular transplanting. Transplanting can be intraportal transplanting.

Transplanting can be of one or more cells from a human. For example, the one or more cells can be from an organ, which can be a brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes or lymph vessels. The one or more cells can also be from a brain, heart, liver, skin, intestine, lung, kidney, eye, small bowel, or pancreas. The one or more cells can be from a pancreas, kidney, eye, liver, small bowel, lung, or heart. The one or more cells can be from a pancreas. The one or more cells can be pancreatic islet cells, for example, pancreatic β cells. The one or more cells can be any blood cells, such as peripheral blood mononuclear cell (PBMC), lymphocytes, monocytes or macrophages. The one or more cells can be any immune cells such as lymphocytes, B cells, or T cells.

The method disclosed herein can also comprise transplanting one or more cells, where the one or more cells can be any types of cells. For example, the one or more cells can be epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, granulosa cells, cumulus cells, epidermal cells, endothelial cells, pancreatic islet cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, dopamiergic cells, squamous epithelial cells, osteocytes, osteoblasts, osteoclasts, dopaminergic cells, embryonic stem cells, fibroblasts and fetal fibroblasts. Further, the one or more cells can be pancreatic islet cells and/or cell clusters or the like, including, but not limited to pancreatic α cells, pancreatic β cells, pancreatic δ cells, pancreatic F cells (e.g., PP cells), or pancreatic E cells. In one instance, the one or more cells can be pancreatic α cells. In another instance, the one or more cells can be pancreatic β cells.

Donor can be at any stage of development including, but not limited to, fetal, neonatal, young and adult. For example, donor T cells can be isolated from adult human Donor human T cells can be under the age of 10, 9, 8, 7, 6, 5, 4, 3, 2, or 1 year(s). For example, T cells can be isolated from a human under the age of 6 years. T cells can also be isolated from a human under the age of 3 years. A donor can be older than 10 years.

a. Transplantation

The method disclosed herein can comprise transplanting. Transplanting can be auto transplanting, allotransplanting, xenotransplanting, or any other transplanting. For example, transplanting can be xenotransplanting. Transplanting can also be allotransplanting.

“Xenotransplantation” and its grammatical equivalents as used herein can encompass any procedure that involves transplantation, implantation, or infusion of cells, tissues, or organs into a recipient, where the recipient and donor are different species. Transplantation of the cells, organs, and/or tissues described herein can be used for xenotransplantation in into humans Xenotransplantation includes but is not limited to vascularized xenotransplant, partially vascularized xenotransplant, unvascularized xenotransplant, xenodressings, xenobandages, and xenostructures.

“Allotransplantation” and its grammatical equivalents (e.g., allogenic transplantation) as used herein can encompass any procedure that involves transplantation, implantation, or infusion of cells, tissues, or organs into a recipient, where the recipient and donor are the same species but different individuals. Transplantation of the cells, organs, and/or tissues described herein can be used for allotransplantation into humans. Allotransplantation includes but is not limited to vascularized allotransplant, partially vascularized allotransplant, unvascularized allotransplant, allodressings, allobandages, and allostructures.

“Autotransplantation” and its grammatical equivalents (e.g., autologous transplantation) as used herein can encompass any procedure that involves transplantation, implantation, or infusion of cells, tissues, or organs into a recipient, where the recipient and donor is the same individual. Transplantation of the cells, organs, and/or tissues described herein can be used for autotransplantation into humans. Autotransplantation includes but is not limited to vascularized autotransplantation, partially vascularized autotransplantation, unvascularized autotransplantation, autodressings, autobandages, and autostructures.

After treatment (e.g., any of the treatment as disclosed herein), transplant rejection can be improved as compared to when one or more wild-type cells is transplanted into a recipient. For example, transplant rejection can be hyperacute rejection. Transplant rejection can also be acute rejection. Other types of rejection can include chronic rejection. Transplant rejection can also be cell-mediated rejection or T cell-mediated rejection. Transplant rejection can also be natural killer cell-mediated rejection.

“Improving” and its grammatical equivalents as used herein can mean any improvement recognized by one of skill in the art. For example, improving transplantation can mean lessening hyperacute rejection, which can encompass a decrease, lessening, or diminishing of an undesirable effect or symptom.

After transplanting, the transplanted cells can be functional in the recipient. Functionality can in some cases determine whether transplantation was successful. For example, the transplanted cells can be functional for at least or at least about 1, 2, 3, 4, 5, 6, 7, 8, 9, 10 or more days. This can indicate that transplantation was successful. This can also indicate that there is no rejection of the transplanted cells, tissues, and/or organs.

In certain instances, transplanted cells can be functional for at least 1 day. Transplanted cells can also functional for at least 7 day. Transplanted cells can be functional for at least 14 day. Transplanted cells can be functional for at least 21 day. Transplanted cells can be functional for at least 28 day. Transplanted cells can be functional for at least 60 days.

Another indication of successful transplantation can be the days a recipient does not require immunosuppressive therapy. For example, after treatment (e.g., transplantation) provided herein, a recipient can require no immunosuppressive therapy for at least or at least about 1, 2, 3, 4, 5, 6, 7, 8, 9, 10 or more days. This can indicate that transplantation was successful. This can also indicate that there is no rejection of the transplanted cells, tissues, and/or organs.

In some cases, a recipient can require no immunosuppressive therapy for at least 1 day. A recipient can also require no immunosuppressive therapy for at least 7 days. A recipient can require no immunosuppressive therapy for at least 14 days. A recipient can require no immunosuppressive therapy for at least 21 days. A recipient can require no immunosuppressive therapy for at least 28 days. A recipient can require no immunosuppressive therapy for at least 60 days. Furthermore, a recipient can require no immunosuppressive therapy for at least 1, 2, 3, 4, 5, 6, 7, 8, 9, 10, or more years.

Another indication of successful transplantation can be the days a recipient requires reduced immunosuppressive therapy. For example, after the treatment provided herein, a recipient can require reduced immunosuppressive therapy for at least 1, 2, 3, 4, 5, 6, 7, 8, 9, 10 or more days. This can indicate that transplantation was successful. This can also indicate that there is no or minimal rejection of the transplanted cells, tissues, and/or organs.

In some cases, a recipient can require no immunosuppressive therapy for at least 1 day. A recipient can also require no immunosuppressive therapy for at least or at least about 7 days. A recipient can require no immunosuppressive therapy for at least or at least about 14 days. A recipient can require no immunosuppressive therapy for at least or at least about 21 days. A recipient can require no immunosuppressive therapy for at least or at least about 28 days. A recipient can require no immunosuppressive therapy for at least or at least about 60 days. Furthermore, a recipient can require no immunosuppressive therapy for at least or at least about 1, 2, 3, 4, 5, 6, 7, 8, 9, 10, or more years.

Another indication of successful transplantation can be the days a recipient requires reduced immunosuppressive therapy. For example, after the treatment provided herein, a recipient can require reduced immunosuppressive therapy for at least or at least about 1, 2, 3, 4, 5, 6, 7, 8, 9, 10 or more days. This can indicate that transplantation was successful. This can also indicate that there is no or minimal rejection of the transplanted cells, tissues, and/or organs.

“Reduced” and its grammatical equivalents as used herein can refer to less immunosuppressive therapy compared to a required immunosuppressive therapy when one or more wild-type cells is transplanted into a recipient.

Immunosuppressive therapy can comprise any treatment that suppresses the immune system. Immunosuppressive therapy can help to alleviate, minimize, or eliminate transplant rejection in a recipient. For example, immunosuppressive therapy can comprise immuno-suppressive drugs Immunosuppressive drugs that can be used before, during and/or after transplant, but are not limited to, MMF (mycophenolate mofetil (Cellcept)), ATG (anti-thymocyte globulin), anti-CD154 (CD4OL), anti-CD40 (2C10, ASKP1240, CCFZ533X2201), alemtuzumab (Campath), anti-CD20 (rituximab), anti-IL-6R antibody (tocilizumab, Actemra), anti-IL-6 antibody (sarilumab, olokizumab), CTLA4-Ig (Abatacept/Orencia), belatacept (LEA29Y), sirolimus (Rapimune), everolimus, tacrolimus (Prograf), daclizumab (Ze-napax), basiliximab (Simulect), infliximab (Remicade), cyclosporin, deoxyspergualin, soluble complement receptor 1, cobra venom factor, compstatin, anti C5 antibody (eculizumab/Soliris), methylprednisolone, FTY720, everolimus, leflunomide, anti-IL-2R-Ab, rapamycin, anti-CXCR3 antibody, anti-ICOS antibody, anti-0X40 antibody, and anti-CD122 antibody. Furthermore, one or more than one immunosuppressive agents/drugs can be used together or sequentially. One or more than one immunosuppressive agents/drugs can be used for induction therapy or for maintenance therapy. The same or different drugs can be used during induction and maintenance stages. In some cases, daclizumab (Zenapax) can be used for induction therapy and tacrolimus (Prograf) and sirolimus (Rapimune) can be used for maintenance therapy. Daclizumab (Zenapax) can also be used for induction therapy and low dose tacrolimus (Prograf) and low dose sirolimus (Rapimune) can be used for maintenance therapy Immunosuppression can also be achieved using non-drug regimens including, but not limited to, whole body irradiation, thymic irradiation, and full and/or partial splenectomy. These techniques can also be used in combination with one or more immuno-suppressive drugs.

### EXAMPLES

### Example 1: Determine the Transfection Efficiency of Various Nucleic Acid Delivery Platforms

Isolation of Peripheral Blood Mononuclear Cells (PBMCs) from a LeukoPak

Leukopaks collected from normal peripheral blood were used herein. Blood cells were diluted 3 to 1 with chilled 1X PBS. The diluted blood was added dropwise (e.g., very slowly) over 15 mLs of LYMPHOPREP (Stem Cell Technologies) in a 50 ml conical. Cells were spun at 400×G for 25 minutes with no brake. The buffy coat was slowly removed and placed into a sterile conical. The cells were washed with chilled 1X PBS and spun for 400×G for 10 minutes. The supernatant was removed, cells resuspended in media, counted and viably frozen in freezing media (45 mLs heat inactivated FBS and 5 mLs DMSO).

### Isolation of CD3+ T Cells

PBMCs were thawed and plated for 1-2 hours in culturing media (RPMI-1640 (with no Phenol red), 20% FBS (heat inactivated), and 1× Gluta-MAX). Cells were collected and counted; the cell density was adjusted to 5×10\hat{ }7 cells/mL and transferred to sterile 14 mL polystyrene round-bottom tube. Using the Easy Sep Human CD3 cell Isolation Kit (Stem Cell Technologies), 50 uL/mL of the Isolation Cocktail was added to the cells. The mixture was mixed by pipetting and incubated for 5 minutes at room temperature. After incubation, the RapidSpheres were vortexed for 30 seconds and added at 50 uL/mL to the sample; mixed by pipetting. Mixture was topped off to 5 mLs for samples less than 4 mLs or topped off to 10 mLs for samples more than 4 mLs. The sterile polystyrene tube was added to the “Big Easy” magnet; incubated at room temperature for 3 minutes. The magnet and tube, in one continuous motion, were inverted, pouring off the enriched cell suspension into a new sterile tube.

### Activation and Stimulation of CD3+ T Cells

Isolated CD3+ T cells were counted and plated out at a density of 2×10\hat{ }6 cells/mL in a 24 well plate. Dynabeads Human T-Activator CD3/CD28 beads (Gibco, Life Technologies) were added 3:1 (beads: cells) to the cells after being washed with 1×PBS with 0.2% BSA using a dynamagnet. IL-2 (Peprotech) was added at a concentration of 300 IU/mL. Cells were incubated for 48 hours and then the beads were removed using a dynamagnet. Cells were cultured for an additional 6-12 hours before electroporation or nucelofection.

### Amaxa Transfection of CD3+ T Cells

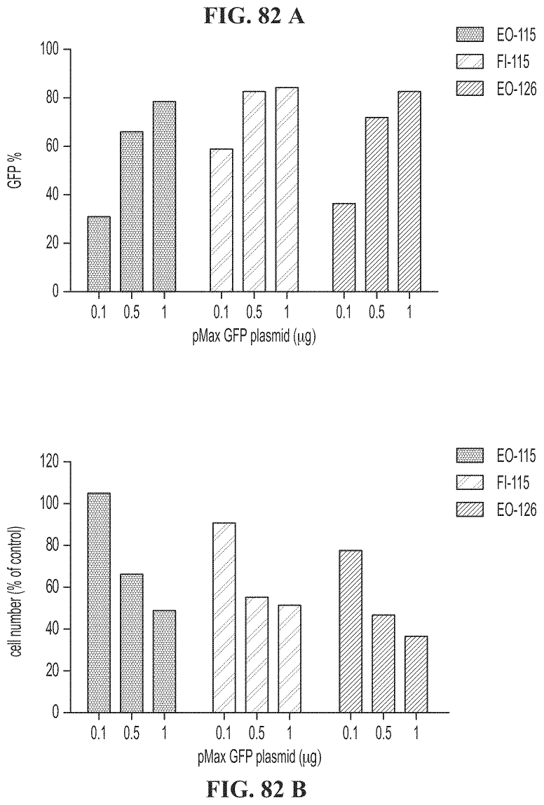

Unstimulated or stimulated T cells were nucleofected using the Amaxa Human T Cell Nucleofector Kit (Lonza, Switzerland), FIG. 82 A. and FIG. 82 B. Cells were counted and resuspended at of density of 1-8×10\hat{ }6 cells in 100 uL of room temperature Amaxa buffer. 1-15 ug of mRNA or plasmids were added to the cell mixture. Cells were nucleofected using the U-014 program. After nucleofection, cells were plated in 2 mLs culturing media in a 6 well plate.

### Neon Transfection of CD3+ T Cells

Unstimulated or stimulated T cells were electroporated using the Neon Transfection System (10 uL Kit, Invitrogen, Life Technologies). Cells were counted and resuspended at a density of 2×10\hat{ }5 cells in 10 uL of T buffer. 1 ug of GFP plasmid or mRNA or 1 ug Cas9 and 1 ug of gRNA plasmid were added to the cell mixture. Cells were electroporated at 1400 V, 10 ms, 3 pulses. After transfection, cells were plated in a 200 uL culturing media in a 48 well plate.

### Lipofection of RNA and Plasmid DNA Transfections of CD3+ T Cells

Unstimulated T cells were plated at a density of 5×10\hat{ }5 cells per mL in a 24 well plate. For RNA transfection, T cells were transfected with 500 ng of mRNA using the TransIT-mRNA Transfection Kit (Minis Bio), according to the manufacturer's protocol. For Plasmid DNA transfection, the T cells were transfected with 500 ng of plasmid DNA using the TransIT-X2 Dynamic Delivery System (Minis Bio), according to the manufacturer's protocol. Cells were incubated at 37° C. for 48 hours before being analyzed by flow cytometry.

### CD3+ T Cell Uptake of Gold Nanoparticle SmartFlares

Unstimulated or stimulated T cells were plated at a density of 1-2×10\hat{ }5 cells per well in a 48 well plate in 200 uL of culturing media. Gold nanoparticle SmartFlared complexed to Cy5 or Cy3 (Millipore, Germany) were vortexed for 30 seconds prior to being added to the cells. 1 uL of the gold nanoparticle SmartFlares was added to each well of cells. The plate was rocked for 1 minute incubated for 24 hours at 37° C. before being analyzed for Cy5 or Cy3 expression by flow cytometry.

### Flow Cytometry

Electroporated and nucleofected T cells were analyzed by flow cytometry 24-48 hours post transfection for expression of GFP. Cells were prepped by washing with chilled 1×PBS with 0.5% FBS and stained with APC anti-human CDR (eBiosciences, San Diego) and Fixable Viability Dye eFlour 780 (eBiosciences, San Diego). Cells were analyzed using a LSR II (BD Biosciences, San Jose) and FlowJo v.9.

### Results

As shown in Table 2, a total of six cell and DNA/RNA combinations were tested using four exemplary transfection platforms. The six cell and DNA/RNA combinations were: adding EGFP plasmid DNA to unstimulated PBMCs; adding EGFP plasmid DNA to unstimulated T cells; adding EGFP plasmid DNA to stimulated T cells; adding EGFP mRNA to unstimulated PBMCs; adding EGFP mRNA to unstimulated T cells; and adding EGFP mRNA to stimulated T cells. The four exemplary transfection platforms were: AMAXA Nucleofection, NEON Eletrophoration, Lipid-based Transfection, and Gold Nanoparticle delivery. The transfection efficiency (% of transfected cells) results under various conditions were listed in Table 1 and adding mRNA to stimulated T cells using AMAXA platform provides the highest efficiency.

TABLE 2
The transfection efficiency of various nucleic acid
delivery platforms.

| | Nucleic Acid Delivery Platforms | | | | | |
|---|---|---|---|---|---|---|
| | DNA or | | | | | Gold |
| Cell type | RNA | Amaxa | NEON | Lipid Based | | Nanoparticle |
| PBMCs loading | EGFP | 8.1% (CD3 T- | | | | |
| (unstimulated) | Plasmid | Cells) | | | | |
| T-Cell loading | EGFP | 28.70% | >0.1% | >0.1% | >0.1% | 54.8% Cy5 Pos. |
| (unstimulated) | Plasmid | | | (DNA) | (RNA) | |
| T-Cell loading | EGFP | | 32.10% | >0.1% | >0.1% | |
| (Stimulated, | Plasmid | | | (DNA) | (RNA) | |
| CD3/CD28) | | | | | | |
| PBMCs loading | EGFP | 28.1% (CD3 T- | | | | |
| (unstimulated) | mRNA | Cells) | | | | |
| T-Cell loading | EGFP | 29.80% | | | | |
| (unstimulated) | mRNA | | | | | |
| T-Cell loading | EGFP | 90.30% | 81.40% | | | 29.1% Cy5 Pos. |
| (Stimulated, | mRNA | | | | | |
| CD3/CD28) | | | | | | |

Other transfection conditions including exosome-mediated transfection will be tested using similar methods in the future. In addition, other delivery combinations including DNA Cas9/DNA gRNA, mRNA Cas9/DNA gRNA, protein Cas9/DNA gRNA, DNA Cas9/PCR product of gRNA, DNA Cas9/PCR product of gRNA, mRNA Cas9/PCR product of gRNA, protein Cas9/PCR product of gRNA, DNA Cas9/modified gRNA, mRNA Cas9/modified gRNA, and protein Cas9/modified gRNA, will also be tested using similar methods. The combinations with high delivery efficiency can be used in the methods disclosed herein.

### Example 2: Determine the Transfection Efficiency of a GFP Plasmid in T Cells

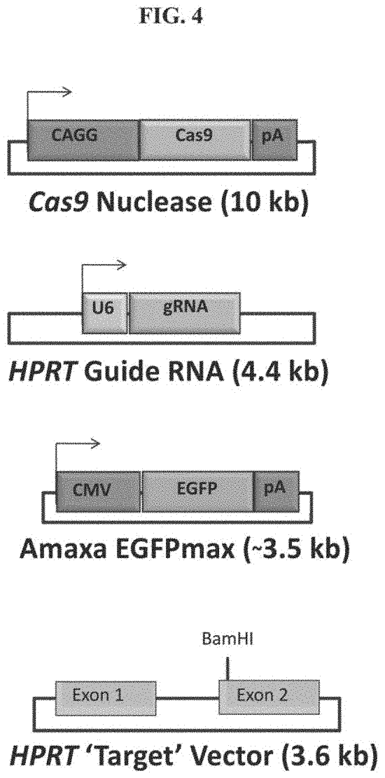

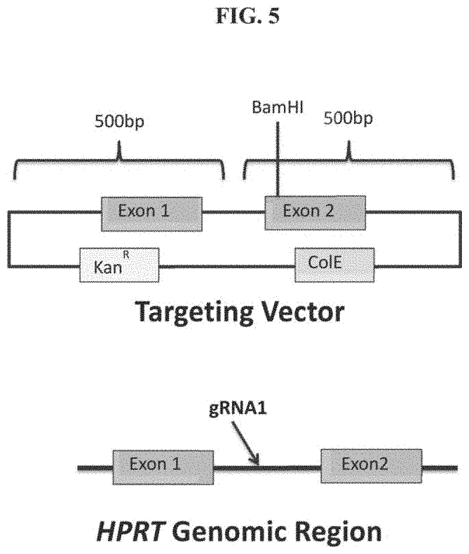

The transfection efficiency of primary T cells with Amaxa Nuclofection using a GFP plasmid. FIG. 4 showed the structures of four plasmids prepared for this experiment: Cas9 nuclease plasmid, HPRT gRNA plasmid (CRISPR gRNA targeting human HPRT gene), Amaxa EGFPmax plasmid and HPRT target vector. The HPRT target vector had targeting arms of 0.5 kb (FIG. 5). The sample preparation, flow cytometry and other methods were similar to experiment 1. The plasmids were prepared using the endotoxin free kit (Qiagen). Different conditions (shown in Table 3) including cell number and plasmid combination were tested.

TABLE 3
The different conditions used in the experiment.

| Sample′ | | | GFP′ | Cas9′ | gRNA′ | target′ |
|---|---|---|---|---|---|---|
| ID | #PBMCs | Plasmids | (ug) | (ug) | (ug) | (ug) |
| 1 | 5 × 10\hat{ }6 | GFP | 5 | 0 | 0 | 0 |
| 2 | 2 × 10\hat{ }7 | Cas9 | 0.1 | 20 | 0 | 0 |
| 3 | 2 × 10\hat{ }7 | Cas9 + gRNA | 0.1 | 10 | 10 | 0 |
| 4 | 2 × 10\hat{ }7 | Cas9 + gRNA + | 0.1 | 5 | 5 | 10 |
| | | Target | | | | |
| 5 | 2 × 10\hat{ }7 | Cas9 + gRNA + | 0.1 | 2.5 | 2.5 | 15 |
| | | Target | | | | |
| 6 | 2 × 10\hat{ }7 | GFP | 5 | 0 | 0 | 0 |

### Results

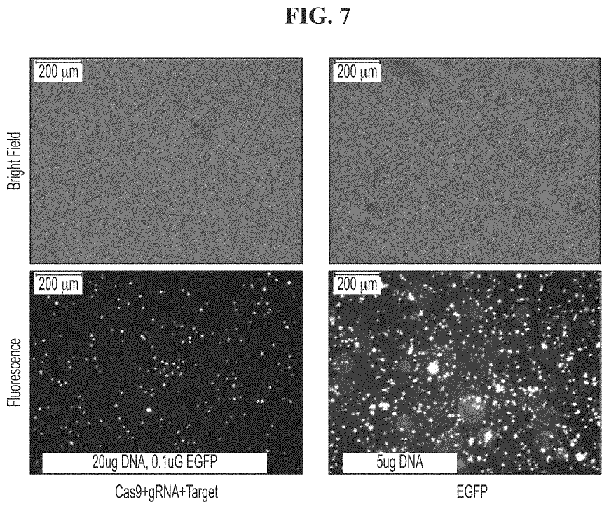

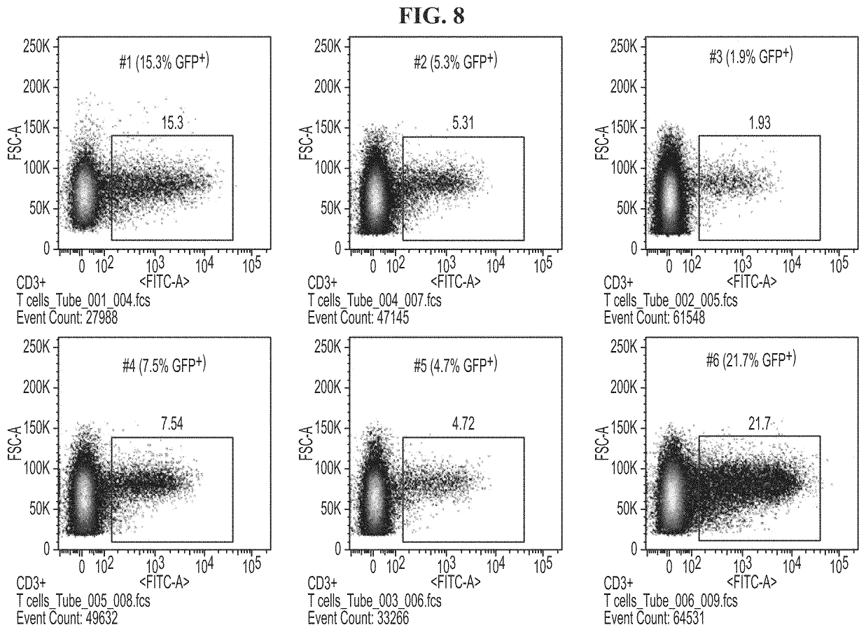

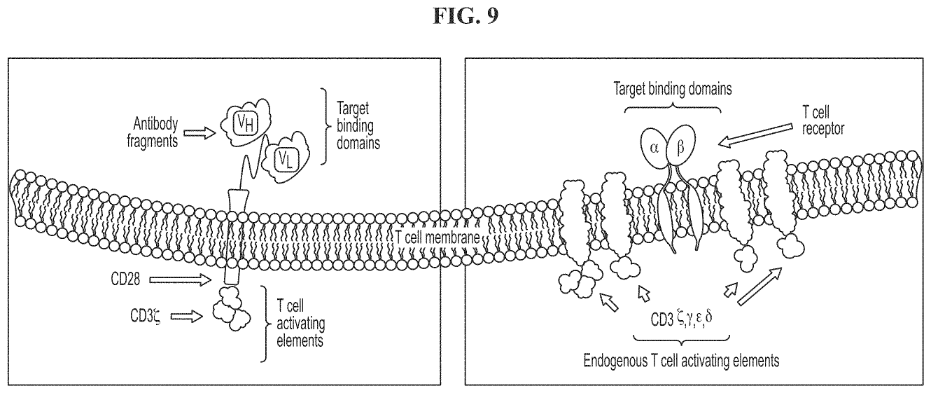

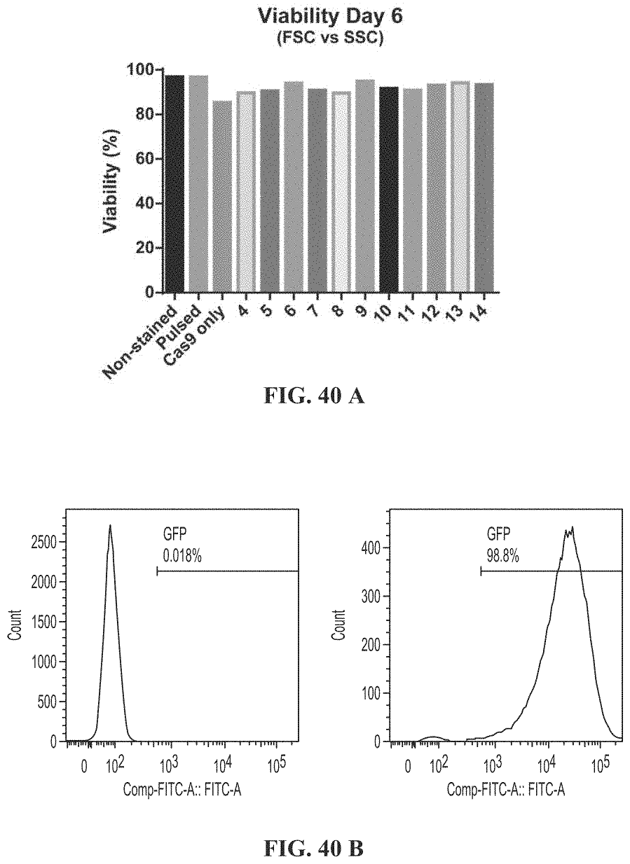

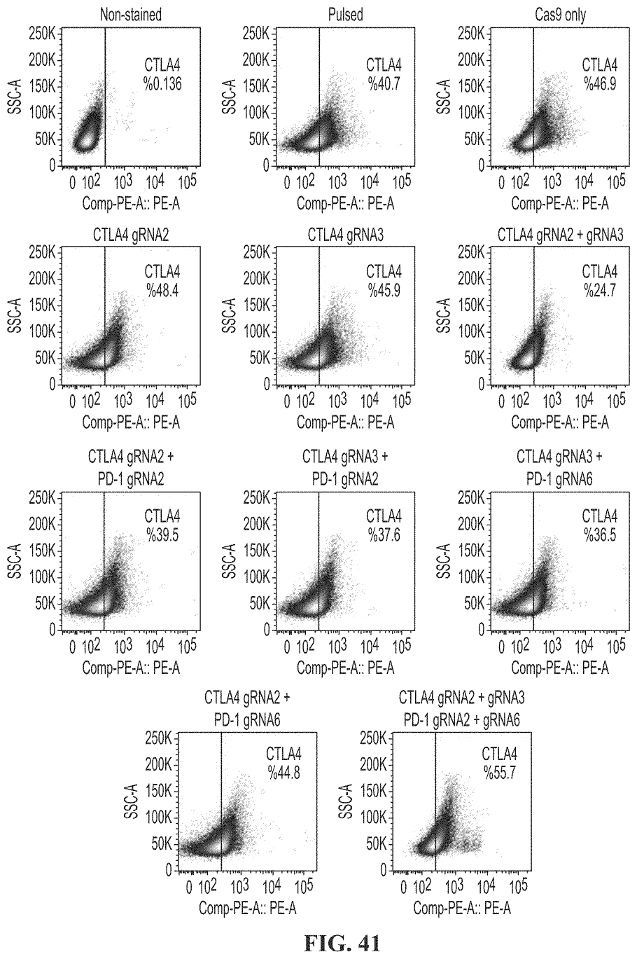

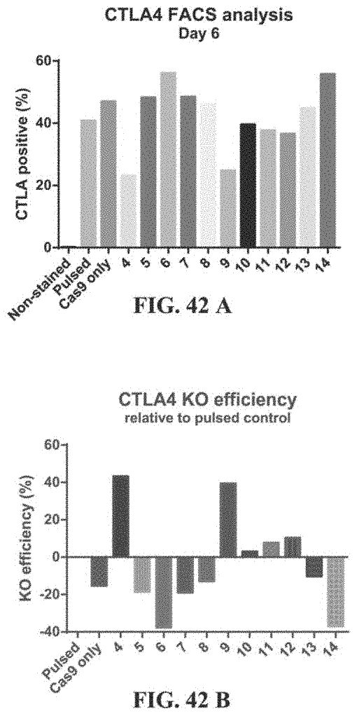

FIG. 7 demonstrated that the Cas9+gRNA+Target plasmids co-transfection had good transfection efficiency in bulk population. FIG. 8 showed the results of the EGFP FACS analysis of CD3+ T cells. Different transfection efficiencies were demonstrated using the above conditions. FIG. 40 A and FIG. 40 B show viability and transfection efficiency on day 6 post CRISPR transfection with a donor transgene (% GFP+).

### Example 3: Identify gRNA with Highest Double Strand Break (DSB) Induction at Each Gene Site

### Design and Construction of Guide RNAs:

Guide RNAs (gRNAs) were designed to the desired region of a gene using the CRISPR Design Program (Zhang Lab, M I T 2015). Multiple primers to generate gRNAs (shown in Table 4) were chosen based on the highest ranked values determined by off-target locations. The gRNAs were ordered in oligonucleotide pairs: 5′-CACCG-gRNA sequence-3′ and 5′-AAAC-reverse complement gRNA sequence-C-3′ (sequences of the oligonucleotide pairs are listed in Table 4).

TABLE 4
Primers used to generate the gRNAs (the sequence
CACCG is added to the sense and AAAC to the
antisense for cloning purposes).

| SEQ | | |
|---|---|---|
| ID | Primer Name | Sequence 5′-3′ |
| 5 | HPRT gRNA 1 Sense | CACCGCACGTGTGAACCAACCCGCC |
| 6 | HPRT gRNA 1 Anti | AAACGGCGGGTTGGTTCACACGTGC |
| 7 | HPRT gRNA 2 Sense | CACCGAAACAACAGGCCGGGCGGGT |
| 8 | HPRT gRNA 2 Anti | AAACACCCGCCCGGCCTGTTGTTTC |
| 9 | HPRT gRNA 3 Sense | CACCGACAAAAAAATTAGCCGGGTG |
| 10 | HPRT gRNA 3 Anti | AAACCACCCGGCTAATTTTTTTGT |
| 11 | HPRT gRNA 4 Sense | CACCGTAAATTTCTCTGATAGACTA |
| 12 | HPRT gRNA 4 Anti | AAACTAGTCTATCAGAGAAATTTAC |
| 13 | HPRT gRNA 5 Sense | CACCGTGTTTCAATGAGAGCATTAC |
| 14 | HPRT gRNA 5 Anti | AAACGTAATGCTCTCATTGAAACAC |
| 15 | HPRT gRNA 6 Sense | CACCGGTCTCGAACTCCTGAGCTC |
| 16 | HPRT gRNA 6 Anti | AAACGAGCTCAGGAGTTCGAGACC |
| 17 | HPRT CelI For | AGTGAAGTGGCGCATTCTTG |
| 18 | HPRT CelI Rev | CACCCTTTCCAAATCCTCAGC |
| 19 | AAVS1 gRNA 1 Sense | CACCGTGGGGGTTAGACCCAATATC |
| 20 | AAVS1 gRNA 1 Anti | AAACGATATTGGGTCTAACCCCCAC |
| 21 | AAVS1 gRNA 2 Sense | CACCGACCCCACAGTGGGGCCACTA |
| 22 | AAVS1 gRNA 2 Anti | AAACTAGTGGCCCCACTGTGGGGTC |
| 23 | AAVS1 gRNA 3 Sense | CACCGAGGGCCGGTTAATGTGGCTC |
| 24 | AAVS1 gRNA 3 Anti | AAACGAGCCACATTAACCGGCCCTC |
| 25 | AAVS1 gRNA 4 Sense | CACCGTCACCAATCCTGTCCCTAG |
| 26 | AAVS1 gRNA 4 Anti | AAACCTAGGGACAGGATTGGTGAC |
| 27 | AAVS1 gRNA 5 Sense | CACCGCCGGCCCTGGGAATATAAGG |
| 28 | AAVS1 gRNA 5 Anti | AAACCCTTATATTCCCAGGGCCGGC |
| 29 | AAVS1 gRNA 6 Sense | CACCGCGGGCCCCTATGTCCACTTC |
| 30 | AAVS1 gRNA 6 Anti | AAACGAAGTGGACATAGGGGCCCGC |
| 31 | AAVS1 CelI For | ACTCCTTTCATTTGGGCAGC |
| 32 | AAVS1 CelI Rev | GGTTCTGGCAAGGAGAGAGA |
| 33 | PD-1 gRNA 1 Sense | CACCGCGGAGAGCTTCGTGCTAAAC |
| 34 | PD-1 gRNA 1 Anti | AAACGTTTAGCACGAAGCTCTCCGC |
| 35 | PD-1 gRNA 2 Sense | CACCGCCTGCTCGTGGTGACCGAAG |
| 36 | PD-1 gRNA 2 Anti | AAACCTTCGGTCACCACGAGCAGGC |
| 37 | PD-1 gRNA 3 Sense | CACCGCAGCAACCAGACGGACAAGC |
| 38 | PD-1 gRNA 3 Anti | AAACGCTTGTCCGTCTGGTTGCTGC |
| 39 | PD-1 gRNA 4 Sense | CACCGAGGCGGCCAGCTTGTCCGTC |
| 40 | PD-1 gRNA 4 Anti | AAACGACGGACAAGCTGGCCGCCTC |
| 41 | PD-1 gRNA 5 Sense | CACCGCGTTGGGCAGTTGTGTGACA |
| 42 | PD-1 gRNA 5 Anti | AAACTGTCACACAACTGCCCAACGC |
| 43 | PD-1 gRNA 6 Sense | CACCGACGGAAGCGGCAGTCCTGGC |
| 44 | PD-1 gRNA 6 Anti | AAACGCCAGGACTGCCGCTTCCGTC |
| 45 | PD-1 CelI For | AGAAGGAAGAGGCTCTGCAG |
| 46 | PD-1 CelI Rev | CTCTTTGATCTGCGCCTTGG |
| 47 | CTLA4 gRNA 1 Sense | CACCGCCGGGTGACAGTGCTTCGGC |
| 48 | CTLA4 gRNA 1 Anti | AAACGCCGAAGCACTGTCACCCGGC |
| 49 | CTLA4 gRNA 2 Sense | CACCGTGCGGCAACCTACATGATG |
| 50 | CTLA4 gRNA 2 Anti | AAACCATCATGTAGGTTGCCGCAC |
| Si | CTLA4 gRNA 3 Sense | CACCGCTAGATGATTCCATCTGCAC |
| 52 | CTLA4 gRNA 3 Anti | AAACGTGCAGATGGAATCATCTAGC |
| 53 | CTLA4 gRNA 4 Sense | CACCGAGGTTCACTTGATTTCCAC |
| 54 | CTLA4 gRNA 4 Anti | AAACGTGGAAATCAAGTGAACCTC |
| 55 | CTLA4 gRNA 5 Sense | CACCGCCGCACAGACTTCAGTCACC |
| 56 | CTLA4 gRNA 5 Anti | AAACGGTGACTGAAGTCTGTGCGGC |
| 57 | CTLA4 gRNA 6 Sense | CACCGCTGGCGATGCCTCGGCTGC |
| 58 | CTLA4 gRNA 6 Anti | AAACGCAGCCGAGGCATCGCCAGC |
| 59 | CTLA4 CelI For | TGGGGATGAAGCTAGAAGGC |
| 60 | CTLA4 CelI Rev | AATCTGGGTTCCGTTGCCTA |
| 61 | CCR5 gRNA 1 Sense | CACCGACAATGTGTCAACTCTTGAC |
| 62 | CCR5 gRNA 1 Anti | AAACGTCAAGAGTTGACACATTGTC |
| 63 | CCR5 gRNA 2 Sense | CACCGTCATCCTCCTGACAATCGAT |
| 64 | CCR5 gRNA 2 Anti | AAACATCGATTGTCAGGAGGATGAC |
| 65 | CCR5 gRNA 3 Sense | CACCGGTGACAAGTGTGATCACTT |
| 66 | CCR5 gRNA 3 Anti | AAACAAGTGATCACACTTGTCACC |
| 67 | CCR5 gRNA 4 Sense | CACCGACACAGCATGGACGACAGCC |
| 68 | CCR5 gRNA 4 Anti | AAACGGCTGTCGTCCATGCTGTGTC |
| 69 | CCR5 gRNA 5 Sense | CACCGATCTGGTAAAGATGATTCC |
| 70 | CCR5 gRNA 5 Anti | AAACGGAATCATCTTTACCAGATC |
| 71 | CCR5 gRNA 6 Sense | CACCGTTGTATTTCCAAAGTCCCAC |
| 72 | CCR5 gRNA 6 Anti | AAACGTGGGACTTTGGAAATACAAC |
| 73 | CCR5 CelI For | CTCAACCTGGCCATCTCTGA |
| 74 | CCR5 CelI Rev | CCCGAGTAGCAGATGACCAT |

The gRNAs were cloned together using the target sequence cloning protocol (Zhang Lab, MIT). Briefly, the oligonucleotide pairs were phosphorylated and annealed together using T4 PNK (NEB) and 10X T4 Ligation Buffer (NEB) in a thermocycler with the following protocol: 37° C. 30 minutes, 95° C. 5 minutes and then ramped down to 25° C. at 5° C./minute. pENTR1-U6-Stuffer-gRNA vector (made in house) was digested with FastDigest BbsI (Fermentas), FastAP (Fermentas) and 10X Fast Digest Buffer were used for the ligation reaction. The digested pENTRl vector was ligated together with the phosphorylated and annealed oligo duplex (dilution 1:200) from the previous step using T4 DNA Ligase and Buffer (NEB). The ligation was incubated at room temperature for 1 hour and then transformed and subsequently mini-prepped using GeneJET Plasmid Miniprep Kit (Thermo Scientific). The plasmids were sequenced to confirm the proper insertion.

TABLE 5
Engineered CISH guide RNA (gRNA) target sequences

| SEQ ID | gRNA No. | Exon | Target 5′-3′ |
|---|---|---|---|
| 75 | 1 | 2 | TTGCTGGCTGTGGAGCGGAC |
| 76 | 2 | 2 | GACTGGCTTGGGCAGTTCCA |
| 77 | 3 | 2 | TGCTGGGGCCTTCCTCGAGG |
| 78 | 4 | 2 | CCGAAGGTAGGAGAAGGTCT |
| 79 | 5 | 2 | ATGCACAGCAGATCCTCCTC |
| 80 | 6 | 2 | AGAGAGTGAGCCAAAGGTGC |
| 81 | 1 | 3 | GGCATACTCAATGCGTACAT |
| 82 | 2 | 3 | GGGTTCCATTACGGCCAGCG |
| 83 | 3 | 3 | AAGGCTGACCACATCCGGAA |
| 84 | 4 | 3 | TGCCGACTCCAGCTTCCGTC |
| 85 | 5 | 3 | CTGTCAGTGAAAACCACTCG |
| 86 | 6 | 3 | CGTACTAAGAACGTGCCTTC |

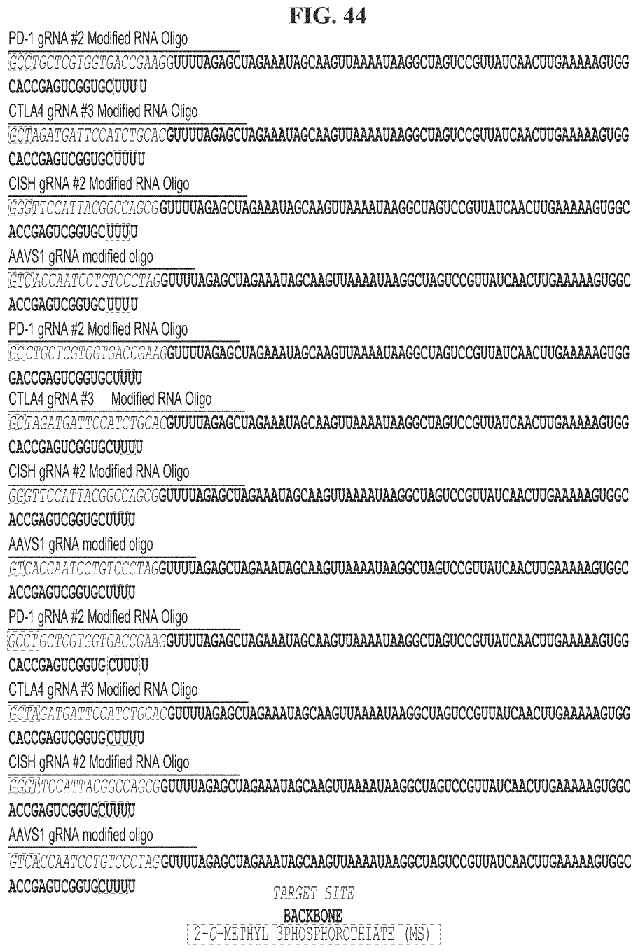

Genomic sequences that are targeted by engineered gRNAs are shown in Table 5 and Table 6. FIG. 44 A and FIG. 44 B show modified gRNA targeting the CISH gene.

TABLE 6
AAVS1 gRNA target sequence

| SEQ ID | Gene | gRNA Sequence (5′ to 3′) |
|---|---|---|
| 87 | AAVS1 | GTCACCAATCCTGTCCCTAG- |

Validation of gRNAs

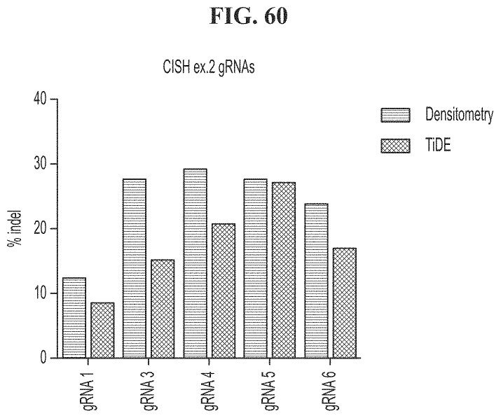

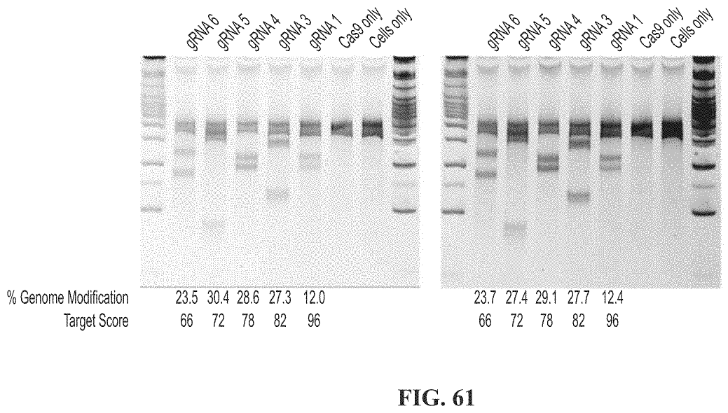

HEK293T cells were plated out at a density of 1×10\hat{ }5 cells per well in a 24 well plate. 150 uL of Opti-MEM medium was combined with 1.5 ug of gRNA plasmid, 1.5 ug of Cas9 plasmid. Another 150 uL of Opti-MEM medium was combined with 5 ul of Lipofectamine 2000 Transfection reagent (Invitrogen). The solutions were combined together and incubated for 15 minutes at room temperature. The DNA-lipid complex was added dropwise to wells of the 24 well plates. Cells were incubated for 3 days at 37° C. and genomic DNA was collected using the GeneJET Genomic DNA Purification Kit (Thermo Scientific). Activity of the gRNAs was quantified by a Surveyor Digest, gel electrophoresis, and densitometry (FIG. 60 and FIG. 61) (Guschin, D. Y., et al., “A Rapid and General Assay for Monitoring Endogenous Gene Modification,” Methods in Molecular Biology, 649: 247-256 (2010)).

### Plasmid Targeting Vector Construction

Sequences of target integration sites were acquired from ensemble database. PCR primers were designed based on these sequences using Primer3 software to generate targeting vectors of carrying lengths, 1kb, 2kb, and 4kb in size. Targeting vector arms were then PCR amplified using Accuprime Taq HiFi (Invitrogen), following manufacturer's instructions. The resultant PCR products were then sub cloned using the TOPO-PCR-Blunt II cloning kit (Invitrogen) and sequence verified. A representative targeting vector construct is shown in FIG. 16.

### Results

The efficiencies of Cas9 in creating double strand break (DSB) with the assistance of different gRNA sequences were listed in Table 7. The percentage numbers in Table 7 indicated the percent of gene modifications in the sample.

TABLE 7
The efficiencies of Cas9/gRNA pair in creating double
strand break (DSB) at each target gene site.

| | HPRT | AAVS1 | CCR5 | PD1 | CTLA4 |
|---|---|---|---|---|---|
| gRNA#1 | 27.85% | 32.99% | 21.47% | 10.83% | 40.96% |
| gRNA#2 | 30.04% | 27.10% | >60% | >60% | 56.10% |
| gRNA#3 | <1% | 39.82% | 55.98% | 37.42% | 39.33% |
| gRNA#4 | <5% | 25.93% | 45.99% | 20.87% | 40.13% |
| gRNA#5 | <1% | 27.55% | 36.07% | 30.60% | 15.90% |
| gRNA#6 | <5% | 39.62% | 33.17% | 25.91% | 36.93% |

DSB were created at all five tested target gene sites. Among them, CCR5, PD1, and CTLA4 provided the highest DSB efficiency. Other target gene sites, including hRosa26, will be tested using the same methods described herein.

The rates of Cas9 in creating double strand break in conjunction with different gRNA sequences is shown in FIG. 15. The percent of double strand break compared to donor control and Cas9 only controls are listed. A three representative target gene sites (i.e., CCR5, PD1, and CTLA4) were tested.

### Example 4: Generation of T Cells Comprising an Engineered TCR that Also Disrupts an Immune Checkpoint Gene

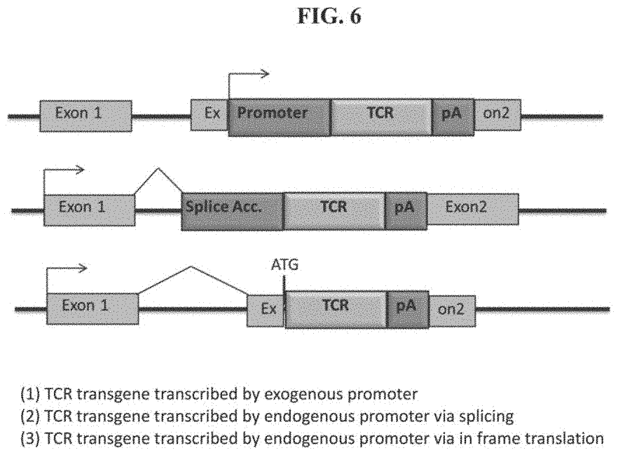

To generate a T cell population that expresses an engineered TCR that also disrupts an immune checkpoint gene, CRISPR, TALEN, transposon-based, ZEN, meganuclease, or Mega-TAL gene editing method will be used. A summary of PD-1 and other endogenous checkpoints is shown in Table 9. Cells (e.g., PBMCs, T cells such as TILs, CD4+ or CD8+ cells) will be purified from a cancer patient (e.g., metastatic melanoma) and cultured and/or expanded according to standard procedures. Cells will be stimulated (e.g., using anti-CD3 and anti-CD28 beads) or unstimulated. Cells will be transfected with a target vector carrying a TCR transgene. For example, TCR transgene sequence of MBVb22 will be acquired and synthesized by IDT as a gBLOCK. The gBLOCK will be designed with flanking attB sequences and cloned into pENTR1 via the LR Clonase reaction (Invitrogen), following manufacturer's instructions, and sequence verified. Three transgene configurations (see FIG. 6) that express a TCR transgene in three different ways will be tested: 1) Exogenous promoter: TCR transgene is transcribed by an exogenous promoter; 2) SA in-frame transcription: TCR transgene is transcribed by endogenous promoter via splicing; and 3) Fusion in frame translation: TCR transgene transcribed by endogenous promoter via in frame translation.

When CRISPR gene editing method is used, a Cas9 nuclease plasmid and a gRNA plasmid (similar to the plasmids shown in FIG. 4) will be also transfected with the DNA plasmid with the target vector carrying a TCR transgene. 10 micrograms of gRNA and 15 micrograms of Cas 9 mRNA can be utilized. The gRNA guides the Cas9 nuclease to an integration site, for example, an endogenous checkpoint gene such as PD-1. Alternatively, PCR product of the gRNA or a modified RNA (as demonstrated in Hendel, Nature biotechnology, 2015) will be used. Another plasmid with both the Cas9 nuclease gene and gRNA will be also tested. The plasmids will be transfected together or separately. Alternatively, Cas9 nuclease or a mRNA encoding Cas9 nuclease will be used to replace the Cas9 nuclease plasmid.

To optimize the rate of homologous recombination to integrate TCR transgene using CRISPR gene editing method, different lengths of target vector arms will be tested, including 0.5 kbp, 1 kbp, and 2 kbp. For example, a target vector with a 0.5 kbp arm length is illustrated in FIG. 5. In addition, the effect of a few CRISPR enhancers such as SCR7 drug and DNA Ligase IV inhibitor (e.g., adenovirus proteins) will be also tested.

In addition to delivering a homologous recombination HR enhancer carrying a transgene using a plasmid, the use of mRNA will be also tested. An optimal reverse transcription platform capable of high efficiency conversion of mRNA homologous recombination HR enhancer to DNA in situ will be identified. The reverse transcription platform for engineering of hematopoietic stem cells and primary T-cells will be also optimized and implemented.

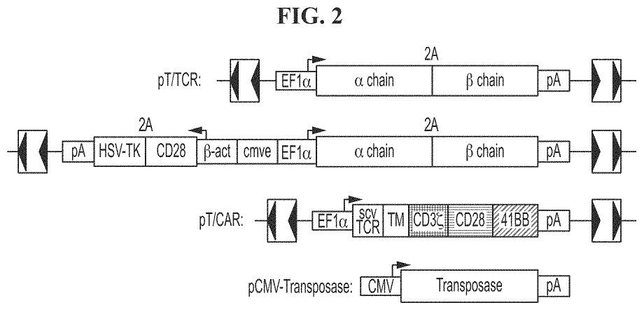

When transposon-based gene editing method (e.g., PiggyBac, Sleeping Beauty) will be used, a transposase plasmid will be also transfected with the DNA plasmid with the target vector carrying a TCR transgene. FIG. 2 illustrates some of the transposon-based constructs for TCR transgene integration and expression.

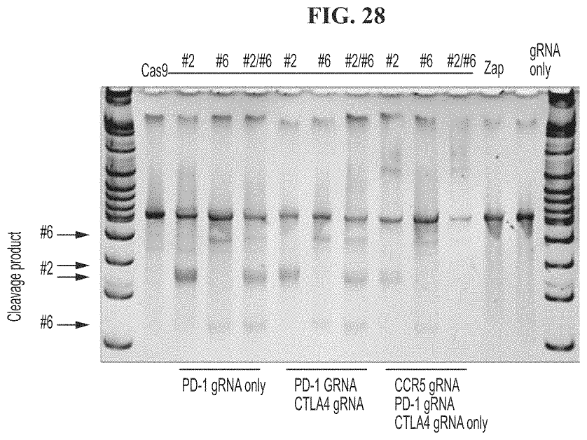

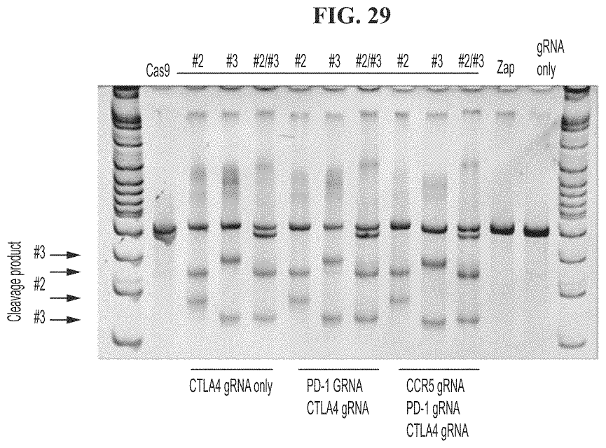

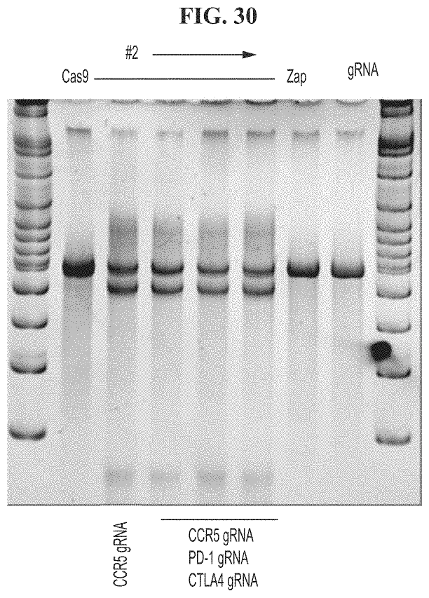

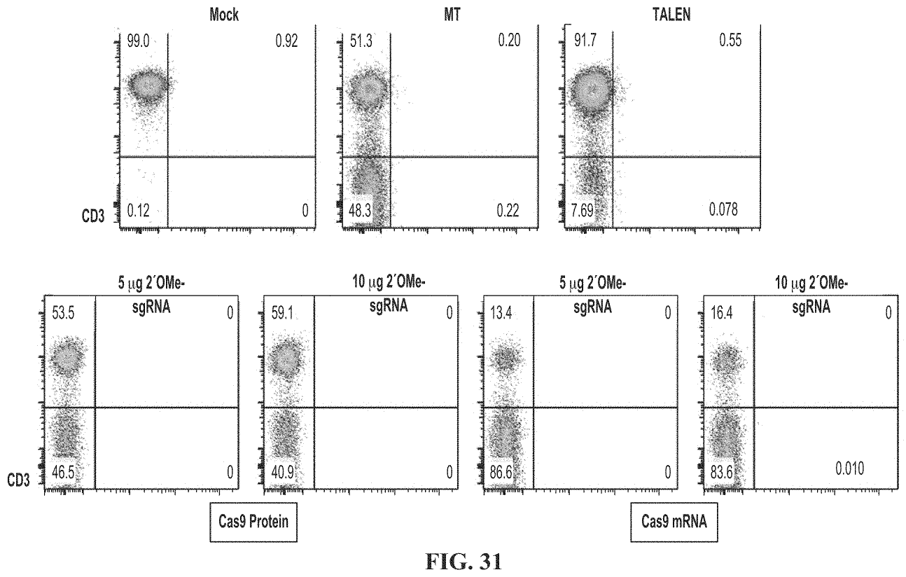

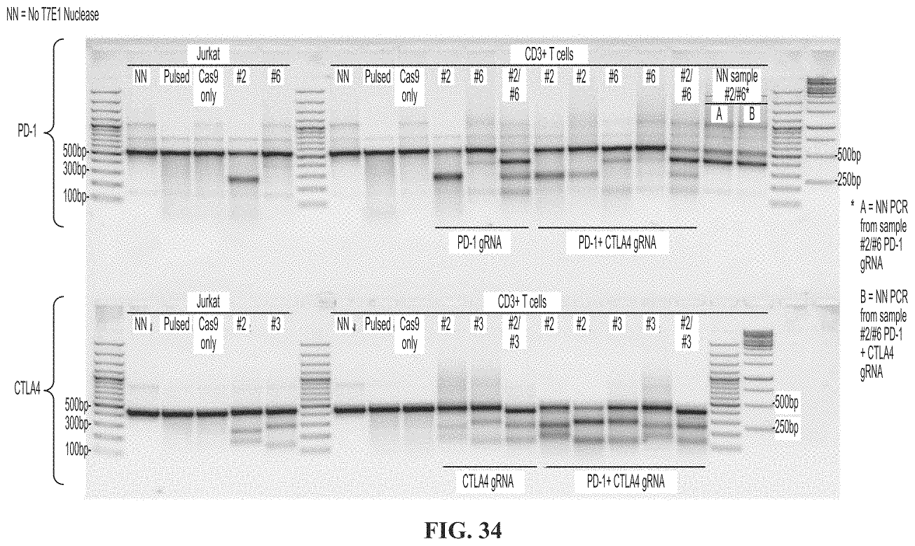

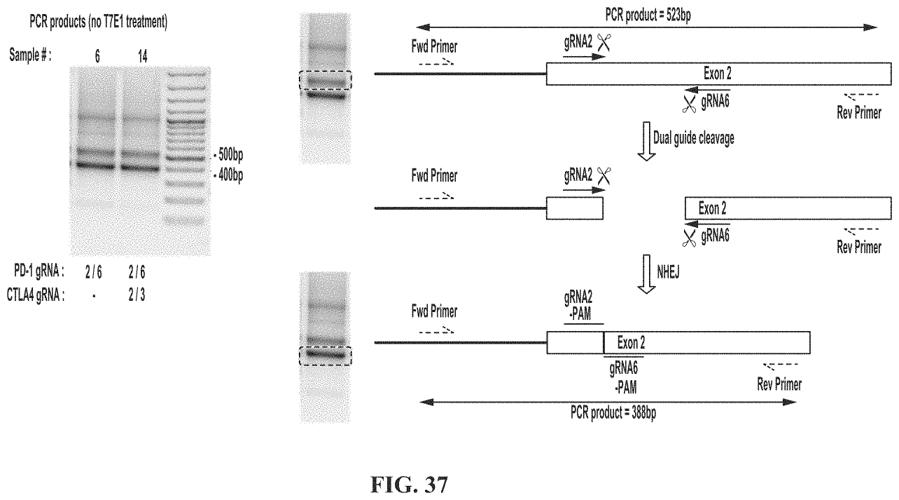

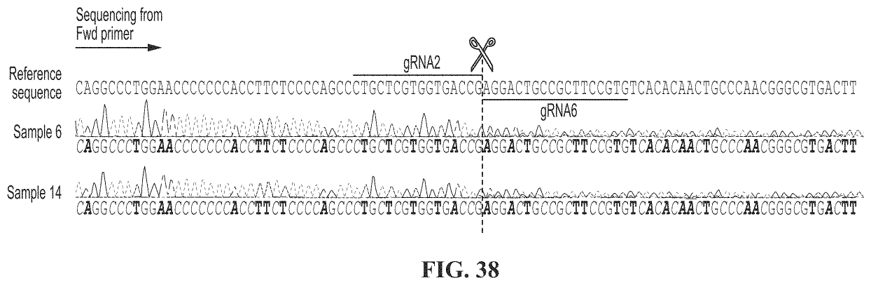

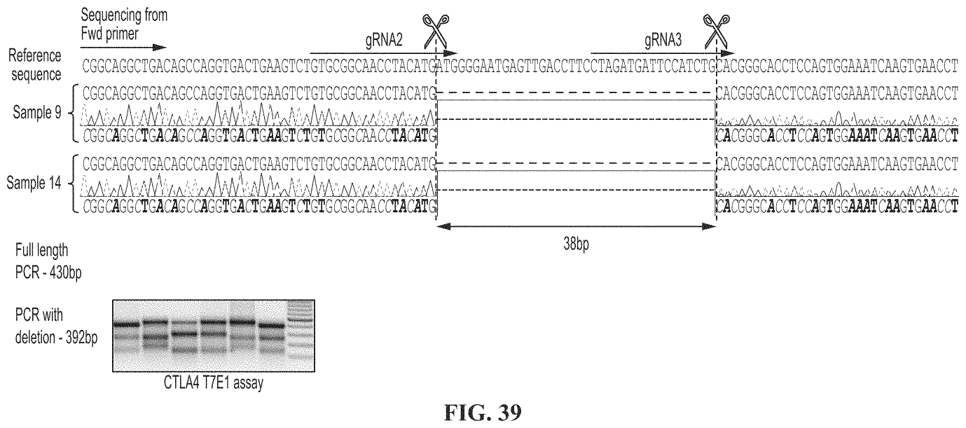

The engineered cells will then be treated with mRNAs encoding PD1-specific nucleases and the population will be analyzed by the Cel-I assay (FIG. 28 to FIG. 30) to verify PD1 disruption and TCR transgene insertion. After the verification, the engineered cells will then be grown and expanded in vitro. The T7 endonuclease I (T7E1) assay can be used to detect on-target CRISPR events in cultured cells, FIG. 34 and FIG. 39. Dual sequencing deletion is shown in FIG. 37 and FIG. 38.

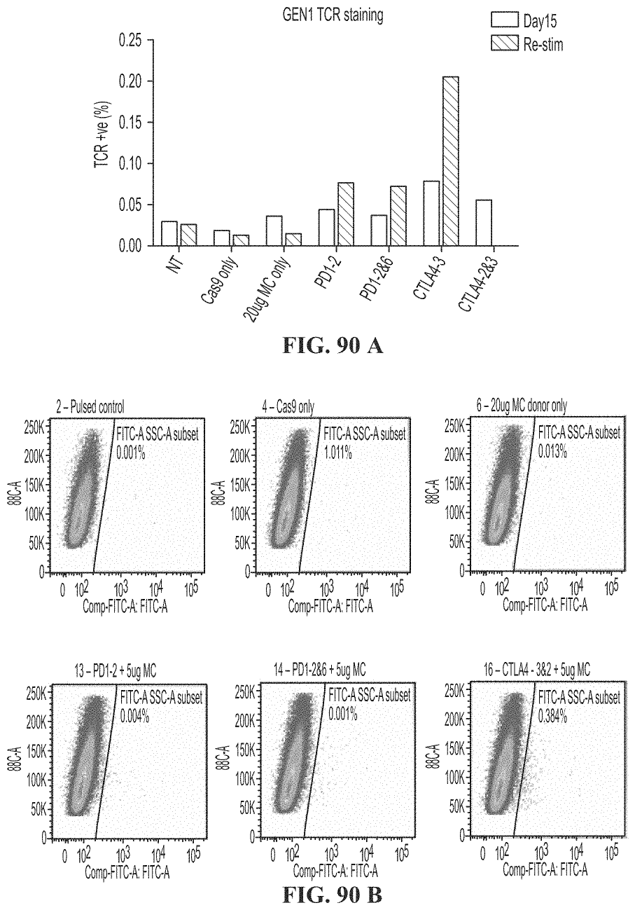

Some engineered cells will be used in autologous transplantation (e.g., administered back to the cancer patient whose cells were used to generate the engineered cells). Some engineered cells will be used in allogenic transplantation (e.g., administered back to a different cancer patient). The efficacy and specificity of the T cells in treating patients will be determined. Cells that have been genetically engineered can be restimulated with antigen or anti-CD3 and anti-CD28 to drive expression of an endogenous checkpoint gene, FIG. 90A and FIG. 90B.

### Results

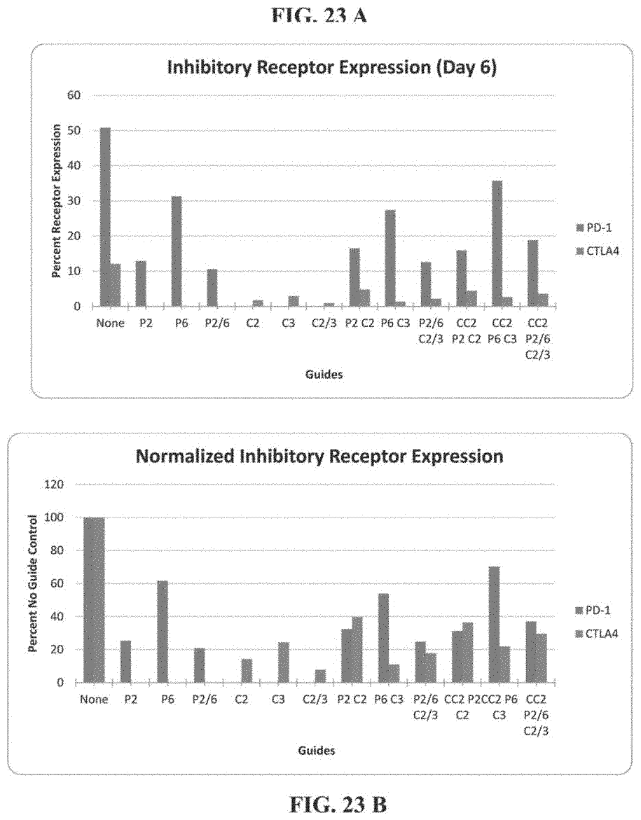

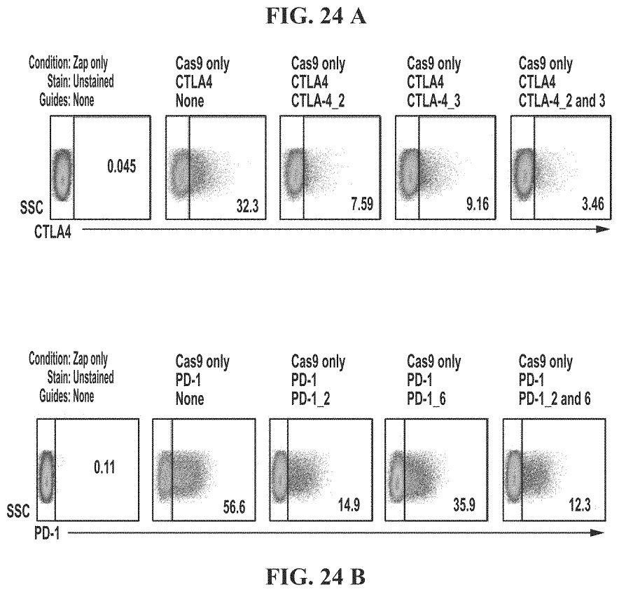

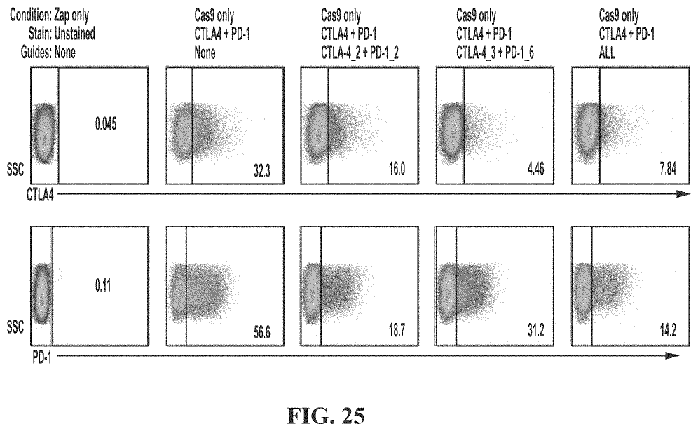

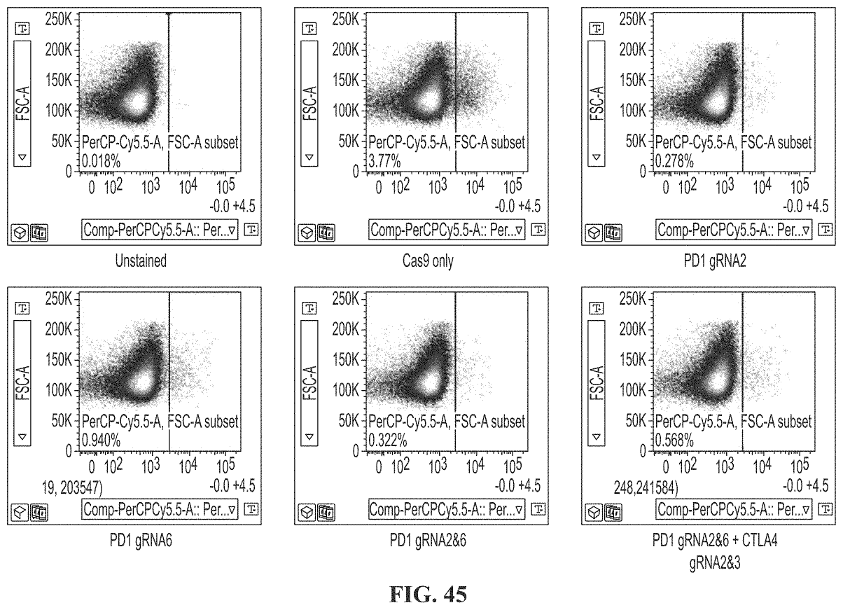

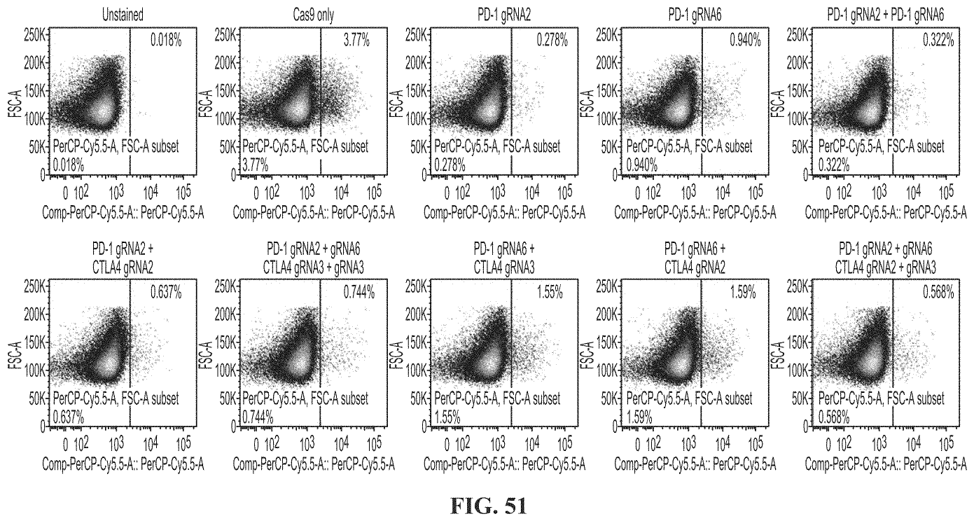

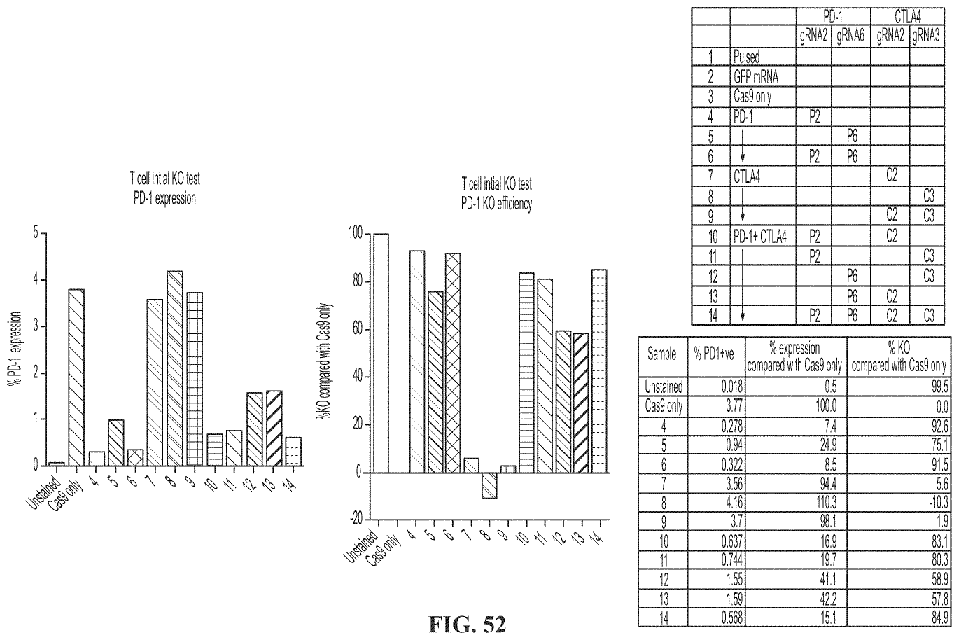

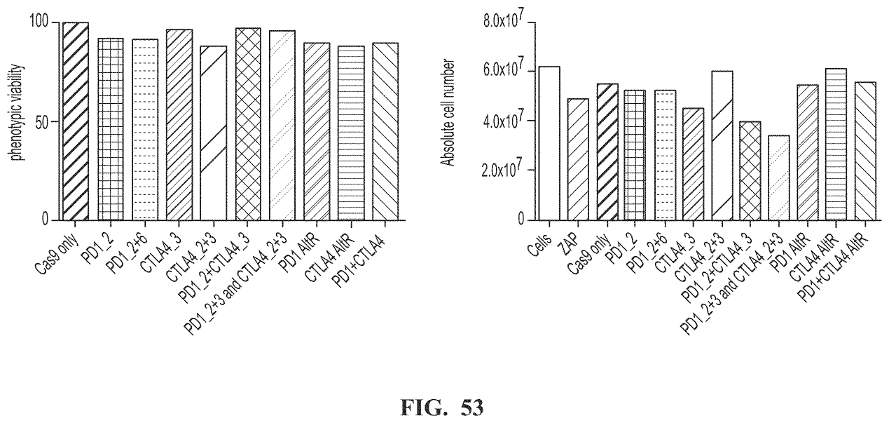

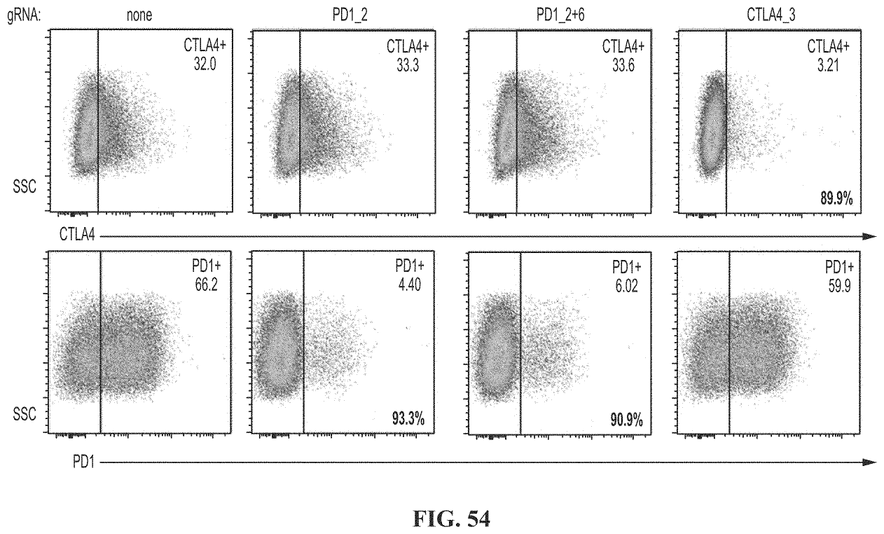

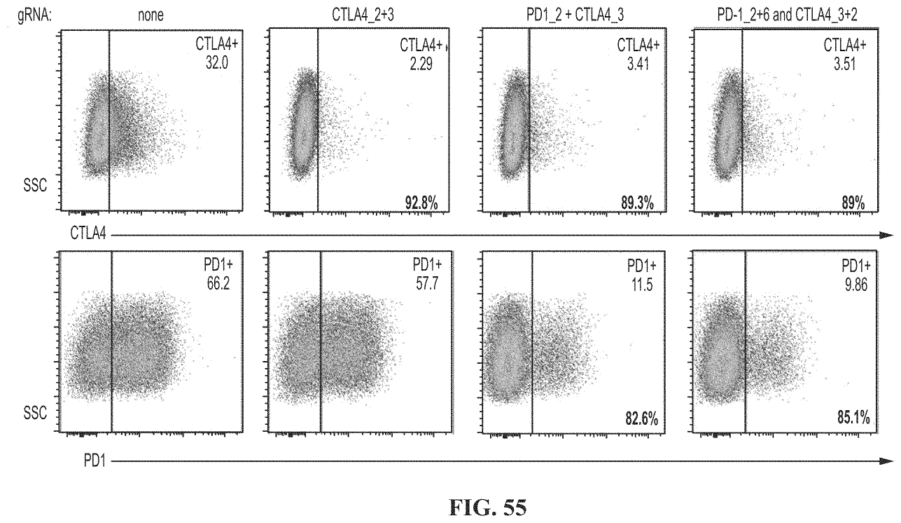

A representative example of the generating a T cell with an engineered TCR and an immune checkpoint gene disruption is shown in FIG. 17. Positive PCR results demonstrate successful recombination at the CCR5 gene. Efficiency of immune checkpoint knock out is shown in a representative experiment in FIG. 23 A, FIG. 23 B, FIG. 24 A, and FIG. 24 B. Flow cytometry data is shown for a representative experiment in FIG. 25. FIG. 26 A and FIG. 26 B show percent double knock out in primary human T cells post treatment with CRISPR. A representative example of flow cytometry results on day 14 post transfection with CRISPR and anti-PD-1 guide RNAs is shown in FIG. 45, FIG. 51, and FIG. 52. Cellular viability and gene editing efficiency 14 days post transfection is shown in FIG. 53, FIG. 54, and FIG. 55 for cells transfected with a CRISPR system and gRNA targeting CTLA-4 and PD-1.

### Example 5: Detection of Homologous Recombination in T Cells

To generate an engineered T cell population that expresses an engineered TCR that also disrupts a gene, CRISPR, TALEN, transposon-based, ZEN, meganuclease, or Mega-TAL gene editing method will be used. To determine if homologous recombination is facilitated with the use of a homologous recombination enhancer the following example embodies a representative experiment. Stimulated CD3+ T cells were electroporated using the NEON transfection system (Invitrogen). Cells were counted and resuspended at a density of 1.0-3.0×10⁶ cells in 100 uL of T buffer. 15 ug mRNA Cas9 (TriLink BioTechnologies), 10 ug mRNA gRNA (TriLink BioTechnologies) and 10 ug of homologous recombination (HR) targeting vector were used for to examine HR, 10 ug of HR targeting vector alone or 15 ug Cas9 with 10 ug mRNA gRNA were used as controls. After electroporation cells were split into four conditions to test two drugs suggested to promote HR.: 1) DMSO only (vehicle control), 2) SCR7 (1 uM), 3) L755507 (5 uM) and 4) SCR7 and L755507. Cells were counted using a Countess II Automated Cell Counter (Thermo Fisher) every three days to monitor growth under these various conditions. In order to monitor for HR, cells were analyzed by flow cytometry and tested by PCR. For flow cytometry, cells were analyzed once a week for three weeks. T cells were stained with APC anti-mouse TCRβ (eBiosciences) and Fixable Viability Dye eFluor 780 (eBiosciences). Cells were analyzed using a LSR II (RD Biosciences) and FlowJo v.9. To test for HR by PCR, gDNA was isolated from T cells and amplified by PCR using accuprime tag DNA polymerase, high fidelity (Thermo Fisher). Primers were designed to both the CCR5 gene and to both ends of the RR targeting vector to look for proper homologous recombination at both the 5′ and 3′ end.

### Example 6: Preventing Toxicity Induced by Exogenous Plasmid DNA

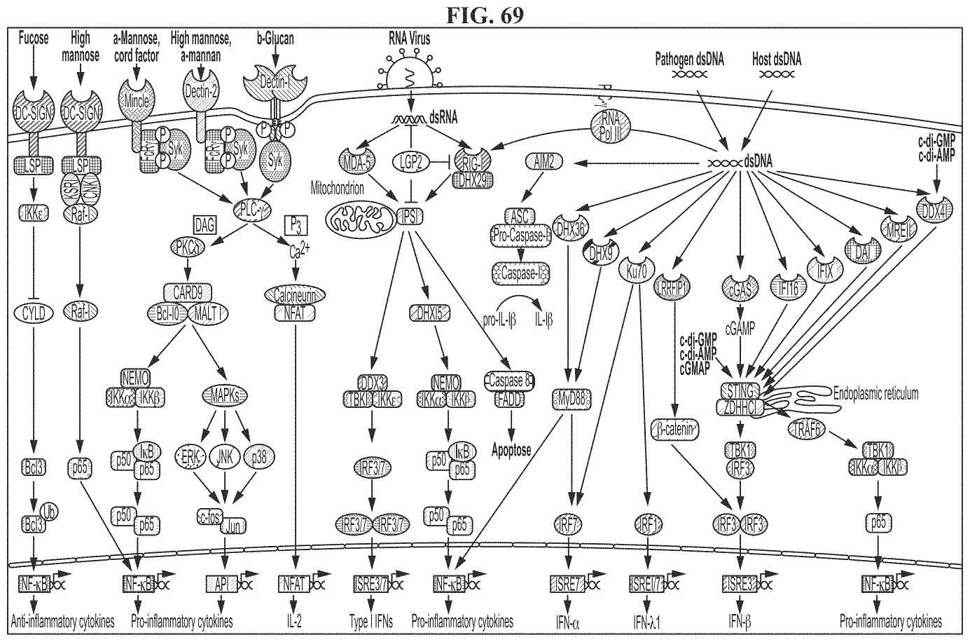

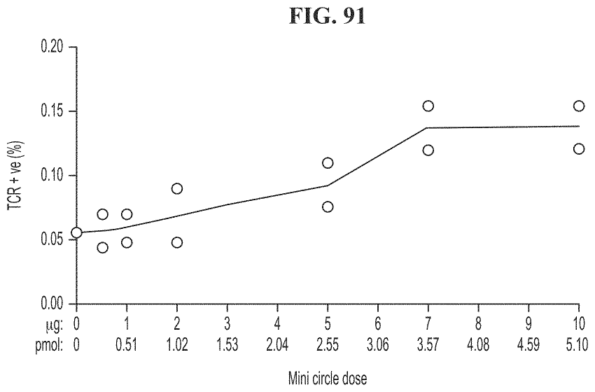

Exogenous plasmid DNA induces toxicity in T cells, The mechanism by which toxicity occurs is described by the innate immune sensing pathway of FIG. 19 and FIG. 69. To determine if cellular toxicity can be reduced by addition of a compound that modifies a response to exogenous polynucleic acids the following representative experiment was completed. CD3+ T cells were electroporated using the NEON transfection system (Invitrogen) with increasing amounts of plasmid DNA (0.1 ug to 40 ug). FIG. 91. After electroporation cells were split into four conditions to test two drugs capable of blocking apoptosis induced by the double stranded DNA: 1) DMSO only (vehicle control), 2) BX795 (1 uM, Invivogen), 3) Z-VAD-FMK (50 uM, R&D Systems) and 4) BX795 and Z-VAD-FMK. Cells were analyzed by flow 48 hours later. T cells were stained with Fixable Viability Dye eFluor 780 (eBiosciences) and were analyzed using a LSR II (BD Biosciences) and FlowJo v.9.

### Results

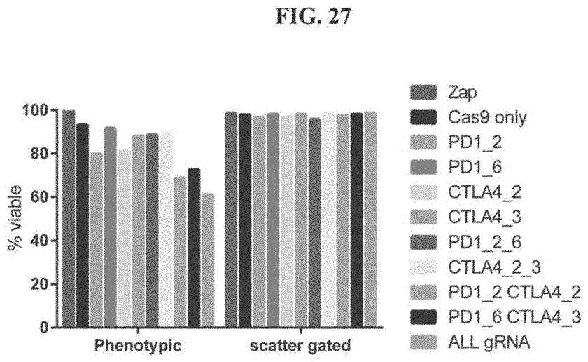

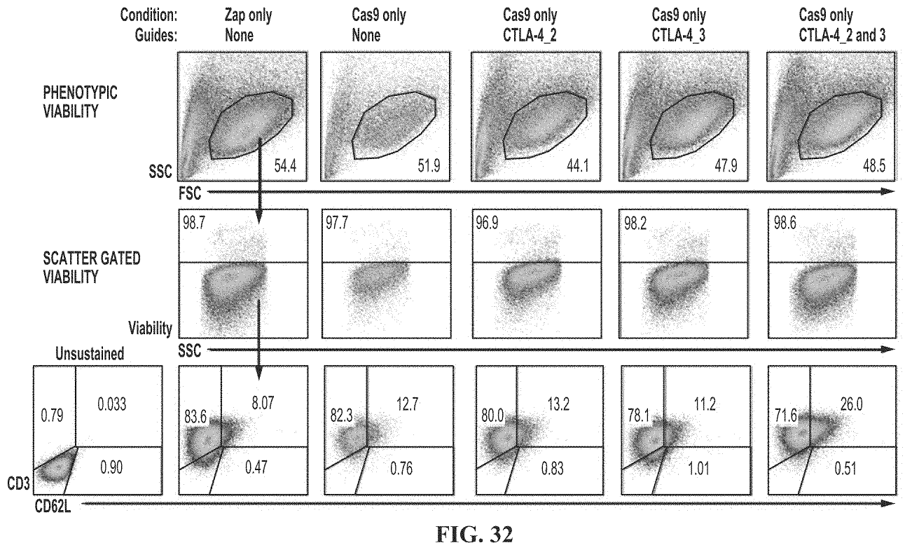

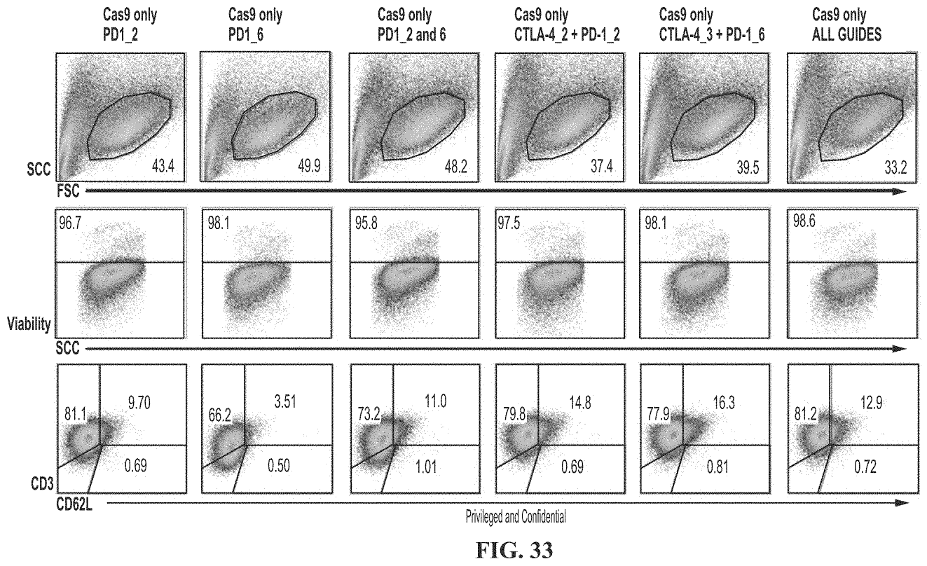

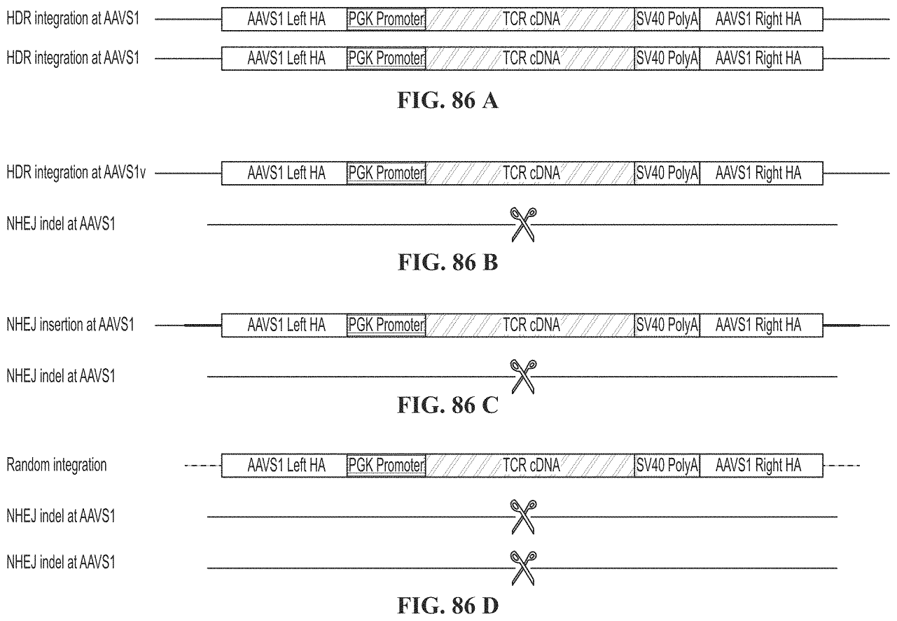

A representative example of toxicity experienced by T cells in transfected with plasmid DNA is shown in FIG. 18, FIG. 27, FIG. 32 and FIG. 33. Viability by cell count is shown in FIG. 86. After the addition of innate immune pathway inhibitors, the percent of T cells undergoing death is reduced. By way of example, FIG. 20 shows a representation of the reduction of apoptosis of T cell cultures treated with two different inhibitors.

### Example 7: An Unmethylated Polynucleic Acid Comprising at Least One Engineered Antigen Receptor with Recombination Arms to a Genomic Region

Modifications to polynucleic acids can be performed as shown in FIG. 21. To determine if an unmethylated polynucleic acid can reduce toxicity induced by exogenous plasmid DNA and improve genomic engineering the following experimental example can be employed. To start the maxi prep, a bacterial colony containing the homologous recombination targeting vector was picked and inoculated in 5 mLs LB broth with kanamycin (1:1000) and grown for 4-6 hours at 37° C. The starter culture was then added to a larger culture of 250 mLs LB broth with kanamycin and grown 12-16 hours in the presence of SssI enzyme at 37° C. The maxi was prepped using the Hi Speed Plasmid. Maxi Kit (Qiagen) following the manufacturers protocol with one exception. After lysis and neutralization of the prep, 2.5 mL of endotoxin toxin removal buffer was added to the prep and incubated for 45 minutes on ice. The prep was finished in a laminar flow hood to maintain sterility. The concentration of the prep was determined using a Nanodrop.

### Example 8: GUIDE-Seq Library Preparation

Genomic DNA was isolated from transfected, control (untransfected and CRISPR transfected cells with minicircle DNA carrying an exogenous TCR, Table 10. Human T cells isolated using solid-phase reversible immobilization magnetic beads (Agencourt DNAdvance), were sheared with a Covaris S200 instrument to an average length of 500 bp, end-repaired, A-tailed, and ligated to half-functional adapters, incorporating a 8-nt random molecular index. Two rounds of nested anchored PCR, with primers complementary to the oligo tag, were used for target enrichment. End Repair Thermocycler Program: 12° C. for 15 min, 37° C. for 15 min; 72° C. for 15 min; hold at 4° C.

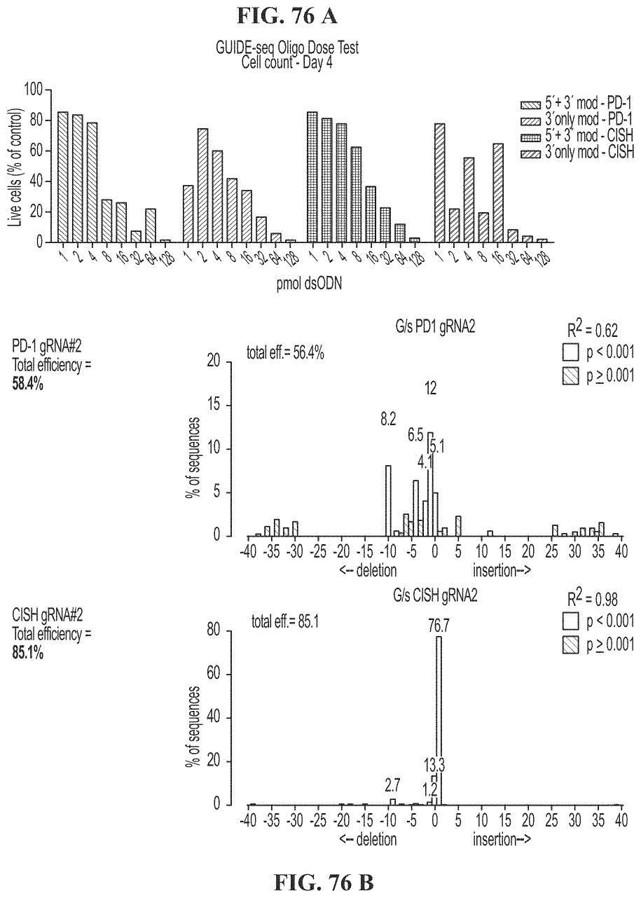

Start sites of GUIDE-Seq reads mapped back to the genome enable localization of the DSB to within a few base pairs. Quantitate library using Kapa Biosystems kit for Illumina Library Quantification kit, according to manufacturer instruction. Using the mean quantity estimate of number of molecules per uL given by the qPCR run for each sample, proceed to normalize the total set of libraries to 1.2×10¹⁰ molecules, divided by the number of libraries to be pooled together for sequencing. This will give a by molecule input for each sample, and also a by volume input for each sample Mapped reads for the on- and off-target sites of the three RGNs directed by truncated gRNAs we assessed by GUIDE-Seq are shown. In all cases, the target site sequence is shown with the protospacer sequence to the left and the PAM sequence to the right on the x-axis. Denature the library and load onto the Miseq according to Illumina's standard protocol for sequencing with an Illumina Miseq Reagent Kit V2-300 cycle (2×150 bp paired end). FIG. 76 A and FIG. 76 B show data for a representative GUIDE-Seq experiment.

### Example 9: Adenoviral Serotype 5 Mutant Protein Generation

Mutant cDNAs, Table 8, were codon optimized and synthesized as gBlock fragments by Integrated DNA technologies (IDT). Synthesized fragments were sub-cloned into an mRNA production vector for in vitro mRNA synthesis.

TABLE 8
Mutant cDNA sequences for adenoviral proteins

| SEQ ID | Mutation | Name | Sequence (5′ to 3′) |
|---|---|---|---|
| 88 | None | Adenovirus | atgacaacaagtggcgtgccattcggcatgactttgcgccccac |
| | | serotype 5 | gagatcacgactgtctcgccgaactccctacagccgggatcgac |
| | | E4orf6 | tccctccctttgagactgaaacacgggccacgatactcgaggac |
| | | | cacccacttctgccggagtgtaacaccttgacgatgcataacgtta |
| | | | gctatgtgagaggtctcccttgttctgtcggctttacccttattcaag |
| | | | agtgggtcgtgccgtgggacatggttctcacgagagaggagctc |
| | | | gttatcctgagaaaatgtatgcacgtttgtctttgctgtgcaaatata |
| | | | gatataatgacttctatgatgattcatgggtacgaatcttgggcctt |
| | | | gcactgccattgtagcagtcctggctccctccaatgcatcgcggg |
| | | | aggccaagttctcgcttcctggtttagaatggtcgtggacggagc |
| | | | aatgttcaaccagcgctttatctggtatcgcgaggtagtcaactata |
| | | | atatgccgaaggaggttatgtttatgtctagtgtgttcatgcgaggg |
| | | | agacatttgatttatcttagactgtggtatgatggccatgtgggaag |
| | | | cgtagttccggcgatgtccttcggttactccgcattgcattgtggg |
| | | | attttgaataacatcgttgtactagagttcatactgcgccgatctgt |
| | | | cagaaataagggtacgatgctgcgcacggcgaacccggaggct |
| | | | catgctgagagccgttcgaataatcgctgaagaaacgacagcaa |
| | | | tgttgtattcatgccgaactgaaaggcgacggcaacagtttatacg |
| | | | cgcactcttgcagcaccacaggccgatcctgatgcatgactacg |
| | | | atagcactccgatgtag |
| 89 | H→A at amino | Adenovirus | atggagagaaggaatcctagtgagaggggagtgcccgccggg |
| | acid 373 | serotype 5 | ttttctggtcacgcctccgtggaatccggatgtgagactcaggagt |
| | | H373A mutant | cccccgccaccgtggtgttccgcccaccaggagacaacactga |
| | | | cggtggcgcggcggctgctgcaggtggaagccaagccgccgc |
| | | | tgctggggccgagccgatggaacccgaatccagacccggtccc |
| | | | tctggcatgaacgttgtgcaggtcgcagaactctaccccgaactc |
| | | | cgcaggatcttgacaatcacggaggacggccagggcctcaagg |
| | | | gagtgaagagagagagaggggcttgtgaggccactgaggaag |
| | | | ctcgcaatctggcgttttcattgatgacaaggcacaggccggaat |
| | | | gcattacattccaacagattaaggacaactgcgcaaacgagctc |
| | | | gatctcctggcccagaagtatagcatcgagcagctgacaacctat |
| | | | tggctgcagcccggcgacgattttgaagaggccatccgcgtgta |
| | | | cgcaaaggtggccctgcgacctgactgcaaatataagatttccaa |
| | | | actggttaacatccggaattgttgttatattagtggaaatggcgcag |
| | | | aagtggagattgacacagaggatcgagtcgctaccggtgctcta |
| | | | tgatcaacatgtggcccggtgtgctcggcatggatggcgtagtca |
| | | | ttatgaatgtgaggttcaccggacctaattttagcggaaccgtcttc |
| | | | ctggcaaacactaatctgatcctgcatggagtttctttctatggattt |
| | | | aataacacctgtgttgaagcttggaccgacgtgcgggttagagg |
| | | | gtgtgctattattgctgctggaaaggcgtcgtgtgtagacccaaa |
| | | | agtagagcttctatcaagaaatgcctgttcgagaggtgtactctgg |
| | | | gcattctcagtgaaggtaatagcagggtcaggcataacgtggcct |
| | | | cagattgcggatgattatgttggttaaatccgtggctgtgatcaag |
| | | | cacaacatggtgtgtggcaattgtgaggaccgggcatctcaaatg |
| | | | ctgacatgttccgatggcaactgtcacctgctcaaaacaattgccg |
| | | | ttgcgagccattctcggaaggcctggccagttttcgagcataacat |
| | | | cctgacgcgctgtagtctccacctgggtaacagacggggcgtttt |
| | | | cctgccatatcagtgtaacctgtcacataccaagatactcctggaa |
| | | | ccagaatctatgagtaaagtgaacctgaatggtgtattcgatatga |
| | | | ccatgaagatatggaaagtcctccgctatgacgaaactaggacta |
| | | | ggtgtaggccctgcgagtgtggcggcaagcatatccgcaacca |
| | | | acccgtgatgctggacgtgaccgaggagctgcgccccgatcac |
| | | | ctggtgctggcctgcaccagagcagaattcgggagctcagacg |
| | | | aagacactgattaa |
| 90 | Amino acid | Adenovirus | atggagagaaggaatcctagtgagaggggagtgcccgccggg |
| | Insertion | serotype 5 | ttttctggtcacgcctccgtggaatccggatgtgagactcaggagt |
| | (AGIPA) | H354 mutant | cccccgccaccgtggtgttccgcccaccaggagacaacactga |
| | | | cggtggcgcggcggctgctgcaggtggaagccaagccgccgc |
| | | | tgctggggccgagccgatggaacccgaatccagacccggtccc |
| | | | tctggcatgaacgttgtgcaggtcgcagaactctaccccgaactc |
| | | | cgcaggatcttgacaatcacggaggacggccagggcctcaagg |
| | | | gagtgaagagagagagaggggcttgtgaggccactgaggaag |
| | | | ctcgcaatctggcgttttcattgatgacaaggcacaggccggaat |
| | | | gcattacattccaacagattaaggacaactgcgcaaacgagctc |
| | | | gatctcctggcccagaagtatagcatcgagcagctgacaacctat |
| | | | tggctgcagcccggcgacgattttgaagaggccatccgcgtgta |
| | | | cgcaaaggtggccctgcgacctgactgcaaatataagatttccaa |
| | | | actggttaacatccggaattgttgttatattagtggaaatggcgcag |
| | | | aagtggagattgacacagaggatcgagtcgctttccggtgctcta |
| | | | tgatcaacatgtggcccggtgtgctcggcatggatggcgtagtca |
| | | | ttatgaatgtgaggttcaccggacctaattttagcggaaccgtcttc |
| | | | ctggcaaacactaatctgatcctgcatggagtttctttctatggattt |
| | | | aataacacctgtgttgaagcttggaccgacgtgcgggttagagg |
| | | | gtgtgctattattgctgctggaaaggcgtcgtgtgtagacccaaa |
| | | | agtagagcttctatcaagaaatgcctgttcgagaggtgtactctgg |
| | | | gcattctcagtgaaggtaatagcagggtcaggcataacgtggcct |
| | | | cagattgcggatgattatgaggttaaatccgtggctgtgatcaag |
| | | | cacaacatggtgtgtggcaattgtgaggaccgggctggaattcc |
| | | | agcatctcaaatgctgacatgttccgatggcaactgtcacctgctc |
| | | | aaaacaattcacgttgcgagccattctcggaaggcctggccagtt |
| | | | ttcgagcataacatcctgacgcgctgtagtctccacctgggtaac |
| | | | agacggggcgttttcctgccatatcagtgtaacctgtcacatacca |
| | | | agatactcctggaaccagaatctatgagtaaagtgaacctgaatg |
| | | | gtgtattcgatatgaccatgaagatatggaaagtcctccgctatga |
| | | | cgaaactaggactaggtgtaggccctgcgagtgtggcggcaag |
| | | | catatccgcaaccaacccgtgatgctggacgtgaccgaggagct |
| | | | gcgccccgatcacctggtgctggcctgcaccagagcagaattcg |
| | | | ggagctcagacgaagacactgattaa′ |

### Example 10. Genomic Engineering of TIL to Knock Out PD-1, CTLA-4, and CISH

Suitable tumors from eligible stage IIIc-IV cancer patients will be resected and cut up into small 3-5 mm² fragments and placed in culture plates or small culture flasks with growth medium and high-dose (HD) IL-2. The TIL will initially be expanded for 3-5 weeks during this “pre-rapid expansion protocol” (pre-REP) phase to at least 50×10⁶ cells. TILs are electroporated using the Neon Transfection System (100 uL or 10 ul Kit, Invitrogen, Life Technologies). TILS will be pelleted and washed once with T buffer. TILs are resuspended at a density of 2×10\hat{ }5 cells in 10 uL of T buffer for 10 ul tip, and 3×10\hat{ }6 cells in 100 ul T buffer for 100 ul tips. TILs are then electroporated at 1400 V, 10 ms, 3 pulses utilizing 15ug Cas9 mRNA, and 10-50ug PD-1, CTLA-4, and CISH gRNA-RNA (100 mcl tip). After transfection, TILs will be plated at 1000 cells/ul in antibiotic free culture media and incubated at 30 C in 5% CO2 for 24 hrs. After 24 hr recovery, TILs can be transferred to antibiotic containing media and cultured at 37 C in 5% CO2.

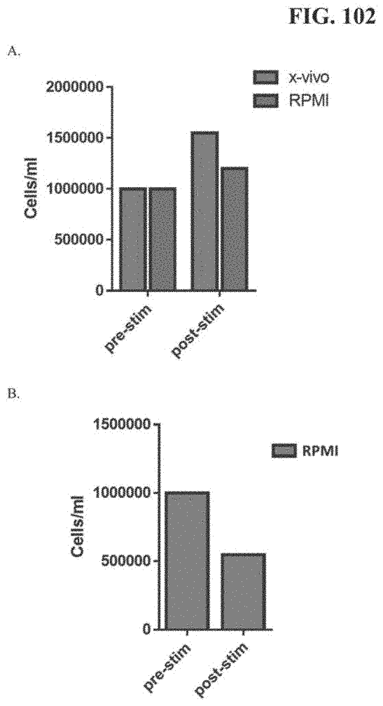

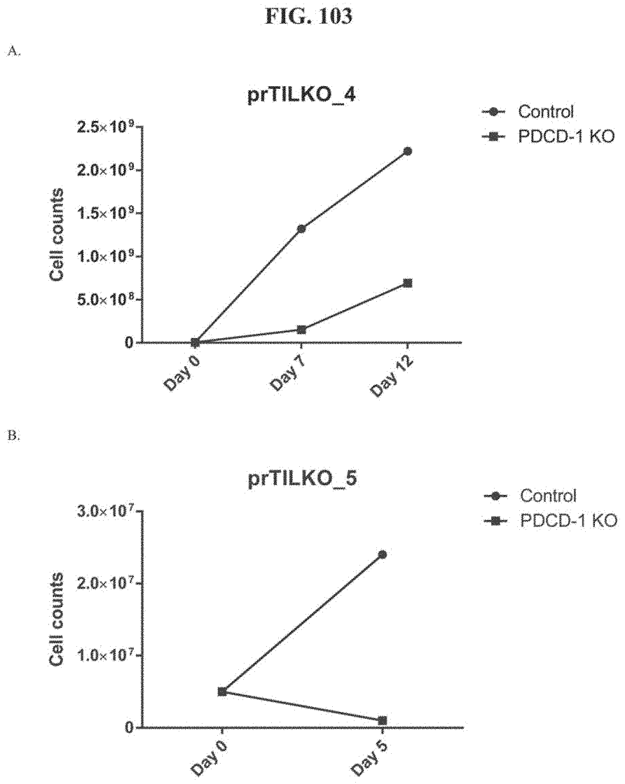

The cells are then subjected to a rapid expansion protocol (REP) over two weeks by stimulating the TILs using anti-CD3 in the presence of PBMC feeder cells and IL-2. The expanded TIL (now billions of cells) will be washed, pooled, and infused into a patient followed by one or two cycles of HD IL-2 therapy. Before TIL transfer, a patient can be treated with a preparative regimen using cyclophosphamide (Cy) and fludaribine (Flu) that transiently depletes host lymphocytes “making room” for the infused TIL and removing cytokine sinks and regulatory T cells in order to facilitate TIL persistence. Subjects will receive an infusion of their own modified TIL cells over 30 minutes and will remain in the hospital to be monitored for adverse events until they have recovered from the treatment. FIG. 102 A and FIG. 102 B show cellular expansion of TIL of two different subjects. FIG. 103 A and FIG. 103 B show cellular expansion of TIL electroporated with a CRISPR system, and anti-PD-1 guides and cultured with the addition of feeders (A) or no addition of feeders (B).

TABLE 9
Endogenous checkpoint summary

| | | | | NCBI number | | | |
|---|---|---|---|---|---|---|---|
| | | | | (GRCh38.p2) | | | |
| SEQ | Gene | | | *AC010327.8 | Original | Original | Location in |
| ID | Symbol | Abbreviation | Name | **GRCh38.p7 | Start | Stop | genome |
| 91 | ADORA2A | A2aR; RDC8; | adenosine | 135 | 24423597 | 24442360 | 22q11.23 |
| | | ADORA2 | A2a receptor | | | | |
| 92 | CD276 | B7H3; B7-H3; | CD276 | 80381 | 73684281 | 73714518 | 15q23-q24 |
| | | B7RP-2; 4Ig- | molecule | | | | |
| | | B7-H3 | | | | | |
| 93 | VTCN1 | B7X; B7H4; | V-set | 79679 | 117143587 | 117270368 | 1p13.1 |
| | | B7S1; B7-H4; | domain | | | | |
| | | B7h.5; VCTN1; | containing T | | | | |
| | | PRO1291 | cell | | | | |
| | | | activation | | | | |
| | | | inhibitor 1 | | | | |
| 94 | BTLA | BTLA1; CD272 | B and T | 151888 | 112463966 | 112499702 | 3q13.2 |
| | | | lymphocyte | | | | |
| | | | associated | | | | |
| 95 | CTLA4 | GSE; GRD4; | cytotoxic T- | 1493 | 203867788 | 203873960 | 2q33 |
| | | ALPS5; CD152; | lymphocyte- | | | | |
| | | CTLA-4; | associated | | | | |
| | | IDDM12; | protein 4 | | | | |
| | | CELIAC3 | | | | | |
| 96 | IDO1 | IDO; INDO; | indoleamine | 3620 | 39913809 | 39928790 | 8p12-p11 |
| | | IDO-1 | 2,3- | | | | |
| | | | dioxygenase 1 | | | | |
| 97 | KIR3DL1 | KIR; NKB1; | killer cell | 3811 | 54816438 | 54830778 | 19q13.4 |
| | | NKAT3; | immunoglobulin- | | | | |
| | | NKB1B; | like | | | | |
| | | NKAT-3; | receptor, | | | | |
| | | CD158E1; | three | | | | |
| | | KIR3DL2; | domains, | | | | |
| | | KIR3DL1/S1 | long | | | | |
| | | | cytoplasmic | | | | |
| | | | tail, 1 | | | | |
| 98 | LAG3 | LAG3; CD223 | lymphocyte- | 3902 | 6772483 | 6778455 | 12p13.32 |
| | | | activation | | | | |
| | | | gene 3 | | | | |
| 99 | PDCD1 | PD1; PD-1; | programmed | 5133 | 241849881 | 241858908 | 2q37.3 |
| | | CD279; SLEB2; | cell death 1 | | | | |
| | | hPD-1; hPD-1; | | | | | |
| | | hSLE1 | | | | | |
| 100 | HAVCR2 | TIM3; CD366; | hepatitis A | 84868 | 157085832 | 157109237 | 5q33.3 |
| | | KIM-3; TIMD3; | virus | | | | |
| | | Tim-3; TIMD-3; | cellular | | | | |
| | | HAVcr-2 | receptor 2 | | | | |
| 101 | VISTA | C10orf54, | V-domain | 64115 | 71747556 | 71773580 | 10q22.1 |
| | | differentiation of | immunoglobulin | | | | |
| | | ESC-1 (Dies1); | suppressor | | | | |
| | | platelet receptor | of T-cell | | | | |
| | | Gi24 precursor; | activation | | | | |
| | | PD1 homolog | | | | | |
| | | (PD1H) B7H5; | | | | | |
| | | GI24; B7-H5; | | | | | |
| | | SISP1; PP2135 | | | | | |
| 102 | CD244 | 2B4; 2B4; | CD244 | 51744 | 160830158 | 160862902 | 1q23.3 |
| | | NAIL; Nmrk; | molecule, | | | | |
| | | NKR2B4; | natural killer | | | | |
| | | SLAMF4 | cell receptor | | | | |
| | | | 2B4 | | | | |
| 103 | CISH | CIS; G18; | cytokine | 1154 | 50606454 | 50611831 | 3p21.3 |
| | | SOCS; CIS-1; | inducible | | | | |
| | | BACTS2 | SH2- | | | | |
| | | | containing | | | | |
| | | | protein | | | | |
| 104 | HPRT1 | HPRT; HGPRT | hypoxanthine | 3251 | 134452842 | 134500668 | Xq26.1 |
| | | | phosphoribosyltransferase 1 | | | | |
| 105 | AAV*S1 | AAV | adeno- | 14 | 7774 | 11429 | 19q13 |
| | | | associated | | | | |
| | | | virus | | | | |
| | | | integration | | | | |
| | | | site 1 | | | | |
| 106 | CCR5 | CKR5; CCR-5; | chemokine | 1234 | 46370142 | 46376206 | 3p21.31 |
| | | CD195; CKR-5; | (C-C motif) | | | | |
| | | CCCKR5; | receptor 5 | | | | |
| | | CMKBR5; | (gene/pseudogene) | | | | |
| | | IDDM22; CC- | | | | | |
| | | CKR-5 | | | | | |
| 107 | CD160 | NK1; BY55; | CD160 | 11126 | 145719433 | 145739288 | 1q21.1 |
| | | NK28 | molecule | | | | |
| 108 | TIGIT | VSIG9; | T-cell | 201633 | 114293986 | 114310288 | 3q13.31 |
| | | VSTM3; | immunoreceptor | | | | |
| | | WUCAM | with Ig | | | | |
| | | | and ITIM | | | | |
| | | | domains | | | | |
| 109 | CD96 | TACTILE | CD96 | 10225 | 111542079 | 111665996 | 3q13.13-q13.2 |
| | | | molecule | | | | |
| 110 | CRTAM | CD355 | cytotoxic | 56253 | 122838431 | 122872643 | 11q24.1 |
| | | | and | | | | |
| | | | regulatory | | | | |
| | | | T-cell | | | | |
| | | | molecule | | | | |
| 111 | LAIR1 | CD305; LAIR-1 | leukocyte | 3903 | 54353624 | 54370556 | 19q13.4 |
| | | | associated | | | | |
| | | | immunoglobulin | | | | |
| | | | like | | | | |
| | | | receptor 1 | | | | |
| 112 | SIGLEC7 | p75; QA79; | sialic acid | 27036 | 51142294 | 51153526 | 19q13.3 |
| | | AIRM1; CD328; | binding Ig | | | | |
| | | CDw328; D- | like lectin 7 | | | | |
| | | siglec; SIGLEC- | | | | | |
| | | 7; SIGLECP2; | | | | | |
| | | SIGLEC19P; | | | | | |
| | | p75/AIRM1 | | | | | |
| 113 | SIGLEC9 | CD329; | sialic acid | 27180 | 51124880 | 51141020 | 19q13.41 |
| | | CDw329; | binding Ig | | | | |
| | | FOAP-9; siglec- | like lectin 9 | | | | |
| | | 9; OBBP-LIKE | | | | | |
| 114 | TNFRSF10B | DR5; CD262; | tumor | 8795 | 23006383 | 23069187 | 8p22-p21 |
| | | KILLER; | necrosis | | | | |
| | | TRICK2; | factor | | | | |
| | | TRICKB; | receptor | | | | |
| | | ZTNFR9; | superfamily | | | | |
| | | TRAILR2; | member 10b | | | | |
| | | TRICK2A; | | | | | |
| | | TRICK2B; | | | | | |
| | | TRAIL-R2; | | | | | |
| | | KILLER/DR5 | | | | | |
| 115 | TNFRSF10A | DR4; APO2; | tumor | 8797 | 23191457 | 23225167 | 8p21 |
| | | CD261; | necrosis | | | | |
| | | TRAILR1; | factor | | | | |
| | | TRAILR-1 | receptor | | | | |
| | | | superfamily | | | | |
| | | | member 10a | | | | |
| 116 | CASP8 | CAP4; MACH; | caspase 8 | 841 | 201233443 | 201287711 | 2q33-q34 |
| | | MCH5; FLICE; | | | | | |
| | | ALPS2B; Casp-8 | | | | | |
| 117 | CASP10 | MCH4; ALPS2; | caspase 10 | 843 | 201182898 | 201229406 | 2q33-q34 |
| | | FLICE2 | | | | | |
| 118 | CASP3 | CPP32; SCA-1; | caspase 3 | 836 | 184627696 | 184649475 | 4q34 |
| | | CPP32B | | | | | |
| 119 | CASP6 | MCH2 | caspase 6 | 839 | 109688628 | 109713904 | 4q25 |
| 120 | CASP7 | MCH3; CMH-1; | caspase 7 | 840 | 113679162 | 113730909 | 10q25 |
| | | LICE2; CASP- | | | | | |
| | | 7; ICE-LAP3 | | | | | |
| 121 | FADD | GIG3; MORT1 | Fas | 8772 | 70203163 | 70207402 | 11q13.3 |
| | | | associated | | | | |
| | | | via death | | | | |
| | | | domain | | | | |
| 122 | FAS | APT1; CD95; | Fas cell | 355 | 88969801 | 89017059 | 10q24.1 |
| | | FAS1; APO-1; | surface | | | | |
| | | FASTM; | death | | | | |
| | | ALPS1A; | receptor | | | | |
| | | TNFRSF6 | | | | | |
| 123 | TGFBRII | AAT3; FAA3; | transforming | 7048 | 30606493 | 30694142 | 3p22 |
| | | LDS2; MFS2; | growth | | | | |
| | | RIIC; LDS1B; | factor beta | | | | |
| | | LDS2B; | receptor II | | | | |
| | | TAAD2; TGFR- | | | | | |
| | | 2; TGFbeta-RII | | | | | |
| 124 | TGFBR1 | AAT5; ALK5; | transforming | 7046 | 99104038 | 99154192 | 9q22 |
| | | ESS1; LDS1; | growth | | | | |
| | | MSSE; SKR4; | factor beta | | | | |
| | | ALK-5; LDS1A; | receptor I | | | | |
| | | LDS2A; TGFR- | | | | | |
| | | 1; ACVRLK4; | | | | | |
| | | tbetaR-I | | | | | |
| 125 | SMAD2 | JV18; MADH2; | SMAD | 4087 | 47833095 | 47931193 | 18q21.1 |
| | | MADR2; JV18- | family | | | | |
| | | 1; hMAD-2; | member 2 | | | | |
| | | hSMAD2 | | | | | |
| 126 | SMAD3 | LDS3; LDS1C; | SMAD | 4088 | 67065627 | 67195195 | 15q22.33 |
| | | MADH3; JV15- | family | | | | |
| | | 2; HSPC193; | member 3 | | | | |
| | | HsT17436 | | | | | |
| 127 | SMAD4 | JIP; DPC4; | SMAD | 4089 | 51030213 | 51085042 | 18q21.1 |
| | | MADH4; | family | | | | |
| | | MYHRS | member 4 | | | | |
| 128 | SKI | SGS; SKV | SKI proto- | 6497 | 2228695 | 2310213 | 1p36.33 |
| | | | oncogene | | | | |
| 129 | SKIL | SNO; SnoA; | SKI-like | 6498 | 170357678 | 170396849 | 3q26 |
| | | SnoI; SnoN | proto- | | | | |
| | | | oncogene | | | | |
| 130 | TGIF1 | HPE4; TGIF | TGFB | 7050 | 3411927 | 3458411 | 18p11.3 |
| | | | induced | | | | |
| | | | factor | | | | |
| | | | homeobox 1 | | | | |
| 131 | IL10RA | CD210; IL10R; | interleukin | 3587 | 117986391 | 118001483 | 11q23 |
| | | CD210a; | 10 receptor | | | | |
| | | CDW210A; | subunit | | | | |
| | | HIL-10R; IL- | alpha | | | | |
| | | 10R1 | | | | | |
| 132 | IL10RB | CRFB4; CRF2- | interleukin | 3588 | 33266360 | 33297234 | 21q22.11 |
| | | 4; D21S58; | 10 receptor | | | | |
| | | D21S66; | subunit beta | | | | |
| | | CDW210B; IL- | | | | | |
| | | 10R2 | | | | | |
| 133 | HMOX2 | HO-2 | heme | 3163 | 4474703 | 4510347 | 16p13.3 |
| | | | oxygenase 2 | | | | |
| 134 | IL6R | IL6Q; gp80; | interleukin 6 | 3570 | 154405193 | 154469450 | 1q21 |
| | | CD126; IL6RA; | receptor | | | | |
| | | IL6RQ; IL-6RA; | | | | | |
| | | IL-6R-1 | | | | | |
| 135 | IL6ST | CD130; GP130; | interleukin 6 | 3572 | 55935095 | 55994993 | 5q11.2 |
| | | CDW130; IL- | signal | | | | |
| | | 6RB | transducer | | | | |
| 136 | CSK | CSK | c-src | 1445 | 74782084 | 74803198 | 15q24.1 |
| | | | tyrosine | | | | |
| | | | kinase | | | | |
| 137 | PAG1 | CBP; PAG | phosphoprotein | 55824 | 80967810 | 81112068 | 8q21.13 |
| | | | membrane | | | | |
| | | | anchor with | | | | |
| | | | glycosphingolipid | | | | |
| | | | microdomains 1 | | | | |
| 138 | SIT1 | SIT1 | signaling | 27240 | 35649298 | 35650950 | 9p13-p12 |
| | | | threshold | | | | |
| | | | regulating | | | | |
| | | | transmembrane | | | | |
| | | | adaptor 1 | | | | |
| 139 | FOXP3 | JM2; AIID; | forkhead | 50943 | 49250436 | 49269727 | Xp11.23 |
| | | IPEX; PIDX; | box P3 | | | | |
| | | XPID; DIETER | | | | | |
| 140 | PRDM1 | BLIMP1; PRDI- | PR domain 1 | 639 | 106086320 | 106109939 | 6q21 |
| | | BF1 | | | | | |
| 141 | BATF | SFA2; B-ATF; | basic leucine | 10538 | 75522441 | 75546992 | 14q24.3 |
| | | BATF1; SFA-2 | zipper | | | | |
| | | | transcription | | | | |
| | | | factor, ATF- | | | | |
| | | | like | | | | |
| 142 | GUCY1A2 | GC-SA2; | guanylate | 2977 | 106674012 | 107018445 | 11q21-q22 |
| | | GUC1A2 | cyclase 1, | | | | |
| | | | soluble, | | | | |
| | | | alpha 2 | | | | |
| 143 | GUCY1A3 | GUCA3; | guanylate | 2982 | 155666568 | 155737062 | 4q32.1 |
| | | MYMY6; GC- | cyclase 1, | | | | |
| | | SA3; GUC1A3; | soluble, | | | | |
| | | GUCSA3; | alpha 3 | | | | |
| | | GUCY1A1 | | | | | |
| 144 | GUCY1B2 | GUCY1B2 | guanylate | 2974 | 50994511 | 51066157 | 13q14.3 |
| | | | cyclase 1, | | | | |
| | | | soluble, beta 2 | | | | |
| | | | (pseudogene) | | | | |
| 145 | GUCY1B3 | GUCB3; GC- | guanylate | 2983 | 15575897 | 15580764 | 4q31.3-q33 |
| | | SB3; GUC1B3; | cyclase 1, | | | | |
| | | GUCSB3; | soluble, beta 3 | | | | |
| | | GUCY1B1; GC- | | | | | |
| | | S-beta-1 | | | | | |
| 146 | TRA | IMD7; TCRA; | T-cell | 6955 | 21621904 | 22552132 | 14q11.2 |
| | | TCRD; | receptor | | | | |
| | | TRAalpha; | alpha locus | | | | |
| | | TRAC | | | | | |
| 147 | TRB | TCRB; TRBbeta | T cell | 6957 | 142299011 | 142813287 | 7q34 |
| | | | receptor beta | | | | |
| | | | locus | | | | |
| 148 | EGLN1 | HPH2; PHD2; | egl-9 family | 54583 | 231363751 | 231425044 | 1q42.1 |
| | | SM20; ECYT3; | hypoxia- | | | | |
| | | HALAH; HPH- | inducible | | | | |
| | | 2; HIFPH2; | factor 1 | | | | |
| | | ZMYND6; | | | | | |
| | | C1orf12; HIF- | | | | | |
| | | PH2 | | | | | |
| 149 | EGLN2 | EIT6; PHD1; | egl-9 family | 112398 | 40799143 | 40808441 | 19q13.2 |
| | | HPH-1; HPH-3; | hypoxia- | | | | |
| | | HIFPH1; HIF- | inducible | | | | |
| | | PH1 | factor 2 | | | | |
| 150 | EGLN3 | PHD3; HIFPH3; | egl-9 family | 112399 | 33924215 | 33951083 | 14q13.1 |
| | | HIFP4H3 | hypoxia- | | | | |
| | | | inducible | | | | |
| | | | factor 3 | | | | |
| 151 | PPP1R12C** | p84; p85; | protein | 54776 | 55090913 | 55117600 | 19q13.42 |
| | | LENG3; MBS85 | phosphatase | | | | |
| | | | 1 regulatory | | | | |
| | | | subunit 12C | | | | |

TABLE 10
Engineered T cell receptor (TCR)

| SEQ ID | Sequence 5′-3′ |
|---|---|
| 152 | atggccttggtaacctctataactgtgctgctcagtctcggga |
| | tcatgggagatgctaagactactcagcctaatagtatggaaag |
| | taatgaggaggagcctgtccacctgccttgtaatcactctacc |
| | ataagcgggacagattacatacattggtatcggcagctccctt |
| | cacaaggtccagagtatgtgattcatggcctcacatcaaatgt |
| | gaacaatcggatggcttctcttgccattgcagaggatcggaaa |
| | agctcaacactcatcctgcatagggcgacactcagagatgcgg |
| | ccgtttatta |

TABLE 11
Streptococcus pyogenes Cas9 (SpCas9)

| SEQ ID | Sequence 5′ to 3′ |
|---|---|
| 153 | atggactataaggaccacgacggagactacaaggatcatgata |
| | ttgattacaaagacgatgacgataagatggccccaaagaagaa |
| | gcggaaggtcggtatccacggagtcccagcagccgacaagaag |
| | tacagcatcggcctggacatcggcaccaactctgtgggctggg |
| | ccgtgatcaccgacg |

### Example 11: gRNA Modification

### Design and Construction of Modified Guide RNAs:

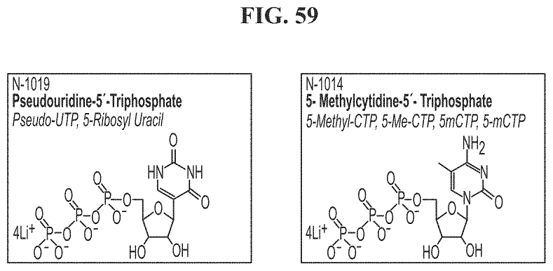

Guide RNAs (gRNAs) were designed to the desired region of a gene using the CRISPR Design Program (Zhang Lab, M I T 2015). Multiple gRNAs (shown in Table 12) were chosen based on the highest ranked values determined by off-target locations. The gRNAs targeting PD-1, CTLA-4, and CISH gene sequences were modified to contain 2-O-Methyl 3phosphorothioate additions, FIG. 44 and FIG. 59.

### Example 12: rAAV Targeting Vector Construction and Virus Production

Targeting vectors described in FIG. 138 were generated by DNA synthesis of the homology arms and PCR amplification of the mTCR expression cassette. The synthesised fragments and mTCR cassette were cloned by restriction enzyme digestion and ligation into the pAAV-MCS backbone plasmid (Agilent) between the two copies of the AAV-2 ITR sequences to facilitate viral packaging. Ligated plasmids were transformed into One Shot TOP10 Chemically Competent E. coli (Thermo fisher). 1 mg of plasmid DNA for each vector was purified from the bacteria using the EndoFree Plasmid Maxi Kit (Qiagen) and sent to Vigene Biosciences, MD USA, for production of Infectious rAAV. The titre of the purified virus, exceeding 1×10′³ viral genome copies per ml, was determined and frozen stocks were made.

### Example 13: T Cell Infection with rAAV

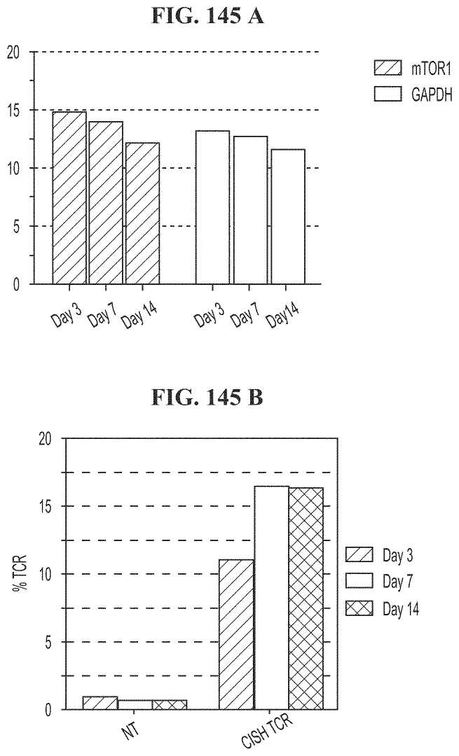

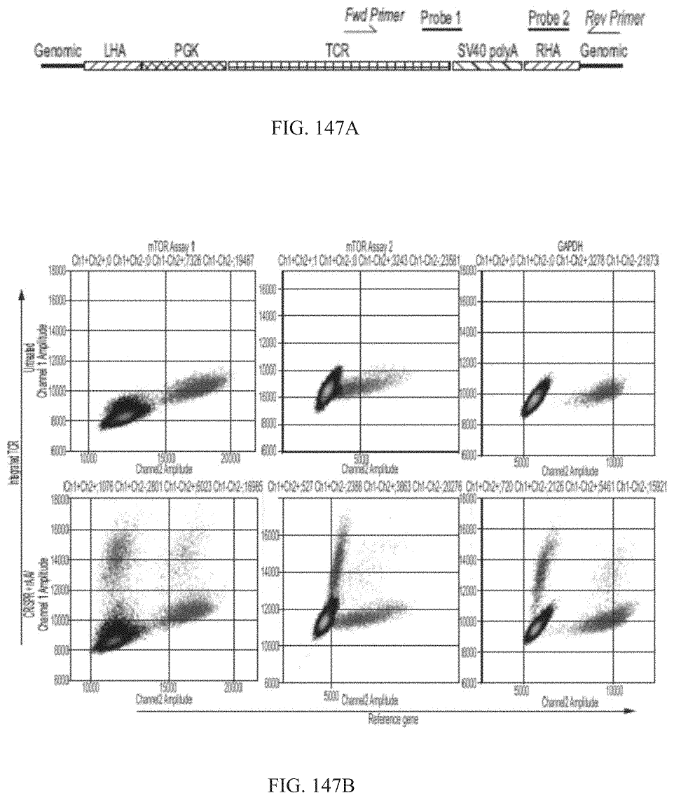

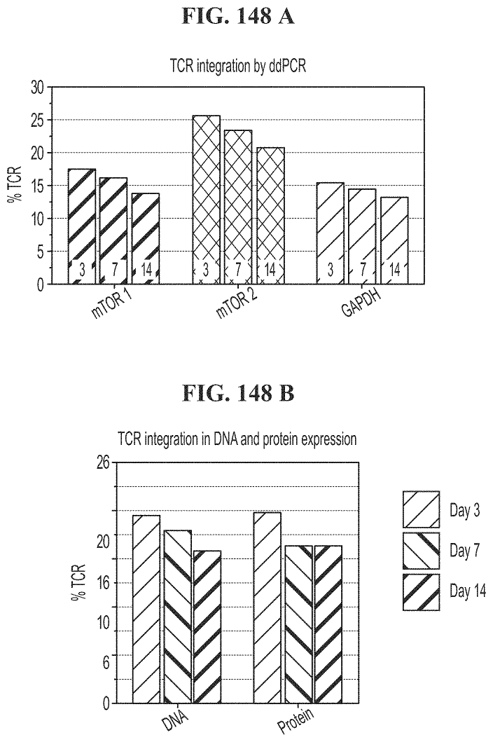

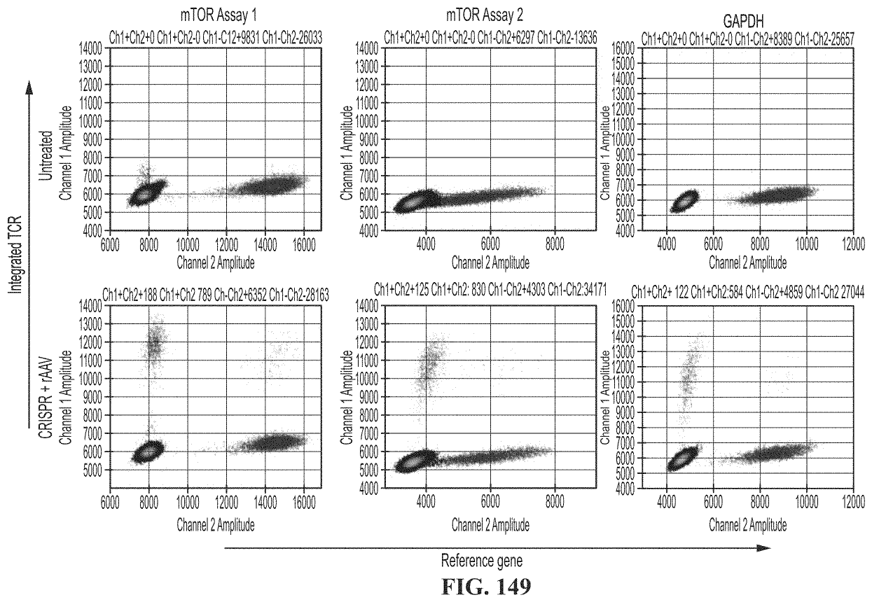

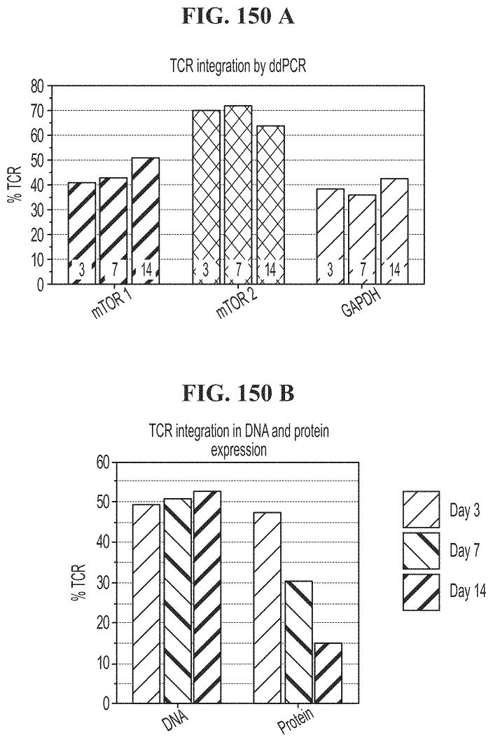

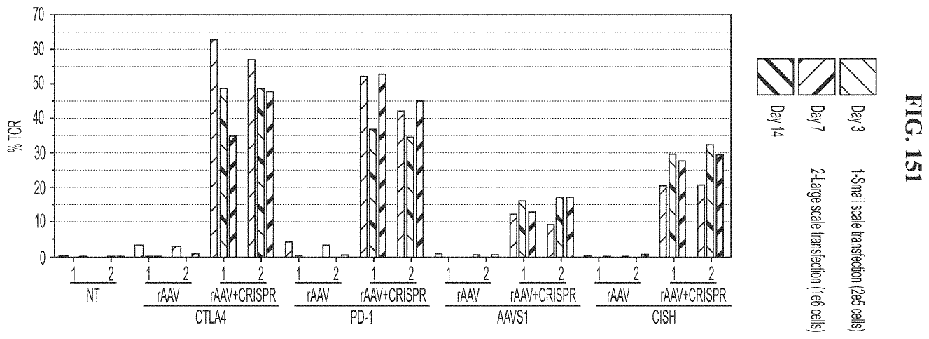

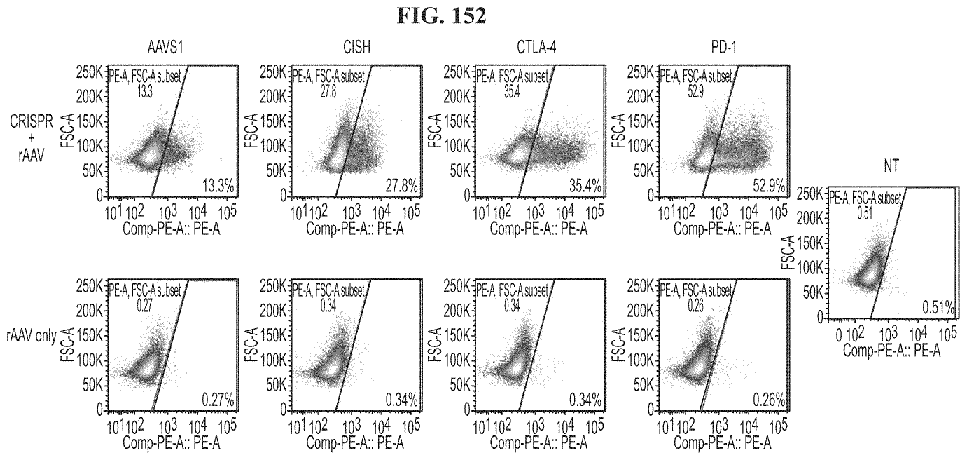

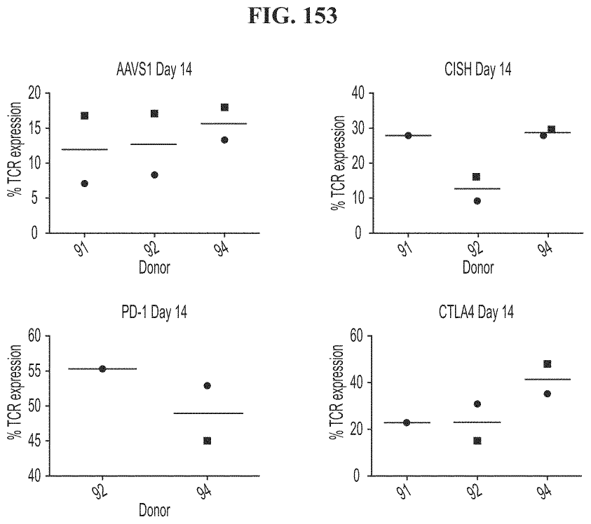

Human T cells were infected with purified rAAV at multiplicity of infection (MOI) of 1×10⁶ genome copies/virus particles per cell. The appropriate volume of virus was diluted in X-VIVO15 culture media (Lonza) containing 10% Human AB Serum (Sigma), 300 units/ml Human Recombinant IL-2, 5 ng/ml Human recombinant IL-7 and 5 ng/ml Human recombinant IL-15 (Peprotech). Diluted virus was added to the T cells in 6-well dishes, 2 hours after electroporation with the CRISPR reagents. Cells were incubated at 30° C. in a humidified incubator with 5% CO₂ for approximately 18 hours before virus containing media was replaced with fresh media as above, without virus. The T cells were returned to culture at 37° C. for a further 14 days, during which the cells were analysed at regular time points to measure mTCR expression by flow cytometry, FIG. 151, FIG. 152, FIG. 153 and integration of the mTCR expression cassette into the T cell DNA by digital droplet PCR (ddPCR), FIG. 145A, FIG. 145B, FIG. 147A, FIG. 147B, FIG. 148A, FIG. 148B, FIG. 149, FIG. 150A, and FIG. 150B.

### Example 14: ddPCR Detection of mTCR Cassette into Human T Cells

Insertion of the mTCR expression cassette into the T cell target loci was detected and quantified by ddPCR using a forward primer situated within the mTCR cassette and a reverse primer situated outside of the right homology arm within the genomic DNA region. All PCR reactions were performed with ddPCR supermix (BIO-RAD, Cat-no#186-3024) using the conditions specified by the manufacturer. PCR reactions were performed within droplets in 20 μl total volume using the following PCR cycling conditions: 1 cycle of 96° C. for 10 minutes; 40 cycles of 96° C. for 30 seconds, 55° C.-61° C. for 30 seconds, 72° C. for 240 seconds; 1 cycle of 98° C. for 10 minutes. Digital PCR data was analysed using Quantasoft (BIO-RAD).

### Example 15: Single Cell RT-PCR

TCR knock-in expression in single T lymphocytes in culture was assessed by single cell real-time RT-PCR. Single cell contents from CRISPR(CISH KO)/rAAV engineered cells were collected. Briefly, presterilized glass electrodes were filled with lysis buffer from an Ambion Single Cell-to-CT kit (Life Technologies, Grand Island, N.Y.) and were then used to obtain whole cell patches of lymphocytes in culture. The intracellular contents (˜4-5 μl) were drawn into the tip of the patch pipette by applying negative pressure and were then transferred to RNase/DNase-free tubes. The volume in each tube was brought up to 10 μl by adding Single Cell DNase I/Single Cell Lysis solution, and then the contents were incubated at room temperature for 5 min. Following cDNA synthesis by performing reverse transcription in a thermal cycler (25° C. for 10 min, 42° C. for 60 min, and 85° C. for 5 min), TCR gene expression primers were mixed with preamplification reaction mix based on the instructions from the kit (95° C. for 10 min, 14 cycles of 95° C. for 15 s, 60° C. for 4 min, and 60° C. for 4 min). The products from the preamplification stage were used for the real-time RT-PCT reaction (50° C. for 2 min, 95° C. 10 min, and 40 cycles of 95° C. for 5 s and 60° C. for 1 min). The products from the real-time RT-PCR were separated by electrophoresis on a 3% agarose gel containing 1 μl/ml ethidium bromide.

### Results

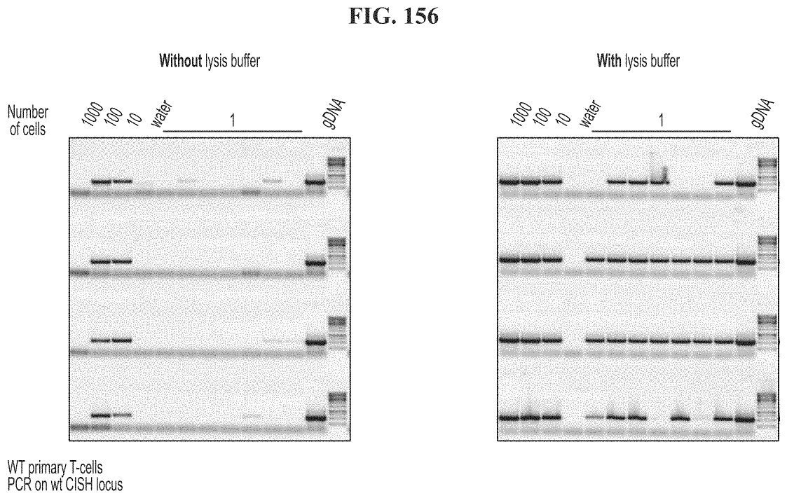

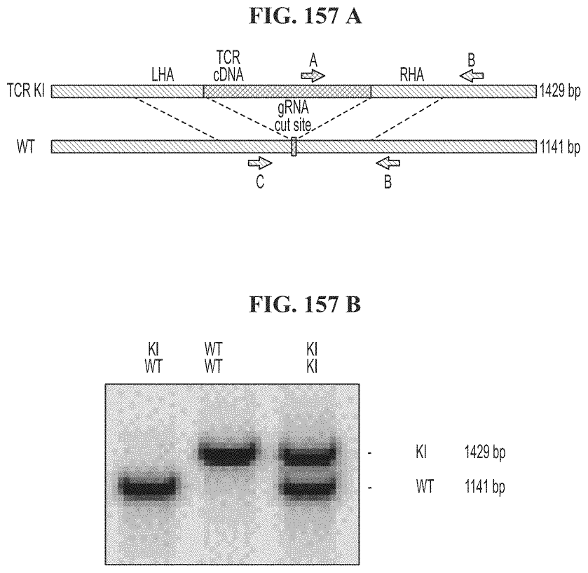

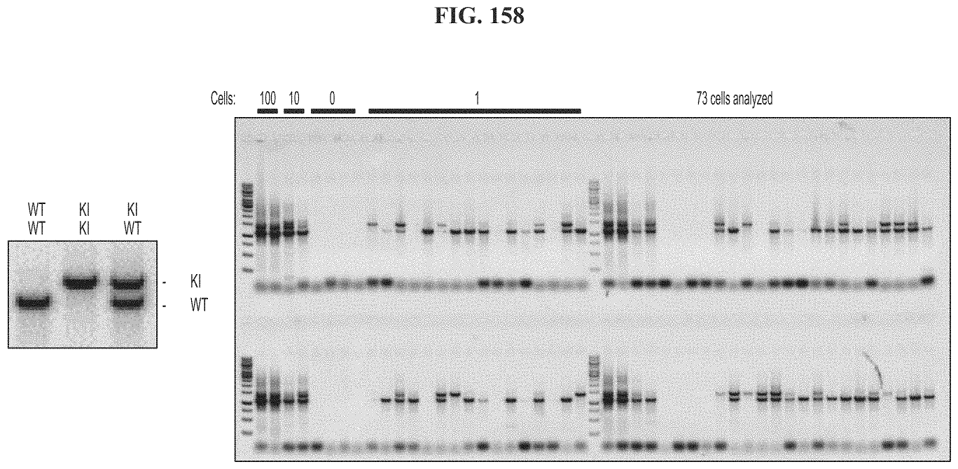

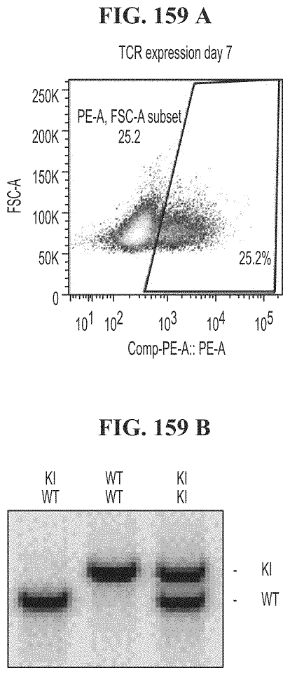

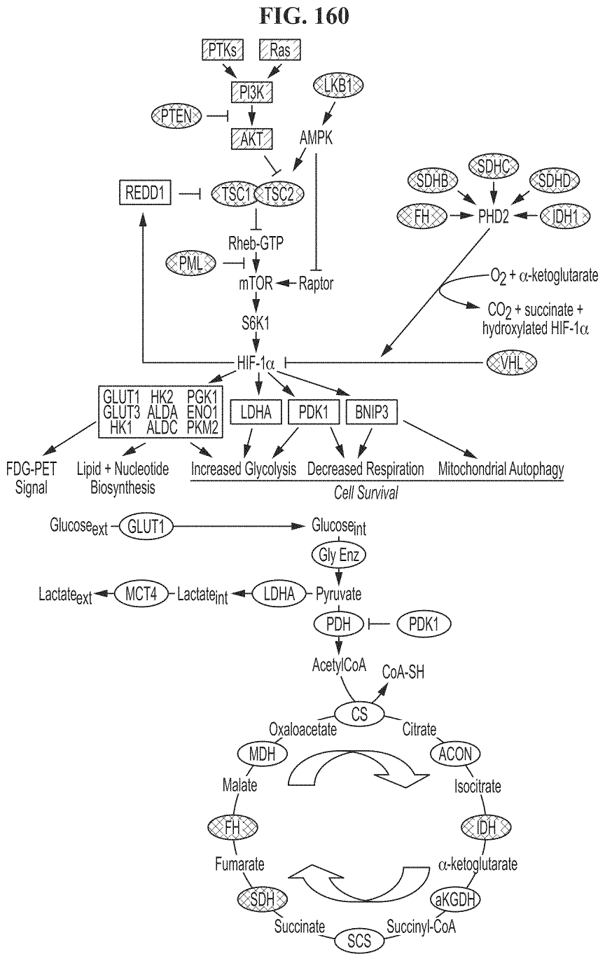

Single cell RT-PCR data showed that following CRISPR and rAAV modification, T lymphocytes expressed an exogenous TCR at 25%, FIG. 159A, on day 7 post electroporation and transduction, FIG. 156, FIG. 157A, FIG. 157B, FIG. 158, and FIG. 159B.

### Example 16: GUIDE-Seq Library Preparation

Genomic DNA was isolated from transfected, control (untransfected and CRISPR transfected cells with rAAV carrying an exogenous TCR. Transductions utilizing 8 pm dsTCR donor or 16 pmol ds TCR donor were compared Human T cells isolated using solid-phase reversible immobilization magnetic beads (Agencourt DNAdvance), were sheared with a Covaris S200 instrument to an average length of 500 bp, end-repaired, A-tailed, and ligated to half-functional adapters, incorporating a 8-nt random molecular index. Two rounds of nested anchored PCR, with primers complementary to the oligo tag, were used for target enrichment. End Repair Thermocycler Program: 12° C. for 15 min, 37° C. for 15 min; 72° C. for 15 min; hold at 4° C.

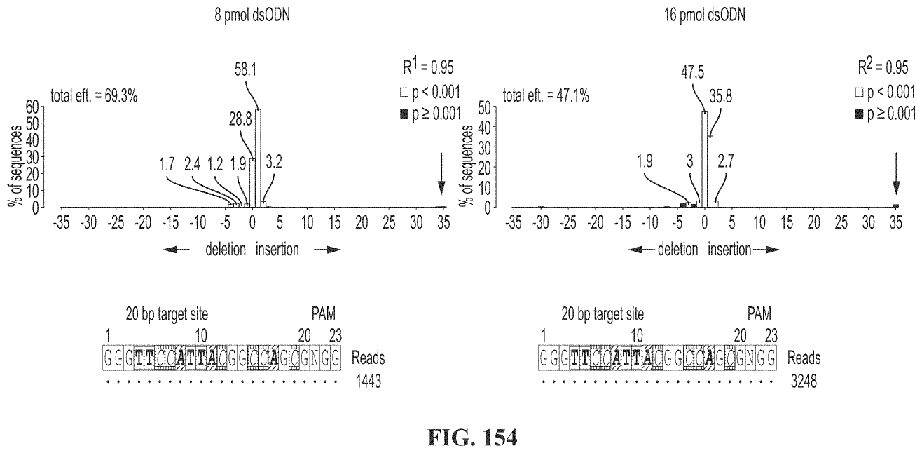

Start sites of GUIDE-Seq reads mapped back to the genome enable localization of the DSB to within a few base pairs. Quantitate library using Kapa Biosystems kit for Illumina Library Quantification kit, according to manufacturer instruction. Using the mean quantity estimate of number of molecules per uL given by the qPCR run for each sample, proceed to normalize the total set of libraries to 1.2×10¹⁰ molecules, divided by the number of libraries to be pooled together for sequencing. This gave a by molecule input for each sample, and also a by volume input for each sample Mapped reads for the on- and off-target sites of the three RGNs directed by truncated gRNAs we assessed by GUIDE-Seq are shown. In all cases, the target site sequence is shown with the protospacer sequence to the left and the PAM sequence to the right on the x-axis. Denature the library and load onto the Miseq according to Illumina's standard protocol for sequencing with an Illumina Miseq Reagent Kit V2-300 cycle (2×150 bp paired end). FIG. 154 shows data for a representative GUIDE-Seq experiment.

TABLE 12
Sequence listings for modified gRNAs targeting the PD-1, CTLA-4,
AAVS1, or CISH genes.

| SEQ ID | gRNA | Sequence 5′-3′ |
|---|---|---|
| 154 | PD-1 gRNA #2 | gcctgctcgtggtgaccgaagguuuuagagcuagaaauagcaaguuaa |
| | | aauaaggcuaguccguuaucaacuugaaaaaguggcaccgagucgg |
| | | ugcuuuu |
| 155 | PD-1 gRNA #6 | gacggaagcggcagtcctggcguuuuagagcuagaaauagcaaguua |
| | | aaauaaggcuaguccguuaucaacuugaaaaaguggcaccgagucg |
| | | gugcuuuu |
| 156 | CTLA4 gRNA #3 | gctagatgattccatctgcac guuuuagagcuagaaauagcaaguuaaa |
| | | auaaggcuaguccguuaucaacuugaaaaaguggcaccgagucggu |
| | | gcuuuu |
| 157 | CTLA4 gRNA #2 | gtgcggcaacctacatgatgguuuuagagcuagaaauagcaaguuaaa |
| | | auaaggcuaguccguuaucaacuugaaaaaguggcaccgagucggu |
| | | gcuuuu |
| 158 | CISH gRNA #2 | gggttccattacggccagcgguuuuagagcuagaaauagcaaguuaaa |
| | | auaaggcuaguccguuaucaacuugaaaaaguggcaccgagucggu |
| | | gcuuuu |
| 159 | AAVS1 | gtcaccaatcctgtccctagguuuuagagcuagaaauagcaaguuaaaa |
| | | uaaggcuaguccguuaucaacuugaaaaaguggcaccgagucggug |
| | | cuuuu |

TABLE 13
Vector constructs

| SED | | |
|---|---|---|
| ID | Construct | Sequence 5′-3′ |
| 174 | pPBSB- | gtggcacttttcggggaaatgtgcgcggaacccctatttgtttatttttctaaatacattcaaatatgtatccgctcatg |
| | Cagg- | agacaataaccctgataaatgcttcaataatattgaaaaaggaagagtatgagtattcaacatttccgtgtcgccctt |
| | RTreporter | attcccttttttgcggcattttgccttcctgtttttgctcacccagaaacgctggtgaaagtaaaagatgctgaagatc |
| | (Puro)() | agttgggtgcacgagtgggttacatcgaactggatctcaacagcggtaagatccttgagagttttcgccccgaag |
| | | aacgttttccaatgatgagcacttttaaagttctgctatgtggcgcggtattatcccgtattgacgccgggcaagag |
| | | caactcggtcgccgcatacactattctcagaatgacttggttgagtactcaccagtcacagaaaagcatcttacgg |
| | | atggcatgacagtaagagaattatgcagtgctgccataaccatgagtgataacactgcggccaacttacttctgac |
| | | aacgatcggaggaccgaaggagctaaccgcttttttgcacaacatgggggatcatgtaactcgccttgatcgttg |
| | | ggaaccggagctgaatgaagccataccaaacgacgagcgtgacaccacgatgcctgtagcaatggcaacaac |
| | | gttgcgcaaactattaactggcgaactacttactctagcttcccggcaacaattaatagactggatggaggcggat |
| | | aaagttgcaggaccacttctgcgctcggcccttccggctggctggtttattgctgataaatctggagccggtgagc |
| | | gtgggtctcgcggtatcattgcagcactggggccagatggtaagccctcccgtatcgtagttatctacacgacgg |
| | | ggagtcaggcaactatggatgaacgaaatagacagatcgctgagataggtgcctcactgattaagcattggtaac |
| | | tgtcagaccaagtttactcatatatactttagattgatttaaaacttcatttttaatttaaaaggatctaggtgaagatcct |
| | | ttttgataatctcatgaccaaaatcccttaacgtgagttttcgttccactgagcgtcagaccccgtagaaaagatcaa |
| | | aggatcttcttgagatcctttttttctgcgcgtaatctgctgcttgcaaacaaaaaaaccaccgctaccagcggtggt |
| | | ttgtttgccggatcaagagctaccaactctttttccgaaggtaactggcttcagcagagcgcagataccaaatactg |
| | | tccttctagtgtagccgtagttaggccaccacttcaagaactctgtagcaccgcctacatacctcgctctgctaatcc |
| | | tgttaccagtggctgctgccagtggcgataagtcgtgtcttaccgggttggactcaagacgatagttaccggataa |
| | | ggcgcagcggtcgggctgaacggggggttcgtgcacacagcccagcttggagcgaacgacctacaccgaac |
| | | tgagatacctacagcgtgagctatgagaaagcgccacgcttcccgaagggagaaaggcggacaggtatccgg |
| | | taagcggcagggtcggaacaggagagcgcacgagggagcttccagggggaaacgcctggtatctttatagtc |
| | | ctgtcgggtttcgccacctctgacttgagcgtcgatttttgtgatgctcgtcaggggggcggagcctatggaaaaa |
| | | cgccagcaacgcggcctttttacggttcctggccttttgctggccttttgctcacatgttctttcctgcgttatcccctg |
| | | attctgtggataaccgtattaccgcctttgagtgagctgataccgctcgccgcagccgaacgaccgagcgcagc |
| | | gagtcagtgagcgaggaagcggaagagcgcccaatacgcaaaccgcctctccccgcgcgttggccgattcatt |
| | | aatgcagctggcacgacaggtttcccgactggaaagcgggcagtgagcgcaacgcaattaatgtgagttagctc |
| | | actcattaggcaccccaggctttacactttatgcttccggctcgtatgttgtgtggaattgtgagcggataacaatttc |
| | | acacaggaaacagctatgaccatgattacgccaagcgcgcccgccgggtaactcacggggtatccatgtccatt |
| | | tctgcggcatccagccaggatacccgtcctcgctgacgtaatatcccagcgccgcaccgctgtcattaatctgca |
| | | caccggcacggcagttccggctgtcgccggtattgttcgggttgctgatgcgcttcgggctgaccatccggaact |
| | | gtgtccggaaaagccgcgacgaactggtatcccaggtggcctgaacgaacagttcaccgttaaaggcgtgcat |
| | | ggccacaccttcccgaatcatcatggtaaacgtgcgttttcgctcaacgtcaatgcagcagcagtcatcctcggca |
| | | aactctttccatgccgcttcaacctcgcgggaaaaggcacgggcttcttcctccccgatgcccagatagcgccag |
| | | cttgggcgatgactgagccggaaaaaagacccgacgatatgatcctgatgcagctagattaaccctagaaagat |
| | | agtctgcgtaaaattgacgcatgcattcttgaaatattgctctctctttctaaatagcgcgaatccgtcgctgtgcattt |
| | | aggacatctcagtcgccgcttggagctcccgtgaggcgtgcttgtcaatgcggtaagtgtcactgattttgaactat |
| | | aacgaccgcgtgagtcaaaatgacgcatgattatcttttacgtgacttttaagatttaactcatacgataattatattgtt |
| | | atttcatgttctacttacgtgataacttattatatatatattttcttgttatagataaatggtaccagatccctatacagttga |
| | | agtcggaagtttacatacaccttagccaaatacatttaaactcactttttcacaattcctgacatttaatcctagtaaaa |
| | | attccctgtcttaggtcagttaggatcaccactttattttaagaatgtgaaatatcagaataatagtagagagaatgatt |
| | | catttcagcttttatttctttcatcacattcccagtgggtcagaagtttacatacactcaattagtatttggtagcattgcc |
| | | tttaaattgtttaacttggtctccctttagtgagggttaattgatatcgaattcagatctgctagttattaatagtaatcaat |
| | | tacggggtcattagttcatagcccatatatggagttccgcgttacataacttacggtaaatggcccgcctggctgac |
| | | cgcccaacgacccccgcccattgacgtcaataatgacgtatgttcccatagtaacgccaatagggactttccattg |
| | | acgtcaatgggtggactatttacggtaaactgcccacttggcagtacatcaagtgtatcatatgccaagtacgccc |
| | | cctattgacgtcaatgacggtaaatggcccgcctggcattatgcccagtacatgaccttatgggactttcctacttg |
| | | gcagtacatctacgtattagtcatcgctattaccatgggtcgaggtgagccccacgttctgcttcactctccccatct |
| | | cccccccctccccacccccaattttgtatttatttattttttaattattttgtgcagcgatgggggcggggggggggg |
| | | gggcgcgcgccaggcggggcggggcggggcgaggggcggggcggggcgaggcggagaggtgcggcg |
| | | gcagccaatcagagcggcgcgctccgaaagtttccttttatggcgaggcggcggcggcggcggccctataaaa |
| | | agcgaagcgcgcggcgggcgggagtcgctgcgttgccttcgccccgtgccccgctccgcgccgcctcgcgc |
| | | cgcccgccccggctctgactgaccgcgttactcccacaggtgagcgggcgggacggcccttctcctccgggct |
| | | gtaattagcgcttggtttaatgacggctcgtttcttttctgtggctgcgtgaaagccttaaagggctccgggagggc |
| | | cctttgtgcgggggggagcggctcggggggtgcgtgcgtgtgtgtgtgcgtggggagcgccgcgtgcggccc |
| | | gcgctgcccggcggctgtgagcgctgcgggcgcggcgcggggctttgtgcgctccgcgtgtgcgcgagggg |
| | | agcgcggccgggggcggtgccccgcggtgcgggggggctgcgaggggaacaaaggctgcgtgcggggtg |
| | | tgtgcgtgggggggtgagcagggggtgtgggcgcggcggtcgggctgtaacccccccctgcacccccctccc |
| | | cgagttgctgagcacggcccggcttcgggtgcggggctccgtgcggggcgtggcgcggggctcgccgtgcc |
| | | gggcggggggtggcggcaggtgggggtgccgggcggggcggggccgcctcgggccggggagggctcgg |
| | | gggaggggcgcggcggccccggagcgccggcggctgtcgaggcgcggcgagccgcagccattgccttttat |
| | | ggtaatcgtgcgagagggcgcagggacttcctttgtcccaaatctggcggagccgaaatctgggaggcgccgc |
| | | cgcaccccctctagcgggcgcgggcgaagcggtgcggcgccggcaggaaggaaatgggcggggagggcc |
| | | ttcgtgcgtcgccgcgccgccgtccccttctccatctccagcctcggggctgccgcagggggacggctgccttc |
| | | gggggggacggggcagggcggggttcggcttctggcgtgtgaccggcggctctagagcctctgctaaccatg |
| | | ttcatgccttcttctttttcctacagctcctgggcaacgtgctggttgagtgctgtctcatcattaggcaaagaattcat |
| | | aacttcgtatagcatacattatacgaagttatgagctctctggctaactagagaacccactgcttactggcttatcga |
| | | aattaatacgactcactatagggagacccaagctggctagttaagctatcaagcctgcttttttgtacaaacttgtgc |
| | | tcttgggctgcaggtcgagggatctccataagagaagagggacagctatgactgggagtagtcaggagaggag |
| | | gaaaaatctggctagtaaaacatgtaaggaaaattttagggatgttaaagaaaaaaataacacaaaacaaaatata |
| | | aaaaaaatctaacctcaagtcaaggcttactatggaataaggaatggacagcagggggctgtttcatatactgatg |
| | | acctctttatagccaacctagttcatggcagccagcatatgggcatatgagccaaactctaaaccaaatactcattc |
| | | tgatgttttaaatgatttgccctcccatatgtccttccgagtgagagacacaaaaaattccaacacactattgcaatg |
| | | aaaataaatacctttattagccagaagtcagatgctcaaggggcttcatgatgtccccataatttttggcagaggga |
| | | aaaagatctcagtggtatttgtgagccagggcattggccacaccagccaccaccttctgataggcagcctgcacc |
| | | tgaggagtgaattatcgaattcctattacacccactcgtgcaggctgcccaggggcttgcccaggctggtcagct |
| | | gggcgatggcggtctcgtgctgctccacgaagccgccgtcctccacgtaggtcttctccaggcggtgctggatg |
| | | aagtggtactcggggaagtccttcaccacgcccttgctcttcatcagggtgcgcatgtggcagctgtagaacttgc |
| | | cgctgttcaggcggtacaccaggatcacctggcccaccagcacgccgtcgttcatgtacaccacctcgaagctg |
| | | ggctgcaggccggtgatggtcttcttcatcacggggccgtcgttggggaagttgcggcccttgtactccacgcgg |
| | | tacacgaacatctcctcgatcaggttgatgtcgctgcggatctccaccaggccgccgtcctcgtagcgcagggtg |
| | | cgctcgtacacgaagccggcggggaagctctggatgaagaagtcgctgatgtcctcggggtacttggtgaagg |
| | | tgcggttgccgtactggaaggcggggctcaggtgagtccaggagatgtttcagcactgttgcctttagtctcgag |
| | | gcaacttagacaactgagtattgatctgagcacagcagggtgtgagctgtttgaagatactggggttgggggtga |
| | | agaaactgcagaggactaactgggctgagacccagtggcaatgttttagggcctaaggaatgcctctgaaaatct |
| | | agatggacaactagactagagaaaagagaggtggaaatgaggaaaatgacttttctttattagatttcggtagaa |
| | | agaactacatctttcccctatttttgttattcgttttaaaacatctatctggaggcaggacaagtatggtcattaaaaag |
| | | atgcaggcagaaggcatatattggctcagtcaaagtgggggaactttggtggccaaacatacattgctaaggcta |
| | | ttcctatatcagctggacacatataaaatgctgctaatgcttcattacaaacttatatcctttaattccagatgggggca |
| | | aagtatgtccaggggtgaggaacaattgaaacatttgggctggagtagattttgaaagtcagctctgtgtgtgtgtg |
| | | tgtgtgtgtgtgtgtgtgtgtgtgcgcgcacgtgtgtttgtgtgtgtgtgagagcgtgtgtttcttttaacgttttcagcc |
| | | tacagcatacagggttcatggtggcaagaagataacaagatttaaattatggccagtgactagtgctgcaagaag |
| | | aacaactacctgcatttaatgggaaagcaaaatctcaggctttgagggaagttaacataggcttgattctgggtgg |
| | | aagctgggtgtgtagttatctggaggccaggctggagctctcagctcactatgggttcatctttattgtctcctttcat |
| | | ctcatcaggatgtcgaaggcgaagggcaggggggcgcccttggtcacgcggatctgcaccagctggttgccg |
| | | aacaggatgttgcccttgccgcagccctccatggtgaacacgtggttgttcaccacgccctccaggttcaccttga |
| | | agctcatgatctcctgcaggccggtgttcttcaggatctgcttgctcaccatggtaattcctcacgacacctgaaat |
| | | ggaagaaaaaaactttgaaccactgtctgaggcttgagaatgaaccaagatccaaactcaaaaagggcaaattc |
| | | caaggagaattacatcaagtgccaagctggcctaacttcagtctccacccactcagtgtggggaaactccatcgc |
| | | ataaaacccctccccccaacctaaagacgacgtactccaaaagctcgagaactaatcgaggtgcctggacggc |
| | | gcccggtactccgtggagtcacatgaagcgacggctgaggacggaaaggcccttttcctttgtgtgggtgactca |
| | | cccgcccgctctcccgagcgccgcgtcctccattttgagctccctgcagcagggccgggaagcggccatctttc |
| | | cgctcacgcaactggtgccgaccgggccagccttgccgcccagggcggggcgatacacggcggcgcgagg |
| | | ccaggcaccagagcaggccggccagcttgagactacccccgtccgattctcggtggccgcgctcgcaggccc |
| | | cgcctcgccgaacatgtgcgctgggacgcacgggccccgtcgccgcccgcggccccaaaaaccgaaatacc |
| | | agtgtgcagatcttggcccgcatttacaagactatcttgccagaaaaaaagccttgccagaaaaaaagcgtcgca |
| | | gcaggtcatcaaaaattttaaatggctagagacttatcgaaagcagcgagacaggcgcgaaggtgccaccagat |
| | | tccgcacgcggcggccccagcgcccaggccaggcctcaactcaagcacgaggcgaaggggctccttaagcg |
| | | caaggcctcgaactctcccacccacttccaacccgaagctcgggatcaagaatcacgtactgcagccaggggc |
| | | gtggaagtaattcaaggcacgcaagggccataacccgtaaagaggccaggcccgcgggaaccacacacggc |
| | | acttacctgtgttctggcggcaaacccgagcgaaaaagaacgttcacggcgactactgcacttatatacggttctc |
| | | ccccaccctcgggaaaaaggcggagccagtacacgacatcactttcccagtttaccccgcgccaccttctctag |
| | | gcaccggttcaattgccgacccctccccccaacttctcggggactgtgggcgatgtgcgctctgcccactgacg |
| | | ggcaccggagcctcacgcatgctcttctccacctcagtgatgacgagagcgggcgggtgagggggcgggaac |
| | | gcagcgatctctgggttctacgttagtgggagtttaacgacggtccctgggattccccaaggcaggggcgagtc |
| | | cttttgtatgaattactctcagctccggtcggggcgggttggggggggtggtgacggggaggccgcctggaag |
| | | ggacgtgcagaatcttccctctaccattgctggcttagctccaaaggttgtattgagattagggtgtaccttcgcctc |
| | | tcaatcagcctcccgtcctcagccttgccatctcgctagtccgggacaaatccctagagcgtcttcctctgcgggt |
| | | ctcagcccagcccggggttggctcctcctccgccccggcttccgcgcccctcccgtgtggcaaggagtaccag |
| | | gcccggggaccccgaggggcttggggcgaagggtcgggactgggggcctccttaacggctcacggacttgc |
| | | gagaggttcggctcgatggccgtgaaagcgacgaatccgctcctgtgctggcctcttggctccttccattcaaag |
| | | ccagctgcttttatggaagcccgtaacacgtcatctccccctggtactccagatgtccaggctacagtttagaatag |
| | | actcagtcctacagttagctttagatctaattctagttagttacgccaaaaagttcctgcgagtgtgtgtgtgtgcctc |
| | | atggtactttttaaattaaaaggtgtacagttatttgattgcaaacataaggaacctaaaatgctacagattaccacat |
| | | gatctcatgtagaggctaagatctacagcatcagcaagtttatccacccagtttcctaaccccaacacttgctatga |
| | | agtcacagcttctcctatttaaataagtgcctattatatttaaataagtgctgtcgttttctgtcatcctatcgattgtaact |
| | | gcattttagcataaatctagggcaagattggatgagcttggcctttttggatggctatcaaggcaggccttgggaaa |
| | | tgctcctctgaggaaagaagaacgtttatttttaatgagctaattactagatcattatgtacttcttccagctgtagaat |
| | | atcattgcccagcttctcgaacaaacttatttattaacaagtatttgagaacctactatgtggccaacgctaagtgac |
| | | ctgcaggcatgcaagctgagcctattctaccaccactttgtacaagaaagctgggttgatctagagggcccgcgg |
| | | ttcgaaggtaagcctatccctaaccctctcctcggtctcgattctacgcgtcaggtgcaggctgcctatcagaaggt |
| | | ggtggctggtgtggccaatgccctggctcacaaataccactgagatctttttccctctgccaaaaattatggggac |
| | | atcatgaacgcagtgaaaaaaatgctttatttgtgaaatttgtgatgctattgctttatttgtaaccattataagctgcaa |
| | | taaacaagttctcgagaagttcctattctctagaaagtataggaacttctggctgcaggtcgtcgaaattctaccgg |
| | | gtaggggaggcgcttttcccaaggcagtctggagcatgcgctttagcagccccgctgggcacttggcgctacac |
| | | aagtggcctctggcctcgcacacattccacatccaccggtaggcgccaaccggctccgttctttggtggcccctt |
| | | cgcgccaccttctactcctcccctagtcaggaagacccccccgccccgcagctcgcgtcgtgcaggacgtgac |
| | | aaatggaagtagcacgtctcactagtctcgtgcagatggacagcaccgctgagcaatggaagcgggtaggcctt |
| | | tggggcagcggccaatagcagctttggctccttcgctttctgggctcagaggctgggaaggggtgggtccggg |
| | | ggcgggctcaggggcgggctcaggggcggggcgggcgcccgaaggtcctccggaggcccggcattctgca |
| | | cgcttcaaaagcgcacgtctgccgcgctgttctcctcttcctcatctccgggcctttcgacctgcatccatctagatc |
| | | tcgagcagctgaagcttaccatgaccgagtacaagcccacggtgcgcctcgccacccgcgacgacgtcccca |
| | | gggccgtacgcaccctcgccgccgcgttcgccgactaccccgccacgcgccacaccgtcgatccagaccgcc |
| | | acatcgagcgggtcaccgagctgcaagaactcttcctcacgcgcgtcgggctcgacatcggcaaggtgtgggt |
| | | cgcggacgacggcgcagcagtggcggtctggaccacgccggagagcgtcgaagcgggggcggtgttcgcc |
| | | gagatcggcccgcgcatggccgagttgagcggacccggctggccgcgcagcaacagatggaaggcctcctg |
| | | gcgccgcaccggcccaaggagcccgcgtggttcctggccaccgtcggcgtctcgcccgaccaccagggcaa |
| | | gggtctgggcagcgccgtcgtgctccccggagtggaggcggccgagcgcgccggggtgcccgccttcctgg |
| | | agacctccgcgccccgcaacctccccttctacgagcggctcggcttcaccgtcaccgccgacgtcgaggtgcc |
| | | cgaaggaccgcgcacttggtgcatgacccgcaagcccggtgcctgacgcccgcccacaagacccgcagcgc |
| | | ccgaccgaaaggagcgcacgaccccatgcatcgatgatctagagctcgctgatcagcctcgactgtgccttcta |
| | | gttgccagccatctgttgtttgcccctcccccgtgccttccttgaccctggaaggtgccactcccactgtcctttcct |
| | | aataaaatgaggaaattgcatcgcattgtctgagtaggtgtcattctattctggggggtggggtggggcaggaca |
| | | gcaagggggaggattgggaagacaatagcaggcatgctggggatgcggtgggctctatggcttctgaggcgg |
| | | aagacctattctctagaaagtataggaacttctcgagtctagaagatgggcgggagtcttctgggcaggcttaaa |
| | | ggctaacctggtgtgtgggcgttgtcctgcaggggaattgaacaggtgattaccctgttatccctagtaatcccgg |
| | | gatctaatacgactcactatagggagaccatcattttctggaattttccaagctgtttaaaggcacagtcaacttagt |
| | | gtatgtaaacttctgacccactggaattgtgatacagtgaattataagtgaaataatctgtctgtaaacaattgttgga |
| | | aaaatgacttgtgtcatgcacaaagtagatgtcctaactgacttgccaaaactattgtttgttaacaagaaatttgtgg |
| | | agtagttgaaaaacgagttttaatgactccaacttaagtgtatgtaaacttccgacttcaactgtatagggatccccc |
| | | gggctgcaggaattcgataaaagttagttactttatagaagaaattagagtttttgtttttttttaataaataaataaaca |
| | | taaataaattgtagttgaatttattattagtatgtaagtgtaaatataataaaacttaatatctattcaaattaataaataaa |
| | | cctcgatatacagaccgataaaacacatgcgtcaattttacgcatgattatctttaacgtacgtcacaatatgattatc |
| | | tttctagggttaatctagctgcgtgttctgcagcgtgtcgagcatcttcatctgctccatcacgctgtaaaacacattt |
| | | gcaccgcgagtctgcccgtcctccacgggttcaaaaacgtgaatgaacgaggcgcgctcactggccgtcgtttt |
| | | acaacgtcgtgactgggaaaaccctggcgttacccaacttaatcgccttgcagcacatccccctttcgccagctg |
| | | gcgtaatagcgaagaggcccgcaccgatcgcccttcccaacagttgcgcagcctgaatggcgaatgggacgc |
| | | gccctgtagcggcgcattaagcgcggcgggtgtggtggttacgcgcagcgtgaccgctacacttgccagcgcc |
| | | ctagcgcccgctcctttcgctttcttcccttcctttctcgccacgttcgccggctttccccgtcaagctctaaatcggg |
| | | ggctccctttagggttccgatttagtgctttacggcacctcgaccccaaaaaacttgattagggtgatggttcacgta |
| | | gtgggccatcgccctgatagacggtttttcgccctagacgaggagtccacgactttaatagtggactcttgacca |
| | | aactggaacaacactcaaccctatctcggtctattcttttgatttataagggattttgccgatttcggcctattggttaa |
| | | aaaatgagctgatttaacaaaaatttaacgcgaattttaacaaaatattaacgcttacaatttag |
| 175 | AMVlarge | gacggatcgggagatctcccgatcccctatggtgcactctcagtacaatctgctctgatgccgcatagttaagcca |
| | pcDNA | gtatctgctccctgcttgtgtgttggaggtcgctgagtagtgcgcgagcaaaatttaagctacaacaaggcaaggc |
| | Dest40 | ttgaccgacaattgcatgaagaatctgcttagggttaggcgttttgcgctgcttcgcgatgtacgggccagatatac |
| | | gcgttgacattgattattgactagttattaatagtaatcaattacggggtcattagttcatagcccatatatggagacc |
| | | gcgttacataacttacggtaaatggcccgcctggctgaccgcccaacgacccccgcccattgacgtcaataatga |
| | | cgtatgttcccatagtaacgccaatagggactttccattgacgtcaatgggtggagtatttacggtaaactgcccac |
| | | ttggcagtacatcaagtgtatcatatgccaagtacgccccctattgacgtcaatgacggtaaatggcccgcctggc |
| | | attatgcccagtacatgaccttatgggactttcctacttggcagtacatctacgtattagtcatcgctattaccatggtg |
| | | atgcggttttggcagtacatcaatgggcgtggatagcggtttgactcacggggatttccaagtctccaccccattg |
| | | acgtcaatgggagtttgttttggcaccaaaatcaacgggactttccaaaatgtcgtaacaactccgccccattgac |
| | | gcaaatgggcggtaggcgtgtacggtgggaggtctatataagcagagctctctggctaactagagaacccactg |
| | | cttactggcttatcgaaattaatacgactcactatagggagacccaagctggctagttaagctatcaacaagtttgta |
| | | caaaaaagctgaacgagaaacgtaaaatgatataaatatcaatatattaaattagattagcataaaaaacagacta |
| | | cataatactgtaaaacacaacatatccagtcactatggctgccaccatggactacaaagacgatgacgacaagag |
| | | cagggctgaccccaagaagaagaggaaggtgactgtcgctctgcacctggcaatacctcttaaatggaaaccta |
| | | atcacactccagtttggatcgatcaatggccacttcctgagggcaagttggtggcattgactcagttggtagagaa |
| | | agaactccaacttgggcacatcgaaccgtccctgtcctgttggaacaccccagtattcgtcataaggaaagcctc |
| | | cggaagttaccgcttgcttcatgacctgagggcggtgaatgcaaagcttgtaccttttggcgccgtccagcaggg |
| | | agctccagtcttgagtgccttgccacggggatggccgcttatggttctcgatttgaaggactgctttttcagcattcc |
| | | gcttgcggaacaggatcgagaggctttcgcctttacgctgcccagcgtcaacaaccaggccccggctagacgct |
| | | tccaatggaaagtcctccctcagggtatgacctgttcacctacaatttgtcaacttattgttggtcaaatcctggaacc |
| | | gcttagattgaagcatccgtcccttagaatgctgcattatatggacgacctgcttctcgcagcgagttctcacgacg |
| | | ggttggaggctgccggagaagaagttattagcacccttgaacgagcagggttcaccatttcaccggataaggta |
| | | cagcgggaacccggcgtacagtacttgggctacaagctcggttcaacatacgtggcccccgtaggactggagc |
| | | cgagccaaggattgcaactctttgggatgtacaaaaactcgttggttcacttcagtggttgaggcccgctctcggc |
| | | attccgccgagacttatgggccctttctatgagcagcttagaggatctgacccgaacgaagcacgagaatggaa |
| | | cctggacatgaaaatggcctggcgagagatcgtacagctctcaacgacggctgctcttgaacggtgggacccc |
| | | gcccttcccctcgaaggggctgtggcacgctgtgaacaaggagctataggggtcctcggtcagggactttccac |
| | | ccatccccgcccatgtctaggcttattcaactcaacccaccaaagcatttacagcgtggctggaggtacttaccct |
| | | tctcattaccaaattgcgagcgtccgcggtccgaactttcgggaaagaagtagatatattgttgctgccagcctgttt |
| | | tagagaagatttgccccttccagaagggattcttcttgccttgagaggtttcgcaggtaagattagaagtagcgaca |
| | | caccgtccatcttcgacatcgcgcgcccgctccacgtgagcctgaaggttagagtcaccgaccatcccgttccg |
| | | ggtcccacagtttttaccgatgcatctagtagtacccacaaaggagtagtagtctggcgcgagggacctcgatgg |
| | | gaaataaaggagatcgcagatttgggggctagtgttcagcagttggaagcacgcgccgtggcgatggctcttct |
| | | cctgtggcccacgacaccaactaatgttgtaaccgactcagctttcgtagctaaaatgctcctgaaaatgggccag |
| | | gaaggggtcccatccactgcagctgcatttatccttgaagacgcactcagccaaaggtcagcaatggctgcggt |
| | | gctccatgtgcggtcccattccgaagtacctggtttctttacagaggggaatgatgtcgccgactctcaagcaacc |
| | | ttccaggcgtatcctcttagggaagctaaagacctccatacagctcttcatataggtccgagagctctgagcaagg |
| | | cgtgtaatattagcatgcagcaagctagggaggtcgtccagacatgtccacactgtaactccgcacctgccctcg |
| | | aggcaggggtaaatccgcgagggttggggccgctccagatctggcaaactgatttcacgttggaaccaaggat |
| | | ggctccgcggagttggctggcagtaaccgtagacacagcgtcttctgcaattgttgtaactcagcatggccgcgt |
| | | gactagcgtggccgcgcagcatcactgggcaacggctatagcggtcctcggacgacctaaagcaataaagac |
| | | ggacaatggcagttgttttacttcaaaatcaaccagagagtggctcgctaggtggggcatagcacacacgactgg |
| | | aatccccggtaatagccaagggcaggctatggtagagagagcaaatcgactgctcaaagataagatccgggtc |
| | | cttgctgaaggggacggctttatgaagcggataccaactagtaaacagggagaacttcttgcaaaggccatgtac |
| | | gcgctcaatcattttgaacgaggggaaaatactaaaaccccgatccaaaaacactggcgacctaccgtgttgacg |
| | | gagggacctccagtaaaaatcaggattgagacgggcgagtgggaaaaaggttggaacgtgctggtctggggg |
| | | cgagggtatgctgcagtaaaaaacagagacactgacaaagtaatatgggttccatctcgcaaggttaaaccgga |
| | | catcgctcaaaaggatgaagtgacaaaaaaagacgaagcgtcaccactctttgcataatgaacccatagtgactg |
| | | gatatgttgtgttttacagtattatgtagtctgttttttatgcaaaatctaatttaatatattgatatttatatcattttacgtttc |
| | | tcgttcagctacttgtacaaagtggttgatctagagggcccgcggttcgaaggtaagcctatccctaaccctctcct |
| | | cggtctcgattctacgcgtaccggtcatcatcaccatcaccattgagtttaaacccgctgatcagcctcgactgtgc |
| | | cttctagttgccagccatctgttgtttgcccctcccccgtgccttccttgaccctggaaggtgccactcccactgtcc |
| | | tttcctaataaaatgaggaaattgcatcgcattgtctgagtaggtgtcattctattctggggggtggggtggggcag |
| | | gacagcaagggggaggattgggaagacaatagcaggcatgctggggatgcggtgggctctatggcttctgag |
| | | gcggaaagaaccagctggggctctagggggtatccccacgcgccctgtagcggcgcattaagcgcggcgggt |
| | | gtggtggttacgcgcagcgtgaccgctacacttgccagcgccctagcgcccgctcctttcgctttcttcccttccttt |
| | | ctcgccacgttcgccggctttccccgtcaagctctaaatcgggggctccctttagggttccgatttagtgctttacg |
| | | gcacctcgaccccaaaaaacttgattagggtgatggttcacgtagtgggccatcgccctgatagacggtattcgc |
| | | cctttgacgttggagtccacgttctttaatagtggactcttgttccaaactggaacaacactcaaccctatctcggtct |
| | | attcttttgatttataagggattttgccgatttcggcctattggttaaaaaatgagctgatttaacaaaaatttaacgcga |
| | | attaattctgtggaatgtgtgtcagttagggtgtggaaagtccccaggctccccagcaggcagaagtatgcaaag |
| | | catgcatctcaattagtcagcaaccaggtgtggaaagtccccaggctccccagcaggcagaagtatgcaaagca |
| | | tgcatctcaattagtcagcaaccatagtcccgcccctaactccgcccatcccgcccctaactccgcccagttccgc |
| | | ccattctccgccccatggctgactaatttatttatttatgcagaggccgaggccgcctctgcctctgagctattccag |
| | | aagtagtgaggaggcttttttggaggcctaggcttagcaaaaagctcccgggagcttgtatatccattacggatct |
| | | gatcaagagacaggatgaggatcgtttcgcatgattgaacaagatggattgcacgcaggttctccggccgcttgg |
| | | gtggagaggctattcggctatgactgggcacaacagacaatcggctgctctgatgccgccgtgttccggctgtca |
| | | gcgcaggggcgcccggttctattgtcaagaccgacctgtccggtgccctgaatgaactgcaggacgaggcag |
| | | cgcggctatcgtggctggccacgacgggcgttccttgcgcagctgtgctcgacgttgtcactgaagcgggaag |
| | | ggactggctgctattgggcgaagtgccggggcaggatctcctgtcatctcaccttgctcctgccgagaaagtatc |
| | | catcatggctgatgcaatgcggcggctgcatacgcttgatccggctacctgcccattcgaccaccaagcgaaac |
| | | atcgcatcgagcgagcacgtactcggatggaagccggtcttgtcgatcaggatgatctggacgaagagcatcag |
| | | gggctcgcgccagccgaactgttcgccaggctcaaggcgcgcatgcccgacggcgaggatctcgtcgtgacc |
| | | catggcgatgcctgcttgccgaatatcatggtggaaaatggccgcttttctggattcatcgactgtggccggctgg |
| | | gtgtggcggaccgctatcaggacatagcgttggctacccgtgatattgctgaagagcttggcggcgaatgggct |
| | | gaccgcttcctcgtgctttacggtatcgccgctcccgattcgcagcgcatcgccttctatcgccttcttgacgagttc |
| | | ttctgagcgggactctggggttcgcgaaatgaccgaccaagcgacgcccaacctgccatcacgagatttcgatt |
| | | ccaccgccgccttctatgaaaggttgggcttcggaatcgttttccgggacgccggctggatgatcctccagcgcg |
| | | gggatctcatgctggagttcttcgcccaccccaacttgtttattgcagcttataatggttacaaataaagcaatagca |
| | | tcacaaatttcacaaataaagcattatttcactgcattctagagtggtagtccaaactcatcaatgtatcttatcatgtc |
| | | tgtataccgtcgacctctagctagagcttggcgtaatcatggtcatagctgtttcctgtgtgaaattgttatccgctca |
| | | caattccacacaacatacgagccggaagcataaagtgtaaagcctggggtgcctaatgagtgagctaactcaca |
| | | ttaattgcgttgcgctcactgcccgctttccagtcgggaaacctgtcgtgccagctgcattaatgaatcggccaac |
| | | gcgcggggagaggcggtttgcgtattgggcgctcttccgcttcctcgctcactgactcgctgcgctcggtcgttc |
| | | ggctgcggcgagcggtatcagctcactcaaaggcggtaatacggttatccacagaatcaggggataacgcagg |
| | | aaagaacatgtgagcaaaaggccagcaaaaggccaggaaccgtaaaaaggccgcgagctggcgtattccat |
| | | aggctccgcccccctgacgagcatcacaaaaatcgacgctcaagtcagaggtggcgaaacccgacaggacta |
| | | taaagataccaggcgtttccccctggaagctccctcgtgcgctctcctgttccgaccctgccgcttaccggatacc |
| | | tgtccgcctttctcccttcgggaagcgtggcgctttctcatagctcacgctgtaggtatctcagttcggtgtaggtcg |
| | | ttcgctccaagctgggctgtgtgcacgaaccccccgttcagcccgaccgctgcgccttatccggtaactatcgtct |
| | | tgagtccaacccggtaagacacgacttatcgccactggcagcagccactggtaacaggattagcagagc gagg |
| | | tatgtaggcggtgctacagagttcttgaagtggtggcctaactacggctacactagaagaacagtataggtatctg |
| | | cgctctgctgaagccagttaccttcggaaaaagagttggtagctcttgatccggcaaacaaaccaccgctggtag |
| | | cggtggtattagtagcaagcagcagattacgcgcagaaaaaaaggatctcaagaagatcctagatcttactac |
| | | ggggtctgacgctcagtggaacgaaaactcacgttaagggattaggtcatgagattatcaaaaaggatcttcacc |
| | | tagatccttttaaattaaaaatgaagttttaaatcaatctaaagtatatatgagtaaacttggtctgacagttaccaatg |
| | | cttaatcagtgaggcacctatctcagcgatctgtctatttcgttcatccatagttgcctgactccccgtcgtgtagata |
| | | actacgatacgggagggcttaccatctggccccagtgctgcaatgataccgcgagacccacgctcaccggctc |
| | | cagatttatcagcaataaaccagccagccggaagggccgagcgcagaagtggtcctgcaactttatccgcctcc |
| | | atccagtctattaattgttgccgggaagctagagtaagtagttcgccagttaatagtagcgcaacgttgagccattg |
| | | ctacaggcatcgtggtgtcacgctcgtcgtttggtatggcttcattcagctccggttcccaacgatcaaggcgagtt |
| | | acatgatcccccatgttgtgcaaaaaagcggttagctccttcggtcctccgatcgttgtcagaagtaagttggccg |
| | | cagtgttatcactcatggttatggcagcactgcataattctcttactgtcatgccatccgtaagatgcttttctgtgact |
| | | ggtgagtactcaaccaagtcattctgagaatagtgtatgcggcgaccgagttgctcttgcccggcgtcaatacgg |
| | | gataataccgcgccacatagcagaactttaaaagtgctcatcattggaaaacgttcttcggggcgaaaactctcaa |
| | | ggatcttaccgctgttgagatccagttcgatgtaacccactcgtgcacccaactgatcttcagcatcttttactttcac |
| | | cagcgtttctgggtgagcaaaaacaggaaggcaaaatgccgcaaaaaagggaataagggcgacacggaaat |
| | | gttgaatactcatactcttcctattcaatattattgaagcatttatcagggttattgtctcatgagcggatacatatttga |
| | | atgtatttagaaaaataaacaaataggggttccgcgcacatttccccgaaaagtgccacctgacgtc |
| 176 | AMVsmall | gacggatcgggagatctcccgatcccctatggtgcactctcagtacaatctgctctgatgccgcatagttaagcca |
| | pcDNA | gtatctgctccctgcttgtgtgttggaggtcgctgagtagtgcgcgagcaaaatttaagctacaacaaggcaaggc |
| | Dest40 | ttgaccgacaattgcatgaagaatctgcttagggttaggcgttttgcgctgcttcgcgatgtacgggccagatatac |
| | | gcgttgacattgattattgactagttattaatagtaatcaattacggggtcattagttcatagcccatatatggagacc |
| | | gcgttacataacttacggtaaatggcccgcctggctgaccgcccaacgacccccgcccattgacgtcaataatga |
| | | cgtatgttcccatagtaacgccaatagggactttccattgacgtcaatgggtggagtatttacggtaaactgcccac |
| | | ttggcagtacatcaagtgtatcatatgccaagtacgccccctattgacgtcaatgacggtaaatggcccgcctggc |
| | | attatgcccagtacatgaccttatgggactttcctacttggcagtacatctacgtattagtcatcgctattaccatggtg |
| | | atgcggttttggcagtacatcaatgggcgtggatagcggtttgactcacggggatttccaagtctccaccccattg |
| | | acgtcaatgggagtttgttttggcaccaaaatcaacgggactttccaaaatgtcgtaacaactccgccccattgac |
| | | gcaaatgggcggtaggcgtgtacggtgggaggtctatataagcagagctctctggctaactagagaacccactg |
| | | cttactggcttatcgaaattaatacgactcactatagggagacccaagctggctagttaagctatcaacaagtttgta |
| | | caaaaaagctgaacgagaaacgtaaaatgatataaatatcaatatattaaattagattagcataaaaaacagacta |
| | | cataatactgtaaaacacaacatatccagtcactatggctgccaccatggactacaaagacgatgacgacaagag |
| | | cagggctgaccccaagaagaagaggaaggtgactgttgcgctccatcttgcgataccgttgaagtggaaaccg |
| | | aatcacactcctgtgtggatcgaccagtggccactcccagaagggaaactggtagcgttgacacaacttgtcgaa |
| | | aaggagcttcaacttggccatatagaacctagtagtcctgttggaacactcctgtgtagtcatcaggaaggcctcc |
| | | gggagttatcgcctgttgcacgaccttcgagctgttaatgcaaaactcgtaccctttggcgcggtgcaacaaggg |
| | | gctccagttagagtgcattgcctcgggggtggccgcttatggtcttggatctgaaggattgcttttttttgtatacctct |
| | | ggcagagcaggatagagaggcctttgccttcacgcttccttcagtgaacaaccaggctccggccaggcggtttc |
| | | aatggaaggttagccccaagggatgacttgctccccgacgatatgtcaactgatcgtgggccagatactggaac |
| | | cactccgattgaagcacccttctttgcgcatgctccattacatggatgacctcttgttggcggccagctcccatgac |
| | | ggtctggaggcggcgggtgaagaagtgataagcaccctggaacgagcgggattcacaatcagcccggacaa |
| | | agtgcaaagagagcccggagtccaatatctgggctacaagttgggttccacatacgtcgcccctgtaggcctggt |
| | | agcggaaccgcgcattgccacgttgtgggatgtgcaaaaactcgttggatctctccaatggttgcgcccggcact |
| | | gggtatcccacccagactgatgggtccattctatgaacaactgaggggctctgacccgaatgaggcgcgggaat |
| | | ggaatttggacatgaagatggcgtggcgcgaaatagtccaactttcaacaacggcggctcttgaacgctgggat |
| | | cctgccttgccgcttgaaggcgcagtagccaggtgcgagcagggggcgataggagtgttgggacaaggtctca |
| | | gcacacacccgaggccgtgcctgtggagttcagtactcaacctacgaaggcttttacagcatggctggaagtcct |
| | | caccttgagattacaaaactcagagcatctgccgtcaggaccttcggcaaggaagtagatatccttcttctgcccg |
| | | cctgcttccgcgaagaccttccactgccagagggaatactgcttgcattgaggggttttgccggtaagatccggtc |
| | | cagcgatactccgagcatatttgacatcgctagacctcttcacgtctcactcaaggttcgcgtgactgaccaccca |
| | | gttccgggacccaccgtattcaccgatgccagtagtagcactcataaaggggtagtcgtctggcgggaaggacc |
| | | tcgctgggagataaaggaaatagcagacttgggtgccagcgtgcaacaactggaggcccgggcggtcgcgat |
| | | ggcactccttttgtggccaaccaccccgacgaacgtagttacagattcagctttcgtagccaaaatgttgttgaaaa |
| | | tgggtcaggaaggtgtcccttccactgccgcagcattcatattggaggatgccctgagtcaaagaagtgcaatgg |
| | | ccgcagttcttcacgtgcgatcccatagcgaagtacctggcttttttttctgagggcaatgatgtggctgactcacag |
| | | gctacatttcaggcttattaatgaacccatagtgactggatatgagtgttttacagtattatgtagtctgtttttttttgca |
| | | aaatctaatttaatatattgatatttatatcattttacgtttctcgttcagctttcttgtacaaagtggttgatctagagggc |
| | | ccgcggttcgaaggtaagcctatccctaaccctctcctcggtctcgattctacgcgtaccggtcatcatcaccatca |
| | | ccattgagtttaaacccgctgatcagcctcgactgtgccttctagttgccagccatctgttgtttgcccctcccccgt |
| | | gccttccttgaccctggaaggtgccactcccactgtcctttcctaataaaatgaggaaattgcatcgcattgtctgag |
| | | taggtgtcattctattctggggggtggggtggggcaggacagcaagggggaggattgggaagacaatagcag |
| | | gcatgctggggatgcggtgggctctatggcttctgaggcggaaagaaccagctggggctctagggggtatccc |
| | | cacgcgccctgtagcggcgcattaagcgcggcgggtgtggtggttacgcgcagcgtgaccgctacacttgcca |
| | | gcgccctagcgcccgctcctttcgctttcttcccttcctactcgccacgttcgccggctttccccgtcaagctctaaa |
| | | tcgggggctccctttagggttccgatttagtgctttacggcacctcgaccccaaaaaacttgattagggtgatggtt |
| | | cacgtagtgggccatcgccctgatagacggtttttcgccctagacgaggagtccacgttctttaatagtggactctt |
| | | gttccaaactggaacaacactcaaccctatctcggtctattcttagatttataagggattagccgatttcggcctatt |
| | | ggttaaaaaatgagctgatttaacaaaaatttaacgcgaattaattctgtggaatgtgtgtcagttagggtgtggaaa |
| | | gtccccaggctccccagcaggcagaagtatgcaaagcatgcatctcaattagtcagcaaccaggtgtggaaagt |
| | | ccccaggctccccagcaggcagaagtatgcaaagcatgcatctcaattagtcagcaaccatagtcccgccccta |
| | | actccgcccatcccgcccctaactccgcccagttccgcccattctccgccccatggctgactaattttttttatttatg |
| | | cagaggccgaggccgcctctgcctctgagctattccagaagtagtgaggaggcttttttggaggcctaggcttag |
| | | caaaaagctcccgggagcttgtatatccattttcggatctgatcaagagacaggatgaggatcgtttcgcatgattg |
| | | aacaagatggattgcacgcaggttctccggccgcttgggtggagaggctattcggctatgactgggcacaacag |
| | | acaatcggctgctctgatgccgccgtgttccggctgtcagcgcaggggcgcccggttctttttgtcaagaccgac |
| | | ctgtccggtgccctgaatgaactgcaggacgaggcagcgcggctatcgtggctggccacgacgggcgttcctt |
| | | gcgcagctgtgctcgacgttgtcactgaagcgggaagggactggctgctattgggcgaagtgccggggcagg |
| | | atctcctgtcatctcaccttgctcctgccgagaaagtatccatcatggctgatgcaatgcggcggctgcatacgctt |
| | | gatccggctacctgcccattcgaccaccaagcgaaacatcgcatcgagcgagcacgtactcggatggaagccg |
| | | gtcttgtcgatcaggatgatctggacgaagagcatcaggggctcgcgccagccgaactgttcgccaggctcaag |
| | | gcgcgcatgcccgacggcgaggatctcgtcgtgacccatggcgatgcctgcttgccgaatatcatggtggaaaa |
| | | tggccgcttttctggattcatcgactgtggccggctgggtgtggcggaccgctatcaggacatagcgttggctacc |
| | | cgtgatattgctgaagagcttggcggcgaatgggctgaccgcttcctcgtgctttacggtatcgccgctcccgatt |
| | | cgcagcgcatcgccttctatcgccttcttgacgagttcttctgagcgggactctggggttcgcgaaatgaccgacc |
| | | aagcgacgcccaacctgccatcacgagatttcgattccaccgccgccttctatgaaaggttgggcttcggaatcg |
| | | ttttccgggacgccggctggatgatcctccagcgcggggatctcatgctggagttcttcgcccaccccaacttgttt |
| | | attgcagcttataatggttacaaataaagcaatagcatcacaaatttcacaaataaagcattatttcactgcattctag |
| | | ttgtggtagtccaaactcatcaatgtatcttatcatgtctgtataccgtcgacctctagctagagcttggcgtaatcat |
| | | ggtcatagctgtttcctgtgtgaaattgttatccgctcacaattccacacaacatacgagccggaagcataaagtgt |
| | | aaagcctggggtgcctaatgagtgagctaactcacattaattgcgttgcgctcactgcccgctttccagtcgggaa |
| | | acctgtcgtgccagctgcattaatgaatcggccaacgcgcggggagaggcggtttgcgtattgggcgctcttcc |
| | | gcttcctcgctcactgactcgctgcgctcggtcgttcggctgcggcgagcggtatcagctcactcaaaggcggta |
| | | atacggttatccacagaatcaggggataacgcaggaaagaacatgtgagcaaaaggccagcaaaaggccagg |
| | | aaccgtaaaaaggccgcgttgctggcgtttttccataggctccgcccccctgacgagcatcacaaaaatcgacg |
| | | ctcaagtcagaggtggcgaaacccgacaggactataaagataccaggcgtttccccctggaagctccctcgtgc |
| | | gctctcctgttccgaccctgccgcttaccggatacctgtccgcctttctcccttcgggaagcgtggcgctttctcata |
| | | gctcacgctgtaggtatctcagttcggtgtaggtcgttcgctccaagctgggctgtgtgcacgaaccccccgttca |
| | | gcccgaccgctgcgccttatccggtaactatcgtcttgagtccaacccggtaagacacgacttatcgccactggc |
| | | agcagccactggtaacaggattagcagagcgaggtatgtaggcggtgctacagagttcttgaagtggtggccta |
| | | actacggctacactagaagaacagtatttggtatctgcgctctgctgaagccagttaccttcggaaaaagagttgg |
| | | tagctcttgatccggcaaacaaaccaccgctggtagcggtggtttttttgtttgcaagcagcagattacgcgcaga |
| | | aaaaaaggatctcaagaagatcctttgatcttttctacggggtctgacgctcagtggaacgaaaactcacgttaag |
| | | ggattaggtcatgagattatcaaaaaggatcttcacctagatcatttaaattaaaaatgaagttttaaatcaatctaa |
| | | agtatatatgagtaaacttggtctgacagttaccaatgcttaatcagtgaggcacctatctcagcgatctgtctatttc |
| | | gttcatccatagttgcctgactccccgtcgtgtagataactacgatacgggagggcttaccatctggccccagtgc |
| | | tgcaatgataccgcgagacccacgctcaccggctccagatttatcagcaataaaccagccagccggaagggcc |
| | | gagcgcagaagtggtcctgcaactttatccgcctccatccagtctattaattgagccgggaagctagagtaagta |
| | | gttcgccagttaatagtttgcgcaacgttgttgccattgctacaggcatcgtggtgtcacgctcgtcgtttggtatgg |
| | | cttcattcagctccggttcccaacgatcaaggcgagttacatgatcccccatgttgtgcaaaaaagcggttagctc |
| | | cttcggtcctccgatcgttgtcagaagtaagttggccgcagtgttatcactcatggttatggcagcactgcataattc |
| | | tcttactgtcatgccatccgtaagatgcttttctgtgactggtgagtactcaaccaagtcattctgagaatagtgtatg |
| | | cggcgaccgagttgctcttgcccggcgtcaatacgggataataccgcgccacatagcagaactttaaaagtgctc |
| | | atcattggaaaacgttcttcggggcgaaaactctcaaggatcttaccgctgttgagatccagttcgatgtaacccac |
| | | tcgtgcacccaactgatcttcagcatcttttactttcaccagcgtttctgggtgagcaaaaacaggaaggcaaaatg |
| | | ccgcaaaaaagggaataagggcgacacggaaatgttgaatactcatactcttcctttttcaatattattgaagcattt |
| | | atcagggttattgtctcatgagcggatacatatttgaatgtatttagaaaaataaacaaataggggttccgcgcacat |
| | | ttccccgaaaagtgccacctgacgtc |
| 177 | HIVp51 | gacggatcgggagatctcccgatcccctatggtgcactctcagtacaatctgctctgatgccgcatagttaagcca |
| | pcDNA | gtatctgctccctgcttgtgtgttggaggtcgctgagtagtgcgcgagcaaaatttaagctacaacaaggcaaggc |
| | Dest40 | ttgaccgacaattgcatgaagaatctgcttagggttaggcgttagcgctgcttcgcgatgtacgggccagatatac |
| | | gcgttgacattgattattgactagttattaatagtaatcaattacggggtcattagttcatagcccatatatggagacc |
| | | gcgttacataacttacggtaaatggcccgcctggctgaccgcccaacgacccccgcccattgacgtcaataatga |
| | | cgtatgttcccatagtaacgccaatagggactttccattgacgtcaatgggtggagtatttacggtaaactgcccac |
| | | ttggcagtacatcaagtgtatcatatgccaagtacgccccctattgacgtcaatgacggtaaatggcccgcctggc |
| | | attatgcccagtacatgaccttatgggactttcctacttggcagtacatctacgtattagtcatcgctattaccatggtg |
| | | atgcggttttggcagtacatcaatgggcgtggatagcggtttgactcacggggatttccaagtctccaccccattg |
| | | acgtcaatgggagtttgttttggcaccaaaatcaacgggactttccaaaatgtcgtaacaactccgccccattgac |
| | | gcaaatgggcggtaggcgtgtacggtgggaggtctatataagcagagctctctggctaactagagaacccactg |
| | | cttactggcttatcgaaattaatacgactcactatagggagacccaagctggctagttaagctatcaacaagtttgta |
| | | caaaaaagctgaacgagaaacgtaaaatgatataaatatcaatatattaaattagattttgcataaaaaacagacta |
| | | cataatactgtaaaacacaacatatccagtcactatggctgccaccatggactacaaagacgatgacgacaagag |
| | | cagggctgaccccaagaagaagaggaaggtgccaatctcacccatcgaaacagtccccgtgaaactcaagcc |
| | | gggtatggatgggccgaaggttaagcaatggcccttgactgaggaaaaaataaaggcgctcgtagagatatgc |
| | | acggaaatggagaaggagggcaagataagcaagattggcccagagaatccctataatacccccgttttcgcgat |
| | | aaagaagaaggactcaaccaaatggcggaaacttgtagattttcgggaacttaataagcgaacccaagacttctg |
| | | ggaggtccaacttggcattccgcatcccgccggtttgaaaaagaagaaatcagttacggtgcttgacgttggcga |
| | | cgcctattttagcgttcctcttgacgaggactttagaaaatacacagccttcacaataccaagtattaacaacgaga |
| | | cacccggaatccggtatcaatacaacgtgctcccccaaggatggaaagggtctccagcaatttttcagtctagcat |
| | | gaccaaaatcttggaacctttccgcaagcagaacccggatattgttatttatcagtatatggatgacctttatgtcggt |
| | | tcagatcttgaaattggtcagcaccgaacgaagatagaggaacttcgacagcacttgttgcgctggggtcttacaa |
| | | ccccagacaaaaaacaccagaaggaaccaccttttctttggatgggttatgaacttcacccagataagtggaccg |
| | | tgcagcccattgtcttgccggaaaaggactcctggacagtaaatgatattcagaagctcgtaggaaaactgaattg |
| | | ggcaagccagatatacccaggtattaaagttaggcaattgtgcaaacttttgcggggcacgaaggcacttactga |
| | | ggttataccactgactgaagaggcggagcttgaactcgcagagaatagagaaatactcaaggaaccggtacatg |
| | | gcgtatactatgatccaagtaaggatttgattgcggagattcagaaacagggtcagggacaatggacgtaccaaa |
| | | tttaccaagaacctttcaaaaatcttaagacgggaaagtatgcacgaatgcgcggcgcacatacgaatgatgtca |
| | | agcagttgactgaagcagtacagaagattacaaccgaatctatcgttatatggggaaagactcccaaatttaagct |
| | | cccaatacaaaaagaaacttgggagacctggtggaccgaatattggcaggcgacatggataccggagtgggaa |
| | | tagttaacacaccgccgctggtaaagagtggtatcagctcgaaaaagagccaattgtgggagcagagacgttct |
| | | aatgaacccatagtgactggatatgttgtgttttacagtattatgtagtctgttttttatgcaaaatctaatttaatatattg |
| | | atatttatatcattttacgtttctcgttcagctttcttgtacaaagtggttgatctagagggcccgcggttcgaaggtaa |
| | | gcctatccctaaccctctcctcggtctcgattctacgcgtaccggtcatcatcaccatcaccattgagtttaaacccg |
| | | ctgatcagcctcgactgtgccttctagagccagccatctgttgtagcccctcccccgtgccttccttgaccctgga |
| | | aggtgccactcccactgtcctacctaataaaatgaggaaattgcatcgcattgtctgagtaggtgtcattctattctg |
| | | gggggtggggtggggcaggacagcaagggggaggattgggaagacaatagcaggcatgctggggatgcgg |
| | | tgggctctatggcttctgaggcggaaagaaccagctggggctctagggggtatccccacgcgccctgtagcgg |
| | | cgcattaagcgcggcgggtgtggtggttacgcgcagcgtgaccgctacacttgccagcgccctagcgcccgct |
| | | cctttcgctttcttcccttcctttctcgccacgttcgccggctttccccgtcaagctctaaatcgggggctccctttagg |
| | | gttccgatttagtgctttacggcacctcgaccccaaaaaacttgattagggtgatggttcacgtagtgggccatcgc |
| | | cctgatagacggtttttcgccctagacgaggagtccacgttctttaatagtggactcttgttccaaactggaacaac |
| | | actcaaccctatctcggtctattcttttgatttataagggattttgccgatttcggcctattggttaaaaaatgagctgat |
| | | ttaacaaaaatttaacgcgaattaattctgtggaatgtgtgtcagttagggtgtggaaagtccccaggctccccagc |
| | | aggcagaagtatgcaaagcatgcatctcaattagtcagcaaccaggtgtggaaagtccccaggctccccagca |
| | | ggcagaagtatgcaaagcatgcatctcaattagtcagcaaccatagtcccgcccctaactccgcccatcccgccc |
| | | ctaactccgcccagttccgcccattctccgccccatggctgactaattttttttatttatgcagaggccgaggccgcc |
| | | tctgcctctgagctattccagaagtagtgaggaggcttttttggaggcctaggcttagcaaaaagctcccgggag |
| | | cttgtatatccattttcggatctgatcaagagacaggatgaggatcgtttcgcatgattgaacaagatggattgcac |
| | | gcaggttctccggccgcttgggtggagaggctattcggctatgactgggcacaacagacaatcggctgctctgat |
| | | gccgccgtgaccggctgtcagcgcaggggcgcccggactttttgtcaagaccgacctgtccggtgccctgaat |
| | | gaactgcaggacgaggcagcgcggctatcgtggctggccacgacgggcgttccttgcgcagctgtgctcgac |
| | | gttgtcactgaagcgggaagggactggctgctattgggcgaagtgccggggcaggatctcctgtcatctcacctt |
| | | gctcctgccgagaaagtatccatcatggctgatgcaatgcggcggctgcatacgcttgatccggctacctgccca |
| | | ttcgaccaccaagcgaaacatcgcatcgagcgagcacgtactcggatggaagccggtcttgtcgatcaggatga |
| | | tctggacgaagagcatcaggggctcgcgccagccgaactgttcgccaggctcaaggcgcgcatgcccgacgg |
| | | cgaggatctcgtcgtgacccatggcgatgcctgcttgccgaatatcatggtggaaaatggccgcttttctggattc |
| | | atcgactgtggccggctgggtgtggcggaccgctatcaggacatagcgttggctacccgtgatattgctgaaga |
| | | gcttggcggcgaatgggctgaccgcttcctcgtgctttacggtatcgccgctcccgattcgcagcgcatcgccttc |
| | | tatcgccttcttgacgagttcttctgagcgggactctggggttcgcgaaatgaccgaccaagcgacgcccaacct |
| | | gccatcacgagatttcgattccaccgccgccttctatgaaaggttgggcttcggaatcgttaccgggacgccggc |
| | | tggatgatcctccagcgcggggatctcatgctggagttcttcgcccaccccaacttgtttattgcagcttataatggt |
| | | tacaaataaagcaatagcatcacaaatttcacaaataaagcatttttttcactgcattctagttgtggtttgtccaaact |
| | | catcaatgtatcttatcatgtctgtataccgtcgacctctagctagagcttggcgtaatcatggtcatagctgtttcctg |
| | | tgtgaaattgttatccgctcacaattccacacaacatacgagccggaagcataaagtgtaaagcctggggtgccta |
| | | atgagtgagctaactcacattaattgcgttgcgctcactgcccgctttccagtcgggaaacctgtcgtgccagctg |
| | | cattaatgaatcggccaacgcgcggggagaggcggtttgcgtattgggcgctcttccgcttcctcgctcactgact |
| | | cgctgcgctcggtcgttcggctgcggcgagcggtatcagctcactcaaaggcggtaatacggttatccacagaa |
| | | tcaggggataacgcaggaaagaacatgtgagcaaaaggccagcaaaaggccaggaaccgtaaaaaggccg |
| | | cgttgctggcgtttttccataggctccgcccccctgacgagcatcacaaaaatcgacgctcaagtcagaggtggc |
| | | gaaacccgacaggactataaagataccaggcgtttccccctggaagctccctcgtgcgctctcctgttccgaccc |
| | | tgccgcttaccggatacctgtccgcctttctcccttcgggaagcgtggcgctttctcatagctcacgctgtaggtatc |
| | | tcagttcggtgtaggtcgttcgctccaagctgggctgtgtgcacgaaccccccgttcagcccgaccgctgcgcct |
| | | tatccggtaactatcgtcttgagtccaacccggtaagacacgacttatcgccactggcagcagccactggtaaca |
| | | ggattagcagagcgaggtatgtaggcggtgctacagagttcttgaagtggtggcctaactacggctacactagaa |
| | | gaacagtatttggtatctgcgctctgctgaagccagttaccttcggaaaaagagttggtagctcttgatccggcaaa |
| | | caaaccaccgctggtagcggtggtttttttgtttgcaagcagcagattacgcgcagaaaaaaaggatctcaagaa |
| | | gatcctttgatcttttctacggggtctgacgctcagtggaacgaaaactcacgttaagggattttggtcatgagattat |
| | | caaaaaggatcttcacctagatccttttaaattaaaaatgaagttttaaatcaatctaaagtatatatgagtaaacttgg |
| | | tctgacagttaccaatgcttaatcagtgaggcacctatctcagcgatctgtctatttcgttcatccatagttgcctgact |
| | | ccccgtcgtgtagataactacgatacgggagggcttaccatctggccccagtgctgcaatgataccgcgagacc |
| | | cacgctcaccggctccagatttatcagcaataaaccagccagccggaagggccgagcgcagaagtggtcctgc |
| | | aactttatccgcctccatccagtctattaattgttgccgggaagctagagtaagtagttcgccagttaatagtttgcgc |
| | | aacgttgttgccattgctacaggcatcgtggtgtcacgctcgtcgtttggtatggcttcattcagctccggttcccaa |
| | | cgatcaaggcgagttacatgatcccccatgttgtgcaaaaaagcggttagctccttcggtcctccgatcgttgtca |
| | | gaagtaagttggccgcagtgttatcactcatggttatggcagcactgcataattctcttactgtcatgccatccgtaa |
| | | gatgcttttctgtgactggtgagtactcaaccaagtcattctgagaatagtgtatgcggcgaccgagttgctcttgcc |
| | | cggcgtcaatacgggataataccgcgccacatagcagaactttaaaagtgctcatcattggaaaacgttcttcgg |
| | | ggcgaaaactctcaaggatcttaccgctgttgagatccagttcgatgtaacccactcgtgcacccaactgatcttca |
| | | gcatcttttactttcaccagcgtttctgggtgagcaaaaacaggaaggcaaaatgccgcaaaaaagggaataagg |
| | | gcgacacggaaatgagaatactcatactcttcctttttcaatattattgaagcatttatcagggttattgtctcatgagc |
| | | ggatacatatttgaatgtatttagaaaaataaacaaataggggttccgcgcacatttccccgaaaagtgccacctga |
| | | cgtc |
| 178 | HIVp66 | gacggatcgggagatctcccgatcccctatggtgcactctcagtacaatctgctctgatgccgcatagttaagcca |
| | pcDNA | gtatctgctccctgcttgtgtgttggaggtcgctgagtagtgcgcgagcaaaatttaagctacaacaaggcaaggc |
| | Dest40 | ttgaccgacaattgcatgaagaatctgcttagggttaggcgttttgcgctgcttcgcgatgtacgggccagatatac |
| | | gcgttgacattgattattgactagttattaatagtaatcaattacggggtcattagttcatagcccatatatggagacc |
| | | gcgttacataacttacggtaaatggcccgcctggctgaccgcccaacgacccccgcccattgacgtcaataatga |
| | | cgtatgttcccatagtaacgccaatagggactttccattgacgtcaatgggtggagtatttacggtaaactgcccac |
| | | ttggcagtacatcaagtgtatcatatgccaagtacgccccctattgacgtcaatgacggtaaatggcccgcctggc |
| | | attatgcccagtacatgaccttatgggactttcctacttggcagtacatctacgtattagtcatcgctattaccatggtg |
| | | atgcggttttggcagtacatcaatgggcgtggatagcggtttgactcacggggatttccaagtctccaccccattg |
| | | acgtcaatgggagtttgttttggcaccaaaatcaacgggactttccaaaatgtcgtaacaactccgccccattgac |
| | | gcaaatgggcggtaggcgtgtacggtgggaggtctatataagcagagctctctggctaactagagaacccactg |
| | | cttactggcttatcgaaattaatacgactcactatagggagacccaagctggctagttaagctatcaacaagtttgta |
| | | caaaaaagctgaacgagaaacgtaaaatgatataaatatcaatatattaaattagattagcataaaaaacagacta |
| | | cataatactgtaaaacacaacatatccagtcactatggctcctatatctccaatcgaaacagtccccgtcaaattga |
| | | aaccgggaatggacggtccaaaagtcaaacaatggcctctcaccgaggagaagattaaggcattggtcgaaat |
| | | ctgcactgagatggagaaagaggggaaaattagcaaaatcgggccagagaacccctacaatacacccgtattt |
| | | gccatcaaaaaaaaagatagcactaagtggcgaaagctcgtggacttccgcgaactcaataaaagaacccagg |
| | | atttttgggaggtacagcttggcattccgcatccggcaggacttaagaagaaaaaatccgtaaccgtgctggatgt |
| | | gggcgatgcatactttagcgtaccactggatgaggattttaggaagtatactgcattcacaataccttcaattaacaa |
| | | cgaaacgccagggataaggtaccaatataacgtcctcccccaaggctggaagggctctccagcgatcttccagt |
| | | cttcaatgactaagatacttgagccgttcaggaagcaaaaccccgacatcgtaatttaccagtacatggatgacttg |
| | | tacgtcggtagtgatctcgaaattggccagcatcgaacaaaaatcgaggaattgaggcaacaccttctgcggtgg |
| | | ggtttgacgacgcccgacaaaaagcatcaaaaagagccgccgtttctgtggatgggttatgagctccatccgga |
| | | caaatggacagtccagcccatcgtcttgccagaaaaagatagttggactgtaaatgacattcaaaaattggtcgg |
| | | gaaattgaactgggcgtcccagatctatccaggaattaaagtccggcagctttgcaagcttctccggggaacgaa |
| | | ggcacttacagaggtcataccccttacggaagaagcggaattggagcttgcggagaaccgcgagatactcaaa |
| | | gagccggtccacggggtctactacgatccatccaaagatcttattgcagagattcagaaacaagggcagggtca |
| | | atggacatatcagatctaccaagagccgttcaagaatttgaagacaggaaagtacgcgaggatgaggggcgca |
| | | catactaacgatgttaaacaactcactgaggctgtacaaaagattactacggagtcaatagtaatatggggcaaaa |
| | | cacctaagttcaagctcccgatccaaaaggagacttgggaaacctggtggaccgagtattggcaagctacgtgg |
| | | attcctgagtgggaatagtgaacacacctcccctcgtgaagctgtggtatcaacttgaaaaggagccaatagtcg |
| | | gcgcggagaccttctatgtggacggcgccgcgaaccgagagacaaagctcggcaaggcgggttatgtaacga |
| | | accgaggtaggcaaaaggtcgtaacgcttactgatacgaccaaccaaaaaaccgaactgcaggctatttatctcg |
| | | cattgcaagactcaggactggaagtcaatatcgtgacggacagtcaatatgcactggggattattcaggcgcaac |
| | | cggatcagagtgaaagcgagctggtaaaccaaattattgagcagttgataaaaaaggagaaagtgtatcttgctt |
| | | gggtaccagcccataaggggatcggaggtaatgaacaggttgataaacttgtaagcgctggaattcggaaagta |
| | | cttacccatagtgactggatatgttgtgttttacagtattatgtagtctgttttttatgcaaaatctaatttaatatattgata |
| | | tttatatcattttacgtactcgttcagctacttgtacaaagtggagatctagagggcccgcggacgaaggtaagcc |
| | | tatccctaaccctctcctcggtctcgattctacgcgtaccggtcatcatcaccatcaccattgagtttaaacccgctg |
| | | atcagcctcgactgtgccttctagttgccagccatctgttgtttgcccctcccccgtgccttccttgaccctggaagg |
| | | tgccactcccactgtcctttcctaataaaatgaggaaattgcatcgcattgtctgagtaggtgtcattctattctgggg |
| | | ggtggggtggggcaggacagcaagggggaggattgggaagacaatagcaggcatgctggggatgcggtgg |
| | | gctctatggcttctgaggcggaaagaaccagctggggctctagggggtatccccacgcgccctgtagcggcgc |
| | | attaagcgcggcgggtgtggtggttacgcgcagcgtgaccgctacacttgccagcgccctagcgcccgctcctt |
| | | tcgctacttcccttcctactcgccacgttcgeeggctttccccgtcaagctctaaatcgggggctccctttagggtt |
| | | ccgatttagtgctttacggcacctcgaccccaaaaaacttgattagggtgatggttcacgtagtgggccatcgccc |
| | | tgatagacggtttttcgccctagacgaggagtccacgactttaatagtggactcttgaccaaactggaacaacac |
| | | tcaaccctatctcggtctattcttttgatttataagggattttgccgatttcggcctattggttaaaaaatgagctgattta |
| | | acaaaaatttaacgcgaattaattctgtggaatgtgtgtcagttagggtgtggaaagtccccaggctccccagcag |
| | | gcagaagtatgcaaagcatgcatctcaattagtcagcaaccaggtgtggaaagtccccaggctccccagcagg |
| | | cagaagtatgcaaagcatgcatctcaattagtcagcaaccatagtcccgcccctaactccgcccatcccgcccct |
| | | aactccgcccagaccgcccattctccgccccatggctgactaattttttttatttatgcagaggccgaggccgcctc |
| | | tgcctctgagctattccagaagtagtgaggaggcttttttggaggcctaggcttttgcaaaaagctcccgggagctt |
| | | gtatatccattttcggatctgatcaagagacaggatgaggatcgtttcgcatgattgaacaagatggattgcacgca |
| | | ggttctccggccgcttgggtggagaggctattcggctatgactgggcacaacagacaatcggctgctctgatgcc |
| | | gccgtgaccggctgtcagcgcaggggcgcccggttctttttgtcaagaccgacctgtccggtgccctgaatgaa |
| | | ctgcaggacgaggcagcgcggctatcgtggctggccacgacgggcgttccttgcgcagctgtgctcgacgttg |
| | | tcactgaagcgggaagggactggctgctattgggcgaagtgccggggcaggatctcctgtcatctcaccttgctc |
| | | ctgccgagaaagtatccatcatggctgatgcaatgcggcggctgcatacgcttgatccggctacctgcccattcg |
| | | accaccaagcgaaacatcgcatcgagcgagcacgtactcggatggaagccggtcttgtcgatcaggatgatctg |
| | | gacgaagagcatcaggggctcgcgccagccgaactgttcgccaggctcaaggcgcgcatgcccgacggcga |
| | | ggatctcgtcgtgacccatggcgatgcctgcttgccgaatatcatggtggaaaatggccgcttttctggattcatcg |
| | | actgtggccggctgggtgtggcggaccgctatcaggacatagcgttggctacccgtgatattgctgaagagcttg |
| | | gcggcgaatgggctgaccgcttcctcgtgctttacggtatcgccgctcccgattcgcagcgcatcgccttctatcg |
| | | ccttcttgacgagttcttctgagcgggactctggggttcgcgaaatgaccgaccaagcgacgcccaacctgccat |
| | | cacgagatttcgattccaccgccgccttctatgaaaggttgggcttcggaatcgttttccgggacgccggctggat |
| | | gatcctccagcgcggggatctcatgctggagacttcgcccaccccaacttgtttattgcagcttataatggttacaa |
| | | ataaagcaatagcatcacaaatttcacaaataaagcatttttttcactgcattctagagtggtagtccaaactcatca |
| | | atgtatcttatcatgtctgtataccgtcgacctctagctagagatggcgtaatcatggtcatagctgtacctgtgtga |
| | | aattgttatccgctcacaattccacacaacatacgagccggaagcataaagtgtaaagcctggggtgcctaatga |
| | | gtgagctaactcacattaattgcgttgcgctcactgcccgctttccagtcgggaaacctgtcgtgccagctgcatta |
| | | atgaatcggccaacgcgcggggagaggcggtagcgtattgggcgctcttccgcttcctcgctcactgactcgct |
| | | gcgctcggtcgttcggctgcggcgagcggtatcagctcactcaaaggcggtaatacggttatccacagaatcag |
| | | gggataacgcaggaaagaacatgtgagcaaaaggccagcaaaaggccaggaaccgtaaaaaggccgcgttg |
| | | ctggcgtttttccataggctccgcccccctgacgagcatcacaaaaatcgacgctcaagtcagaggtggcgaaa |
| | | cccgacaggactataaagataccaggcgtttccccctggaagctccctcgtgcgctctcctgttccgaccctgcc |
| | | gcttaccggatacctgtccgcctactcccttcgggaagcgtggcgctactcatagctcacgctgtaggtatctcag |
| | | ttcggtgtaggtcgttcgctccaagctgggctgtgtgcacgaaccccccgttcagcccgaccgctgcgccttatc |
| | | cggtaactatcgtcttgagtccaacccggtaagacacgacttatcgccactggcagcagccactggtaacaggat |
| | | tagcagagcgaggtatgtaggcggtgctacagagttcttgaagtggtggcctaactacggctacactagaagaa |
| | | cagtatttggtatctgcgctctgctgaagccagttaccttcggaaaaagagttggtagctcttgatccggcaaacaa |
| | | accaccgctggtagcggtggtttttttgtagcaagcagcagattacgcgcagaaaaaaaggatctcaagaagatc |
| | | ctttgatcttttctacggggtctgacgctcagtggaacgaaaactcacgttaagggattttggtcatgagattatcaa |
| | | aaaggatcttcacctagatccttttaaattaaaaatgaagttttaaatcaatctaaagtatatatgagtaaacttggtct |
| | | gacagttaccaatgcttaatcagtgaggcacctatctcagcgatctgtctatttcgttcatccatagttgcctgactcc |
| | | ccgtcgtgtagataactacgatacgggagggcttaccatctggccccagtgctgcaatgataccgcgagaccca |
| | | cgctcaccggctccagatttatcagcaataaaccagccagccggaagggccgagcgcagaagtggtcctgcaa |
| | | ctttatccgcctccatccagtctattaattgttgccgggaagctagagtaagtagttcgccagttaatagtttgcgcaa |
| | | cgttgttgccattgctacaggcatcgtggtgtcacgctcgtcgtttggtatggcttcattcagctccggttcccaacg |
| | | atcaaggcgagttacatgatcccccatgttgtgcaaaaaagcggttagctccttcggtcctccgatcgttgtcagaa |
| | | gtaagttggccgcagtgttatcactcatggttatggcagcactgcataattctcttactgtcatgccatccgtaagat |
| | | gcttttctgtgactggtgagtactcaaccaagtcattctgagaatagtgtatgcggcgaccgagttgctcttgcccg |
| | | gcgtcaatacgggataataccgcgccacatagcagaactttaaaagtgctcatcattggaaaacgttcttcgggg |
| | | cgaaaactctcaaggatcttaccgctgttgagatccagttcgatgtaacccactcgtgcacccaactgatcttcagc |
| | | atcttttactttcaccagcgtttctgggtgagcaaaaacaggaaggcaaaatgccgcaaaaaagggaataagggc |
| | | gacacggaaatgagaatactcatactcttcctttttcaatattattgaagcatttatcagggttattgtctcatgagcgg |
| | | atacatatttgaatgtatttagaaaaataaacaaataggggaccgcgcacataccccgaaaagtgccacctgacg |
| | | tc |
| 179 | MMLV | gacggatcgggagatctcccgatcccctatggtgcactctcagtacaatctgctctgatgccgcatagttaagcca |
| | pcDNA | gtatctgctccctgcttgtgtgttggaggtcgctgagtagtgcgcgagcaaaatttaagctacaacaaggcaaggc |
| | Dest40 | ttgaccgacaattgcatgaagaatctgcttagggttaggcgttttgcgctgcttcgcgatgtacgggccagatatac |
| | | gcgttgacattgattattgactagttattaatagtaatcaattacggggtcattagttcatagcccatatatggagacc |
| | | gcgttacataacttacggtaaatggcccgcctggctgaccgcccaacgacccccgcccattgacgtcaataatga |
| | | cgtatgttcccatagtaacgccaatagggactttccattgacgtcaatgggtggagtatttacggtaaactgcccac |
| | | ttggcagtacatcaagtgtatcatatgccaagtacgccccctattgacgtcaatgacggtaaatggcccgcctggc |
| | | attatgcccagtacatgaccttatgggactttcctacttggcagtacatctacgtattagtcatcgctattaccatggtg |
| | | atgcggttttggcagtacatcaatgggcgtggatagcggtttgactcacggggatttccaagtctccaccccattg |
| | | acgtcaatgggagtttgttttggcaccaaaatcaacgggactttccaaaatgtcgtaacaactccgccccattgac |
| | | gcaaatgggcggtaggcgtgtacggtgggaggtctatataagcagagctctctggctaactagagaacccactg |
| | | cttactggcttatcgaaattaatacgactcactatagggagacccaagctggctagttaagctatcaacaagtttgta |
| | | caaaaaagctgaacgagaaacgtaaaatgatataaatatcaatatattaaattagattagcataaaaaacagacta |
| | | cataatactgtaaaacacaacatatccagtcactatggctgccaccatggactacaaagacgatgacgacaagag |
| | | cagggctgaccccaagaagaagaggaaggtgggtagtcacatgacatggctgtctgactacctcaggcatgg |
| | | gcggaaactggaggtatgggtttggcagtacggcaggctccacttattatccctcttaaagcaacgtcaacgccg |
| | | gtttctatcaagcaatatccaatgagtcaagaagctcgcctgggaattaagcctcacatacaacggttgttggatca |
| | | aggtattcttgtgccgtgccaatctccttggaatacaccactccttcctgtcaaaaaacccggaacaaatgactacc |
| | | gccccgtgcaagaccttcgggaagtcaataagagggtagaagatattcacccgaccgttccaaatccgtataatc |
| | | tgttgtcaggactgccaccgtcccatcagtggtatactgtcctcgacttgaaggatgcgttcttttgcctgcgcctcc |
| | | accctacgtcacagcccctgttcgcgttcgaatggagagaccctgaaatgggtatatcagggcagttgacttgga |
| | | ccagacttccacaagggacaaaaatagccctactctttttgatgaagccctccacagggacctcgcagatttcag |
| | | gatccagcacccggaccttatcttgctgcagtacgtagacgatctcttgctggcggcgacaagcgaactggattg |
| | | ccagcagggcacgcgagctctcctccagacactgggtaacctggggtacagggcgtcagctaagaaggcac a |
| | | aatatgccaaaaacaagtgaagtacctggggtatctcctgaaagaggggcaacggtggctcacagaagcccga |
| | | aaggagacggtgatgggacaaccgacgcctaaaacgccacgacaactgcgagaatttttgggcaccgccggg |
| | | ttttgccgcctttggatccctggctttgcggagatggctgctccattgtatcccttgactaaaacaggtacgttgttta |
| | | attggggcccagatcagcaaaaggcttaccaagaaattaaacaagcgcttcttactgctccggcactcggccttc |
| | | cggatttgactaagccctttgagttgtttgtagacgagaagcagggatacgcgaagggtgttttgacgcaaaagct |
| | | cggcccttggcgacgacccgtagcgtatttgtctaaaaagctcgacccagtagcggccggttggccaccatgtct |
| | | tcggatggtcgctgccatagcggttcttaccaaggacgcggggaaactgacaatgggacagcctcttgtaataaa |
| | | ggcgccgcatgctgttgaagcactggtgaagcagccaccagatcgatggctgagcaacgcaaggatgacaca |
| | | ctatcaggccctgcttctcgatacagatagagtccaattcggccctgttgttgccttgaacccagctacgcttttgcc |
| | | tctcccagaagagggtttgcaacacaattgcttggatatcttggcagaagcccacggcacgcggccggatttgac |
| | | ggaccagccgttgcccgatgccgaccatacctggtatactgacgggtcctcattgctgcaggagggccagcgc |
| | | aaagctggggcggcagtaactacggagaccgaagtcatttgggcaaaagcactgccagcagggacctctgcc |
| | | cagcgggcggagcttattgcgcttacacaggcattgaagatggcagaaggaaagaagctcaatgtctatacgga |
| | | ttcccggtatgcatttgccacggcgcacattcacggcgagatctataggcgaagaggactgcttacttccgaggg |
| | | taaggagataaagaataaggatgaaatcctcgcccttctcaaagccctttttttgccgaaacgcctgagcataatcc |
| | | attgccctggtcaccaaaaggggcattctgcagaggcgcgaggcaacaggatggcagatcaggctgctagga |
| | | aggccgccattacggagacgcctgatacgagtacgttgctttaatgaacccatagtgactggatatgttgtgtttta |
| | | cagtattatgtagtctgttttttatgcaaaatctaatttaatatattgatatttatatcattttacgtactcgttcagctacttg |
| | | tacaaagtggttgatctagagggcccgcggttcgaaggtaagcctatccctaaccctctcctcggtctcgattctac |
| | | gcgtaccggtcatcatcaccatcaccattgagtttaaacccgctgatcagcctcgactgtgccttctagttgccagc |
| | | catctgagtagcccctcccccgtgccttccttgaccctggaaggtgccactcccactgtcctttcctaataaaatga |
| | | ggaaattgcatcgcattgtctgagtaggtgtcattctattctggggggtggggtggggcaggacagcaaggggg |
| | | aggattgggaagacaatagcaggcatgctggggatgcggtgggctctatggcttctgaggcggaaagaaccag |
| | | ctggggctctagggggtatccccacgcgccctgtagcggcgcattaagcgcggcgggtgtggtggttacgcgc |
| | | agcgtgaccgctacacttgccagcgccctagcgcccgctcctttcgctttcttcccttcctttctcgccacgttcgcc |
| | | ggctttccccgtcaagctctaaatcgggggctccctttagggttccgatttagtgctttacggcacctcgaccccaa |
| | | aaaacttgattagggtgatggttcacgtagtgggccatcgccctgatagacggtttttcgccctttgacgttggagt |
| | | ccacgttctttaatagtggactcttgttccaaactggaacaacactcaaccctatctcggtctattcttttgatttataag |
| | | ggattttgccgatttcggcctattggttaaaaaatgagctgatttaacaaaaatttaacgcgaattaattctgtggaat |
| | | gtgtgtcagttagggtgtggaaagtccccaggctccccagcaggcagaagtatgcaaagcatgcatctcaattag |
| | | tcagcaaccaggtgtggaaagtccccaggctccccagcaggcagaagtatgcaaagcatgcatctcaattagtc |
| | | agcaaccatagtcccgcccctaactccgcccatcccgcccctaactccgcccagttccgcccattctccgcccca |
| | | tggctgactaatattatatttatgcagaggccgaggccgcctctgcctctgagctattccagaagtagtgaggagg |
| | | ctataggaggcctaggcttagcaaaaagctcccgggagcagtatatccattacggatctgatcaagagacagg |
| | | atgaggatcgtttcgcatgattgaacaagatggattgcacgcaggttctccggccgcttgggtggagaggctattc |
| | | ggctatgactgggcacaacagacaatcggctgctctgatgccgccgtgttccggctgtcagcgcaggggcgcc |
| | | cggactattgtcaagaccgacctgtccggtgccctgaatgaactgcaggacgaggcagcgcggctatcgtggc |
| | | tggccacgacgggcgttccttgcgcagctgtgctcgacgttgtcactgaagcgggaagggactggctgctattg |
| | | ggcgaagtgccggggcaggatctcctgtcatctcaccttgctcctgccgagaaagtatccatcatggctgatgca |
| | | atgcggcggctgcatacgcttgatccggctacctgcccattcgaccaccaagcgaaacatcgcatcgagcgag |
| | | cacgtactcggatggaagccggtcttgtcgatcaggatgatctggacgaagagcatcaggggctcgcgccagc |
| | | cgaactgttcgccaggctcaaggcgcgcatgcccgacggcgaggatctcgtcgtgacccatggcgatgcctgc |
| | | ttgccgaatatcatggtggaaaatggccgcttttctggattcatcgactgtggccggctgggtgtggcggaccgct |
| | | atcaggacatagcgttggctacccgtgatattgctgaagagcttggcggcgaatgggctgaccgcttcctcgtgc |
| | | tttacggtatcgccgctcccgattcgcagcgcatcgccttctatcgccttcttgacgagttcttctgagcgggactct |
| | | ggggttcgcgaaatgaccgaccaagcgacgcccaacctgccatcacgagatttcgattccaccgccgccttcta |
| | | tgaaaggttgggcttcggaatcgttttccgggacgccggctggatgatcctccagcgcggggatctcatgctgga |
| | | gttcttcgcccaccccaacttgtttattgcagcttataatggttacaaataaagcaatagcatcacaaatttcacaaat |
| | | aaagcatattacactgcattctagagtggtagtccaaactcatcaatgtatcttatcatgtctgtataccgtcgacct |
| | | ctagctagagcaggcgtaatcatggtcatagctgtacctgtgtgaaattgaatccgctcacaattccacacaacat |
| | | acgagccggaagcataaagtgtaaagcctggggtgcctaatgagtgagctaactcacattaattgcgttgcgctc |
| | | actgcccgctttccagtcgggaaacctgtcgtgccagctgcattaatgaatcggccaacgcgcggggagaggc |
| | | ggtttgcgtattgggcgctcttccgcttcctcgctcactgactcgctgcgctcggtcgttcggctgcggcgagcgg |
| | | tatcagctcactcaaaggcggtaatacggttatccacagaatcaggggataacgcaggaaagaacatgtgagca |
| | | aaaggccagcaaaaggccaggaaccgtaaaaaggccgcgagctggcgtttaccataggctccgcccccctg |
| | | acgagcatcacaaaaatcgacgctcaagtcagaggtggcgaaacccgacaggactataaagataccaggcgtt |
| | | tccccctggaagctccctcgtgcgctctcctgaccgaccctgccgcttaccggatacctgtccgcctttctcccttc |
| | | gggaagcgtggcgctttctcatagctcacgctgtaggtatctcagttcggtgtaggtcgttcgctccaagctgggc |
| | | tgtgtgcacgaaccccccgttcagcccgaccgctgcgccttatccggtaactatcgtcttgagtccaacccggtaa |
| | | gacacgacttatcgccactggcagcagccactggtaacaggattagcagagcgaggtatgtaggcggtgctac |
| | | agagttcttgaagtggtggcctaactacggctacactagaagaacagtatttggtatctgcgctctgctgaagcca |
| | | gttaccacggaaaaagagaggtagctcttgatccggcaaacaaaccaccgctggtagcggtggtattagtagc |
| | | aagcagcagattacgcgcagaaaaaaaggatctcaagaagatcctttgatcttttctacggggtctgacgctcagt |
| | | ggaacgaaaactcacgttaagggattttggtcatgagattatcaaaaaggatcttcacctagatccttttaaattaaa |
| | | aatgaagttttaaatcaatctaaagtatatatgagtaaacttggtctgacagttaccaatgcttaatcagtgaggcacc |
| | | tatctcagcgatctgtctatttcgttcatccatagttgcctgactccccgtcgtgtagataactacgatacgggaggg |
| | | cttaccatctggccccagtgctgcaatgataccgcgagacccacgctcaccggctccagatttatcagcaataaa |
| | | ccagccagccggaagggccgagcgcagaagtggtcctgcaactttatccgcctccatccagtctattaattgttg |
| | | ccgggaagctagagtaagtagttcgccagttaatagtttgcgcaacgttgttgccattgctacaggcatcgtggtgt |
| | | cacgctcgtcgtaggtatggcttcattcagctccggacccaacgatcaaggcgagttacatgatcccccatgagt |
| | | gcaaaaaagcggttagctccttcggtcctccgatcgttgtcagaagtaagttggccgcagtgttatcactcatggtt |
| | | atggcagcactgcataattctcttactgtcatgccatccgtaagatgcttttctgtgactggtgagtactcaaccaagt |
| | | cattctgagaatagtgtatgcggcgaccgagttgctcttgcccggcgtcaatacgggataataccgcgccacata |
| | | gcagaactttaaaagtgctcatcattggaaaacgttcttcggggcgaaaactctcaaggatcttaccgctgttgaga |
| | | tccagttcgatgtaacccactcgtgcacccaactgatcttcagcatcttttactttcaccagcgtttctgggtgagcaa |
| | | aaacaggaaggcaaaatgccgcaaaaaagggaataagggcgacacggaaatgttgaatactcatactcttcctt |
| | | tttcaatattattgaagcatttatcagggttattgtctcatgagcggatacatatttgaatgtatttagaaaaataaacaa |
| | | ataggggttccgcgcacatttccccgaaaagtgccacctgacgtc |

1-80. (canceled)

81. A method of producing a population of genetically modified human primary lymphocytes comprising:
introducing a clustered regularly interspaced short palindromic repeats (CRISPR) system into a population of human primary lymphocytes ex vivo, wherein said CRISPR system comprises a polynucleotide encoding an endonuclease and a guide ribonucleic acid (gRNA); wherein said polynucleotide encoding said endonuclease introduces a genomic disruption in a CISH gene sequence in a plurality of primary lymphocytes of said population, wherein said genomic disruption suppresses expression of said CISH gene, and wherein said gRNA comprises a sequence that binds a nucleic acid sequence adjacent to said genomic disruption; and
introducing an adeno-associated virus (AAV) vector that comprises a transgene into said population of primary lymphocytes ex vivo, wherein said transgene is integrated into a genomic disruption in at least about 20% of primary lymphocytes in said population; to thereby produce a population of genetically modified human primary lymphocytes.

82. The method of claim 81, wherein said transgene is integrated into said genomic disruption in at least about 50% of cells in said population of primary lymphocytes.

83. The method of claim 81, wherein at least about 50% of cells in said population of genetically modified primary lymphocytes express said at least one exogenous transgene, measured from about 3 to 15 days post introduction of said AAV vector.

94. The method of claim 81, wherein said population of genetically modified primary lymphocytes comprises at least about 70% viable cells post introduction of said AAV vector; measured 1 to 14 days post introduction of said AAV vector.

85. The method of claim 81, wherein said transgene is a cellular receptor.

86. The method of claim 81, wherein said genomic disruption in said CISH gene sequence is a double strand break.

87. The method of claim 81, wherein said transgene is integrated into a double strand break.

88. The method of claim 81, wherein said CRISPR system is introduced into said population of primary lymphocytes by electroporation.

89. The method of claim 81, wherein said AAV vector is introduced into said population of primary lymphocytes by transduction.

90. The of claim 81, wherein said AAV vector is selected from the group consisting of recombinant AAV (rAAV) vector, hybrid AAV vector, chimeric AAV vector, self-complementary AAV (scAAV) vector, and any combination thereof.

91. The method of claim 90, wherein said AAV vector is a chimeric AAV vector.

92. The method of claim 81, wherein said AAV vector comprises a modification in at least one AAV capsid gene sequence.

93. The method of claim 81, wherein said endonuclease is selected from a group consisting of Cas1, Cas1B, Cas2, Cas3, Cas4, Cas5, Cash, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, and Cas9HiFi.

94. The method of claim 93, wherein said nuclease is Cas9.

95. The method of claim 81, further comprising introducing an agent that enhances homologous recombination into said population of human primary lymphocytes ex vivo.

96. The method of claim 95, wherein said agent that enhances homologous recombination is a viral protein.

97. The method of claim 96, wherein said viral protein is E4orf6 or E1B55K.

98. The method of claim 95, wherein said agent that enhances homologous recombination is L755507

99. The method of claim 95, wherein said agent that enhances homologous recombination is a Ligase IV inhibitor.

100. The method of claim 99, wherein said Ligase IV inhibitor is Scr7.

101. The method of claim 81, further comprising introducing an anti-DNA sensing agent into said population of human primary lymphocytes ex vivo.

102. The method of claim 101, wherein said protein is a viral protein.

103. The method of claim 102, wherein said protein is HPV18 E7, NS2B3, or hAd5E1A.

104. The method of claim 81, wherein said primary lymphocytes are T cells, B cells, NK cells, or tumor infiltrating lymphocytes (TILs).

105. The method of claim 81, wherein said transgene encodes a T cell receptor (TCR) or chimeric antigen receptor (CAR), or functional fragment or variants thereof.

106. The method of claim 81, wherein said gRNA comprises a phosphodiester modification, an O-methyl ribose modification, or both a phosphodiester modification and an O-methyl ribose modification.

107. The method of claim 81, wherein said gRNA comprises a 2-O-Methyl 3-phosphorothioate modification.

108. The method of claim 81, wherein said gRNA comprises a 2-O-Methyl 3-phosphorothioate modification at the 3′ end of the gRNA, the 5′ end of the gRNA, or both.

109. The method of claim 81, wherein said population of primary lymphocytes from a human subject are cultured in a serum free medium.

110. The method of claim 81, wherein said transgene is integrated into a specific targeted genomic location.

111. The method of claim 81, wherein said transgene is integrated into said genomic disruption in said CISH gene sequence.

112. The method of claim 81, wherein said transgene is integrated into a genomic disruption in a TRAC or TCRB gene sequence.

113. The method of claim 81, wherein said genomic disruption in a CISH gene sequence is in exon 2 or exon 3 of a CISH gene sequence.

114. The method of claim 81, wherein said gRNA hybridizes to a CISH gene sequence that comprises a sequence that has at least 80% identity to one of SEQ ID NOS: 75-86.

115. The method of claim 81, wherein said gRNA hybridizes to a CISH gene sequence that comprises a sequence that has at least 80% identity to SEQ ID NO: 82.

116. A method of treating a human subject with cancer, the method comprising:
administering to said subject a population of genetically modified human primary lymphocytes that comprise:
a) a genomic disruption in a CISH gene sequence, wherein said genomic disruption is introduced by a clustered regularly interspaced short palindromic repeats (CRISPR) system that comprises a polynucleotide encoding an endonuclease and a guide ribonucleic acid (gRNA); wherein said genomic disruption suppresses expression of said CISH gene, and wherein said gRNA comprises a sequence that binds a nucleic acid sequence adjacent to said genomic disruption; and
b) a transgene integrated into a gene sequence, and wherein said transgene is integrated into a genomic disruption in at least about 20% of cells in said population; to thereby treat a human subject with cancer.

117. A population of isolated genetically modified human primary lymphocytes that comprise:
a genomic disruption in a CISH gene sequence, wherein said genomic disruption is introduced by a clustered regularly interspaced short palindromic repeats (CRISPR) system that comprises a polynucleotide encoding an endonuclease and a guide ribonucleic acid (gRNA); wherein said genomic disruption suppresses expression of said CISH gene, and wherein said gRNA comprises a sequence that binds a nucleic acid sequence adjacent to said genomic disruption; and
b) a transgene integrated into a gene sequence, and wherein said transgene is integrated into a genomic disruption in at least about 20% of cells in said population.


SEQUENCE LISTING (190 sequences)

SEQ ID NO: 1-4 - skipped (4 entries)

SEQ ID NO: 5 - DNA, 25 bp, Artificial Sequence
  Description of Artificial Sequence: Synthetic primer
caccgcacgtgtgaaccaacccgcc

SEQ ID NO: 6 - DNA, 25 bp, Artificial Sequence
  Description of Artificial Sequence: Synthetic primer
aaacggcgggttggttcacacgtgc

SEQ ID NO: 7 - DNA, 25 bp, Artificial Sequence
  Description of Artificial Sequence: Synthetic primer
caccgaaacaacaggccgggcgggt

SEQ ID NO: 8 - DNA, 25 bp, Artificial Sequence
  Description of Artificial Sequence: Synthetic primer
aaacacccgcccggcctgttgtttc

SEQ ID NO: 9 - DNA, 25 bp, Artificial Sequence
  Description of Artificial Sequence: Synthetic primer
caccgacaaaaaaattagccgggtg

SEQ ID NO: 10 - DNA, 24 bp, Artificial Sequence
  Description of Artificial Sequence: Synthetic primer
aaaccacccggctaatttttttgt

SEQ ID NO: 11 - DNA, 25 bp, Artificial Sequence
  Description of Artificial Sequence: Synthetic primer
caccgtaaatttctctgatagacta

SEQ ID NO: 12 - DNA, 25 bp, Artificial Sequence
  Description of Artificial Sequence: Synthetic primer
aaactagtctatcagagaaatttac

SEQ ID NO: 13 - DNA, 25 bp, Artificial Sequence
  Description of Artificial Sequence: Synthetic primer
caccgtgtttcaatgagagcattac

SEQ ID NO: 14 - DNA, 25 bp, Artificial Sequence
  Description of Artificial Sequence: Synthetic primer
aaacgtaatgctctcattgaaacac

SEQ ID NO: 15 - DNA, 24 bp, Artificial Sequence
  Description of Artificial Sequence: Synthetic primer
caccggtctcgaactcctgagctc

SEQ ID NO: 16 - DNA, 24 bp, Artificial Sequence
  Description of Artificial Sequence: Synthetic primer
aaacgagctcaggagttcgagacc

SEQ ID NO: 17 - DNA, 20 bp, Artificial Sequence
  Description of Artificial Sequence: Synthetic primer
agtgaagtggcgcattcttg

SEQ ID NO: 18 - DNA, 21 bp, Artificial Sequence
  Description of Artificial Sequence: Synthetic primer
caccctttccaaatcctcagc

SEQ ID NO: 19 - DNA, 25 bp, Artificial Sequence
  Description of Artificial Sequence: Synthetic primer
caccgtgggggttagacccaatatc

SEQ ID NO: 20 - DNA, 25 bp, Artificial Sequence
  Description of Artificial Sequence: Synthetic primer
aaacgatattgggtctaacccccac

SEQ ID NO: 21 - DNA, 25 bp, Artificial Sequence
  Description of Artificial Sequence: Synthetic primer
caccgaccccacagtggggccacta

SEQ ID NO: 22 - DNA, 25 bp, Artificial Sequence
  Description of Artificial Sequence: Synthetic primer
aaactagtggccccactgtggggtc

SEQ ID NO: 23 - DNA, 25 bp, Artificial Sequence
  Description of Artificial Sequence: Synthetic primer
caccgagggccggttaatgtggctc

SEQ ID NO: 24 - DNA, 25 bp, Artificial Sequence
  Description of Artificial Sequence: Synthetic primer
aaacgagccacattaaccggccctc

SEQ ID NO: 25 - DNA, 24 bp, Artificial Sequence
  Description of Artificial Sequence: Synthetic primer
caccgtcaccaatcctgtccctag

SEQ ID NO: 26 - DNA, 24 bp, Artificial Sequence
  Description of Artificial Sequence: Synthetic primer
aaacctagggacaggattggtgac

SEQ ID NO: 27 - DNA, 25 bp, Artificial Sequence
  Description of Artificial Sequence: Synthetic primer
caccgccggccctgggaatataagg

SEQ ID NO: 28 - DNA, 25 bp, Artificial Sequence
  Description of Artificial Sequence: Synthetic primer
aaacccttatattcccagggccggc

SEQ ID NO: 29 - DNA, 25 bp, Artificial Sequence
  Description of Artificial Sequence: Synthetic primer
caccgcgggcccctatgtccacttc

SEQ ID NO: 30 - DNA, 25 bp, Artificial Sequence
  Description of Artificial Sequence: Synthetic primer
aaacgaagtggacataggggcccgc

SEQ ID NO: 31 - DNA, 20 bp, Artificial Sequence
  Description of Artificial Sequence: Synthetic primer
actcctttcatttgggcagc

SEQ ID NO: 32 - DNA, 20 bp, Artificial Sequence
  Description of Artificial Sequence: Synthetic primer
ggttctggcaaggagagaga

SEQ ID NO: 33 - DNA, 25 bp, Artificial Sequence
  Description of Artificial Sequence: Synthetic primer
caccgcggagagcttcgtgctaaac

SEQ ID NO: 34 - DNA, 25 bp, Artificial Sequence
  Description of Artificial Sequence: Synthetic primer
aaacgtttagcacgaagctctccgc

SEQ ID NO: 35 - DNA, 25 bp, Artificial Sequence
  Description of Artificial Sequence: Synthetic primer
caccgcctgctcgtggtgaccgaag

SEQ ID NO: 36 - DNA, 25 bp, Artificial Sequence
  Description of Artificial Sequence: Synthetic primer
aaaccttcggtcaccacgagcaggc

SEQ ID NO: 37 - DNA, 25 bp, Artificial Sequence
  Description of Artificial Sequence: Synthetic primer
caccgcagcaaccagacggacaagc

SEQ ID NO: 38 - DNA, 25 bp, Artificial Sequence
  Description of Artificial Sequence: Synthetic primer
aaacgcttgtccgtctggttgctgc

SEQ ID NO: 39 - DNA, 25 bp, Artificial Sequence
  Description of Artificial Sequence: Synthetic primer
caccgaggcggccagcttgtccgtc

SEQ ID NO: 40 - DNA, 25 bp, Artificial Sequence
  Description of Artificial Sequence: Synthetic primer
aaacgacggacaagctggccgcctc

SEQ ID NO: 41 - DNA, 25 bp, Artificial Sequence
  Description of Artificial Sequence: Synthetic primer
caccgcgttgggcagttgtgtgaca

SEQ ID NO: 42 - DNA, 25 bp, Artificial Sequence
  Description of Artificial Sequence: Synthetic primer
aaactgtcacacaactgcccaacgc

SEQ ID NO: 43 - DNA, 25 bp, Artificial Sequence
  Description of Artificial Sequence: Synthetic primer
caccgacggaagcggcagtcctggc

SEQ ID NO: 44 - DNA, 25 bp, Artificial Sequence
  Description of Artificial Sequence: Synthetic primer
aaacgccaggactgccgcttccgtc

SEQ ID NO: 45 - DNA, 20 bp, Artificial Sequence
  Description of Artificial Sequence: Synthetic primer
agaaggaagaggctctgcag

SEQ ID NO: 46 - DNA, 20 bp, Artificial Sequence
  Description of Artificial Sequence: Synthetic primer
ctctttgatctgcgccttgg

SEQ ID NO: 47 - DNA, 25 bp, Artificial Sequence
  Description of Artificial Sequence: Synthetic primer
caccgccgggtgacagtgcttcggc

SEQ ID NO: 48 - DNA, 25 bp, Artificial Sequence
  Description of Artificial Sequence: Synthetic primer
aaacgccgaagcactgtcacccggc

SEQ ID NO: 49 - DNA, 24 bp, Artificial Sequence
  Description of Artificial Sequence: Synthetic primer
caccgtgcggcaacctacatgatg

SEQ ID NO: 50 - DNA, 24 bp, Artificial Sequence
  Description of Artificial Sequence: Synthetic primer
aaaccatcatgtaggttgccgcac

SEQ ID NO: 51 - DNA, 25 bp, Artificial Sequence
  Description of Artificial Sequence: Synthetic primer
caccgctagatgattccatctgcac

SEQ ID NO: 52 - DNA, 25 bp, Artificial Sequence
  Description of Artificial Sequence: Synthetic primer
aaacgtgcagatggaatcatctagc

SEQ ID NO: 53 - DNA, 24 bp, Artificial Sequence
  Description of Artificial Sequence: Synthetic primer
caccgaggttcacttgatttccac

SEQ ID NO: 54 - DNA, 24 bp, Artificial Sequence
  Description of Artificial Sequence: Synthetic primer
aaacgtggaaatcaagtgaacctc

SEQ ID NO: 55 - DNA, 25 bp, Artificial Sequence
  Description of Artificial Sequence: Synthetic primer
caccgccgcacagacttcagtcacc

SEQ ID NO: 56 - DNA, 25 bp, Artificial Sequence
  Description of Artificial Sequence: Synthetic primer
aaacggtgactgaagtctgtgcggc

SEQ ID NO: 57 - DNA, 24 bp, Artificial Sequence
  Description of Artificial Sequence: Synthetic primer
caccgctggcgatgcctcggctgc

SEQ ID NO: 58 - DNA, 24 bp, Artificial Sequence
  Description of Artificial Sequence: Synthetic primer
aaacgcagccgaggcatcgccagc

SEQ ID NO: 59 - DNA, 20 bp, Artificial Sequence
  Description of Artificial Sequence: Synthetic primer
tggggatgaagctagaaggc

SEQ ID NO: 60 - DNA, 20 bp, Artificial Sequence
  Description of Artificial Sequence: Synthetic primer
aatctgggttccgttgccta

SEQ ID NO: 61 - DNA, 25 bp, Artificial Sequence
  Description of Artificial Sequence: Synthetic primer
caccgacaatgtgtcaactcttgac

SEQ ID NO: 62 - DNA, 25 bp, Artificial Sequence
  Description of Artificial Sequence: Synthetic primer
aaacgtcaagagttgacacattgtc

SEQ ID NO: 63 - DNA, 25 bp, Artificial Sequence
  Description of Artificial Sequence: Synthetic primer
caccgtcatcctcctgacaatcgat

SEQ ID NO: 64 - DNA, 25 bp, Artificial Sequence
  Description of Artificial Sequence: Synthetic primer
aaacatcgattgtcaggaggatgac

SEQ ID NO: 65 - DNA, 24 bp, Artificial Sequence
  Description of Artificial Sequence: Synthetic primer
caccggtgacaagtgtgatcactt

SEQ ID NO: 66 - DNA, 24 bp, Artificial Sequence
  Description of Artificial Sequence: Synthetic primer
aaacaagtgatcacacttgtcacc

SEQ ID NO: 67 - DNA, 25 bp, Artificial Sequence
  Description of Artificial Sequence: Synthetic primer
caccgacacagcatggacgacagcc

SEQ ID NO: 68 - DNA, 25 bp, Artificial Sequence
  Description of Artificial Sequence: Synthetic primer
aaacggctgtcgtccatgctgtgtc

SEQ ID NO: 69 - DNA, 24 bp, Artificial Sequence
  Description of Artificial Sequence: Synthetic primer
caccgatctggtaaagatgattcc

SEQ ID NO: 70 - DNA, 24 bp, Artificial Sequence
  Description of Artificial Sequence: Synthetic primer
aaacggaatcatctttaccagatc

SEQ ID NO: 71 - DNA, 25 bp, Artificial Sequence
  Description of Artificial Sequence: Synthetic primer
caccgttgtatttccaaagtcccac

SEQ ID NO: 72 - DNA, 25 bp, Artificial Sequence
  Description of Artificial Sequence: Synthetic primer
aaacgtgggactttggaaatacaac

SEQ ID NO: 73 - DNA, 20 bp, Artificial Sequence
  Description of Artificial Sequence: Synthetic primer
ctcaacctggccatctctga

SEQ ID NO: 74 - DNA, 20 bp, Artificial Sequence
  Description of Artificial Sequence: Synthetic primer
cccgagtagcagatgaccat

SEQ ID NO: 75 - DNA, 20 bp, Artificial Sequence
  Description of Artificial Sequence: Synthetic oligonucleotide
ttgctggctgtggagcggac

SEQ ID NO: 76 - DNA, 20 bp, Artificial Sequence
  Description of Artificial Sequence: Synthetic oligonucleotide
gactggcttgggcagttcca

SEQ ID NO: 77 - DNA, 20 bp, Artificial Sequence
  Description of Artificial Sequence: Synthetic oligonucleotide
tgctggggccttcctcgagg

SEQ ID NO: 78 - DNA, 20 bp, Artificial Sequence
  Description of Artificial Sequence: Synthetic oligonucleotide
ccgaaggtaggagaaggtct

SEQ ID NO: 79 - DNA, 20 bp, Artificial Sequence
  Description of Artificial Sequence: Synthetic oligonucleotide
atgcacagcagatcctcctc

SEQ ID NO: 80 - DNA, 20 bp, Artificial Sequence
  Description of Artificial Sequence: Synthetic oligonucleotide
agagagtgagccaaaggtgc

SEQ ID NO: 81 - DNA, 20 bp, Artificial Sequence
  Description of Artificial Sequence: Synthetic oligonucleotide
ggcatactcaatgcgtacat

SEQ ID NO: 82 - DNA, 20 bp, Artificial Sequence
  Description of Artificial Sequence: Synthetic oligonucleotide
gggttccattacggccagcg

SEQ ID NO: 83 - DNA, 20 bp, Artificial Sequence
  Description of Artificial Sequence: Synthetic oligonucleotide
aaggctgaccacatccggaa

SEQ ID NO: 84 - DNA, 20 bp, Artificial Sequence
  Description of Artificial Sequence: Synthetic oligonucleotide
tgccgactccagcttccgtc

SEQ ID NO: 85 - DNA, 20 bp, Artificial Sequence
  Description of Artificial Sequence: Synthetic oligonucleotide
ctgtcagtgaaaaccactcg

SEQ ID NO: 86 - DNA, 20 bp, Artificial Sequence
  Description of Artificial Sequence: Synthetic oligonucleotide
cgtactaagaacgtgccttc

SEQ ID NO: 87 - DNA, 20 bp, Artificial Sequence
  Description of Artificial Sequence: Synthetic oligonucleotide
gtcaccaatcctgtccctag

SEQ ID NO: 88 - DNA, 885 bp, Artificial Sequence
  Description of Artificial Sequence: Synthetic polynucleotide
atgacaacaagtggcgtgccattcggcatgactttgcgccccacgagatcacgactgtctcgccgaactccctacagccgggatcgactccctccctttgagactgaaacacgggccacgatactcgaggaccacccacttctgccggagtgtaacaccttgacgatgcataacgttagctatgtgagaggtctcccttgttctgtcggctttacccttattcaagagtgggtcgtgccgtgggacatggttctcacgagagaggagctcgttatcctgagaaaatgtatgcacgtttgtctttgctgtgcaaatatagatataatgacttctatgatgattcatgggtacgaatcttgggccttgcactgccattgtagcagtcctggctccctccaatgcatcgcgggaggccaagttctcgcttcctggtttagaatggtcgtggacggagcaatgttcaaccagcgctttatctggtatcgcgaggtagtcaactataatatgccgaaggaggttatgtttatgtctagtgtgttcatgcgagggagacatttgatttatcttagactgtggtatgatggccatgtgggaagcgtagttccggcgatgtccttcggttactccgcattgcattgtgggattttgaataacatcgttgtactttgttgttcatactgcgccgatctgtcagaaataagggtacgatgctgcgcacggcgaacccggaggctcatgctgagagccgttcgaataatcgctgaagaaacgacagcaatgttgtattcatgccgaactgaaaggcgacggcaacagtttatacgcgcactcttgcagcaccacaggccgatcctgatgcatgactacgatagcactccgatgtag

SEQ ID NO: 89 - DNA, 1491 bp, Artificial Sequence
  Description of Artificial Sequence: Synthetic polynucleotide
atggagagaaggaatcctagtgagaggggagtgcccgccgggttttctggtcacgcctccgtggaatccggatgtgagactcaggagtcccccgccaccgtggtgttccgcccaccaggagacaacactgacggtggcgcggcggctgctgcaggtggaagccaagccgccgctgctggggccgagccgatggaacccgaatccagacccggtccctctggcatgaacgttgtgcaggtcgcagaactctaccccgaactccgcaggatcttgacaatcacggaggacggccagggcctcaagggagtgaagagagagagaggggcttgtgaggccactgaggaagctcgcaatctggcgttttcattgatgacaaggcacaggccggaatgcattacattccaacagattaaggacaactgcgcaaacgagctcgatctcctggcccagaagtatagcatcgagcagctgacaacctattggctgcagcccggcgacgattttgaagaggccatccgcgtgtacgcaaaggtggccctgcgacctgactgcaaatataagatttccaaactggttaacatccggaattgttgttatattagtggaaatggcgcagaagtggagattgacacagaggatcgagtcgctttccggtgctctatgatcaacatgtggcccggtgtgctcggcatggatggcgtagtcattatgaatgtgaggttcaccggacctaattttagcggaaccgtcttcctggcaaacactaatctgatcctgcatggagtttctttctatggatttaataacacctgtgttgaagcttggaccgacgtgcgggttagagggtgtgctttttattgctgctggaaaggcgtcgtgtgtagacccaaaagtagagcttctatcaagaaatgcctgttcgagaggtgtactctgggcattctcagtgaaggtaatagcagggtcaggcataacgtggcctcagattgcggatgttttatgttggttaaatccgtggctgtgatcaagcacaacatggtgtgtggcaattgtgaggaccgggcatctcaaatgctgacatgttccgatggcaactgtcacctgctcaaaacaattgccgttgcgagccattctcggaaggcctggccagttttcgagcataacatcctgacgcgctgtagtctccacctgggtaacagacggggcgttttcctgccatatcagtgtaacctgtcacataccaagatactcctggaaccagaatctatgagtaaagtgaacctgaatggtgtattcgatatgaccatgaagatatggaaagtcctccgctatgacgaaactaggactaggtgtaggccctgcgagtgtggcggcaagcatatccgcaaccaacccgtgatgctggacgtgaccgaggagctgcgccccgatcacctggtgctggcctgcaccagagcagaattcgggagctcagacgaagacactgattaa

SEQ ID NO: 90 - DNA, 1503 bp, Artificial Sequence
  Description of Artificial Sequence: Synthetic polynucleotide
atggagagaaggaatcctagtgagaggggagtgcccgccgggttttctggtcacgcctccgtggaatccggatgtgagactcaggagtcccccgccaccgtggtgttccgcccaccaggagacaacactgacggtggcgcggcggctgctgcaggtggaagccaagccgccgctgctggggccgagccgatggaacccgaatccagacccggtccctctggcatgaacgttgtgcaggtcgcagaactctaccccgaactccgcaggatcttgacaatcacggaggacggccagggcctcaagggagtgaagagagagagaggggcttgtgaggccactgaggaagctcgcaatctggcgttttcattgatgacaaggcacaggccggaatgcattacattccaacagattaaggacaactgcgcaaacgagctcgatctcctggcccagaagtatagcatcgagcagctgacaacctattggctgcagcccggcgacgattttgaagaggccatccgcgtgtacgcaaaggtggccctgcgacctgactgcaaatataagatttccaaactggttaacatccggaattgttgttatattagtggaaatggcgcagaagtggagattgacacagaggatcgagtcgctttccggtgctctatgatcaacatgtggcccggtgtgctcggcatggatggcgtagtcattatgaatgtgaggttcaccggacctaattttagcggaaccgtcttcctggcaaacactaatctgatcctgcatggagtttctttctatggatttaataacacctgtgttgaagcttggaccgacgtgcgggttagagggtgtgctttttattgctgctggaaaggcgtcgtgtgtagacccaaaagtagagcttctatcaagaaatgcctgttcgagaggtgtactctgggcattctcagtgaaggtaatagcagggtcaggcataacgtggcctcagattgcggatgttttatgttggttaaatccgtggctgtgatcaagcacaacatggtgtgtggcaattgtgaggaccgggctggaattccagcatctcaaatgctgacatgttccgatggcaactgtcacctgctcaaaacaattcacgttgcgagccattctcggaaggcctggccagttttcgagcataacatcctgacgcgctgtagtctccacctgggtaacagacggggcgttttcctgccatatcagtgtaacctgtcacataccaagatactcctggaaccagaatctatgagtaaagtgaacctgaatggtgtattcgatatgaccatgaagatatggaaagtcctccgctatgacgaaactaggactaggtgtaggccctgcgagtgtggcggcaagcatatccgcaaccaacccgtgatgctggacgtgaccgaggagctgcgccccgatcacctggtgctggcctgcaccagagcagaattcgggagctcagacgaagacactgattaa

SEQ ID NO: 91-151 - skipped (61 entries)

SEQ ID NO: 152 - DNA, 311 bp, Artificial Sequence
  Description of Artificial Sequence: Synthetic polynucleotide
atggccttggtaacctctataactgtgctgctcagtctcgggatcatgggagatgctaagactactcagcctaatagtatggaaagtaatgaggaggagcctgtccacctgccttgtaatcactctaccataagcgggacagattacatacattggtatcggcagctcccttcacaaggtccagagtatgtgattcatggcctcacatcaaatgtgaacaatcggatggcttctcttgccattgcagaggatcggaaaagctcaacactcatcctgcatagggcgacactcagagatgcggccgtttatta

SEQ ID NO: 153 - DNA, 187 bp, Streptococcus pyogenes
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacg

SEQ ID NO: 154 - DNA, 101 bp, Artificial Sequence
  Description of Artificial Sequence: Synthetic oligonucleotide
  Description of Combined DNA/RNA Molecule: Synthetic oligonucleotide
gcctgctcgtggtgaccgaagguuuuagagcuagaaauagcaaguuaaaauaaggcuaguccguuaucaacuugaaaaaguggcaccgagucggugcuuuu

SEQ ID NO: 155 - DNA, 101 bp, Artificial Sequence
  Description of Artificial Sequence: Synthetic oligonucleotide
  Description of Combined DNA/RNA Molecule: Synthetic oligonucleotide
gacggaagcggcagtcctggcguuuuagagcuagaaauagcaaguuaaaauaaggcuaguccguuaucaacuugaaaaaguggcaccgagucggugcuuuu

SEQ ID NO: 156 - DNA, 101 bp, Artificial Sequence
  Description of Artificial Sequence: Synthetic oligonucleotide
  Description of Combined DNA/RNA Molecule: Synthetic oligonucleotide
gctagatgattccatctgcacguuuuagagcuagaaauagcaaguuaaaauaaggcuaguccguuaucaacuugaaaaaguggcaccgagucggugcuuuu

SEQ ID NO: 157 - DNA, 100 bp, Artificial Sequence
  Description of Artificial Sequence: Synthetic oligonucleotide
  Description of Combined DNA/RNA Molecule: Synthetic oligonucleotide
gtgcggcaacctacatgatgguuuuagagcuagaaauagcaaguuaaaauaaggcuaguccguuaucaacuugaaaaaguggcaccgagucggugcuuuu

SEQ ID NO: 158 - DNA, 100 bp, Artificial Sequence
  Description of Artificial Sequence: Synthetic oligonucleotide
  Description of Combined DNA/RNA Molecule: Synthetic oligonucleotide
gggttccattacggccagcgguuuuagagcuagaaauagcaaguuaaaauaaggcuaguccguuaucaacuugaaaaaguggcaccgagucggugcuuuu

SEQ ID NO: 159 - DNA, 100 bp, Artificial Sequence
  Description of Artificial Sequence: Synthetic oligonucleotide
  Description of Combined DNA/RNA Molecule: Synthetic oligonucleotide
gtcaccaatcctgtccctagguuuuagagcuagaaauagcaaguuaaaauaaggcuaguccguuaucaacuugaaaaaguggcaccgagucggugcuuuu

SEQ ID NO: 160-173 - skipped (14 entries)

SEQ ID NO: 174 - DNA, 13064 bp, Artificial Sequence
  Description of Artificial Sequence: Synthetic polynucleotide
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcccgccgggtaactcacggggtatccatgtccatttctgcggcatccagccaggatacccgtcctcgctgacgtaatatcccagcgccgcaccgctgtcattaatctgcacaccggcacggcagttccggctgtcgccggtattgttcgggttgctgatgcgcttcgggctgaccatccggaactgtgtccggaaaagccgcgacgaactggtatcccaggtggcctgaacgaacagttcaccgttaaaggcgtgcatggccacaccttcccgaatcatcatggtaaacgtgcgttttcgctcaacgtcaatgcagcagcagtcatcctcggcaaactctttccatgccgcttcaacctcgcgggaaaaggcacgggcttcttcctccccgatgcccagatagcgccagcttgggcgatgactgagccggaaaaaagacccgacgatatgatcctgatgcagctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagataaatggtaccagatccctatacagttgaagtcggaagtttacatacaccttagccaaatacatttaaactcactttttcacaattcctgacatttaatcctagtaaaaattccctgtcttaggtcagttaggatcaccactttattttaagaatgtgaaatatcagaataatagtagagagaatgattcatttcagcttttatttctttcatcacattcccagtgggtcagaagtttacatacactcaattagtatttggtagcattgcctttaaattgtttaacttggtctccctttagtgagggttaattgatatcgaattcagatctgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcataacttcgtatagcatacattatacgaagttatgagctctctggctaactagagaacccactgcttactggcttatcgaaattaatacgactcactatagggagacccaagctggctagttaagctatcaagcctgcttttttgtacaaacttgtgctcttgggctgcaggtcgagggatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccacaccagccaccaccttctgataggcagcctgcacctgaggagtgaattatcgaattcctattacacccactcgtgcaggctgcccaggggcttgcccaggctggtcagctgggcgatggcggtctcgtgctgctccacgaagccgccgtcctccacgtaggtcttctccaggcggtgctggatgaagtggtactcggggaagtccttcaccacgcccttgctcttcatcagggtgcgcatgtggcagctgtagaacttgccgctgttcaggcggtacaccaggatcacctggcccaccagcacgccgtcgttcatgtacaccacctcgaagctgggctgcaggccggtgatggtcttcttcatcacggggccgtcgttggggaagttgcggcccttgtactccacgcggtacacgaacatctcctcgatcaggttgatgtcgctgcggatctccaccaggccgccgtcctcgtagcgcagggtgcgctcgtacacgaagccggcggggaagctctggatgaagaagtcgctgatgtcctcggggtacttggtgaaggtgcggttgccgtactggaaggcggggctcaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtgggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgtgtgtgtgcgcgcacgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcatcaggatgtcgaaggcgaagggcaggggggcgcccttggtcacgcggatctgcaccagctggttgccgaacaggatgttgcccttgccgcagccctccatggtgaacacgtggttgttcaccacgccctccaggttcaccttgaagctcatgatctcctgcaggccggtgttcttcaggatctgcttgctcaccatggtaattcctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagccttgccagaaaaaaagcgtcgcagcaggtcatcaaaaattttaaatggctagagacttatcgaaagcagcgagacaggcgcgaaggtgccaccagattccgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgcatgctcttctccacctcagtgatgacgagagcgggcgggtgagggggcgggaacgcagcgatctctgggttctacgttagtgggagtttaacgacggtccctgggattccccaaggcaggggcgagtccttttgtatgaattactctcagctccggtcggggcgggttggggggggtggtgacggggaggccgcctggaagggacgtgcagaatcttccctctaccattgctggcttagctccaaaggttgtattgagattagggtgtaccttcgcctctcaatcagcctcccgtcctcagccttgccatctcgctagtccgggacaaatccctagagcgtcttcctctgcgggtctcagcccagcccggggttggctcctcctccgccccggcttccgcgcccctcccgtgtggcaaggagtaccaggcccggggaccccgaggggcttggggcgaagggtcgggactgggggcctccttaacggctcacggacttgcgagaggttcggctcgatggccgtgaaagcgacgaatccgctcctgtgctggcctcttggctccttccattcaaagccagctgcttttatggaagcccgtaacacgtcatctccccctggtactccagatgtccaggctttcagtttagaatagactcagtcctacagttagctttagatctaattctagttttgttacgccaaaaagttcctgcgagtgtgtgtgtgtgcctcatggtactttttaaattaaaaggtgtacagttatttgattgcaaacataaggaacctaaaatgctttcagattttccacatgatctcatgtagaggctaagatctacagcatcagcaagtttatccacccagtttcctaaccccaacacttgctatgaagtcacagcttctcctatttaaataagtgcctattatatttaaataagtgctgtcgttttctgtcatcctatcgattgtaactgcattttagcataaatctagggcaagattggatgagcttggcctttttggatggctatcaaggcaggccttgggaaatgctcctctgaggaaagaagaacgtttatttttaatgagctaattactagatcattatgtttcttcttccagctgtagaatatcattgcccagcttctcgaacaaacttatttattaacaagtatttgagaacctactatgtggccaacgctaagtgacctgcaggcatgcaagctgagcctattctaccaccactttgtacaagaaagctgggttgatctagagggcccgcggttcgaaggtaagcctatccctaaccctctcctcggtctcgattctacgcgtcaggtgcaggctgcctatcagaaggtggtggctggtgtggccaatgccctggctcacaaataccactgagatctttttccctctgccaaaaattatggggacatcatgaacgcagtgaaaaaaatgctttatttgtgaaatttgtgatgctattgctttatttgtaaccattataagctgcaataaacaagttctcgagaagttcctattctctagaaagtataggaacttctggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatctcgagcagctgaagcttaccatgaccgagtacaagcccacggtgcgcctcgccacccgcgacgacgtccccagggccgtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgcagcagtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacttggtgcatgacccgcaagcccggtgcctgacgcccgcccacaagacccgcagcgcccgaccgaaaggagcgcacgaccccatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaagttcctattctctagaaagtataggaacttctcgagtctagaagatgggcgggagtcttctgggcaggcttaaaggctaacctggtgtgtgggcgttgtcctgcaggggaattgaacaggtgattaccctgttatccctagtaatcccgggatctaatacgactcactatagggagaccatcattttctggaattttccaagctgtttaaaggcacagtcaacttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactgtatagggatcccccgggctgcaggaattcgataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatctagctgcgtgttctgcagcgtgtcgagcatcttcatctgctccatcacgctgtaaaacacatttgcaccgcgagtctgcccgtcctccacgggttcaaaaacgtgaatgaacgaggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 175 - DNA, 8340 bp, Artificial Sequence
  Description of Artificial Sequence: Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatggctgccaccatggactacaaagacgatgacgacaagagcagggctgaccccaagaagaagaggaaggtgactgtcgctctgcacctggcaatacctcttaaatggaaacctaatcacactccagtttggatcgatcaatggccacttcctgagggcaagttggtggcattgactcagttggtagagaaagaactccaacttgggcacatcgaaccgtccctgtcctgttggaacaccccagtattcgtcataaggaaagcctccggaagttaccgcttgcttcatgacctgagggcggtgaatgcaaagcttgtaccttttggcgccgtccagcagggagctccagtcttgagtgccttgccacggggatggccgcttatggttctcgatttgaaggactgctttttcagcattccgcttgcggaacaggatcgagaggctttcgcctttacgctgcccagcgtcaacaaccaggccccggctagacgcttccaatggaaagtcctccctcagggtatgacctgttcacctacaatttgtcaacttattgttggtcaaatcctggaaccgcttagattgaagcatccgtcccttagaatgctgcattatatggacgacctgcttctcgcagcgagttctcacgacgggttggaggctgccggagaagaagttattagcacccttgaacgagcagggttcaccatttcaccggataaggtacagcgggaacccggcgtacagtacttgggctacaagctcggttcaacatacgtggcccccgtaggactggttgccgagccaaggattgcaactctttgggatgtacaaaaactcgttggttcacttcagtggttgaggcccgctctcggcattccgccgagacttatgggccctttctatgagcagcttagaggatctgacccgaacgaagcacgagaatggaacctggacatgaaaatggcctggcgagagatcgtacagctctcaacgacggctgctcttgaacggtgggaccccgcccttcccctcgaaggggctgtggcacgctgtgaacaaggagctataggggtcctcggtcagggactttccacccatccccgcccatgtctttggcttttttcaactcaacccaccaaagcatttacagcgtggctggaggtacttacccttctcattaccaaattgcgagcgtccgcggtccgaactttcgggaaagaagtagatatattgttgctgccagcctgttttagagaagatttgccccttccagaagggattcttcttgccttgagaggtttcgcaggtaagattagaagtagcgacacaccgtccatcttcgacatcgcgcgcccgctccacgtgagcctgaaggttagagtcaccgaccatcccgttccgggtcccacagtttttaccgatgcatctagtagtacccacaaaggagtagtagtctggcgcgagggacctcgatgggaaataaaggagatcgcagatttgggggctagtgttcagcagttggaagcacgcgccgtggcgatggctcttctcctgtggcccacgacaccaactaatgttgtaaccgactcagctttcgtagctaaaatgctcctgaaaatgggccaggaaggggtcccatccactgcagctgcatttatccttgaagacgcactcagccaaaggtcagcaatggctgcggtgctccatgtgcggtcccattccgaagtacctggtttctttacagaggggaatgatgtcgccgactctcaagcaaccttccaggcgtatcctcttagggaagctaaagacctccatacagctcttcatataggtccgagagctctgagcaaggcgtgtaatattagcatgcagcaagctagggaggtcgtccagacatgtccacactgtaactccgcacctgccctcgaggcaggggtaaatccgcgagggttggggccgctccagatctggcaaactgatttcacgttggaaccaaggatggctccgcggagttggctggcagtaaccgtagacacagcgtcttctgcaattgttgtaactcagcatggccgcgtgactagcgtggccgcgcagcatcactgggcaacggctatagcggtcctcggacgacctaaagcaataaagacggacaatggcagttgttttacttcaaaatcaaccagagagtggctcgctaggtggggcatagcacacacgactggaatccccggtaatagccaagggcaggctatggtagagagagcaaatcgactgctcaaagataagatccgggtccttgctgaaggggacggctttatgaagcggataccaactagtaaacagggagaacttcttgcaaaggccatgtacgcgctcaatcattttgaacgaggggaaaatactaaaaccccgatccaaaaacactggcgacctaccgtgttgacggagggacctccagtaaaaatcaggattgagacgggcgagtgggaaaaaggttggaacgtgctggtctgggggcgagggtatgctgcagtaaaaaacagagacactgacaaagtaatatgggttccatctcgcaaggttaaaccggacatcgctcaaaaggatgaagtgacaaaaaaagacgaagcgtcaccactctttgcataatgaacccatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 176 - DNA, 7482 bp, Artificial Sequence
  Description of Artificial Sequence: Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatggctgccaccatggactacaaagacgatgacgacaagagcagggctgaccccaagaagaagaggaaggtgactgttgcgctccatcttgcgataccgttgaagtggaaaccgaatcacactcctgtgtggatcgaccagtggccactcccagaagggaaactggtagcgttgacacaacttgtcgaaaaggagcttcaacttggccatatagaacctagtttgtcctgttggaacactcctgtgtttgtcatcaggaaggcctccgggagttatcgcctgttgcacgaccttcgagctgttaatgcaaaactcgtaccctttggcgcggtgcaacaaggggctccagttttgagtgcattgcctcgggggtggccgcttatggtcttggatctgaaggattgcttttttagtatacctctggcagagcaggatagagaggcctttgccttcacgcttccttcagtgaacaaccaggctccggccaggcggtttcaatggaaggttttgccccaagggatgacttgctccccgacgatatgtcaactgatcgtgggccagatactggaaccactccgattgaagcacccttctttgcgcatgctccattacatggatgacctcttgttggcggccagctcccatgacggtctggaggcggcgggtgaagaagtgataagcaccctggaacgagcgggattcacaatcagcccggacaaagtgcaaagagagcccggagtccaatatctgggctacaagttgggttccacatacgtcgcccctgtaggcctggtagcggaaccgcgcattgccacgttgtgggatgtgcaaaaactcgttggatctctccaatggttgcgcccggcactgggtatcccacccagactgatgggtccattctatgaacaactgaggggctctgacccgaatgaggcgcgggaatggaatttggacatgaagatggcgtggcgcgaaatagtccaactttcaacaacggcggctcttgaacgctgggatcctgccttgccgcttgaaggcgcagtagccaggtgcgagcagggggcgataggagtgttgggacaaggtctcagcacacacccgaggccgtgcctgtggttgttcagtactcaacctacgaaggcttttacagcatggctggaagtcctcaccttgttgattacaaaactcagagcatctgccgtcaggaccttcggcaaggaagtagatatccttcttctgcccgcctgcttccgcgaagaccttccactgccagagggaatactgcttgcattgaggggttttgccggtaagatccggtccagcgatactccgagcatatttgacatcgctagacctcttcacgtctcactcaaggttcgcgtgactgaccacccagttccgggacccaccgtattcaccgatgccagtagtagcactcataaaggggtagtcgtctggcgggaaggacctcgctgggagataaaggaaatagcagacttgggtgccagcgtgcaacaactggaggcccgggcggtcgcgatggcactccttttgtggccaaccaccccgacgaacgtagttacagattcagctttcgtagccaaaatgttgttgaaaatgggtcaggaaggtgtcccttccactgccgcagcattcatattggaggatgccctgagtcaaagaagtgcaatggccgcagttcttcacgtgcgatcccatagcgaagtacctggcttttttactgagggcaatgatgtggctgactcacaggctacatttcaggcttattaatgaacccatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 177 - DNA, 7086 bp, Artificial Sequence
  Description of Artificial Sequence: Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatggctgccaccatggactacaaagacgatgacgacaagagcagggctgaccccaagaagaagaggaaggtgccaatctcacccatcgaaacagtccccgtgaaactcaagccgggtatggatgggccgaaggttaagcaatggcccttgactgaggaaaaaataaaggcgctcgtagagatatgcacggaaatggagaaggagggcaagataagcaagattggcccagagaatccctataatacccccgttttcgcgataaagaagaaggactcaaccaaatggcggaaacttgtagattttcgggaacttaataagcgaacccaagacttctgggaggtccaacttggcattccgcatcccgccggtttgaaaaagaagaaatcagttacggtgcttgacgttggcgacgcctattttagcgttcctcttgacgaggactttagaaaatacacagccttcacaataccaagtattaacaacgagacacccggaatccggtatcaatacaacgtgctcccccaaggatggaaagggtctccagcaatttttcagtctagcatgaccaaaatcttggaacctttccgcaagcagaacccggatattgttatttatcagtatatggatgacctttatgtcggttcagatcttgaaattggtcagcaccgaacgaagatagaggaacttcgacagcacttgttgcgctggggtcttacaaccccagacaaaaaacaccagaaggaaccaccttttctttggatgggttatgaacttcacccagataagtggaccgtgcagcccattgtcttgccggaaaaggactcctggacagtaaatgatattcagaagctcgtaggaaaactgaattgggcaagccagatatacccaggtattaaagttaggcaattgtgcaaacttttgcggggcacgaaggcacttactgaggttataccactgactgaagaggcggagcttgaactcgcagagaatagagaaatactcaaggaaccggtacatggcgtatactatgatccaagtaaggatttgattgcggagattcagaaacagggtcagggacaatggacgtaccaaatttaccaagaacctttcaaaaatcttaagacgggaaagtatgcacgaatgcgcggcgcacatacgaatgatgtcaagcagttgactgaagcagtacagaagattacaaccgaatctatcgttatatggggaaagactcccaaatttaagctcccaatacaaaaagaaacttgggagacctggtggaccgaatattggcaggcgacatggataccggagtgggaatttgttaacacaccgccgctggtaaagttgtggtatcagctcgaaaaagagccaattgtgggagcagagacgttctaatgaacccatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 178 - DNA, 7374 bp, Artificial Sequence
  Description of Artificial Sequence: Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatggctcctatatctccaatcgaaacagtccccgtcaaattgaaaccgggaatggacggtccaaaagtcaaacaatggcctctcaccgaggagaagattaaggcattggtcgaaatctgcactgagatggagaaagaggggaaaattagcaaaatcgggccagagaacccctacaatacacccgtatttgccatcaaaaaaaaagatagcactaagtggcgaaagctcgtggacttccgcgaactcaataaaagaacccaggatttttgggaggtacagcttggcattccgcatccggcaggacttaagaagaaaaaatccgtaaccgtgctggatgtgggcgatgcatactttagcgtaccactggatgaggattttaggaagtatactgcattcacaataccttcaattaacaacgaaacgccagggataaggtaccaatataacgtcctcccccaaggctggaagggctctccagcgatcttccagtcttcaatgactaagatacttgagccgttcaggaagcaaaaccccgacatcgtaatttaccagtacatggatgacttgtacgtcggtagtgatctcgaaattggccagcatcgaacaaaaatcgaggaattgaggcaacaccttctgcggtggggtttgacgacgcccgacaaaaagcatcaaaaagagccgccgtttctgtggatgggttatgagctccatccggacaaatggacagtccagcccatcgtcttgccagaaaaagatagttggactgtaaatgacattcaaaaattggtcgggaaattgaactgggcgtcccagatctatccaggaattaaagtccggcagctttgcaagcttctccggggaacgaaggcacttacagaggtcataccccttacggaagaagcggaattggagcttgcggagaaccgcgagatactcaaagagccggtccacggggtctactacgatccatccaaagatcttattgcagagattcagaaacaagggcagggtcaatggacatatcagatctaccaagagccgttcaagaatttgaagacaggaaagtacgcgaggatgaggggcgcacatactaacgatgttaaacaactcactgaggctgtacaaaagattactacggagtcaatagtaatatggggcaaaacacctaagttcaagctcccgatccaaaaggagacttgggaaacctggtggaccgagtattggcaagctacgtggattcctgagtgggaatttgtgaacacacctcccctcgtgaagctgtggtatcaacttgaaaaggagccaatagtcggcgcggagaccttctatgtggacggcgccgcgaaccgagagacaaagctcggcaaggcgggttatgtaacgaaccgaggtaggcaaaaggtcgtaacgcttactgatacgaccaaccaaaaaaccgaactgcaggctatttatctcgcattgcaagactcaggactggaagtcaatatcgtgacggacagtcaatatgcactggggattattcaggcgcaaccggatcagagtgaaagcgagctggtaaaccaaattattgagcagttgataaaaaaggagaaagtgtatcttgcttgggtaccagcccataaggggatcggaggtaatgaacaggttgataaacttgtaagcgctggaattcggaaagtacttacccatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 179 - DNA, 7722 bp, Artificial Sequence
  Description of Artificial Sequence: Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagctgaacgagaaacgtaaaatgatataaatatcaatatattaaattagattttgcataaaaaacagactacataatactgtaaaacacaacatatccagtcactatggctgccaccatggactacaaagacgatgacgacaagagcagggctgaccccaagaagaagaggaaggtgggtagtcacatgacatggctgtctgactttcctcaggcatgggcggaaactggaggtatgggtttggcagtacggcaggctccacttattatccctcttaaagcaacgtcaacgccggtttctatcaagcaatatccaatgagtcaagaagctcgcctgggaattaagcctcacatacaacggttgttggatcaaggtattcttgtgccgtgccaatctccttggaatacaccactccttcctgtcaaaaaacccggaacaaatgactaccgccccgtgcaagaccttcgggaagtcaataagagggtagaagatattcacccgaccgttccaaatccgtataatctgttgtcaggactgccaccgtcccatcagtggtatactgtcctcgacttgaaggatgcgttcttttgcctgcgcctccaccctacgtcacagcccctgttcgcgttcgaatggagagaccctgaaatgggtatatcagggcagttgacttggaccagacttccacaagggttcaaaaatagccctactctttttgatgaagccctccacagggacctcgcagatttcaggatccagcacccggaccttatcttgctgcagtacgtagacgatctcttgctggcggcgacaagcgaactggattgccagcagggcacgcgagctctcctccagacactgggtaacctggggtacagggcgtcagctaagaaggcacaaatatgccaaaaacaagtgaagtacctggggtatctcctgaaagaggggcaacggtggctcacagaagcccgaaaggagacggtgatgggacaaccgacgcctaaaacgccacgacaactgcgagaatttttgggcaccgccgggttttgccgcctttggatccctggctttgcggagatggctgctccattgtatcccttgactaaaacaggtacgttgtttaattggggcccagatcagcaaaaggcttaccaagaaattaaacaagcgcttcttactgctccggcactcggccttccggatttgactaagccctttgagttgtttgtagacgagaagcagggatacgcgaagggtgttttgacgcaaaagctcggcccttggcgacgacccgtagcgtatttgtctaaaaagctcgacccagtagcggccggttggccaccatgtcttcggatggtcgctgccatagcggttcttaccaaggacgcggggaaactgacaatgggacagcctcttgtaataaaggcgccgcatgctgttgaagcactggtgaagcagccaccagatcgatggctgagcaacgcaaggatgacacactatcaggccctgcttctcgatacagatagagtccaattcggccctgttgttgccttgaacccagctacgcttttgcctctcccagaagagggtttgcaacacaattgcttggatatcttggcagaagcccacggcacgcggccggatttgacggaccagccgttgcccgatgccgaccatacctggtatactgacgggtcctcattgctgcaggagggccagcgcaaagctggggcggcagtaactacggagaccgaagtcatttgggcaaaagcactgccagcagggacctctgcccagcgggcggagcttattgcgcttacacaggcattgaagatggcagaaggaaagaagctcaatgtctatacggattcccggtatgcatttgccacggcgcacattcacggcgagatctataggcgaagaggactgcttacttccgagggtaaggagataaagaataaggatgaaatcctcgcccttctcaaagccctttttttgccgaaacgcctgagcataatccattgccctggtcaccaaaaggggcattctgcagaggcgcgaggcaacaggatggcagatcaggctgctaggaaggccgccattacggagacgcctgatacgagtacgttgctttaatgaacccatagtgactggatatgttgtgttttacagtattatgtagtctgttttttatgcaaaatctaatttaatatattgatatttatatcattttacgtttctcgttcagctttcttgtacaaagtggttgatctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 180 - protein, 4 aa, Unknown
  Description of Unknown: DEAD box helicase
DEAD

SEQ ID NO: 181 - protein, 4 aa, Unknown
  Description of Unknown: DEAH box helicase
DEAH

SEQ ID NO: 182 - protein, 600 aa, Artificial Sequence
  Description of Artificial Sequence: Synthetic polypeptide
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 183 - DNA, 96 bp, Artificial Sequence
  Description of Artificial Sequence: Synthetic oligonucleotide
caggccctggaacccccccaccttctccccagccctgctcgtggtgaccgaggactgccgcttccgtgtcacacaactgcccaacgggcgtgactt

SEQ ID NO: 184 - DNA, 119 bp, Artificial Sequence
  Description of Artificial Sequence: Synthetic polynucleotide
cggcaggctgacagccaggtgactgaagtctgtgcggcaacctacatgatggggaatgagttgaccttcctagatgattccatctgcacgggcacctccagtggaaatcaagtgaacct

SEQ ID NO: 185 - DNA, 81 bp, Artificial Sequence
  Description of Artificial Sequence: Synthetic oligonucleotide
cggcaggctgacagccaggtgactgaagtctgtgcggcaacctacatgcacgggcacctccagtggaaatcaagtgaacct

SEQ ID NO: 186 - DNA, 7520 bp, Artificial Sequence
  Description of Artificial Sequence: Synthetic polynucleotide
acattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactagtgaattcgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttgctagcccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtgaacacgctaccggtgccaccatggccttggtaacctctataactgtgctgctcagtctcgggatcatgggagatgctaagactactcagcctaatagtatggaaagtaatgaggaggagcctgtccacctgccttgtaatcactctaccataagcgggacagattacatacattggtatcggcagctcccttcacaaggtccagagtatgtgattcatggcctcacatcaaatgtgaacaatcggatggcttctcttgccattgcagaggatcggaaaagctcaacactcatcctgcatagggcgacactcagagatgcggccgtttattactgtatactccggagactgaacgactataagctgtcctttggagcagggaccaccgtaacagtaagagccgacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaagagccaaaagaagtggttctggcgcgacgaattttagtttgcttaagcaagccggagatgtggaggaaaatcctggaccgatggacacctggctcgtctgttgggctatcttttctctcctcaaggccggactcacggagccggaagttacacagacaccatcacaccaagtcacacaaatggggcaggaagtgattcttaggtgcgtaccgatttctaaccacctctacttctactggtataggcagattctgggccagaaggtggagttcctcgtttcattttacaataacgagatcagcgagaaaagtgagatcttcgacgaccaattcagtgtcgaaagacccgatggaagtaacttcacactgaagattcgcagtactaaattggaggattccgcgatgtacttttgtgcctctagcctcggagatcgggggaatgagaaactgttttttggcagtggtactcagctgtccgtactggaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaagttaactgagcggccgcgtttcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcaaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaagatatctctagagtcgacccatgggggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgat

SEQ ID NO: 187 - DNA, 127 bp, Artificial Sequence
  Description of Artificial Sequence: Synthetic polynucleotide
tctctcagactccccagacaggccctggaacccccccaccttctccccagccctgctcgtggtgaccgaagggggaattcgagcacgctagttttagtaactcgcgttgttttcttcacctttaata

SEQ ID NO: 188 - DNA, 127 bp, Artificial Sequence
  Description of Artificial Sequence: Synthetic polynucleotide
atagctactccaccacttgttcctaagcggtcagctcctgcttcaatcattttttgagcatcttcaaatgttctaactccaccagctgctttgctagcctaccgggtaggggaggcgcttttcccaa

SEQ ID NO: 189 - DNA, 23 bp, Artificial Sequence
  Description of Artificial Sequence: Synthetic oligonucleotide
  modified_base (21)..(21): a, c, t, g, unknown or other
gggttccattacggccagcgngg

SEQ ID NO: 190 - protein, 6 aa, Artificial Sequence
  Description of Artificial Sequence: Synthetic 6xHis tag
HHHHHH